SEQUENCE LISTING (304 sequences)

SEQ ID NO: 1 - DNA, 342 bp, synthetic construct
  source 1..342: mol_type = other DNA
gccgagtactgcctgagctacgacaccgagatctggaccgtggagtacggcgccatgcctatcggcaagatcgtggaggagaagatcgagtgttctgtgtacaccgtggatgagaacggcttcgtgtacacccagcctatcgcccagtggcaccctagaggccagcaggagatcatcgagtacaccctggaggacggcagaaagatcagagccaccaaggaccacaagatgatgaccgagagcggcgagatgctgcctatcgaggagatcttccagagagagctggacctgaaggtggagacattccacgagatgagcctgctgagaagaggcgccaagtaa

SEQ ID NO: 2 - DNA, 117 bp, synthetic construct
  source 1..117: mol_type = other DNA
atggtgaagatcatcaagagacagagcctgggcagacagaacgtgtacgacgtgtgcgtggagacagaccacaacttcgtgctggccaacggctgcgtggccagcaactgcttcaac

SEQ ID NO: 3 - DNA, 315 bp, synthetic construct
  source 1..315: mol_type = other DNA
gccgagtactgcctgagcgccgataccgagatcctgaccgtggagtacggcttcctgcctatcggcgagatcgtgggcaaggccatcgagtgtagagtgtactctgtggacggaaacggaaacatctacacccagtctatcgcccagtggcacaacagaggcgagcaggaggtgttcgagtacaccctggaggatggctctatcatcagagccaccaaggatcacaagttcatgaccaccgacggcgagatgctgcctatcgacgagatcttcgccagacagctggacctgatgcaggtgcagggcctgcactaa

SEQ ID NO: 4 - DNA, 117 bp, synthetic construct
  source 1..117: mol_type = other DNA
atggtaaagatcaccgctagaaaattcgttggccgggaaaacgtgtatgatattggtgtggaacatcaccacaatttcgccattaagaatggactcatagcttccaattgcttcaac

SEQ ID NO: 5 - DNA, 318 bp, synthetic construct
  source 1..318: mol_type = other DNA
gccgagtactgcctgagctacgataccgaggtgctgaccgtggagtacggcttcgtgcctatcggcgagatcgtggagaagggcatcgagtgtagcgtgttctctatcaacaacaacggcatcgtgtacacccagcctatcgcccagtggcaccacagaggcaagcaggaggtgttcgagtactgtctggaggacggcagcatcatcaaggccaccaaggaccacaagttcatgacccaggacggcaagatgctgcctatcgacgagatcttcgagcaggagctggacctgctgcaggtgaagggcctgcctgagtaa

SEQ ID NO: 6 - DNA, 117 bp, synthetic construct
  source 1..117: mol_type = other DNA
atgatcaagatcgccagcagaaagttcctgggcgtggagaacgtgtacgacatcggcgtgagaagagatcacaacttcttcatcaagaacggcctgatcgccagcaactgcttcaac

SEQ ID NO: 7 - DNA, 318 bp, synthetic construct
  source 1..318: mol_type = other DNA
gccgagtactgcctgagctacgacaccgaggtgctgaccgtggagtacggcttcgtgcctatcggcgagatcgtggacaagggcatcgagtgttctgtgttcagcatcgacagcaacggcatcgtgtacacccagcctatcgcccagtggcaccacagaggcaagcaggaggtgttcgagtactgtctggaggacggctctatcatcaaggccaccaaggaccacaagttcatgacccaggatggcaagatgctgcctatcgatgagatcttcgagcaggagctggacctgctgcaggtgaagggcctgcctgagtaa

SEQ ID NO: 8 - DNA, 117 bp, synthetic construct
  source 1..117: mol_type = other DNA
atgatcaagatcgccagcagaaagttcctgggcgtggagaacgtgtacgacatcggcgtgggcagagatcacaacttcttcgtgaagaacggcctgatcgccagcaactgcttcaac

SEQ ID NO: 9 - DNA, 366 bp, synthetic construct
  source 1..366: mol_type = other DNA
gccgagtactgcctgagctacgagacagaggtgctgaccctggagtacggcttcgtgcctatcggcgagatcgtgaacaagcagatggtgtgtaccgtgttctctctgaacgactctggcaacgtgtacacccagcctatcggccagtggcacgatagaggcgtgcaggacctgtacgagtactgcctggacgacggcagcaccatcagagccaccaaggaccacaagttcatgaccacccagggcgagatggtgcctatcgatgagatcttccaccagggctgggagctggtgcaggtgtctggcatcagcaagctggtgcagcagagaaccctgcctttcatcatcgtggacagaaagctgtaa

SEQ ID NO: 10 - DNA, 117 bp, synthetic construct
  source 1..117: mol_type = other DNA
atggtgaagatcgtgagcagaagatacctgggcaaggccgacgtgtacgacatcggcgtggccaaggaccacaacttcatcatcaagaacggcctggtggccagcaactgcttcaac

SEQ ID NO: 11 - DNA, 309 bp, synthetic construct
  source 1..309: mol_type = other DNA
gccgagtactgcctgagctacgacaccgagatcctgaccgtggagtacggccctatgcctatcggcaagatcgtggaggagaacatcaactgcagcgtgtacaccgtgaacaagaacggcttcgtgtacacccagagcatcgcccagtggcaccacagaggcgagcaggaggtgttcgagtactacctggaggacggcgagacaatcagagccaccaaggaccacaagttcatgaccaccgagggcaagatgctgcctatcgacgagatcttcgagaacaacctggacctgaagaagctgaccgtgtaa

SEQ ID NO: 12 - DNA, 117 bp, synthetic construct
  source 1..117: mol_type = other DNA
atggtgaagatcatcgagagaagaagcctgggcaagcagaacgtgtacgacatcggcgtggagaaggaccacaacttcctgctgagcaacaacctgatcgccagcaactgcttcaac

SEQ ID NO: 13 - DNA, 312 bp, synthetic construct
  source 1..312: mol_type = other DNA
gccgagtactgcctgagctacgagacacagatcatgaccgtggagtacggcctgatgcctatcggcaagatcgtggaggagcagatcgactgtaccgtgtacaccgtgaacaagaacggcttcgtgtacacccagcctatcgcccagtggcactacagaggcgagcaggaggtgttcgagtactgcctggaggacggcagcaccatcagagccacaaaggaccacaagttcatgaccaccgacggccagatgctgcctatcgacgagatcttcgagcagggcctggagctgaagcagatccacctgtcttaa

SEQ ID NO: 14 - DNA, 117 bp, synthetic construct
  source 1..117: mol_type = other DNA
atggtgaagatcatcagcagacagagcctgggcatccagcctgtgtacgacatcggcgtggagaaggaccacaacttcctgatcagcgacggcctgatcgccagcaactgcttcaac

SEQ ID NO: 15 - DNA, 309 bp, synthetic construct
  source 1..309: mol_type = other DNA
gccgagtactgcctgagctacgacaccgagatcctgaccgtggagtacggcgccatccctatcggcaaggtggtggaggagaacatcgactgtaccgtgtacaccgtggacaagaacggcttcgtgtacacccagaacatcgcccagtggcacctgagaggccagcaggaggtgttcgagtactacctggacgacggctctatcctgagagccaccaaggaccaccagttcatgaccctggagggcgagatgctgcctatccacgagatcttcgagagaggcctggagctgaagaagatcaagatctaa

SEQ ID NO: 16 - DNA, 117 bp, synthetic construct
  source 1..117: mol_type = other DNA
atggtgaagatcgtgagctacagaagcctgggcaagcagttcgtgtacgacatcggcgtggcccaggaccacaacttcctgctggccaacggcagcatcgccagcaactgcttcaac

SEQ ID NO: 17 - DNA, 330 bp, synthetic construct
  source 1..330: mol_type = other DNA
gccgagtactgcctgagctacgacaccgagatcctgaccgtggagtacggcgccatgtacatcggcaagatcgtggaggagaacatcaactgcaccgtgtacaccgtggacaagaacggcttcgtgtacacccagaccatcgcccagtggcacaacagaggcgaacaggaaattttcgagtacgacctggaggacggcagcaagatcaaggccaccaaggaccacaagttcatgaccatcgacggcgagatgctgcctatcgacgagatcttcgagaagaacctggacctgaagcaggtggtgagccaccctgacgactacctggtgtaa

SEQ ID NO: 18 - DNA, 117 bp, synthetic construct
  source 1..117: mol_type = other DNA
atggtgaagatcatcggctgcagaagcctgggcacccagaaggtgtacgacatcggcgtggagaaggaccacaacttcctgctggccaacggcagcatcgccagcaactgcttcaac

SEQ ID NO: 19 - DNA, 276 bp, synthetic construct
  source 1..276: mol_type = other DNA
agcggctactgtctggacctcaagacccaggtgcagacccctcagggcatgaaggagatcagcaacatccaggtgggagatctggtgctgagcaacaccggctacaacgaggtgctgaacgtgttccctaagagcaagaagaagtcttacaagatcaccctggaggacggcaaggagatcatctgcagcgaggagcacctgttccctacccagaccggcgagatgaacatcagcggcggcctgaaggagggcatgtgcctgtacgtgaaggagtaa

SEQ ID NO: 20 - DNA, 120 bp, synthetic construct
  source 1..120: mol_type = other DNA
atgatgctgaagaagatcctgaagatcgaggagctggacgagagagagctgatcgacatcgaggtgagcggcaaccacctgttctacgccaacgacatcctgacccacaacagcagcagc

SEQ ID NO: 21 - DNA, 279 bp, synthetic construct
  source 1..279: mol_type = other DNA
ctgaacagatgtctgtctctggataccatggtggtgacaaacggcaaggccatcgagattagagatgtcaaagtgggcgactggctggagagcgagtgtggccctgtgcaggtgacagaggtgctgcctatcatcaagcagcctgtgttcgagatcgtgctgaagtctggcaagaagatcagagtgtctgccaaccacaagttccctacaaaggacggcctcaagacaatcaacagcggactgaaggtgggcgacttcctgagaagcagagccaagtaa

SEQ ID NO: 22 - DNA, 144 bp, synthetic construct
  source 1..144: mol_type = other DNA
atgtgcgagatcttcgagaacgagatcgactgggacgagatcgccagcatcgagtacgtgggcgtggaggagacaatcgacatcaacgtgaccaacgacagactgttcttcgccaacggcatcctgacccacaacagcgccgtg

SEQ ID NO: 23 - DNA, 315 bp, synthetic construct
  source 1..315: mol_type = other DNA
ggcggcggctgcttcgtgcctggcaccctggtgaacaccgagaacggcctgaagaagatcgaggagatcaaggtgggcgacaaggtgttcagccacaccggcaagctgcaggaggtggtggacaccctgatcttcgacagagatgaggagatcatcagcatcaacggcatcgactgcaccaagaaccacgagttctacgtgatcgacaaggagaacgccaacagagtgaacgaggacaacatccacctgttcgccagatgggtgcacgccgaggagctggacatgaagaagcacctgctgatcgagctggagtaa

SEQ ID NO: 24 - DNA, 129 bp, synthetic construct
  source 1..129: mol_type = other DNA
atgaagttcaagctgaaggagatcaccagcatcgagacaaagcactacaagggcaaggtgcacgacctgaccgtgaaccaggaccacagctacaacgtgagaggcaccgtggtgcacaacagcatctgc

SEQ ID NO: 25 - DNA, 312 bp, synthetic construct
  source 1..312: mol_type = other DNA
gccgagtactgcctgggcggcgagacactgatcctgacagaggagtacggcctgctgcctatcgccaagatcgtgagcgaggagatcaactgcacagtgtacaccgtggaccagaacggcttcgtgtactctcagcctatcagccagtggcacgagagaggcctgcaggaggtgttcgagtacacactggagaacggccagaccatccaggccaccaaggaccacaagttcatgacatctgatggcgagatgctggccatcgacacaatctttgagagaggcctggacctgaagtctagcgacttctcttaa

SEQ ID NO: 26 - DNA, 117 bp, synthetic construct
  source 1..117: mol_type = other DNA
atggtgaagatcatcggcagacagagcctgggcagaaagcctgtgtacgacatcggcgtggagaaggaccacaacttcctgctgggcaacggcctgatcgccagcaactgcttcaac

SEQ ID NO: 27 - DNA, 324 bp, synthetic construct
  source 1..324: mol_type = other DNA
gccgagtactgcctgagctacgacacccagatcctgaccgtggagtacggcgccgtggccatcggcgagatcgtggagaagcagatcgagtgtaccgtgtactctgtggatgagaacggctacgtgtacacccagcctatcgcccagtggcacaacagaggcgagcaggaggtgttcgagtacctgctggaggatggcgccacaatcagagccaccaaggatcacaagttcatgaccgacgaggatcagatgctgcctatcgaccagattttcgagcagggactggaactgaagcaggtggaggtgctgcagcctgtgttctaa

SEQ ID NO: 28 - DNA, 117 bp, synthetic construct
  source 1..117: mol_type = other DNA
atggtgaagatcgtgagaaggcagagcctgggcgtgcagaacgtgtacgacatcggcgtggagaaggaccacaacttctgcctggccagcggcgagatcgccagcaactgcttcaac

SEQ ID NO: 29 - DNA, 318 bp, synthetic construct
  source 1..318: mol_type = other DNA
gccgagtactgcctgagctacgagacagagatcctgacagtggagtacggcctgctgcctatcggcaagatcgtggagaagagaatcgagtgtaccgtgtactctgtggacaacaacggcaacatctatacccagcctgtggcccagtggcacgacagaggcgagcaggaggtgttcgagtactgtctggaggacggctctctgatcagagccaccaaggaccacaagttcatgaccgtggacggccagatgctgcctatcgacgagatcttcgagagagagctggacctgatgagagtggacaacctgcctaactaa

SEQ ID NO: 30 - DNA, 117 bp, synthetic construct
  source 1..117: mol_type = other DNA
atgatcaagatcgccaccagaaagtacctgggcaagcagaacgtgtacgacatcggcgtggagagggaccacaacttcgccctgaagaacggcttcatcgccagcaactgcttcaac

SEQ ID NO: 31 - DNA, 327 bp, synthetic construct
  source 1..327: mol_type = other DNA
aacccttgttgtctggtgggcagctctgagatcatcacaagaaactacggcaagaccaccatcaaggaggtggtggagatcttcgacaacgacaagaacatccaggtgctggccttcaacacccacaccgacaacatcgagtgggcccctatcaaggccgcccagctgacaagacctaacgccgagctggtggagctggagatcgacaccctgcacggcgtcaagaccatcagatgtacccctgaccaccctgtgtacaccaagaacagaggctacgtgagagccgacgagctgaccgacgacgacgagctggtggtggccatctaa

SEQ ID NO: 32 - DNA, 129 bp, synthetic construct
  source 1..129: mol_type = other DNA
atggaggccaagacctacatcggcaagctgaagagcagaaagatcgtgagcaacgaggacacctacgacatccagaccagcacccacaacttcttcgccaacgacatcctggtgcacaacagcgagatc

SEQ ID NO: 33 - DNA, 348 bp, synthetic construct
  source 1..348: mol_type = other DNA
gccgagtactgcctgagctacaacacagaggtgctgaccgtggagtacggccctctgcctatcggcaagatcgtggacgagcagattcactgtagagtgtactctgtggacgagaacggcttcgtgtacacccaggccattgctcagtggcacgatagaggctaccaggagatcttcgcctacgagctggccgatggctctgtgatcagagccaccaaggatcaccagttcatgaccgaggatggccagatgttccctatcgatgagatctgggagaagggcctggacctgaagaagctgcctaccgtgcaggacctgcctgccgccgtgggctacaccgtgtcttaa

SEQ ID NO: 34 - skipped

SEQ ID NO: 35 - DNA, 351 bp, synthetic construct
  source 1..351: mol_type = other DNA
gccgagtactgcctggccgccgacacagaggtgctgacagtggagtacggccctatcgccatcggcaagctggtggaggagaacatcagatgtcaggtgtactgttgcaatcctgacggatacatctactctcagcctatcggacagtggcaccagagaggcgagcaggaggtgatcgagtacgagctgtctgatggcagaatcatcagagccaccgccgatcacagattcatgacagaggagggcgagatgctgagcctggacgagatcttcgagagatctctggagctgaagcagattcctacccctctgctggccattgcccagccttctcctctggccaccgcctaa

SEQ ID NO: 36 - DNA, 117 bp, synthetic construct
  source 1..117: mol_type = other DNA
atggtgaagatcgtgagaagaagaagcctgggcgtgcagcctgtgtacgacctgggcgtggccaccgtgcacaacttcgtgctggccaacggcctggtggccagcaactgcttcaac

SEQ ID NO: 37 - DNA, 381 bp, synthetic construct
  source 1..381: mol_type = other DNA
gccgagtactgcctgagcttcggcacagagattctgaccgtggagtacggccctctgcctattggcaagattgtgagcgaggagatcaactgttctgtgtactctgtggaccctgagggaagagtgtacacccaggccatcgcccagtggcacgacagaggcgagcaggaggtgctggagtacgagctggaggacggctctgtgatcagagccaccagcgaccacagattcctgacaacagactaccagctgctggccattgaggagatcttcgccagacagctggacctgctgacactggagaacattaagcagaccgaggaggccctggacaaccacagactgcctttccctctgctggacgccggcaccattaagtaa

SEQ ID NO: 38 - DNA, 117 bp, synthetic construct
  source 1..117: mol_type = other DNA
atggtgaaggtgatcggcagaagaagcctgggcgtgcagagaatcttcgacatcggcctgcctcaggaccacaacttcctgctggccaacggcgccatcgccgccaactgcttcaac

SEQ ID NO: 39 - DNA, 333 bp, synthetic construct
  source 1..333: mol_type = other DNA
gccgagtactgcctggccggaggaacacctgttgtgacagtggaatacggagtgctgcctattcagaccattgtggagcaggagctgctgtgtcacgtgtactctgtggatgcccagggcctgatctacgcccagctgattgagcagtggcaccagagaggagatagactgctgtacgagtacgagctggagaacggccagatgatcagagccacacctgatcacagattcctgaccaccaccggcgagctgctgcctatcgatgagatcttcacccagaacctggatctggccgcctgggccgtgcctgattctctgcctagaaccgcctaa

SEQ ID NO: 40 - DNA, 117 bp, synthetic construct
  source 1..117: mol_type = other DNA
atggtgaagatcatcagaagaaagttcatcggccacgcccctacctacgacatcggcctgagccaggaccacaacttcctgctgggccagggcctgatcgccgccaactgcttcaac

SEQ ID NO: 41 - DNA, 363 bp, synthetic construct
  source 1..363: mol_type = other DNA
gccgagtactgcctgagcggcgaaaccgccgtgatgaccgtggagtacggcgccgtgcctatcagaaggctggtgcaggaaagactgagctgccacgtgtactctctggatggccagggccacctgtacacacagcctatcgcccagtggcacttccagggcttcagacctgtgtacgaataccagctggaagatggctctaccatctgcgccacacctgatcacagattcatgaccaccagaggccagatgctgcctatcgaacagatcttccaggaaggcctggaactgtggcaggtggctattgctcctagacaggctctgctgcagggactgaagcctgctgtgcagatgtctggataa

SEQ ID NO: 42 - DNA, 114 bp, synthetic construct
  source 1..114: mol_type = other DNA
atgaagatcgtgggcaggagactgatgggctggcaggccgtgtacgacatcggcctggccgccgaccacaacttcgtgctggccaacggcgccatcgccgccaactgcttcaac

SEQ ID NO: 43 - DNA, 315 bp, synthetic construct
  source 1..315: mol_type = other DNA
gccgagtactgcctgacctacgagacagagatcatgaccgtggagtacggccctctgcctatcggcaagatcgtggagtacagaatcgagtgtaccgtgtacaccgtggacaagaacggctacatctacacacagcctatcgcccagtggcacaacagaggcatgcaggaggtgtacgagtacagcctggaggacggcaccgtgatcagagccacccctgagcacaagttcatgaccgaggacggccagatgctgcctatcgatgagatcttcgagagaaacctggacctgaagtgtctgggcaccctggagtaa

SEQ ID NO: 44 - DNA, 117 bp, synthetic construct
  source 1..117: mol_type = other DNA
atggtgaagatcgtgagcagaaagctggccaagaccgagaacgtgtacgacatcggcgtgaccaaggaccacaacttcgtgctggccaacggcctgatcgccagcaactgcttcaac

SEQ ID NO: 45 - DNA, 363 bp, synthetic construct
  source 1..363: mol_type = other DNA
gccgagtactgcctgagcggcgagacagccgtgatgaccgtggagtacggcgccatccctatcagaaggctggtgcaggagagactgatttgccaggtgtacagcctggaccctcagggccacctgtacacacagcctattgcccagtggcacttccagggcttcagacctgtgtacgcttaccagctggaggacggctctaccatttgtgctacacctgaccacagattcatgacaacaagcggccagatgctgcctatcgagcagatcttcagagagggactggagctgtggcaggtggctattgctcctcctggagctctggctcagggactgaagcctgctgtgcagatgtcttgttaa

SEQ ID NO: 46 - DNA, 114 bp, synthetic construct
  source 1..114: mol_type = other DNA
atgaagatcgtgggcaggagactggtgggctggcaggccgtgtacgacatcggcctggccggcgaccacaacttcctgctggccaacggcgccatcgccgccaactgcttcaac

SEQ ID NO: 47-81 - skipped (35 entries)

SEQ ID NO: 82 - protein, 1319 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYSYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 83 - protein, 1329 aa, synthetic construct
MMLKKILKIEELDERELIDIEVSGNHLFYANDILTHNSATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 84 - protein, 1317 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYSYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 85 - protein, 1328 aa, synthetic construct
MMLKKILKIEELDERELIDIEVSGNHLFYANDILTHNSTQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 86 - protein, 1354 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYSYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 87 - protein, 1293 aa, synthetic construct
MMLKKILKIEELDERELIDIEVSGNHLFYANDILTHNSLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 88 - protein, 1359 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYSYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 89 - protein, 1286 aa, synthetic construct
MMLKKILKIEELDERELIDIEVSGNHLFYANDILTHNSPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 90 - protein, 1181 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGGCFVPGTLVNTENGLKKIEEIKVGDKVFSHTGKLQEVVDTLIFDRDEEIISINGIDCTKNHEFYVIDKENANRVNEDNIHLFARWVHAEELDMKKHLLIELE

SEQ ID NO: 91 - protein, 1442 aa, synthetic construct
MKFKLKEITSIETKHYKGKVHDLTVNQDHSYNVRGTVVHNSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 92 - protein, 1328 aa, synthetic construct; identical to SEQ ID NO: 85

SEQ ID NO: 93 - protein, 1180 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGCFVPGTLVNTENGLKKIEEIKVGDKVFSHTGKLQEVVDTLIFDRDEEIISINGIDCTKNHEFYVIDKENANRVNEDNIHLFARWVHAEELDMKKHLLIELE

SEQ ID NO: 94 - protein, 1442 aa, synthetic construct; identical to SEQ ID NO: 91

SEQ ID NO: 95 - protein, 1328 aa, synthetic construct; identical to SEQ ID NO: 85

SEQ ID NO: 96 - protein, 1289 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYGCFVPGTLVNTENGLKKIEEIKVGDKVFSHTGKLQEVVDTLIFDRDEEIISINGIDCTKNHEFYVIDKENANRVNEDNIHLFARWVHAEELDMKKHLLIELE

SEQ ID NO: 97 - protein, 1378 aa, synthetic construct
MKFKLKEITSIETKHYKGKVHDLTVNQDHSYNVRGTVVHNSKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 98 - protein, 1286 aa, synthetic construct; identical to SEQ ID NO: 89

SEQ ID NO: 99 - protein, 1288 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEGCFVPGTLVNTENGLKKIEEIKVGDKVFSHTGKLQEVVDTLIFDRDEEIISINGIDCTKNHEFYVIDKENANRVNEDNIHLFARWVHAEELDMKKHLLIELE

SEQ ID NO: 100 - protein, 1377 aa, synthetic construct
MKFKLKEITSIETKHYKGKVHDLTVNQDHSYNVRGTVVHNSTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 101 - protein, 1286 aa, synthetic construct; identical to SEQ ID NO: 89

SEQ ID NO: 102 - protein, 1185 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGCCLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAI

SEQ ID NO: 103 - protein, 1442 aa, synthetic construct
MEAKTYIGKLKSRKIVSNEDTYDIQTSTHNFFANDILVHNSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 104 - protein, 1328 aa, synthetic construct; identical to SEQ ID NO: 85

SEQ ID NO: 105 - protein, 1186 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDCCLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAI

SEQ ID NO: 106 - protein, 1442 aa, synthetic construct; identical to SEQ ID NO: 103

SEQ ID NO: 107 - protein, 1328 aa, synthetic construct; identical to SEQ ID NO: 85

SEQ ID NO: 108 - protein, 1293 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYCCLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAI

SEQ ID NO: 109 - protein, 1378 aa, synthetic construct
MEAKTYIGKLKSRKIVSNEDTYDIQTSTHNFFANDILVHNSKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 110 - protein, 1286 aa, synthetic construct; identical to SEQ ID NO: 89

SEQ ID NO: 111 - protein, 1292 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFECCLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAI

SEQ ID NO: 112 - protein, 1377 aa, synthetic construct
MEAKTYIGKLKSRKIVSNEDTYDIQTSTHNFFANDILVHNSTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 113 - protein, 1286 aa, synthetic construct; identical to SEQ ID NO: 89

SEQ ID NO: 114 - protein, 850 aa, synthetic construct
MLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVPLFPPPTPLEPSPTLPDLDVVADTGGEEDTEDQGLTGDEAEPFLDQSGGPGAPTTPRKLPSRPPPHYPGIKRKRSAPTSRKLLSDKPQDFQIRVQVIEGRQLPGVNIKPVVKVTAAGQTKRTRIHKGNSPLFNETLFFNLFDSPGELFDEPIFITVVDSRSLRTDALLGEFRMDVGTIYREPRHAYLRKWLLLSDPDDFSAGARGYLKTSLCVLGPGDEAPLERKDPSEDKEDIESNLLRPTGVALRGAHFCLKVFRAEDLPQMDDAVMDNVKQIFGFESNKKNLVDPFVEVSFAGKMLCSKILEKTANPQWNQNITLPAMFPSMCEKMRIRIIDWDRLTHNDIVATTYLSMSKISAPGGEIEEEPAGAVKPSKASDLDDYLGFLPTFGPCYINLYGSPREFTGFPDPYTELNTGKGEGVAYRGRLLLSLETKLVEHSEQKVEDLPADDILRVEKYLRRRKYSLFAAFYSATMLQDVDDAIQFEVSIGNYGNKFDMTCLPLASTTQYSRAVFDGCHYYYLPWGNVKPVVVLSSYWEDISHRIETQNQLLGIADRLEAGLEQVHLALKAQCSTEDVDSLVAQLTDELIAGCSQPLGDIHETPSATHLDQYLYQLRTHHLSQITEAALALKLGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 115 - protein, 1355 aa, synthetic construct
MMLKKILKIEELDERELIDIEVSGNHLFYANDILTHNSELPAALEQAEDWLLRLRALAEEPQNSLPDIVIWMLQGDKRVAYQRVPAHQVLFSRRGANYCGKNCGKLQTIFLKYPMEKVPGARMPVQIRVKLWFGLSVDEKEFNQFAEGKLSVFAETYENETKLALVGNWGTTGLTYPKFSDVTGKIKLPKDSFRPSAGWTWAGDWFVCPEKTLLHDMDAGHLSFVEEVFENQTRLPGGQWIYMSDNYTDVNGEKVLPKDDIECPLGWKWEDEEWSTDLNRAVDEQGWEYSITIPPERKPKHWVPAEKMYYTHRRRRWVRLRRRDLSQMEALKRHRQAEAEGEGWEYASLFGWKFHLEYRKTDAFRRRRWRRRMEPLEKTGPAAVFALEGALGGVMDDKSEDSMSVSTLSFGVNRPTISCIFDYGNRYHLRCYMYQARDLAAMDKDSFSDPYAIVSFLHQSQKTVVVKNTLNPTWDQTLIFYEIEIFGEPATVAEQPPSIVVELYDHDTYGADEFMGRCICQPSLERMPRLAWFPLTRGSQPSGELLASFELIQREKPAIHHIPGFEVQETSRILDESEDTDLPYPPPQREANIYMVPQNIKPALQRTAIEILAWGLRNMKSYQLANISSPSLVVECGGQTVQSCVIRNLRKNPNFDICTLFMEVMLPREELYCPPITVKVIDNRQFGRRPVVGQCTIRSLESFLCDPYSAESPSPQGGPDDVSLLSPGEDVLIDIDDKEPLIPIQEEEFIDWWSKFFASIGEREKCGSYLEKDFDTLKVYDTQLENVEAFEGLSDFCNTFKLYRGKTQEETEDPSVIGEFKGLFKIYPLPEDPAIPMPPRQFHQLAAQGPQECLVRIYIVRAFGLQPKDPNGKCDPYIKISIGKKSVSDQDNYIPCTLEPVFGKMFELTCTLPLEKDLKITLYDYDLLSKDEKIGETVVDLENRLLSKFGARCGLPQTYCVSGPNQWRDQLRPSQLLHLFCQQHRVKAPVYRTDRVMFQDKEYSIEEIEAGRIPNPHLGPVEERLALHVLQQQGLVPEHVESRPLYSPLQPDIEQGKLQMWVDLFPKALGRPGPPFNITPRRARRFFLRCIIWNTRDVILDDLSLTGEKMSDIYVKGWMIGFEEHKQKTDVHYRSLGGEGNFNWRFIFPFDYLPAEQVCTIAKKDAFWRLDKTESKIPARVVFQIWDNDKFSFDDFLGSLQLDLNRMPKPAKTAKKCSLDQLDDAFHPEWFVSLFEQKTVKGWWPCVAEEGEKKILAGKLEMTLEIVAESEHEERPAGQGRDEPNMNPKLEDPRRPDTSFLWFTSPYKTMKFILWRRFRWAIILFIILFILLLFLAIFIYAFPNYAAMKLVKPFS

SEQ ID NO: 116 - protein, 948 aa, synthetic construct
MLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVPLFPPPTPLEPSPTLPDLDVVADTGGEEDTEDQGLTGDEAEPFLDQSGGPGAPTTPRKLPSRPPPHYPGIKRKRSAPTSRKLLSDKPQDFQIRVQVIEGRQLPGVNIKPVVKVTAAGQTKRTRIHKGNSPLFNETLFFNLFDSPGELFDEPIFITVVDSRSLRTDALLGEFRMDVGTIYREPRHAYLRKWLLLSDPDDFSAGARGYLKTSLCVLGPGDEAPLERKDPSEDKEDIESNLLRPTGVALRGAHFCLKVFRAEDLPQMDDAVMDNVKQIFGFESNKKNLVDPFVEVSFAGKMLCSKILEKTANPQWNQNITLPAMFPSMCEKMRIRIIDWDRLTHNDIVATTYLSMSKISAPGGEIEEEPAGAVKPSKASDLDDYLGFLPTFGPCYINLYGSPREFTGFPDPYTELNTGKGEGVAYRGRLLLSLETKLVEHSEQKVEDLPADDILRVEKYLRRRKYSLFAAFYSATMLQDVDDAIQFEVSIGNYGNKFDMTCLPLASTTQYSRAVFDGCHYYYLPWGNVKPVVVLSSYWEDISHRIETQNQLLGIADRLEAGLEQVHLALKAQCSTEDVDSLVAQLTDELIAGCSQPLGDIHETPSATHLDQYLYQLRTHHLSQITEAALALKLGHSELPAALEQAEDWLLRLRALAEEPQNSLPDIVIWMLQGDKRVAYQRVPAHQVLFSRRGANYCGKNCGKLQTIFLKYPMEKVPGARMPVQIRVKLWFGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 117 - protein, 1257 aa, synthetic construct
MMLKKILKIEELDERELIDIEVSGNHLFYANDILTHNSVDEKEFNQFAEGKLSVFAETYENETKLALVGNWGTTGLTYPKFSDVTGKIKLPKDSFRPSAGWTWAGDWFVCPEKTLLHDMDAGHLSFVEEVFENQTRLPGGQWIYMSDNYTDVNGEKVLPKDDIECPLGWKWEDEEWSTDLNRAVDEQGWEYSITIPPERKPKHWVPAEKMYYTHRRRRWVRLRRRDLSQMEALKRHRQAEAEGEGWEYASLFGWKFHLEYRKTDAFRRRRWRRRMEPLEKTGPAAVFALEGALGGVMDDKSEDSMSVSTLSFGVNRPTISCIFDYGNRYHLRCYMYQARDLAAMDKDSFSDPYAIVSFLHQSQKTVVVKNTLNPTWDQTLIFYEIEIFGEPATVAEQPPSIVVELYDHDTYGADEFMGRCICQPSLERMPRLAWFPLTRGSQPSGELLASFELIQREKPAIHHIPGFEVQETSRILDESEDTDLPYPPPQREANIYMVPQNIKPALQRTAIEILAWGLRNMKSYQLANISSPSLVVECGGQTVQSCVIRNLRKNPNFDICTLFMEVMLPREELYCPPITVKVIDNRQFGRRPVVGQCTIRSLESFLCDPYSAESPSPQGGPDDVSLLSPGEDVLIDIDDKEPLIPIQEEEFIDWWSKFFASIGEREKCGSYLEKDFDTLKVYDTQLENVEAFEGLSDFCNTFKLYRGKTQEETEDPSVIGEFKGLFKIYPLPEDPAIPMPPRQFHQLAAQGPQECLVRIYIVRAFGLQPKDPNGKCDPYIKISIGKKSVSDQDNYIPCTLEPVFGKMFELTCTLPLEKDLKITLYDYDLLSKDEKIGETVVDLENRLLSKFGARCGLPQTYCVSGPNQWRDQLRPSQLLHLFCQQHRVKAPVYRTDRVMFQDKEYSIEEIEAGRIPNPHLGPVEERLALHVLQQQGLVPEHVESRPLYSPLQPDIEQGKLQMWVDLFPKALGRPGPPFNITPRRARRFFLRCIIWNTRDVILDDLSLTGEKMSDIYVKGWMIGFEEHKQKTDVHYRSLGGEGNFNWRFIFPFDYLPAEQVCTIAKKDAFWRLDKTESKIPARVVFQIWDNDKFSFDDFLGSLQLDLNRMPKPAKTAKKCSLDQLDDAFHPEWFVSLFEQKTVKGWWPCVAEEGEKKILAGKLEMTLEIVAESEHEERPAGQGRDEPNMNPKLEDPRRPDTSFLWFTSPYKTMKFILWRRFRWAIILFIILFILLLFLAIFIYAFPNYAAMKLVKPFS

SEQ ID NO: 118 - protein, 1364 aa, synthetic construct
MLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVPLFPPPTPLEPSPTLPDLDVVADTGGEEDTEDQGLTGDEAEPFLDQSGGPGAPTTPRKLPSRPPPHYPGIKRKRSAPTSRKLLSDKPQDFQIRVQVIEGRQLPGVNIKPVVKVTAAGQTKRTRIHKGNSPLFNETLFFNLFDSPGELFDEPIFITVVDSRSLRTDALLGEFRMDVGTIYREPRHAYLRKWLLLSDPDDFSAGARGYLKTSLCVLGPGDEAPLERKDPSEDKEDIESNLLRPTGVALRGAHFCLKVFRAEDLPQMDDAVMDNVKQIFGFESNKKNLVDPFVEVSFAGKMLCSKILEKTANPQWNQNITLPAMFPSMCEKMRIRIIDWDRLTHNDIVATTYLSMSKISAPGGEIEEEPAGAVKPSKASDLDDYLGFLPTFGPCYINLYGSPREFTGFPDPYTELNTGKGEGVAYRGRLLLSLETKLVEHSEQKVEDLPADDILRVEKYLRRRKYSLFAAFYSATMLQDVDDAIQFEVSIGNYGNKFDMTCLPLASTTQYSRAVFDGCHYYYLPWGNVKPVVVLSSYWEDISHRIETQNQLLGIADRLEAGLEQVHLALKAQCSTEDVDSLVAQLTDELIAGCSQPLGDIHETPSATHLDQYLYQLRTHHLSQITEAALALKLGHSELPAALEQAEDWLLRLRALAEEPQNSLPDIVIWMLQGDKRVAYQRVPAHQVLFSRRGANYCGKNCGKLQTIFLKYPMEKVPGARMPVQIRVKLWFGLSVDEKEFNQFAEGKLSVFAETYENETKLALVGNWGTTGLTYPKFSDVTGKIKLPKDSFRPSAGWTWAGDWFVCPEKTLLHDMDAGHLSFVEEVFENQTRLPGGQWIYMSDNYTDVNGEKVLPKDDIECPLGWKWEDEEWSTDLNRAVDEQGWEYSITIPPERKPKHWVPAEKMYYTHRRRRWVRLRRRDLSQMEALKRHRQAEAEGEGWEYASLFGWKFHLEYRKTDAFRRRRWRRRMEPLEKTGPAAVFALEGALGGVMDDKSEDSMSVSTLSFGVNRPTISCIFDYGNRYHLRCYMYQARDLAAMDKDSFSDPYAIVSFLHQSQKTVVVKNTLNPTWDQTLIFYEIEIFGEPATVAEQPPSIVVELYDHDTYGADEFMGRCICQPSLERMPRLAWFPLTRGCFVPGTLVNTENGLKKIEEIKVGDKVFSHTGKLQEVVDTLIFDRDEEIISINGIDCTKNHEFYVIDKENANRVNEDNIHLFARWVHAEELDMKKHLLIELE

SEQ ID NO: 119 - protein, 857 aa, synthetic construct
MKFKLKEITSIETKHYKGKVHDLTVNQDHSYNVRGTVVHNSQPSGELLASFELIQREKPAIHHIPGFEVQETSRILDESEDTDLPYPPPQREANIYMVPQNIKPALQRTAIEILAWGLRNMKSYQLANISSPSLVVECGGQTVQSCVIRNLRKNPNFDICTLFMEVMLPREELYCPPITVKVIDNRQFGRRPVVGQCTIRSLESFLCDPYSAESPSPQGGPDDVSLLSPGEDVLIDIDDKEPLIPIQEEEFIDWWSKFFASIGEREKCGSYLEKDFDTLKVYDTQLENVEAFEGLSDFCNTFKLYRGKTQEETEDPSVIGEFKGLFKIYPLPEDPAIPMPPRQFHQLAAQGPQECLVRIYIVRAFGLQPKDPNGKCDPYIKISIGKKSVSDQDNYIPCTLEPVFGKMFELTCTLPLEKDLKITLYDYDLLSKDEKIGETVVDLENRLLSKFGARCGLPQTYCVSGPNQWRDQLRPSQLLHLFCQQHRVKAPVYRTDRVMFQDKEYSIEEIEAGRIPNPHLGPVEERLALHVLQQQGLVPEHVESRPLYSPLQPDIEQGKLQMWVDLFPKALGRPGPPFNITPRRARRFFLRCIIWNTRDVILDDLSLTGEKMSDIYVKGWMIGFEEHKQKTDVHYRSLGGEGNFNWRFIFPFDYLPAEQVCTIAKKDAFWRLDKTESKIPARVVFQIWDNDKFSFDDFLGSLQLDLNRMPKPAKTAKKCSLDQLDDAFHPEWFVSLFEQKTVKGWWPCVAEEGEKKILAGKLEMTLEIVAESEHEERPAGQGRDEPNMNPKLEDPRRPDTSFLWFTSPYKTMKFILWRRFRWAIILFIILFILLLFLAIFIYAFPNYAAMKLVKPFS

SEQ ID NO: 120 - protein, 1328 aa, synthetic construct
MAKYGEHEASPDNGQNEFSDIIKSRSDEHNDVQKKTFTKWINARFSKSGKPPINDMFTDLKDGRKLLDLLEGLTGTSLPKERGSTRVHALNNVNRVLQVLHQNNVELVNIGGTDIVDGNHKLTLGLLWSIILHWQVKDVMKDVMSDLQQTNSEKILLSWVRQTTRPYSQVNVLNFTTSWTDGLAFNAVLHRHKPDLFSWDKVVKMSPIERLEHAFSKAQTYLGIEKLLDPEDVAVQLPDKKSIIMYLTSLFEVLPQQVTIDAIREVETLPRKYKKECEEEAINIQSTAPEEEHESPRAETPSTVTEVDMDLDSYQIALEEVLTWLLSAEDTFQEQDDISDDVEEVKDQFATHEAFMMELTAHQSSVGSVLQAGNQLITQGTLSDEEEFEIQEQMTLLNARWEALRVESMDRQSRLHDVLMELQKKQLQQLSAWLTLTEERIQKMETCPLDDDVKSLQKLLEEHKSLQSDLEAEQVKVNSLTHMVVIVDENSGESATAILEDQLQKLGERWTAVCRWTEERWNRLQEINILWQELLEEQCLLKAWLTEKEEALNKVQTSNFKDQKELSVSVRRLAILKEDMEMKRQTLDQLSEIGQDVGQLLDNSKASKKINSDSEELTQRWDSLVQRLEDSSNQVTQAVAKLGMSQIPQKDLLETVRVREQAITKKSKQELPPPPPPKKRQIHVDIEAKKKFDAISAELLNWILKWKTAIQTTEIKEYMKMQDTSEMKKKLKALEKEQRERIPRADELNQTGQILVEQMGKEGLPTEEIKNVLEKVSSEWKNVSQHLEDLERKIQLEEWRQFHCDLNDLTQWITEAEELLVDTCAPGGSLDLEKARIHQQELEVGISSHQPSFAALNRTGDGIVQKLSQADGSFLKEKLAGLNQRWDAIVAEVKDRQPRLKGESKQVMKYRHQLDEIICWLTKAEHAMQKRSTTELGENLQELRDLTQEMEVHAEKLKWLNRTELEMLSDKSLSLPERDKISESLRTVNMTWNKICREVPTTLKECIQEPSSVSQTRIAAHPNVQKVVLVSSASDIPVQSHRTSEISIPADLDKTITELADWLVLIDQMLKSNIVTVGDVEEINKTVSRMKITKADLEQRHPQLDYVFTLAQNLKNKASSSDMRTAITEKLERVKNQWDGTQHGVELRQQQLEDMIIDSLQWDDHREETEELMRKYEARLYILQQARRDPLTKQISDNQILLQELGPGDGIVMAFDNVLQKLLEEYGCFVPGTLVNTENGLKKIEEIKVGDKVFSHTGKLQEVVDTLIFDRDEEIISINGIDCTKNHEFYVIDKENANRVNEDNIHLFARWVHAEELDMKKHLLIELE

SEQ ID NO: 121 - protein, 1067 aa, synthetic construct
MKFKLKEITSIETKHYKGKVHDLTVNQDHSYNVRGTVVHNSDDTRNVKETTEYLKTSWINLKQSIADRQNALEAEWRTVQASRRDLENFLKWIQEAETTVNVLVDASHRENALQDSILARELKQQMQDIQAEIDAHNDIFKSIDGNRQKMVKALGNSEEATMLQHRLDDMNQRWNDLKAKSASIRAHLEASAEKWNRLLMSLEELIKWLNMKDEELKKQMPIGGDVPALQLQYDHCKALRRELKEKEYSVLNAVDQARVFLADQPIEAPEEPRRNLQSKTELTPEERAQKIAKAMRKQSSEVKEKWESLNAVTSNWQKQVDKALEKLRDLQGAMDDLDADMKEAESVRNGWKPVGDLLIDSLQDHIEKIMAFREEIAPINFKVKTVNDLSSQLSPLDLHPSLKMSRQLDDLNMRWKLLQVSVDDRLKQLQEAHRDFGPSSQHFLSTSVQLPWQRSISHNKVPYYINHQTQTTCWDHPKMTELFQSLADLNNVRFSAYRTAIKIRRLQKALCLDLLELSTTNEIFKQHKLNQNDQLLSVPDVINCLTTTYDGLEQMHKDLVNVPLCVDMCLNWLLNVYDTGRTGKIRVQSLKIGLMSLSKGLLEEKYRYLFKEVAGPTEMCDQRQLGLLLHDAIQIPRQLGEVAAFGGSNIEPSVRSCFQQNNNKPEISVKEFIDWMHLEPQSMVWLPVLHRVAAAETAKHQAKCNICKECPIVGFRYRSLKHFNYDVCQSCFFSGRTAKGHKLHYPMVEYCIPTTSGEDVRDFTKVLKNKFRSKKYFAKHPRLGYLPVQTVLEGDNLETPITLISMWPEHYDPSQSPQLFHDDTHSRIEQYATRLAQMERTNGSFLTDSSSTTGSVEDEHALIQQYCQTLGGESPVSQPQSPAQILKSVEREERGELERIIADLEEEQRNLQVEYEQLKDQHLRRGLPVGSPPESIISPHHTSEDSELIAEAKLLRQHKGRLEARMQILEDHNKQLESQLHRLRQLLEQPESDSRINGVSPWASPQHSALSYSLDPDASGPQFHQAAGEDLLAPPHDTSTDLTEVMEQIHSTFPSCCPNVPSRPQAM

SEQ ID NO: 122 - protein, 1367 aa, synthetic construct
MAKYGEHEASPDNGQNEFSDIIKSRSDEHNDVQKKTFTKWINARFSKSGKPPINDMFTDLKDGRKLLDLLEGLTGTSLPKERGSTRVHALNNVNRVLQVLHQNNVELVNIGGTDIVDGNHKLTLGLLWSIILHWQVKDVMKDVMSDLQQTNSEKILLSWVRQTTRPYSQVNVLNFTTSWTDGLAFNAVLHRHKPDLFSWDKVVKMSPIERLEHAFSKAQTYLGIEKLLDPEDVAVQLPDKKSIIMYLTSLFEVLPQQVTIDAIREVETLPRKYKKECEEEAINIQSTAPEEEHESPRAETPSTVTEVDMDLDSYQIALEEVLTWLLSAEDTFQEQDDISDDVEEVKDQFATHEAFMMELTAHQSSVGSVLQAGNQLITQGTLSDEEEFEIQEQMTLLNARWEALRVESMDRQSRLHDVLMELQKKQLQQLSAWLTLTEERIQKMETCPLDDDVKSLQKLLEEHKSLQSDLEAEQVKVNSLTHMVVIVDENSGESATAILEDQLQKLGERWTAVCRWTEERWNRLQEINILWQELLEEQCLLKAWLTEKEEALNKVQTSNFKDQKELSVSVRRLAILKEDMEMKRQTLDQLSEIGQDVGQLLDNSKASKKINSDSEELTQRWDSLVQRLEDSSNQVTQAVAKLGMSQIPQKDLLETVRVREQAITKKSKQELPPPPPPKKRQIHVDIEAKKKFDAISAELLNWILKWKTAIQTTEIKEYMKMQDTSEMKKKLKALEKEQRERIPRADELNQTGQILVEQMGKEGLPTEEIKNVLEKVSSEWKNVSQHLEDLERKIQLQEDINAYFKQLDELEKVIKTKEEWVKHTSISESSRQSLPSLKDSCQRELTNLLGLHPKIEMARASCSALMSQPSAPDFVQRGFDSFLGRYQAVQEAVEDRQQHLENELKLLPTDYLVEINKILLCMDDVELSLNVPELNTAIYEDFSFQEDSLKNIKDQLDKLGEQIAVIHEKQPDVILEASGPEAIQIRDTLTQLNAKWDRINRMYSDRKGCFDRAMEEWRQFHCDLNDLTQWITEAEELLVDTCAPGGSLDLEKARIHQQELEVGISSHQPSFAALNRTGDGIVQKLSQADGSFLKEKLAGLNQRWDAIVAEVKDRQPRLKGESKQVMKYRHQLDEIICWLTKAEHAMQKRSTTELGENLQELRDLTQEMEVHAEKLKWLNRTELEMLSDKSLSLPERDKISESLRTVNMTWNKICREVPTTLKECIQEPSSVSQTRIAAHPNVQKVVLVSSASDIPVQSHRTCCLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAI

SEQ ID NO: 123 - protein, 1251 aa, synthetic construct
MEAKTYIGKLKSRKIVSNEDTYDIQTSTHNFFANDILVHNSEISIPADLDKTITELADWLVLIDQMLKSNIVTVGDVEEINKTVSRMKITKADLEQRHPQLDYVFTLAQNLKNKASSSDMRTAITEKLERVKNQWDGTQHGVELRQQQLEDMIIDSLQWDDHREETEELMRKYEARLYILQQARRDPLTKQISDNQILLQELGPGDGIVMAFDNVLQKLLEEYGSDDTRNVKETTEYLKTSWINLKQSIADRQNALEAEWRTVQASRRDLENFLKWIQEAETTVNVLVDASHRENALQDSILARELKQQMQDIQAEIDAHNDIFKSIDGNRQKMVKALGNSEEATMLQHRLDDMNQRWNDLKAKSASIRAHLEASAEKWNRLLMSLEELIKWLNMKDEELKKQMPIGGDVPALQLQYDHCKALRRELKEKEYSVLNAVDQARVFLADQPIEAPEEPRRNLQSKTELTPEERAQKIAKAMRKQSSEVKEKWESLNAVTSNWQKQVDKALEKLRDLQGAMDDLDADMKEAESVRNGWKPVGDLLIDSLQDHIEKIMAFREEIAPINFKVKTVNDLSSQLSPLDLHPSLKMSRQLDDLNMRWKLLQVSVDDRLKQLQEAHRDFGPSSQHFLSTSVQLPWQRSISHNKVPYYINHQTQTTCWDHPKMTELFQSLADLNNVRFSAYRTAIKIRRLQKALCLDLLELSTTNEIFKQHKLNQNDQLLSVPDVINCLTTTYDGLEQMHKDLVNVPLCVDMCLNWLLNVYDTGRTGKIRVQSLKIGLMSLSKGLLEEKYRYLFKEVAGPTEMCDQRQLGLLLHDAIQIPRQLGEVAAFGGSNIEPSVRSCFQQNNNKPEISVKEFIDWMHLEPQSMVWLPVLHRVAAAETAKHQAKCNICKECPIVGFRYRSLKHFNYDVCQSCFFSGRTAKGHKLHYPMVEYCIPTTSGEDVRDFTKVLKNKFRSKKYFAKHPRLGYLPVQTVLEGDNLETPITLISMWPEHYDPSQSPQLFHDDTHSRIEQYATRLAQMERTNGSFLTDSSSTTGSVEDEHALIQQYCQTLGGESPVSQPQSPAQILKSVEREERGELERIIADLEEEQRNLQVEYEQLKDQHLRRGLPVGSPPESIISPHHTSEDSELIAEAKLLRQHKGRLEARMQILEDHNKQLESQLHRLRQLLEQPESDSRINGVSPWASPQHSALSYSLDPDASGPQFHQAAGEDLLAPPHDTSTDLTEVMEQIHSTFPSCCPNVPSRPQAM

SEQ ID NO: 124 - DNA, 342 bp, synthetic construct
  source 1..342: mol_type = other DNA
ttacttggcgcctcttctcagcaggctcatctcgtggaatgtctccaccttcaggtccagctctctctggaagatctcctcgataggcagcatctcgccgctctcggtcatcatcttgtggtccttggtggctctgatctttctgccgtcctccagggtgtactcgatgatctcctgctggcctctagggtgccactgggcgataggctgggtgtacacgaagccgttctcatccacggtgtacacagaacactcgatcttctcctccacgatcttgccgataggcatggcgccgtactccacggtccagatctcggtgtcgtagctcaggcagtactcggc

SEQ ID NO: 125 - DNA, 117 bp, synthetic construct
  source 1..117: mol_type = other DNA
gttgaagcagttgctggccacgcagccgttggccagcacgaagttgtggtctgtctccacgcacacgtcgtacacgttctgtctgcccaggctctgtctcttgatgatcttcaccat

SEQ ID NO: 126 - DNA, 315 bp, synthetic construct
  source 1..315: mol_type = other DNA
ttagtgcaggccctgcacctgcatcaggtccagctgtctggcgaagatctcgtcgataggcagcatctcgccgtcggtggtcatgaacttgtgatccttggtggctctgatgatagagccatcctccagggtgtactcgaacacctcctgctcgcctctgttgtgccactgggcgatagactgggtgtagatgtttccgtttccgtccacagagtacactctacactcgatggccttgcccacgatctcgccgataggcaggaagccgtactccacggtcaggatctcggtatcggcgctcaggcagtactcggc

SEQ ID NO: 127 - DNA, 117 bp, synthetic construct
  source 1..117: mol_type = other DNA
gttgaagcaattggaagctatgagtccattcttaatggcgaaattgtggtgatgttccacaccaatatcatacacgttttcccggccaacgaattttctagcggtgatctttaccat

SEQ ID NO: 128 - DNA, 318 bp, synthetic construct
  source 1..318: mol_type = other DNA
ttactcaggcaggcccttcacctgcagcaggtccagctcctgctcgaagatctcgtcgataggcagcatcttgccgtcctgggtcatgaacttgtggtccttggtggccttgatgatgctgccgtcctccagacagtactcgaacacctcctgcttgcctctgtggtgccactgggcgataggctgggtgtacacgatgccgttgttgttgatagagaacacgctacactcgatgcccttctccacgatctcgccgataggcacgaagccgtactccacggtcagcacctcggtatcgtagctcaggcagtactcggc

SEQ ID NO: 129 - DNA, 117 bp, synthetic construct
  source 1..117: mol_type = other DNA
gttgaagcagttgctggcgatcaggccgttcttgatgaagaagttgtgatctcttctcacgccgatgtcgtacacgttctccacgcccaggaactttctgctggcgatcttgatcat

SEQ ID NO: 130 - DNA, 318 bp, synthetic construct
  source 1..318: mol_type = other DNA
ttactcaggcaggcccttcacctgcagcaggtccagctcctgctcgaagatctcatcgataggcagcatcttgccatcctgggtcatgaacttgtggtccttggtggccttgatgatagagccgtcctccagacagtactcgaacacctcctgcttgcctctgtggtgccactgggcgataggctgggtgtacacgatgccgttgctgtcgatgctgaacacagaacactcgatgcccttgtccacgatctcgccgataggcacgaagccgtactccacggtcagcacctcggtgtcgtagctcaggcagtactcggc

SEQ ID NO: 131 - DNA, 117 bp, synthetic construct
  source 1..117: mol_type = other DNA
gttgaagcagttgctggcgatcaggccgttcttcacgaagaagttgtgatctctgcccacgccgatgtcgtacacgttctccacgcccaggaactttctgctggcgatcttgatcat

SEQ ID NO: 132 - DNA, 366 bp, synthetic construct
  source 1..366: mol_type = other DNA
ttacagctttctgtccacgatgatgaaaggcagggttctctgctgcaccagcttgctgatgccagacacctgcaccagctcccagccctggtggaagatctcatcgataggcaccatctcgccctgggtggtcatgaacttgtggtccttggtggctctgatggtgctgccgtcgtccaggcagtactcgtacaggtcctgcacgcctctatcgtgccactggccgataggctgggtgtacacgttgccagagtcgttcagagagaacacggtacacaccatctgcttgttcacgatctcgccgataggcacgaagccgtactccagggtcagcacctctgtctcgtagctcaggcagtactcggc

SEQ ID NO: 133 - DNA, 117 bp, synthetic construct
  source 1..117: mol_type = other DNA
gttgaagcagttgctggccaccaggccgttcttgatgatgaagttgtggtccttggccacgccgatgtcgtacacgtcggccttgcccaggtatcttctgctcacgatcttcaccat

SEQ ID NO: 134 - DNA, 309 bp, synthetic construct
  source 1..309: mol_type = other DNA
ttacacggtcagcttcttcaggtccaggttgttctgcaagatctcgtcgataggcagcatcttgccctcggtggtcatgaacttgtggtccttggtggctctgattgtctcgccgtcctccaggtagtactcgaacacctcctgctcgcctctgtggtgccactgggcgatgctctgggtgtacacgaagccgttcttgttcacggtgtacacgctgcagttgatgttctcctccacgatcttgccgataggcatagggccgtactccacggtcaggatctcggtgtcgtagctcaggcagtactcggc

SEQ ID NO: 135 - DNA, 117 bp, synthetic construct
  source 1..117: mol_type = other DNA
gttgaagcagttgctggcgatcaggttgttgctcagcaggaagttgtggtccttctccacgccgatgtcgtacacgttctgcttgcccaggcttcttctctcgatgatcttcaccat

SEQ ID NO: 136 - DNA, 312 bp, synthetic construct
  source 1..312: mol_type = other DNA
ttaagacaggtggatctgcttcagctccaggccctgctcgaagatctcgtcgataggcagcatctggccgtcggtggtcatgaacttgtggtcctttgtggctctgatggtgctgccgtcctccaggcagtactcgaacacctcctgctcgcctctgtagtgccactgggcgataggctgggtgtacacgaagccgttcttgttcacggtgtacacggtacagtcgatctgctcctccacgatcttgccgataggcatcaggccgtactccacggtcatgatctgtgtctcgtagctcaggcagtactcggc

SEQ ID NO: 137 - DNA, 117 bp, synthetic construct
  source 1..117: mol_type = other DNA
gttgaagcagttgctggcgatcaggccgtcgctgatcaggaagttgtggtccttctccacgccgatgtcgtacacaggctggatgcccaggctctgtctgctgatgatcttcaccat

SEQ ID NO: 138 - DNA, 309 bp, synthetic construct
  source 1..309: mol_type = other DNA
ttagatcttgatcttcttcagctccaggcctctctcgaagatctcgtggataggcagcatctcgccctccagggtcatgaactggtggtccttggtggctctcaggatagagccgtcgtccaggtagtactcgaacacctcctgctggcctctcaggtgccactgggcgatgttctgggtgtacacgaagccgttcttgtccacggtgtacacggtacagtcgatgttctcctccaccaccttgccgatagggatggcgccgtactccacggtcaggatctcggtgtcgtagctcaggcagtactcggc

SEQ ID NO: 139 - DNA, 117 bp, synthetic construct
  source 1..117: mol_type = other DNA
gttgaagcagttgctggcgatgctgccgttggccagcaggaagttgtggtcctgggccacgccgatgtcgtacacgaactgcttgcccaggcttctgtagctcacgatcttcaccat

SEQ ID NO: 140 - DNA, 330 bp, synthetic construct
  source 1..330: mol_type = other DNA
ttacaccaggtagtcgtcagggtggctcaccacctgcttcaggtccaggttcttctcgaagatctcgtcgataggcagcatctcgccgtcgatggtcatgaacttgtggtccttggtggccttgatcttgctgccgtcctccaggtcgtactcgaaaatttcctgttcgcctctgttgtgccactgggcgatggtctgggtgtacacgaagccgttcttgtccacggtgtacacggtgcagttgatgttctcctccacgatcttgccgatgtacatggcgccgtactccacggtcaggatctcggtgtcgtagctcaggcagtactcggc

SEQ ID NO: 141 - DNA, 117 bp, synthetic construct
  source 1..117: mol_type = other DNA
gttgaagcagttgctggcgatgctgccgttggccagcaggaagttgtggtccttctccacgccgatgtcgtacaccttctgggtgcccaggcttctgcagccgatgatcttcaccat

SEQ ID NO: 142 - DNA, 276 bp, synthetic construct
  source 1..276: mol_type = other DNA
ttactccttcacgtacaggcacatgccctccttcaggccgccgctgatgttcatctcgccggtctgggtagggaacaggtgctcctcgctgcagatgatctccttgccgtcctccagggtgatcttgtaagacttcttcttgctcttagggaacacgttcagcacctcgttgtagccggtgttgctcagcaccagatctcccacctggatgttgctgatctccttcatgccctgaggggtctgcacctgggtcttgaggtccagacagtagccgct

SEQ ID NO: 143 - DNA, 120 bp, synthetic construct
  source 1..120: mol_type = other DNA
gctgctgctgttgtgggtcaggatgtcgttggcgtagaacaggtggttgccgctcacctcgatgtcgatcagctctctctcgtccagctcctcgatcttcaggatcttcttcagcatcat

SEQ ID NO: 144 - DNA, 279 bp, synthetic construct
  source 1..279: mol_type = other DNA
ttacttggctctgcttctcaggaagtcgcccaccttcagtccgctgttgattgtcttgaggccgtcctttgtagggaacttgtggttggcagacactctgatcttcttgccagacttcagcacgatctcgaacacaggctgcttgatgataggcagcacctctgtcacctgcacagggccacactcgctctccagccagtcgcccactttgacatctctaatctcgatggccttgccgtttgtcaccaccatggtatccagagacagacatctgttcag

SEQ ID NO: 145 - DNA, 144 bp, synthetic construct
  source 1..144: mol_type = other DNA
cacggcgctgttgtgggtcaggatgccgttggcgaagaacagtctgtcgttggtcacgttgatgtcgattgtctcctccacgcccacgtactcgatgctggcgatctcgtcccagtcgatctcgttctcgaagatctcgcacat

SEQ ID NO: 146 - DNA, 315 bp, synthetic construct
  source 1..315: mol_type = other DNA
ttactccagctcgatcagcaggtgcttcttcatgtccagctcctcggcgtgcacccatctggcgaacaggtggatgttgtcctcgttcactctgttggcgttctccttgtcgatcacgtagaactcgtggttcttggtgcagtcgatgccgttgatgctgatgatctcctcatctctgtcgaagatcagggtgtccaccacctcctgcagcttgccggtgtggctgaacaccttgtcgcccaccttgatctcctcgatcttcttcaggccgttctcggtgttcaccagggtgccaggcacgaagcagccgccgcc

SEQ ID NO: 147 - DNA, 129 bp, synthetic construct
  source 1..129: mol_type = other DNA
gcagatgctgttgtgcaccacggtgcctctcacgttgtagctgtggtcctggttcacggtcaggtcgtgcaccttgcccttgtagtgctttgtctcgatgctggtgatctccttcagcttgaacttcat

SEQ ID NO: 148 - DNA, 312 bp, synthetic construct
  source 1..312: mol_type = other DNA
ttaagagaagtcgctagacttcaggtccaggcctctctcaaagattgtgtcgatggccagcatctcgccatcagatgtcatgaacttgtggtccttggtggcctggatggtctggccgttctccagtgtgtactcgaacacctcctgcaggcctctctcgtgccactggctgataggctgagagtacacgaagccgttctggtccacggtgtacactgtgcagttgatctcctcgctcacgatcttggcgataggcagcaggccgtactcctctgtcaggatcagtgtctcgccgcccaggcagtactcggc

SEQ ID NO: 149 - DNA, 117 bp, synthetic construct
  source 1..117: mol_type = other DNA
gttgaagcagttgctggcgatcaggccgttgcccagcaggaagttgtggtccttctccacgccgatgtcgtacacaggctttctgcccaggctctgtctgccgatgatcttcaccat

SEQ ID NO: 150 - DNA, 324 bp, synthetic construct
  source 1..324: mol_type = other DNA
ttagaacacaggctgcagcacctccacctgcttcagttccagtccctgctcgaaaatctggtcgataggcagcatctgatcctcgtcggtcatgaacttgtgatccttggtggctctgattgtggcgccatcctccagcaggtactcgaacacctcctgctcgcctctgttgtgccactgggcgataggctgggtgtacacgtagccgttctcatccacagagtacacggtacactcgatctgcttctccacgatctcgccgatggccacggcgccgtactccacggtcaggatctgggtgtcgtagctcaggcagtactcggc

SEQ ID NO: 151 - DNA, 117 bp, synthetic construct
  source 1..117: mol_type = other DNA
gttgaagcagttgctggcgatctcgccgctggccaggcagaagttgtggtccttctccacgccgatgtcgtacacgttctgcacgcccaggctctgccttctcacgatcttcaccat

SEQ ID NO: 152 - DNA, 318 bp, synthetic construct
  source 1..318: mol_type = other DNA
ttagttaggcaggttgtccactctcatcaggtccagctctctctcgaagatctcgtcgataggcagcatctggccgtccacggtcatgaacttgtggtccttggtggctctgatcagagagccgtcctccagacagtactcgaacacctcctgctcgcctctgtcgtgccactgggccacaggctgggtatagatgttgccgttgttgtccacagagtacacggtacactcgattctcttctccacgatcttgccgataggcagcaggccgtactccactgtcaggatctctgtctcgtagctcaggcagtactcggc

SEQ ID NO: 153 - DNA, 117 bp, synthetic construct
  source 1..117: mol_type = other DNA
gttgaagcagttgctggcgatgaagccgttcttcagggcgaagttgtggtccctctccacgccgatgtcgtacacgttctgcttgcccaggtactttctggtggcgatcttgatcat

SEQ ID NO: 154 - DNA, 327 bp, synthetic construct
  source 1..327: mol_type = other DNA
ttagatggccaccaccagctcgtcgtcgtcggtcagctcgtcggctctcacgtagcctctgttcttggtgtacacagggtggtcaggggtacatctgatggtcttgacgccgtgcagggtgtcgatctccagctccaccagctcggcgttaggtcttgtcagctgggcggccttgataggggcccactcgatgttgtcggtgtgggtgttgaaggccagcacctggatgttcttgtcgttgtcgaagatctccaccacctccttgatggtggtcttgccgtagtttcttgtgatgatctcagagctgcccaccagacaacaagggtt

SEQ ID NO: 155 - DNA, 129 bp, synthetic construct
  source 1..129: mol_type = other DNA
gatctcgctgttgtgcaccaggatgtcgttggcgaagaagttgtgggtgctggtctggatgtcgtaggtgtcctcgttgctcacgatctttctgctcttcagcttgccgatgtaggtcttggcctccat

SEQ ID NO: 156 - DNA, 348 bp, synthetic construct
  source 1..348: mol_type = other DNA
ttaagacacggtgtagcccacggcggcaggcaggtcctgcacggtaggcagcttcttcaggtccaggcccttctcccagatctcatcgatagggaacatctggccatcctcggtcatgaactggtgatccttggtggctctgatcacagagccatcggccagctcgtaggcgaagatctcctggtagcctctatcgtgccactgagcaatggcctgggtgtacacgaagccgttctcgtccacagagtacactctacagtgaatctgctcgtccacgatcttgccgataggcagagggccgtactccacggtcagcacctctgtgttgtagctcaggcagtactcggc

SEQ ID NO: 157 - DNA, 351 bp, synthetic construct
  source 1..351: mol_type = other DNA
ttaggcggtggccagaggagaaggctgggcaatggccagcagaggggtaggaatctgcttcagctccagagatctctcgaagatctcgtccaggctcagcatctcgccctcctctgtcatgaatctgtgatcggcggtggctctgatgattctgccatcagacagctcgtactcgatcacctcctgctcgcctctctggtgccactgtccgataggctgagagtagatgtatccgtcaggattgcaacagtacacctgacatctgatgttctcctccaccagcttgccgatggcgatagggccgtactccactgtcagcacctctgtgtcggcggccaggcagtactcggc

SEQ ID NO: 158 - DNA, 117 bp, synthetic construct
  source 1..117: mol_type = other DNA
gttgaagcagttgctggccaccaggccgttggccagcacgaagttgtgcacggtggccacgcccaggtcgtacacaggctgcacgcccaggcttcttcttctcacgatcttcaccat

SEQ ID NO: 159 - DNA, 381 bp, synthetic construct
  source 1..381: mol_type = other DNA
ttacttaatggtgccggcgtccagcagagggaaaggcagtctgtggttgtccagggcctcctcggtctgcttaatgttctccagtgtcagcaggtccagctgtctggcgaagatctcctcaatggccagcagctggtagtctgttgtcaggaatctgtggtcgctggtggctctgatcacagagccgtcctccagctcgtactccagcacctcctgctcgcctctgtcgtgccactgggcgatggcctgggtgtacactcttccctcagggtccacagagtacacagaacagttgatctcctcgctcacaatcttgccaataggcagagggccgtactccacggtcagaatctctgtgccgaagctcaggcagtactcggc

SEQ ID NO: 160 - DNA, 117 bp, synthetic construct
  source 1..117: mol_type = other DNA
gttgaagcagttggcggcgatggcgccgttggccagcaggaagttgtggtcctgaggcaggccgatgtcgaagattctctgcacgcccaggcttcttctgccgatcaccttcaccat

SEQ ID NO: 161 - DNA, 333 bp, synthetic construct
  source 1..333: mol_type = other DNA
ttaggcggttctaggcagagaatcaggcacggcccaggcggccagatccaggttctgggtgaagatctcatcgataggcagcagctcgccggtggtggtcaggaatctgtgatcaggtgtggctctgatcatctggccgttctccagctcgtactcgtacagcagtctatctcctctctggtgccactgctcaatcagctgggcgtagatcaggccctgggcatccacagagtacacgtgacacagcagctcctgctccacaatggtctgaataggcagcactccgtattccactgtcacaacaggtgttcctccggccaggcagtactcggc

SEQ ID NO: 162 - DNA, 117 bp, synthetic construct
  source 1..117: mol_type = other DNA
gttgaagcagttggcggcgatcaggccctggcccagcaggaagttgtggtcctggctcaggccgatgtcgtaggtaggggcgtggccgatgaactttcttctgatgatcttcaccat

SEQ ID NO: 163 - DNA, 363 bp, synthetic construct
  source 1..363: mol_type = other DNA
ttatccagacatctgcacagcaggcttcagtccctgcagcagagcctgtctaggagcaatagccacctgccacagttccaggccttcctggaagatctgttcgataggcagcatctggcctctggtggtcatgaatctgtgatcaggtgtggcgcagatggtagagccatcttccagctggtattcgtacacaggtctgaagccctggaagtgccactgggcgataggctgtgtgtacaggtggccctggccatccagagagtacacgtggcagctcagtctttcctgcaccagccttctgataggcacggcgccgtactccacggtcatcacggcggtttcgccgctcaggcagtactcggc

SEQ ID NO: 164 - DNA, 114 bp, synthetic construct
  source 1..114: mol_type = other DNA
gttgaagcagttggcggcgatggcgccgttggccagcacgaagttgtggtcggcggccaggccgatgtcgtacacggcctgccagcccatcagtctcctgcccacgatcttcat

SEQ ID NO: 165 - DNA, 315 bp, synthetic construct
  source 1..315: mol_type = other DNA
ttactccagggtgcccagacacttcaggtccaggtttctctcgaagatctcatcgataggcagcatctggccgtcctcggtcatgaacttgtgctcaggggtggctctgatcacggtgccgtcctccaggctgtactcgtacacctcctgcatgcctctgttgtgccactgggcgataggctgtgtgtagatgtagccgttcttgtccacggtgtacacggtacactcgattctgtactccacgatcttgccgataggcagagggccgtactccacggtcatgatctctgtctcgtaggtcaggcagtactcggc

SEQ ID NO: 166 - DNA, 117 bp, synthetic construct
  source 1..117: mol_type = other DNA
gttgaagcagttgctggcgatcaggccgttggccagcacgaagttgtggtccttggtcacgccgatgtcgtacacgttctcggtcttggccagctttctgctcacgatcttcaccat

SEQ ID NO: 167 - DNA, 363 bp, synthetic construct
  source 1..363: mol_type = other DNA
ttaacaagacatctgcacagcaggcttcagtccctgagccagagctccaggaggagcaatagccacctgccacagctccagtccctctctgaagatctgctcgataggcagcatctggccgcttgttgtcatgaatctgtggtcaggtgtagcacaaatggtagagccgtcctccagctggtaagcgtacacaggtctgaagccctggaagtgccactgggcaataggctgtgtgtacaggtggccctgagggtccaggctgtacacctggcaaatcagtctctcctgcaccagccttctgatagggatggcgccgtactccacggtcatcacggctgtctcgccgctcaggcagtactcggc

SEQ ID NO: 168 - DNA, 114 bp, synthetic construct
  source 1..114: mol_type = other DNA
gttgaagcagttggcggcgatggcgccgttggccagcaggaagttgtggtcgccggccaggccgatgtcgtacacggcctgccagcccaccagtctcctgcccacgatcttcat

SEQ ID NO: 169 - protein, 113 aa, synthetic construct
AEYCLSYDTEIWTVEYGAMPIGKIVEEKIECSVYTVDENGFVYTQPIAQWHPRGQQEIIEYTLEDGRKIRATKDHKMMTESGEMLPIEEIFQRELDLKVETFHEMSLLRRGAK

SEQ ID NO: 170 - protein, 39 aa, synthetic construct
MVKIIKRQSLGRQNVYDVCVETDHNFVLANGCVASNCFN

SEQ ID NO: 171 - protein, 104 aa, synthetic construct
AEYCLSADTEILTVEYGFLPIGEIVGKAIECRVYSVDGNGNIYTQSIAQWHNRGEQEVFEYTLEDGSIIRATKDHKFMTTDGEMLPIDEIFARQLDLMQVQGLH

SEQ ID NO: 172 - protein, 39 aa, synthetic construct
MVKITARKFVGRENVYDIGVEHHHNFAIKNGLIASNCFN

SEQ ID NO: 173 - protein, 105 aa, synthetic construct
AEYCLSYDTEVLTVEYGFVPIGEIVEKGIECSVFSINNNGIVYTQPIAQWHHRGKQEVFEYCLEDGSIIKATKDHKFMTQDGKMLPIDEIFEQELDLLQVKGLPE

SEQ ID NO: 174 - protein, 39 aa, synthetic construct
MIKIASRKFLGVENVYDIGVRRDHNFFIKNGLIASNCFN

SEQ ID NO: 175 - protein, 105 aa, synthetic construct
AEYCLSYDTEVLTVEYGFVPIGEIVDKGIECSVFSIDSNGIVYTQPIAQWHHRGKQEVFEYCLEDGSIIKATKDHKFMTQDGKMLPIDEIFEQELDLLQVKGLPE

SEQ ID NO: 176 - protein, 39 aa, synthetic construct
MIKIASRKFLGVENVYDIGVGRDHNFFVKNGLIASNCFN

SEQ ID NO: 177 - protein, 121 aa, synthetic construct
AEYCLSYETEVLTLEYGFVPIGEIVNKQMVCTVFSLNDSGNVYTQPIGQWHDRGVQDLYEYCLDDGSTIRATKDHKFMTTQGEMVPIDEIFHQGWELVQVSGISKLVQQRTLPFIIVDRKL

SEQ ID NO: 178 - protein, 39 aa, synthetic construct
MVKIVSRRYLGKADVYDIGVAKDHNFIIKNGLVASNCFN

SEQ ID NO: 179 - protein, 102 aa, synthetic construct
AEYCLSYDTEILTVEYGPMPIGKIVEENINCSVYTVNKNGFVYTQSIAQWHHRGEQEVFEYYLEDGETIRATKDHKFMTTEGKMLPIDEIFENNLDLKKLTV

SEQ ID NO: 180 - protein, 39 aa, synthetic construct
MVKIIERRSLGKQNVYDIGVEKDHNFLLSNNLIASNCFN

SEQ ID NO: 181 - protein, 103 aa, synthetic construct
AEYCLSYETQIMTVEYGLMPIGKIVEEQIDCTVYTVNKNGFVYTQPIAQWHYRGEQEVFEYCLEDGSTIRATKDHKFMTTDGQMLPIDEIFEQGLELKQIHLS

SEQ ID NO: 182 - protein, 39 aa, synthetic construct
MVKIISRQSLGIQPVYDIGVEKDHNFLISDGLIASNCFN

SEQ ID NO: 183 - protein, 102 aa, synthetic construct
AEYCLSYDTEILTVEYGAIPIGKVVEENIDCTCYTVDKNGFVYTQNIAQWHLRGQQEVHEYYLDDGSILRATKDHQFMTLEGEMLPIHEIFERGLELKKIKI

SEQ ID NO: 184 - protein, 39 aa, synthetic construct
MVKIVSYRSLGKQFVYDIGVAQDHNFLLANGSIASNCFN

SEQ ID NO: 185 - protein, 109 aa, synthetic construct
AEYCLSYDTEILTVEYGAMYIGKIVEENINCTVYTVDKNGFVYTQTIAQWHNRGEQEIFEYDLEDGSKIKATKDHKFMTIDGEMLPIDEIFEKNLDLKQVVSHPDDYLV

SEQ ID NO: 186 - protein, 39 aa, synthetic construct
MVKIIGCRSLGTQKVYDIGVEKDHNFLLANGSIASNCFN

SEQ ID NO: 187 - protein, 91 aa, synthetic construct
SGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 188 - protein, 40 aa, synthetic construct
MMLKKILKIEELDERELIDIEVSGNHLFYANDILTHNSSS

SEQ ID NO: 189 - protein, 92 aa, synthetic construct
LNRCLSLDTMVVTNGKAIEIRDVKVGDWLESECGPVQVTEVLPIIKQPVFEIVLKSGKKIRVSANHKFPTKDGLKTINSGLKVGDFLRSRAK

SEQ ID NO: 190 - protein, 48 aa, synthetic construct
MCEIFENEIDWDEIASIEYVGVEETIDINVTNDRLFFANGILTHNSAV

SEQ ID NO: 191 - protein, 104 aa, synthetic construct
GGGCFVPGTLVNTENGLKKIEEIKVGDKVFSHTGKLQEVVDTLIFDRDEEIISINGIDCTKNHEFYVIDKENANRVNEDNIHLFARWVHAEELDMKKHLLIELE

SEQ ID NO: 192 - protein, 43 aa, synthetic construct
MKFKLKEITSIETKHYKGKVHDLTVNQDHSYNVRGTVVHNSIC

SEQ ID NO: 193 - protein, 103 aa, synthetic construct
AEYCLGGETLILTEEYGLLPIAKIVSEEINCTVYTVDQNGFVYSQPISQWHERGLQEVFEYTLENGQTIQATKDHKFMTSDGEMLAIDTIFERGLDLKSSDFS

SEQ ID NO: 194 - protein, 39 aa, synthetic construct
MVKIIGRQSLGRKPVYDIGVEKDHNFLLGNGLIASNCFN

SEQ ID NO: 195 - protein, 107 aa, synthetic construct
AEYCLSYDTQILTVEYGAVAIGEIVEKQIECTVYSVDENGYVYTQPIAQWHNRGEQEVFEYLLEDGATIRATKDHKFMTDEDQMLPIDQIFEQGLELKQVEVLQPVF

SEQ ID NO: 196 - protein, 39 aa, synthetic construct
MVKIVRRQSLGVQNVYDIGVEKDHNFCLASGEIASNCFN

SEQ ID NO: 197 - protein, 105 aa, synthetic construct
AEYCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 198 - protein, 39 aa, synthetic construct
MIKIATRKYLGKQNVYDIGVERDHNFALKNGFIASNCFN

SEQ ID NO: 199 - protein, 108 aa, synthetic construct
NPCCLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAI

SEQ ID NO: 200 - protein, 43 aa, synthetic construct
MEAKTYIGKLKSRKIVSNEDTYDIQTSTHNFFANDILVHNSEI

SEQ ID NO: 201 - protein, 115 aa, synthetic construct
AEYCLSYNTEVLTVEYGPLPIGKIVDEQIHCRVYSVDENGFVYTQAIAQWHDRGYQEIFAYELADGSVIRATKDHQFMTEDGQMFPIDEIWEKGLDLKKLPTVQDLPAAVGYTVS

SEQ ID NO: 202 - protein, 116 aa, synthetic construct
AEYCLAADTEVLTVEYGPIAIGKLVEENIRCQVYCCNPDGYIYSQPIGQWHQRGEQEVIEYELSDGRIIRATADHRFMTEEGEMLSLDEIFERSLELKQIPTPLLAIAQPSPLATA

SEQ ID NO: 203 - protein, 39 aa, synthetic construct
MVKIVRRRSLGVQPVYDLGVATVHNFVLANGLVASNCFN

SEQ ID NO: 204 - protein, 126 aa, synthetic construct
AEYCLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 205 - protein, 39 aa, synthetic construct
MVKVIGRRSLGVQRIFDIGLPQDHNFLLANGAIAANCFN

SEQ ID NO: 206 - protein, 110 aa, synthetic construct
AEYCLAGGTPVVTVEYGVLPIQTIVEQELLCHVYSVDAQGLIYAQLIEQWHQRGDRLLYEYELENGQMIRATPDHRFLTTTGELLPIDEIFTQNLDLAAWAVPDSLPRTA

SEQ ID NO: 207 - protein, 39 aa, synthetic construct
MVKIIRRKFIGHAPTYDIGLSQDHNFLLGQGLIAANCFN

SEQ ID NO: 208 - protein, 120 aa, synthetic construct
AEYCLSGETAVMTVEYGAVPIRRLVQERLSCHVYSLDGQGHLYTQPIAQWHFQGFRPVYEYQLEDGSTICATPDHRFMTTRGQMLPIEQIFQEGLELWQVAIAPRQALLQGLKPAVQMSG

SEQ ID NO: 209 - protein, 38 aa, synthetic construct
MKIVGRRLMGWQAVYDIGLAADHNFVLANGAIAANCFN

SEQ ID NO: 210 - protein, 104 aa, synthetic construct
AEYCLTYETEIMTVEYGPLPIGKIVEYRIECTVYTVDKNGYIYTQPIAQWHNRGMQEVYEYSLEDGTVIRATPEHKFMTEDGQMLPIDEIFERNLDLKCLGTLE

SEQ ID NO: 211 - protein, 39 aa, synthetic construct
MVKIVSRKLAKTENVYDIGVTKDHNFVLANGLIASNCFN

SEQ ID NO: 212 - protein, 120 aa, synthetic construct
AEYCLSGETAVMTVEYGAIPIRRLVQERLICQVYSLDPQGHLYTQPIAQWHFQGFRPVYAYQLEDGSTICATPDHRFMTTSGQMLPIEQIFREGLELWQVAIAPPGALAQGLKPAVQMSC

SEQ ID NO: 213 - protein, 38 aa, synthetic construct
MKIVGRRLVGWQAVYDIGLAGDHNFLLANGAIAANCFN

SEQ ID NO: 214 - protein, 6 aa, synthetic construct
AEYCFN

SEQ ID NO: 215 - protein, 5 aa, synthetic construct
AEYCF

SEQ ID NO: 216 - protein, 4 aa, synthetic construct
AEYC

SEQ ID NO: 217 - protein, 8 aa, synthetic construct
AEYEYCFN

SEQ ID NO: 218 - protein, 4 aa, synthetic construct
EYCF

SEQ ID NO: 219 - protein, 4 aa, synthetic construct
YCFN

SEQ ID NO: 220 - protein, 6 aa, synthetic construct
SGYSSS

SEQ ID NO: 221 - protein, 5 aa, synthetic construct
SGYSS

SEQ ID NO: 222 - protein, 4 aa, synthetic construct
SGYS

SEQ ID NO: 223 - protein, 8 aa, synthetic construct
SGYGYSSS

SEQ ID NO: 224 - protein, 4 aa, synthetic construct
GYSS

SEQ ID NO: 225 - protein, 6 aa, synthetic construct
GYYSSS

SEQ ID NO: 226 - protein, 4 aa, synthetic construct
YSSS

SEQ ID NO: 227 - protein, 6 aa, synthetic construct
GGGSIC

SEQ ID NO: 228 - protein, 5 aa, synthetic construct
GGGSI

SEQ ID NO: 229 - protein, 4 aa, synthetic construct
GGGS

SEQ ID NO: 230 - protein, 8 aa, synthetic construct
GGGGGSIC

SEQ ID NO: 231 - protein, 4 aa, synthetic construct
GGSI

SEQ ID NO: 232 - protein, 4 aa, synthetic construct
GSIC

SEQ ID NO: 233 - protein, 6 aa, synthetic construct
NPCSEI

SEQ ID NO: 234 - protein, 5 aa, synthetic construct
NPCSE

SEQ ID NO: 235 - protein, 4 aa, synthetic construct
NPCS

SEQ ID NO: 236 - protein, 8 aa, synthetic construct
NPCPCSEI

SEQ ID NO: 237 - protein, 4 aa, synthetic construct
PCSE

SEQ ID NO: 238 - protein, 6 aa, synthetic construct
PCCSEI

SEQ ID NO: 239 - protein, 4 aa, synthetic construct
CSEI

SEQ ID NO: 240 - protein, 6 aa, synthetic construct
AEYSSS

SEQ ID NO: 241 - protein, 6 aa, synthetic construct
AEYSIC

SEQ ID NO: 242 - protein, 6 aa, synthetic construct
SGYCFN

SEQ ID NO: 243 - protein, 6 aa, synthetic construct
SGYSIC

SEQ ID NO: 244 - protein, 6 aa, synthetic construct
GGGCFN

SEQ ID NO: 245 - protein, 6 aa, synthetic construct
GGGSSS

SEQ ID NO: 246 - protein, 6 aa, synthetic construct
AEYSEI

SEQ ID NO: 247 - protein, 6 aa, synthetic construct
GGGSEI

SEQ ID NO: 248 - protein, 6 aa, synthetic construct
NPCCFN

SEQ ID NO: 249 - protein, 6 aa, synthetic construct
NPCSIC

SEQ ID NO: 250 - protein, 6 aa, synthetic construct
NPCSSS

SEQ ID NO: 251 - protein, 6 aa, synthetic construct
SGYSEI

SEQ ID NO: 252 - protein, 6 aa, synthetic construct
AEYGYS

SEQ ID NO: 253 - protein, 11 aa, synthetic construct
MAEYVDVFLKE

SEQ ID NO: 254 - protein, 10 aa, synthetic construct
EWPALGDSEI

SEQ ID NO: 255 - protein, 6 aa, synthetic construct
AEYPEF

SEQ ID NO: 256 - protein, 9 aa, synthetic construct
AEYEAEYLE

SEQ ID NO: 257 - protein, 11 aa, synthetic construct
RDFAEYKTPDE

SEQ ID NO: 258 - protein, 5 aa, synthetic construct
APPSA

SEQ ID NO: 259 - protein, 4 aa, synthetic construct
YNQP

SEQ ID NO: 260 - protein, 6 aa, synthetic construct
KPGYSA

SEQ ID NO: 261 - protein, 10 aa, synthetic construct
YSLTTIGASA

SEQ ID NO: 262 - protein, 8 aa, synthetic construct
LKLGHSEL

SEQ ID NO: 263 - protein, 5 aa, synthetic construct
LKLGY

SEQ ID NO: 264 - protein, 8 aa, synthetic construct
LKLGYSEL

SEQ ID NO: 265 - protein, 8 aa, synthetic construct
WFGLSVDE

SEQ ID NO: 266 - protein, 4 aa, synthetic construct
WFGY

SEQ ID NO: 267 - protein, 4 aa, synthetic construct
SVDE

SEQ ID NO: 268 - protein, 8 aa, synthetic construct
WFGYSVDE

SEQ ID NO: 269 - protein, 6 aa, synthetic construct
TRGSQP

SEQ ID NO: 270 - protein, 7 aa, synthetic construct
EWPALGG

SEQ ID NO: 271 - protein, 10 aa, synthetic construct
EWPALGGSEI

SEQ ID NO: 272 - protein, 5 aa, synthetic construct
AEPEF

SEQ ID NO: 273 - protein, 6 aa, synthetic construct
EWPALG

SEQ ID NO: 274 - protein, 9 aa, synthetic construct
EQPALGSEI

SEQ ID NO: 275 - protein, 6 aa, synthetic construct
RDFEYG

SEQ ID NO: 276 - protein, 6 aa, synthetic construct
SKTPDE

SEQ ID NO: 277 - protein, 12 aa, synthetic construct
RDFEYGSKTPDE

SEQ ID NO: 278 - protein, 5 aa, synthetic construct
RDFEG

SEQ ID NO: 279 - protein, 5 aa, synthetic construct
STPDE

SEQ ID NO: 280 - protein, 10 aa, synthetic construct
RDFEGSTPDE

SEQ ID NO: 281 - protein, 7 aa, synthetic construct
EWPALGC

SEQ ID NO: 282 - protein, 10 aa, synthetic construct
EWPALGCSEI

SEQ ID NO: 283 - protein, 8 aa, synthetic construct
EWPALGDC

SEQ ID NO: 284 - protein, 11 aa, synthetic construct
EWPALGDCSEI

SEQ ID NO: 285 - protein, 6 aa, synthetic construct
RDFEYC

SEQ ID NO: 286 - protein, 5 aa, synthetic construct
SKTPD

SEQ ID NO: 287 - protein, 12 aa, synthetic construct
RDFEYCSKTPDE

SEQ ID NO: 288 - protein, 5 aa, synthetic construct
RDFEC

SEQ ID NO: 289 - protein, 4 aa, synthetic construct
STPD

SEQ ID NO: 290 - protein, 5 aa, synthetic construct
APGYS

SEQ ID NO: 291 - protein, 8 aa, synthetic construct
LTTIGASA

SEQ ID NO: 292 - protein, 8 aa, synthetic construct
LTTIGYSA

SEQ ID NO: 293 - protein, 13 aa, synthetic construct
APGYSLTTIGASA

SEQ ID NO: 294 - protein, 13 aa, synthetic construct
APGYSLTTIGYSA

SEQ ID NO: 295 - protein, 9 aa, synthetic construct
EWPALGSEI

SEQ ID NO: 296 - protein, 5 aa, synthetic construct
AEYEF

SEQ ID NO: 297 - protein, 10 aa, synthetic construct
RDFEYKTPDE

SEQ ID NO: 298 - protein, 11 aa, synthetic construct
RDAFEYKTPDE

SEQ ID NO: 299 - protein, 10 aa, synthetic construct
RDAEYKTPDE

SEQ ID NO: 300 - protein, 10 aa, synthetic construct
RDFECSTPDE

SEQ ID NO: 301 - protein, 4 aa, synthetic construct
KPGY

SEQ ID NO: 302 - protein, 4 aa, synthetic construct
APGY

SEQ ID NO: 303 - protein, 6 aa, synthetic construct
LTTIGY

SEQ ID NO: 304 - protein, 11 aa, synthetic construct
EWFALGDCSEI

(19) United States
(12) Patent Application Publication
(10) Pub. No.: US 2025/0099616 A1
(43) Pub. Date: Mar. 27, 2025

(54) GENERATION OF LARGE PROTEINS BY CO-DELIVERY OF MULTIPLE VECTORS

(71) Applicant: UNIVERSITY OF WASHINGTON, Seattle, WA (US)

(72) Inventors: Jeffrey S. CHAMBERLAIN, Seattle, WA (US); Hichem TASFAOUT, Seattle, WA (US)

(73) Assignee: UNIVERSITY OF WASHINGTON, Seattle, WA (US)

(21) Appl. No.: 18/291,018

(22) PCT Filed: Jul. 22, 2022
(86) PCT No.: PCT/US2022/038032

§ 371 Date: Jan. 22, 2024

Related U.S. Application Data
(60) Provisional application No. 63/256,819, filed on Oct. 18, 2021.
(60) Provisional application No. 63/225,212, filed on Jul. 23, 2021.

(51) Int. Cl. A61K 48/00; A61K 38/17; A61P 21/00; C12N 15/86

(52) U.S. Cl. CPC A61K 48/0058; A61K 38/1709; A61P 21/00; C12N 15/86; C12N 2750/14143; C12N 2830/42

(57) ABSTRACT

Provided herein are methods and compositions for delivering large proteins to a subject in need thereof for treatment of a disease or disorder. In certain embodiments, the methods and compositions described herein are useful in the delivery of large proteins to subjects using a protein expression system comprising at first and second AAV vector for the treatment of muscular or neuromuscular disease or disorders.

### CROSS-REFERENCE TO RELATED APPLICATIONS

This application is a 35 U.S.C. § 371 National Phase Entry application of International Patent Application No. PCT/US2022/038032 filed on Jul. 22, 2022, which designated the U.S., and which claims benefit under 35 U.S.C. § 119 (e) to U.S. Provisional Application No. 63/225,212 filed Jul. 23, 2021 and U.S. Provisional Application No. 63/256,819 filed Oct. 18, 2021, the contents of each which are incorporated herein by reference in their entireties.

### SEQUENCE LISTING

The instant application contains a Sequence Listing which has been submitted in XML format via Patent Center and is hereby incorporated by reference in its entirety. Said XML copy, created on Sep. 15, 2022, is named 034186-190540WOPT.xml and is 316,676 bytes in size.

### TECHNICAL FIELD

The field of the invention relates to methods of delivering or inducing the production of large therapeutic proteins using multiple vectors.

### BACKGROUND

Gene therapies using AAV vectors hold a promising future for treating different loss-of-function genetic disorders (Li 2020). However, this therapeutic modality has been challenged by the small packaging capacity of this viral vector (˜5 kb).

### SUMMARY

The methods and compositions described herein are based, in part, on the discovery that split inteins can permit the delivery of large polypeptides, including but not limited to dystrophin, using AAV vectors.

In one aspect, described herein is a method for delivering an exogenous polypeptide to a cell, the method comprising contacting the cell with: a first adeno-associated virus (AAV) vector particle comprising a first nucleic acid encoding a first fusion polypeptide comprising a first portion of an exogenous polypeptide fused to a first portion of a split intein; and a second AAV vector particle comprising a second nucleic acid encoding a second fusion polypeptide comprising a second portion of the exogenous polypeptide fused to a second portion of the split intein; wherein the first and second fusion polypeptides are produced in the cell from the first and second nucleic acids, and wherein the first and second portions of the split intein promote joining of the first portion of the exogenous polypeptide to the second portion of the exogenous polypeptide, thereby delivering the exogenous polypeptide to the cell; wherein the exogenous polypeptide delivered is larger than can be encoded by a single AAV vector particle.

In one embodiment of this and all other aspects described herein, the first and second nucleic acids comprise a muscle-specific expression cassette (MSEC).

In another embodiment of this and all other aspects described herein, the split intein is a naturally-occurring split intein.

In another embodiment of this and all other aspects described herein, the split intein is a genetically modified split intein. In another embodiment, the genetic modification of the split intein is selected from codon optimization for expression and/or stability in mammalian cells, shortening or lengthening of the split intein, or changing encoded amino acids in the split intein to more closely match the sequence of the exogenous protein to be delivered.

In another embodiment of this and all other aspects described herein, the first and second portions of the exogenous polypeptide are substantially the same size.

In another embodiment of this and all other aspects described herein, the first and second portions of the exogenous polypeptide differ in size by no more than 50 amino acids.

In another embodiment of this and all other aspects described herein, the exogenous polypeptide comprises a footprint of less than four amino acids from the split intein.

In another embodiment of this and all other aspects described herein, the exogenous polypeptide comprises a footprint of 3 or fewer amino acids from the split intein.

In another embodiment of this and all other aspects described herein, the split site separating the first and second portions of the exogenous polypeptide is selected at a site having the same sequence as the split intein footprint, thereby producing the exogenous polypeptide without extra amino acids from the split intein.

In another embodiment of this and all other aspects described herein, the exogenous polypeptide is a therapeutic polypeptide.

In another embodiment of this and all other aspects described herein, the therapeutic polypeptide is selected from dystrophin, mini-dystrophin, utrophin and dysferlin, nebulin, titin, myosin, spectrin repeat containing nuclear envelope protein 1 (Syne-1), dystroglycan, ATP synthase, clotting factor IIX, lamin A/C, thyroglobulin, epidermal growth factor receptor (EGFR), alpha- and/or beta spectrin, muscle target of rapamycin (mTOR), and ryanodine receptor 1. In another embodiment, the mini-dystrophin is greater than 160 kDa and smaller than full-length dystrophin.

In another embodiment of this and all other aspects described herein, the therapeutic polypeptide is dystrophin and the N-terminal portion of the dystrophin extein is joined to the N-terminal portion of a split intein within or adjacent to a dystrophin hinge domain.

In another embodiment of this and all other aspects described herein, the hinge domain comprises hinge 1, 2, 3, or 4 of dystrophin.

In another embodiment of this and all other aspects described herein, the therapeutic polypeptide is dystrophin and the N-terminal portion of the dystrophin extein is joined to a loop domain joining helix b to helix c, or helix c to helix a′ within one of the 24 dystrophin spectrin-like repeat domains.

In another embodiment of this and all other aspects described herein, the therapeutic polypeptide is dystrophin and the C-terminal portion of the dystrophin extein is joined to the C-terminal portion of the split intein within or adjacent to a dystrophin hinge domain or to a loop domain joining helix b to helix c, or helix c to helix a′ within one of the 24 dystrophin spectrin-like repeat domains. In another embodiment of this and all other aspects described herein, the hinge domain comprises hinge 1, 2, 3, or 4 of dystrophin.

In another embodiment of this and all other aspects described herein, the exogenous polypeptide is functional in the cell.

In another aspect, described herein is a method for delivering an exogenous polypeptide to a cell, the method comprising contacting the cell with: a first adeno-associated virus (AAV) vector particle comprising a first nucleic acid encoding a first fusion polypeptide comprising a first portion of an exogenous polypeptide fused to a first portion of a first split intein, wherein the first portion of the split intein is fused to the carboxy terminus of the first portion of the exogenous polypeptide; a second AAV vector particle comprising a second nucleic acid encoding a second fusion polypeptide comprising a second portion of the exogenous polypeptide fused to (i) a second portion of the first split intein at the amino terminus of the second portion of the exogenous polypeptide and (ii) a first portion of a second split intein at the carboxy terminus of the second portion of the exogenous polypeptide; and a third AAV vector particle comprising a third nucleic encoding a third fusion polypeptide comprising a third portion of the exogenous polypeptide fused to a second portion of the second split intein at the amino terminus of the third portion of the exogenous polypeptide, wherein the first, second, and third fusion polypeptides are produced in the cell from the first, second and third nucleic acids, and wherein the respective portions of the first and second split inteins promote joining of (a) the carboxy terminus of the first portion of the exogenous polypeptide to the amino terminus of the second portion of the exogenous polypeptide and (b) the carboxy terminus of the second portion of the exogenous polypeptide to the amino terminus of the third portion of the exogenous polypeptide, thereby delivering the exogenous polypeptide to the cell; wherein the exogenous polypeptide delivered is larger than can be encoded by a single AAV vector particle. In one embodiment of this and all other aspects described herein, the first and second split inteins do not cross-splice.

In another aspect, described herein is a protein expression system comprising a set of AAV vector particles comprising a first and second AAV particle, wherein the first AAV vector particle comprises a first nucleic acid encoding a first fusion polypeptide comprising a first portion of an exogenous polypeptide fused to a first portion of a split intein; and wherein the second AAV vector particle comprises a second nucleic acid encoding a second fusion polypeptide comprising a second portion of the exogenous polypeptide fused to a second portion of the split intein.

In one embodiment of this and all other aspects described herein, co-infection of a cell with the first and second AAV vector particles promotes joining of the first portion of the exogenous polypeptide to the second portion of the exogenous polypeptide, with removal of the first and second portions of the split intein.

In another embodiment of this and all other aspects described herein, joining of the first portion of the exogenous polypeptide to the second portion of the exogenous polypeptide, with removal of the first and second portions of the split intein generates an exogenous polypeptide larger than can be encoded in a single AAV particle.

In another aspect, described herein is a protein expression system comprising a set of AAV vector particles comprising a first, second, and third AAV particle, wherein the first AAV vector particle comprises a first nucleic acid encoding a first fusion polypeptide comprising a first portion of an exogenous polypeptide fused to a first portion of a first split intein, wherein the first portion of the split intein is fused to the carboxy terminus of the first portion of the exogenous polypeptide; wherein the second AAV vector particle comprises a second nucleic acid encoding a second fusion polypeptide comprising a second portion of the exogenous polypeptide fused to (i) a second portion of the first split intein at the amino terminus of the second portion of the exogenous polypeptide and (ii) a first portion of a second split intein at the carboxy terminus of the second portion of the exogenous polypeptide; and wherein the third AAV vector particle comprises a third nucleic encoding a third fusion polypeptide comprising a third portion of the exogenous polypeptide fused to a second portion of the second split intein at the amino terminus of the third portion of the exogenous polypeptide.

In one embodiment of this and all other aspects described herein, co-infection of a cell with the first, second and third AAV vector particles promotes joining of the first portion of the exogenous polypeptide to the second portion of the exogenous polypeptide, with removal of the first and second portions of the first split intein, and joining of the second portion of the exogenous polypeptide to the third portion of the exogenous polypeptide, with removal of the first and second portions of the second split intein.

In another embodiment of this and all other aspects described herein, joining of the first portion of the exogenous polypeptide to the second portion of the exogenous polypeptide, with removal of the first and second portions of the first split intein, and joining of the second portion of the exogenous polypeptide to the third portion of the exogenous polypeptide, with removal of the first and second portions of the second split intein generates an exogenous polypeptide larger than can be encoded in a single AAV particle.

In another embodiment of this and all other aspects described herein, expression of the first and second, or first, second and third fusion polypeptides is driven by a muscle-specific expression cassette.

In another aspect, described herein is a method of treating a disease or disorder in a subject in need thereof, the method comprising administering a protein expression system as described herein, thereby treating the subject.

In one embodiment of this and all other aspects described herein, the subject in need thereof has a muscular or neuromuscular disease or disorder.

In another embodiment, of this and all other aspects described herein, the exogenous polypeptide is dystrophin or mini-dystrophin and the subject in need thereof has Duchenne muscular dystrophy (DMD) or Becker muscular dystrophy (BMD).

In another embodiment, of this and all other aspects described herein, the dystrophin or mini-dystrophin increases the strength of dystrophic muscles by at least 10%.

In another embodiment, of this and all other aspects described herein, expression of the first and second, or first, second and third fusion polypeptides is driven by a muscle-specific expression cassette.

In another embodiment, of this and all other aspects described herein, the protein expression system is administered by infusion into the vasculature, or by direct injection into a tissue.

In another aspect, described herein is a method for inducing the production of an exogenous polypeptide in a cell, the method comprising contacting the cell with: a first adeno-associated virus (AAV) vector particle comprising a first nucleic acid encoding a first fusion polypeptide comprising a first portion of an exogenous polypeptide fused to a first portion of a split intein; and a second AAV vector particle comprising a second nucleic acid encoding a second fusion polypeptide comprising a second portion of the exogenous polypeptide fused to a second portion of the split intein; wherein the first and second fusion polypeptides are produced in the cell from the first and second nucleic acids, and wherein the first and second portions of the split intein promote joining of the first portion of the exogenous polypeptide to the second portion of the exogenous polypeptide, thereby inducing the production of the exogenous polypeptide in the cell; wherein the exogenous polypeptide produced is larger than can be encoded by a single AAV vector particle.

In one embodiment of this and all other aspects described herein, the first and second nucleic acids comprise a muscle-specific expression cassette (MSEC).

In another embodiment of this and all other aspects described herein, the split intein is a naturally-occurring split intein.

In another embodiment of this and all other aspects described herein, the split intein is a genetically modified split intein. In another embodiment, the genetic modification of the split intein is selected from codon optimization for expression and/or stability in mammalian cells, shortening or lengthening of the split intein, or changing encoded amino acids in the split intein to more closely match the sequence of the exogenous protein to be produced.

In another embodiment of this and all other aspects described herein, the first and second portions of the exogenous polypeptide are substantially the same size.

In another embodiment of this and all other aspects described herein, the first and second portions of the exogenous polypeptide differ in size by no more than 50 amino acids.

In another embodiment of this and all other aspects described herein, the exogenous polypeptide comprises a footprint of less than four amino acids from the split intein.

In another embodiment of this and all other aspects described herein, the exogenous polypeptide comprises a split intein footprint of 3 or fewer amino acids.

In another embodiment of this and all other aspects described herein, the split site separating the first and second portions of the exogenous polypeptide is selected at a site having the same sequence as the split intein footprint, thereby producing the exogenous polypeptide without extra amino acids from the split intein.

In another embodiment of this and all other aspects described herein, the exogenous polypeptide is a therapeutic polypeptide.

In another embodiment of this and all other aspects described herein, the therapeutic polypeptide is selected from dystrophin, mini-dystrophin, utrophin and dysferlin, nebulin, titin, myosin, spectrin repeat containing nuclear envelope protein 1 (Syne-1), dystroglycan, ATP synthase, clotting factor IIX, lamin A/C, thyroglobulin, epidermal growth factor receptor (EGFR), alpha- and/or beta spectrin, muscle target of rapamycin (mTOR), and ryanodine receptor 1.

In another embodiment of this and all other aspects described herein, the mini-dystrophin is greater than 160 kDa and smaller than full-length dystrophin.

In another embodiment of this and all other aspects described herein, the therapeutic polypeptide is dystrophin and the N-terminal portion of the dystrophin extein is joined to the N-terminal portion of a split intein within or adjacent to a dystrophin hinge domain.

In another embodiment of this and all other aspects described herein, the hinge domain comprises hinge 1, 2, 3, or 4 of dystrophin.

In another embodiment of this and all other aspects described herein, the therapeutic polypeptide is dystrophin and the N-terminal portion of the dystrophin extein is joined to a loop domain joining helix b to helix c, or helix c to helix a′ within one of the 24 dystrophin spectrin-like repeat domains.

In another embodiment of this and all other aspects described herein, the therapeutic polypeptide is dystrophin and the C-terminal portion of the dystrophin extein is joined to the C-terminal portion of the split intein within or adjacent to a dystrophin hinge domain or to a loop domain joining helix b to helix c, or helix c to helix a′ within one of the 24 dystrophin spectrin-like repeat domains.

In another embodiment of this and all other aspects described herein, the hinge domain comprises hinge 1, 2, 3, or 4 of dystrophin.

In another embodiment of this and all other aspects described herein, the exogenous polypeptide is functional in the cell.

In another aspect, described herein is a method for inducing the production of an exogenous polypeptide in a cell, the method comprising contacting the cell with: a first adeno-associated virus (AAV) vector particle comprising a first nucleic acid encoding a first fusion polypeptide comprising a first portion of an exogenous polypeptide fused to a first portion of a first split intein, wherein the first portion of the split intein is fused to the carboxy terminus of the first portion of the exogenous polypeptide; a second AAV vector particle comprising a second nucleic acid encoding a second fusion polypeptide comprising a second portion of the exogenous polypeptide fused to (i) a second portion of the first split intein at the amino terminus of the second portion of the exogenous polypeptide and (ii) a first portion of a second split intein at the carboxy terminus of the second portion of the exogenous polypeptide; and a third AAV vector particle comprising a third nucleic encoding a third fusion polypeptide comprising a third portion of the exogenous polypeptide fused to a second portion of the second split intein at the amino terminus of the third portion of the exogenous polypeptide, wherein the first, second, and third fusion polypeptides are produced in the cell from the first, second and third nucleic acids, and wherein the respective portions of the first and second split inteins promote joining of (a) the carboxy terminus of the first portion of the exogenous polypeptide to the amino terminus of the second portion of the exogenous polypeptide and (b) the carboxy terminus of the second portion of the exogenous polypeptide to the amino terminus of the third portion of the exogenous polypeptide, thereby producing the exogenous polypeptide in the cell; wherein the exogenous polypeptide produced is larger than can be encoded by a single AAV vector particle. In one embodiment of this and all other aspects described herein, the first and second split inteins do not cross-splice.

In another aspect, provided herein is a composition(s) as described herein for use in the treatment of a disease or disorder in a subject in need thereof (e.g., a subject having a muscular or neuromuscular disorder).

### BRIEF DESCRIPTION OF DRAWINGS

FIGS. 1A-1B. (FIG. 1A) Schematic representation of the DMD coding sequences (top) encoding the full-length “muscle-specific” isoform of dystrophin (bottom), which consists of an amino-terminal globular domain that binds the actin cytoskeleton, followed by a flexible and elastic rod domain composed of 24 Spectrin-like repeats interspersed with four proline-rich “hinge” regions. A dystroglycan-binding domain (DgBD) is located after the rod domain, followed by the carboxy-terminal (CT) domain that contains binding sites for the syntrophin and dystrobrevin protein families. The DgBD and the CT domain nucleate the assembly of the dystrophin-glycoprotein protein complex (DGC). (FIG. 1B) Different μDys constructs being evaluated in clinical trials by Pfizer, Sarepta Therapeutics, and Solid Biosciences.

FIG. 2. Dual AAV vector homologous recombination strategy to reconstitute mini-Dys (ΔH2-SR19). Two AAV vectors encode either N-(top) or C-terminal (bottom) mini-Dys fragments. Both vectors carry a recombinant sequence (exon 51 to 53) which allows the formation of larger and functional mini-Dys (ΔH2-SR19).

FIG. 3. Schematic representation of protein trans-splicing mediated by contiguous (more common) or split inteins.

FIGS. 4A-4B Example of GFP reconstitution using split Npu intein in HEK293 cells. (FIG. 4A) Brightfield and fluorescent microscopy pictures of living HEK293 cells transfected with Wild-type (WT) GFP, N-terminal and/or C-terminal GFP/Npu intein plasmids. (FIG. 4B) The GFP fluorescence intensity was measured using a spectrophotometer. Values are represented as mean+s.e.m (n=3).

FIGS. 5A-5C in vitro validation of mini-Dys reconstitution. (FIG. 5A) Schematic representation of intein-mediated mini-Dys reconstitution. Left: N-terminal vector encoding human DMD sequences from exons 1 to 50, but lacking exons 21 to 41. Right: C-terminal vector encoding human DMD sequences from exons 51 to 79. The mini-Dys sequences are fused to N- or C-terminal halves of the selected intein. (FIG. 5B) Western blot analysis of HEK293 cells lysates showing the 290 kDa mini-Dys. In control mini-dys, cells were transfected with plasmid expressing the entire mini-Dys ΔSR5-15. In split mini-Dys/intein, cells were co-transfected with both N- and C-terminal vectors. Each lane represents a selected split site between SR19 and Hinge3. (FIG. 5C) Densitometry quantification of mini-Dys normalized to loading control GAPDH (n=4-5 independent experiments). Data are shown as mean+s.e.m. M.W: molecular weight. kDa: kiloDalton.

FIGS. 6A-6B Schematic representation of AAV-based Dystrophin replacement using SIMPL-GT (Split Intein-Mediated Protein Ligation for Gene Therapy) approach. (FIG. 6A) Dual vector strategy which consists of simultaneous administration of two AAV vectors that express two halves of a mini-Dys (ΔSR5-15) fused to split intein. Following in-frame transcription and translation with the N- or C-terminal mini-Dys fragments, the intein polypeptides are self-excised and join the adjacent peptides, thus expressing a highly functional mini-Dys (ΔSR5-15). (FIG. 6B) Expression of full-length Dystrophin via triple AAV vectors administration. The 1^{st} AAV vector encodes proteins from N-terminus to SR8 of Dystrophin fused to N-terminal fragment of split intein1. The 2^{nd} AAV vector encodes a middle fragment of Dystrophin (SR9-19) flanked by both C-terminal half of intein1 and N-terminal half of intein2. While the 3rd AAV vector encodes for C-terminal fragment of Dystrophin which is fused to the C-terminal half of intein2. The double trans-splicing of intein1 and 2, respectively, will lead to the ligation of three Dystrophin fragments into full-length protein.

FIG. 7 Split intein screening using the split GFP system. N- or the C-terminal half of GFP were cloned in-frame with the N- or the C-terminal half of our codon optimized split inteins. The human embryonic kidney 293 (HEK293) cells were co-transfected with both N- and C-terminal GFP/intein plasmids. 24 hours later, The GFP fluorescence intensity was measured using a spectrophotometer. Values are represented as mean±s.e.m of percentage versus the WT GFP (n=5-6). The protein ligation efficiency of each split intein (GFP fluorescence of a given intein/internal control) is labeled on the bar.

FIG. 8 Split intein specificity and cross-reactivity using split GFP system. To test the cross-reactivity of a given N-terminal split intein with a C-terminal of another split intein, N- and C-terminal split GFP-inteins were tested on HEK293 cells. As split inteins from the 1st group present amino-acid similarities, they showed poor specificity and cross-reacted with different inteins of the same group. However, split inteins from the 2^{nd} group, i.e. gp41.1, IMPDH and Nrdj1, were more specific toward the other half of the same intein and did not cross-react with any other split-intein. Values are represented as mean #s.e.m of percentage versus the WT GFP (n=3-4). These observations are very important specially for triple vector strategy, where 2 very specific split inteins are needed to ligate 3 dystrophin fragments into full-length protein.

FIGS. 9A-9C. Split intein footprint importance and optimization for Dystrophin reconstitution. (FIG. 9A) The intein-mediated protein trans-splicing is highly dependent on juxtaposed amino acids that are found in native bacterial extein proteins. When N- and C-terminal split intein fragments fuse and splice out, these native extein amino-acids (AEY and CFN for both Aha and Sel; and SGY and SSS for gp41.1; GGG and SIC for IMPDH; NPC and SEI for Nrdj1) are left as a footprint in the reconstituted protein. Here, the inventors tested several combinations to reduce this footprint to a minimum. These data show that with Aha split intein, the same splicing efficiency is achievable with AEY as AEY/CFN when tested on split GFP system, while with gp41.1 GY/S are sufficient for efficient GFP ligation. This footprint was even shorter with IMPDH and Nrdj1 with only G/S and C/S, respectively. Values are represented as mean+s.e.m of percentage versus the WT GFP (n=4). Schematic example of protein trans-splicing using Aha and/or gp41.1 with minimal footprint leftover for full-length (FIG. 9B) or mini-Dystrophin (FIG. 9C) reconstitution.

FIG. 10 Identification of several split sites in human Dystrophin protein where some native amino acids can be used as part of the intein footprint.

FIGS. 11A-11B. (FIG. 11A) Western blot analysis of HEK293 cells lysates showing the 290 kDa mini-Dys. In control mini-dys, cells were transfected with plasmid expressing the entire mini-Dys ΔSR5-15. In split mini-Dys/intein, cells were co-transfected with both N- and C-terminal vectors. Each lane represents a selected split site between SR19 and Hinge3. (FIG. 11B) Densitometry quantification of mini-Dys normalized to loading control GAPDH (n=4-5 independent experiments). Data are shown as mean+s.e.m. M.W: molecular weight. kDa: kiloDalton. These data show that inserting split gp41.1 in 4 different splitting sites efficiently reconstitutes the mini-Dystrophin ΔSR-5-15.

FIGS. 12A-12C. in vivo mini-Dystrophin ΔSR-5-15 expression after AAV intramuscular injections. The split mini-Dystrophin/intein clones were inserted into pAAV plasmid containing the muscle-specific creatine kinase 8 (CK8) regulatory cassette and small synthetic polyA flanked by two AAV serotype 2 inverted terminal repeats (ITRs). The final pAAV plasmids were co-transfected with the pDG6 packaging plasmid into HEK293 cells to generate recombinant AAV2/6 vectors and purified via heparin-affinity chromatography then concentrated using sucrose gradient centrifugation. A dose of 5×10¹⁰ viral genome (v.g) of AAV encoding the N- and/or C-terminal split mini-Dystrophin/intein was administrated into tibialis anterior muscles (T.A) of three-week-old C57BL/6-mdx^{4cv}. Four weeks post-injection, the injected muscles were harvested, and total proteins were extracted and separated on SDS gel for western blotting (FIG. 12A). A strong expression of mini-Dystrophin ΔSR5-15 was detected in 4 T.A muscle tested, highlighting the efficacy of SIMPLI-GT approach. Muscles were cryo-sectioned and immunostained for dystrophin (FIG. 12B) or stained with Hematoxylin and Eosin, and myofiber size and nuclei position were measured (FIG. 12C). The reconstituted mini-Dystrophin ΔSR5-15 was correctly localized at the myofiber sarcolemma of mdx^{4cv} injected with dual AAV N- and C-terminal vectors. These muscles exhibit a general muscle histology improvement with absence of inflammation.

FIG. 13. in vitro proof-of-concept of full-length Dystrophin expression via triple vector strategy. Western blot analysis of HEK293 cells lysates transfected with 3 plasmids expressing either N-, C- or middle fragments of human Dystrophin. Split intein gp41.1 was used to ligate the middle with the C-terminal fragment, while 6 different split inteins were tested for N-terminal and middle fragment ligation.

FIGS. 14A-14B. in vitro proof-of-concept of full-length Dysferlin expression. (FIG. 14A) Western blot analysis of HEK293 cells lysates transfected with plasmid expressing either the full-length human Dysferlin or split Dysferlin/gp41.1 intein or Dysferlin/IMPDH. 3 splitting sites were tested. (FIG. 14B) Densitometry quantification of full-length Dysferlin expression normalized to loading control GAPDH (n=5 independent experiments). Data are shown as mean+s.e.m. These data show that inserting split gp41.1 in 2 different splitting sites efficiently reconstitutes the full-length Dysferlin.

FIGS. 15A-15W. Split intein DNA and protein sequences. FIG. 15A, Aha (SEQ ID Nos: 1 & 2). FIG. 15B, Aov (SEQ ID Nos: 3 & 4). FIG. 15C, Asp (SEQ ID Nos: 5 & 6). FIG. 15D, Ava (SEQ ID Nos: 7 & 8). FIG. 15E, Cra (SEQ ID Nos: 9 & 10). FIG. 15F, Csp-CCY (SEQ ID Nos: 11 & 12). FIG. 15G, Csp-PCC7424 (SEQ ID Nos: 13 & 14). FIG. 15H, Csp-PCC8801 (SEQ ID Nos: 15 & 16). FIG. 15I, Cwa (SEQ ID Nos: 17 & 18). FIG. 15J, gp41.1 (SEQ ID Nos: 19 & 20). FIG. 15K, gp41.8 (SEQ ID Nos: 21 & 22). FIG. 15L, IMPDH (SEQ ID Nos: 23 & 24). FIG. 15M, Maer (SEQ ID Nos: 25 & 26). FIG. 15N, Mcht (SEQ ID Nos: 27 & 28). FIG. 15O, Npu (SEQ ID Nos: 29 & 30). FIG. 15P, Nrdj (SEQ ID Nos: 31 & 32). FIG. 15Q, Oli (SEQ ID Nos: 33 & 34). FIG. 15R, Sel (SEQ ID Nos: 35 & 36). FIG. 15S, Ssp-PCC6803 (SEQ ID Nos: 37 & 38). FIG. 15T, Ssp-PCC7002 (SEQ ID Nos: 39 & 40). FIG. 15U, Tel (SEQ ID Nos: 41 & 42). FIG. 15V, Ter (SEQ ID Nos: 43 & 44). FIG. 15W, Tvu (SEQ ID Nos: 45 & 46).

FIG. 16. Full-length dysferlin split sites.

FIG. 17. Full-length dystrophin split sites (IMPDH intein).

FIG. 18. Full-length dystrophin split sites (Nrdj intein).

FIG. 19. Full-length dystrophin split sites.

FIG. 20. Full-length dystrophin split sites (gp41.1 intein).

FIG. 21. Mini-dystrophin ΔSR5-15 split sites.

FIGS. 22A-22D in vivo expression of full-length dystrophin following intramuscular administration of 3 intein vectors. Split dystrophin/intein clones for each combination were packaged into an AAV6 vector using the CK8e promoter, and were administrated locally into TA muscles of 3-week-old mdx^{4cv} mice at 5×10¹⁰ v.g per construct. Four weeks after the injection, total proteins were analyzed by western blot using an antibody that recognizes the C-terminal end of dystrophin (FIG. 22A). Similar to in vitro observations, a 427 kDa band was detected with both combinations with a higher expression detected with split Nrdj1 & split gp41.1 combination (2 to 4-fold more versus WT expression). More importantly, a dramatic reduction of centrally nucleated myofiber was observed at this early time point, from ˜65% in untreated mdx^{4cv} TA muscles versus 30 to 40% in muscles treated with this triple vector strategy, with remarkable improvements of the general muscle histology in this short time point (FIGS. 22B, 22C). The reconsituted full-length dystrophin was correctly localized at the sarcolema membrane when assessed using any of three different antibodies that recognize the N-, C-terminal or middle fragments of the full-length dystrophin protein. FIG. 22A, Western blot (above) showing the expression of full-length dystrophin following triple vector administration in mdx^{4cv} TA muscles. FIG. 22B, Visualization of centrally-nucleated myofibers in cross-sections of mdx^{4cv} TA muscles treated with different vector combinations (or saline) and stained with Hematoxylin and Eosin. Also shown are untreated wild-type (WT) or mdx^{4cv} TA muscles from age-matched mice. N-ter only: muscles injected with only a single vector, in this case the N-terminal vector; middle only: muscles injected with only a single vector, in this case the middle vector; C-ter only: muscles injected with only a single vector, in this case the C-terminal vector. Other panels show muscles injected with combinations of two vectors or all 3 (triple). FIG. 22C, Quantification of centrally-nucleated myofibers in cross-sections of mdx^{4cv} TA muscles treated with the indicated triple vector combinations (or saline) and stained with Hematoxylin and Eosin. Also shown are values from untreated wild-type (WT) mouse TA muscles. Data is from counting ˜400 myofibers from various muscles. FIG. 22D, Triple immunolabeling of TA muscle cryosections using antibodies against the N-, C-terminal or middle fragments of dystrophin. Wild-type muscles were uninjected; the mdx^{4cv} muscles were injected with saline or 2 triple vector combinations as in FIG. 22A.

FIGS. 23A-23C in vivo expression of mini-Dys and full-length dystrophin following intravenous infusion of dual or triple vectors. 8-week-old mdx^{4cv} were systemically treated with a total dose of 2×10¹⁴ vg/kg for three months of treatment. Both hindlimb and diaphragm muscle contractile properties were assessed using a muscle force transducer (FIG. 23A, FIG. 23B). Mice treated with Dual or triple vector exhibited significant improvements of muscle specific force development of the tibialis anterior and diaphragm muscles versus saline-treated mdx^{4cv} and wild-type mouse muscles. Using western blot, these muscles showed strong expression of mini-Dys and full-length dystrophin (FIG. 23C). FIG. 23A, in vivo specific force of tibialis anterior muscles. FIG. 23B, in vitro specific force of isolated diaphragm muscle strips. FIG. 23C, Western blot showing expression of mini-Dys and full-length dystrophin in tibialis anterior muscles following systemic administration of dual or triple vectors.

### DETAILED DESCRIPTION

Provided herein are methods and compositions useful for the delivery of exogenous polypeptides that are too large to fit in a single adenoviral, adeno-associated, lentiviral or retroviral vector. The methods and compositions described herein employ the use of split inteins, which mediate the fusion of a first and second portion of a large exogenous polypeptide delivered using at least two viral vectors (e.g., AAV vectors), thereby permitting delivery of a large exogenous polypeptide to a cell (e.g., a muscle cell). The methods and compositions also relate to muscle-specific cell expression of such exogenous polypeptides (e.g., dystrophin, utrophin and dysferlin).

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein, the term “splice” or “splices” means to excise an internal portion of a polypeptide, with joinder of the portions flanking the internal portion to form two or more smaller polypeptide molecules (e.g., an excised polypeptide and a spliced polypeptide. In some cases, splicing also includes the step of fusing together two or more of the smaller polypeptides to form a new polypeptide. Splicing can also refer to the joining of two polypeptides encoded on two separate nucleic acid sequences or in two separate vectors through the action of a split intein.

As used herein, the term “cleave” or “cleaves” means to divide a single polypeptide to form two or more smaller polypeptide molecules. In some cases, cleavage is mediated by the addition of an extrinsic endopeptidase, which is often referred to as “proteolytic cleavage.” In other cases, cleaving can be mediated by the intrinsic activity of one or both of the cleaved peptide sequences, which is often referred to as “self-cleavage.” Cleavage can also refer to the self-cleavage of two polypeptides that is induced by the addition of a non-proteolytic third peptide, as in the action of a split intein system as described herein.

By the term “fused” is meant covalently bonded to. For example, a first peptide is fused to a second peptide when the two peptides are covalently bonded to each other (e.g., via a peptide bond).

As used herein, the term “intein” refers to a naturally occurring, self-splicing protein subdomain that is capable of excising out its own protein subdomain from a larger protein structure while simultaneously joining the two formerly flanking peptide regions (“exteins”) together to form a mature host protein. In some inteins, the precursor protein comes from two genes, which is referred to as a ‘split intein.’

As used herein, the term “split intein” refers to an intein that is comprised of two or more separate components not fused to one another. Split inteins can occur naturally, or can be engineered by splitting contiguous inteins. Typically, the term “split intein” refers to any intein in which one or more peptide bond breaks exists between the N-terminal intein segment and the C-terminal intein segment such that the N-terminal and C-terminal intein segments become separate molecules that can non-covalently reassociate, or reconstitute, into an intein that is functional for splicing or cleaving reactions. Any catalytically active intein, or fragment thereof, can be used to derive a split intein for use in the systems and methods disclosed herein. For example, in one aspect the split intein can be derived from a eukaryotic intein. In another aspect, the split intein can be derived from a bacterial intein. In another aspect, the split intein can be derived from an archaeal intein. Preferably, the split intein so-derived will possess only the amino acid sequences essential for catalyzing splicing reactions.

As used herein, the “N-terminal intein segment” refers to any intein sequence that comprises an N-terminal amino acid sequence that is functional for splicing and/or cleaving reactions when combined with a corresponding C-terminal intein segment. An N-terminal intein segment thus also comprises a sequence that is spliced out when splicing occurs. An N-terminal intein segment can comprise a sequence that is a modification of the N-terminal portion of a naturally occurring (native) intein sequence. For example, an N-terminal intein segment can comprise additional amino acid residues and/or mutated residues so long as the inclusion of such additional and/or mutated residues does not render the intein non-functional for splicing or cleaving. Preferably, the inclusion of the additional and/or mutated residues improves or enhances the splicing activity and/or controllability of the intein. Non-intein residues can also be genetically fused to intein segments to provide additional functionality, such as the ability to be affinity purified or to be covalently immobilized.

As used herein, the “C-terminal intein segment” refers to any intein sequence that comprises a C-terminal amino acid sequence that is functional for splicing or cleaving reactions when combined with a corresponding N-terminal intein segment. In one aspect, the C-terminal intein segment comprises a sequence that is spliced out when splicing occurs. In another aspect, the C-terminal intein segment is cleaved from a peptide sequence fused to its C-terminus. The sequence which is cleaved from the C-terminal intein's C-terminus is a protein for the treatment of a muscular disorder, such as dystrophin, utrophin, dysferlin, mini-dystrophin, or the like. A C-terminal intein segment can comprise a sequence that is a modification of the C-terminal portion of a naturally occurring (native) intein sequence. For example, a C terminal intein segment can comprise additional amino acid residues and/or mutated residues so long as the inclusion of such additional and/or mutated residues does not render the C-terminal intein segment non-functional for splicing or cleaving. Preferably, the inclusion of the additional and/or mutated residues improves or enhances the splicing and/or cleaving activity of the intein.

As used herein, the term “larger than can be encoded by a single AAV vector particle” refers to a polypeptide for which nucleic acid encoding it exceeds the packaging limits of an AAV vector particle. While exact packaging limits can vary slightly with serotype or variant of AAV vector used, the maximum genome-packaging capacity of AAV vectors that efficiently infect and transduce target cells is about 5 kb (the wild-type AAV genome is about 4.7 kb; larger genomes up to 5.5 kb or more can be packaged under certain conditions, but they do not efficiently infect and transduce target cells). Excluding ITRs, this permits the incorporation of about 3.5 kb of DNA for the promoter, transgene coding region, polyadenylation sequence and other regulatory elements for a transgene construct to be carried by a single AAV vector particle. Thus, transgenes requiring more than about 3.5 kb to direct expression of a desired protein are larger than can be encoded by a single AAV vector particle as the term is used herein. In some embodiments, the protein that is larger than can be encoded by a single vector particle requires at least 4 kb, at least 4.5 kb, at least 5 kb, at least 5.5 kb, at least 6 kb, at least 6.5 kb, at least 7 kb, at least 7.5 kb, at least 8 kb, at least 8.5 kb, at least 9 kb, at least 9.5 kb, at least 10 kb, at least 10.5 kb, at least 11 kb, at least 11.5 kb, at least 12 kb, at least 12.5 kb, at least 13 kb, at least 13.5 kb, at least 14 kb or more to encode the transgene polypeptide. As also discussed elsewhere herein, when the target protein requires more nucleic acid sequence than will fit into two separate viral vectors to generate a full length target polypeptide (or sub-full length polypeptide with functional improvements over a more truncated mini- or microgene construct), the polypeptide can be split over separate vectors including three or potentially more split intein constructs. In this instance co-infection with the set of vectors can generate the full length or improved sub-full length polypeptide.

As used herein, the terms “first portion of an exogenous polypeptide fused to a first portion of a split intein” and “second portion of the exogenous polypeptide fused to a second portion of the split intein” as used in regard to methods for delivering an exogenous polypeptide to a cell, producing an exogenous polypeptide in a cell or methods of treatment or prophylaxis based on such delivery or production or compositions therefor as described herein refer to fragments of a target polypeptide that is larger than can be encoded by a single AAV vector particle. The first portion and second portion fragments of the target polypeptide are fused respectively to amino and carboxy-terminal portions of a split intein in a manner that permits excision of the intein and covalent joining of the first and second portion (engineered extein) polypeptides to reconstitute the target protein when both fusion protein are expressed in a cell. The sizes of the first portion and second portion of the target protein can vary, e.g., with the amino-terminal fragment being shorter than, approximately the same size as or larger than the carboxy-terminal fragment (and the corresponding carboxy-terminal fragment varying such that it is longer than, approximately the same size as or shorter than the amino-terminal fragment, respectively), but it is preferred, where a target is divided into two fragments, that the target is split approximately near the middle of the target protein. Where a target protein is divided into three fragments as described herein, the sizes can vary, but it is preferred that the three fragments are also approximately the same length. It can be considered to split the target protein between or at the junction of structural domains, rather than within them, e.g., between alpha helices, beta sheets, or between any two such structural domains. As a non-limiting example, in the context of a dystrophin or utrophin polypeptide, it is contemplated that the protein be split between spectrin-like repeat domains, or between a spectrin-like repeat domain and a hinge domain. The various domains of exemplary large proteins dystrophin, utrophin and dysferlin are discussed further herein below. The boundaries of various domains for dystrophin and dysferlin polypeptides are also described herein below, and one of ordinary skill in the art can determine boundaries between domains in other proteins.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

### Muscular Dystrophies

Muscular dystrophy is a group of inherited disorders characterized by progressive muscle weakness and loss of muscle tissue.

Muscular dystrophies include many inherited disorders, including Becker muscular dystrophy and Duchenne muscular dystrophy, which are both caused by mutations in the dystrophin gene (i.e., DMD). Both of the disorders have similar symptoms, although Becker muscular dystrophy is a slower progressing form of the disease. Duchenne muscular dystrophy is a rapidly progressive form of muscular dystrophy.

Both disorders are characterized by progressive muscle weakness of the legs and pelvis which is associated with a loss of muscle mass (wasting). Muscle weakness also occurs in the arms, neck, and other areas, but not as severely as in the lower half of the body. Calf muscles initially enlarge (an attempt by the body to compensate for loss of muscle strength), the enlarged muscle tissue is eventually replaced by fat and connective tissue (pseudohypertrophy). Muscle contractions occur in the legs and heels, causing inability to use the muscles because of shortening of muscle fibers and fibrosis of connective tissue. Bones develop abnormally, causing skeletal deformities of the chest and other areas. Cardiomyopathy occurs in almost all cases. A mouse model for DMD exists, and is proving useful for furthering understanding of both the normal function of dystrophin and the pathology of the disease. In particular, experiments that enhance the production of utrophin, a dystrophin relative, in order to compensate for the loss of dystrophin are promising, and may lead to the development of effective therapies for this devastating disease.

Dysferlinopathy is a muscular dystrophy that is caused by mutations in the dysferlin gene. The symptoms of dysferlinopathy vary significantly between individuals. Clinical presentations most commonly associated with dysferlinopathy include limb girdle muscular dystrophy (LGMD2B), Miyoshi myopathy, distal myopathy with anterior tibial onset (DMAT), proximodistal weakness, pseudometabolic myopathy, and hyperCKemia. Most commonly, patients report distal muscle weakness in the second decade of life with loss of distal motor function within the ensuing decade. Patients generally require a wheelchair for motility with varying degrees of overall body control. As dysferlinopathy is often misdiagnosed, its incidence has not been determined. To date, there is no effective treatment to slow the loss of muscle function or reverse/improve the dystrophic phenotype.

### Exogenous Polypeptides

A distinct advantage of the methods and compositions described herein is the ability to encode and deliver large proteins to a cell, e.g., a muscle cell, among others. Vectors, such as adenoviral associated vectors (AAV), are limited in their capacity to package nucleic acids and, as such, large proteins cannot be encoded and delivered on a single AAV vector. The methods and compositions described herein utilize split inteins, where an N-terminal region of a split intein and a portion of a desired exogenous polypeptide are encoded on a first AAV vector, and a C-terminal region of the split intein and a second portion of the desired exogenous polypeptide is encoded on a second AAV vector. Expression of the products from each AAV together in a cell permit the first and second portions of the split intein to promote joining of the first portion of the exogenous polypeptide to the second portion of the exogenous polypeptide. As one of skill in the art will appreciate, the methods and compositions described herein can be utilized for any large gene products, which need not be limited by function.

The methods and compositions described herein are exemplified in the working examples using muscle proteins including dystrophin, utrophin, and dysferlin.

Dystrophin: Dystrophin is a 427 kDa cytoskeletal protein and is a member of the spectrin/α-actinin superfamily (See e.g., Blake et al., Brain Pathology, 6:37 (1996); Winder, J. Muscle Res. Cell. Motil., 18:617 (1997); and Tinsley el al., PNAS, 91:8307 (1994)). The N-terminus of dystrophin binds to actin, having a higher affinity for non-muscle actin than for sarcomeric actin. Dystrophin is involved in the submembranous network of non-muscle actin underlying the plasma membrane. Dystrophin is associated with an oligomeric, membrane spanning complex of proteins and glycoproteins, the dystrophin-associated protein complex (DPC). The C-terminus of dystrophin binds to the cytoplasmic tail of β-dystroglycan, and in concert with actin, anchors dystrophin to the sarcolemma. Also bound to the C-terminus of dystrophin are the cytoplasmic members of the DPC. Dystrophin thereby provides a link between the actin-based cytoskeleton of the muscle fiber and the extracellular matrix. It is this link that is disrupted in muscular dystrophy.

The central rod domain of dystrophin is composed of a series of 24 weakly repeating units of approximately 110 amino acids, similar to those found in spectrin (i.e., spectrin-like repeats). This domain constitutes the majority of dystrophin and gives dystrophin a flexible rod-like structure. The rod-domain is interrupted by four hinge regions that are rich in proline. It is contemplated that the rod-domain provides a structural link between members of the DPC.

TABLE 1
Domains of Full-length dystrophin cDNA

| Nucleotides | Domain | SEQ ID NO: |
|---|---|---|
| 1-208 | 5′ untranslated region | SEQ ID NO: 47 |
| 209-211 | Start codon (ATC) | — |
| 209-964 | N terminus | SEQ ID NO: 48 |
| 965-1219 | Hinge 1 | SEQ ID NO: 49 |
| 1220-1546 | Spectrin-like repeat No. 1 | SEQ ID NO: 50 |
| 1547-1879 | Spectrin-like repeat No. 2 | SEQ ID NO: 51 |
| 1880-2212 | Spectrin-like repeat No. 3 | SEQ ID NO: 52 |
| 2213-2359 | Hinge 2 | SEQ ID NO: 53 |
| 2360-2692 | Spectrin-like repeat No. 4 | SEQ ID NO: 54 |
| 2693-3019 | Spectrin-like repeat No. 5 | SEQ ID NO: 55 |
| 3020-3346 | Spectrin-like repeat No. 6 | SEQ ID NO: 56 |
| 3347-3673 | Spectrin-like repeat No. 7 | SEQ ID NO: 57 |
| 3674-4000 | Spectrin-like repeat No. 8 | SEQ ID NO: 58 |
| 4001-4312 | Spectrin-like repeat No. 9 | SEQ ID NO: 59 |
| 4313-4588 | Spectrin-like repeat No. 10 | SEQ ID NO: 60 |
| 4589-4915 | Spectrin-like repeat No. 11 | SEQ ID NO: 61 |
| 4916-5239 | Spectrin-like repeat No. 12 | SEQ ID NO: 62 |
| 5340-5551 | Spectrin-like repeat No. 13 | SEQ ID NO: 63 |
| 5552-5833 | Spectrin-like repeat No. 14 | SEQ ID NO: 64 |
| 5834-6127 | Spectrin-like repeat No. 15 | SEQ ID NO: 65 |
| 6128-6187 | 20 amino acid insert (not | SEQ ID NO: 66 |
| | hinge) | |
| 6188-6514 | Spectrin-like repeat No. 16 | SEQ ID NO: 67 |
| 6515-6835 | Spectrin-like repeat No. 17 | SEQ ID NO: 68 |
| 6836-7186 | Spectrin-like repeat No. 18 | SEQ ID NO: 69 |
| 7187-7489 | Spectrin-like repeat No. 19 | SEQ ID NO: 70 |
| 7490-7612 | Hinge 3 | SEQ ID NO: 71 |
| 7613-7942 | Spectrin-like repeat No. 20 | SEQ ID NO: 72 |
| 7943-8269 | Spectrin-like repeat No. 21 | SEQ ID NO: 73 |
| 8270-8617 | Spectrin-like repeat No. 22 | SEQ ID NO: 74 |
| 8618-9004 | Spectrin-like repeat No. 23 | SEQ ID NO: 75 |
| 9005-9328 | Spectrin-like repeat No. 24 | SEQ ID NO: 76 |
| 9329-9544 | Hinge 4 | SEQ ID NO: 77 |
| 9545-10431 | Start of C terminus | SEQ ID NO: 78 |
| 10432-11254 | Alternatively spliced exons | SEQ ID NO: 79 |
| | 71-78 | |
| 11255-11266 | End of Coding Region | SEQ ID NO: 80 |
| 11267-13957 | 3′ untranslated region | SEQ ID NO: 81 |

Homologs of dystrophin have been identified in a variety of organisms, including mouse (Genbank accession number M68859); dog (Genbank accession number AF070485); and chicken (Genbank accession number X13369). Similar comparisons can be generated with homologs from other species, including but not limited to those described above, by using any of a variety of available computer programs (e.g., BLAST, from NCBI). Candidate homologs can be screened for biological activity using any suitable assay, including, but not limited to those described herein.

Utrophin: Utrophin is an autosomally-encoded homolog of dystrophin and it has been postulated that the proteins play a similar physiological role (For a recent review, See e.g., Blake et al., Brain Pathology, 6:37 [1996]). Human utrophin shows substantial homology to dystrophin, with the major difference occurring in the rod domain, where utrophin lacks repeats 15 and 19 and two hinge regions (See e.g., Love et al., Nature 339:55 [1989]; Winder et al., FEBS Lett., 369:27 [1995]). Utrophin thus contains 22 spectrin-like repeats and two hinge regions.

Dysferlin: Dysferlin comprises the following domains: C2A, C2B, C2C, FerA, DysF, C2D, C2E, C2F, C2G, and TM. The exact boundaries of each domain may vary among orthologs and variants. The approximate amino acid range for each domain in human dysferlin is shown in Table 2. The listed domain boundaries may vary by up to about 20 residues, e.g., about 5, 10, 15, or 20 residues.

TABLE 2
Dysferlin domains

| Domain | Amino Acid Range |
|---|---|
| C2A | 1-124 |
| C2B | 219-352 |
| C2C | 366-515 |
| FerA | 670-782 |
| DysF | 864-1097 |
| C2D | 1137-1281 |
| C2E | 1314-1465 |
| C2F | 1579-1696 |
| C2G | 1789-1994 |
| TM | 2045-2067 |

Protein Variants: Moreover, as described above, variant forms (e.g., mutants) of an exogenous polypeptide, such as dystrophin, utrophin, a mini-dystrophin or dysferlin, are also contemplated for use with the methods and compositions described herein. For example, it is contemplated that an isolated replacement of a leucine with an isoleucine or valine, an aspartate with a glutamate, a threonine with a serine, or a similar replacement of an amino acid with a structurally related amino acid (i.e., conservative mutations) will not necessarily have a major effect on the biological activity of the resulting molecule. Accordingly, in some embodiments, the exogenous polypeptide can comprise one or more conservative amino acid replacements. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine) (See e.g., Stryer (ed.), Biochemistry, 2nd ed, W H Freeman and Co. [1981]). Whether a change in the amino acid sequence of a peptide results in a functional homolog can be readily determined by assessing the ability of the variant peptide to function in a fashion similar to the wild-type protein. Peptides in which more than one replacement has taken place can readily be tested in the same manner.

In some embodiments, a variant of an exogenous polypeptide is engineered to comprise an enhanced biological activity. Such polypeptides, when expressed from recombinant DNA constructs, can be used in therapeutic embodiments as described herein.

In some embodiments, a variant of an exogenous polypeptide can comprise an increased intracellular half-life as compared to the corresponding wild-type protein. For example, such variant protein can be more stable or less stable to proteolytic degradation or other cellular process that result in destruction of, or otherwise inactivation of the variant. Such variants, and the genes that encode them, can be utilized to alter the pharmaceutical activity of constructs expressing variant exogenous polypeptides by modulating the half-life of the protein. For instance, a short half-life can give rise to more transient biological effects. As above, such proteins find use in pharmaceutical applications or for the treatment of a muscular disease or disorder.

A wide range of techniques are known in the art for screening gene products of combinatorial libraries made by point mutations, and for screening cDNA libraries for gene products having a certain property. Such techniques are generally adaptable for rapid screening of the gene libraries generated by the combinatorial mutagenesis of a given exogenous polypeptide. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

In some embodiments, the exogenous polypeptide comprises a mini-dystrophin or micro-dystrophin. As used herein, a “mini-dystrophin” comprises an amino terminal actin-binding domain, a β-dystroglycan binding domain and a plurality (e.g, at least 2) spectrin-like repeat domains.

### Adenoviral Associated Vectors (AAVs)

AAV is a small virus that presents very low immunogenicity and is not associated with any known human disease, making it attractive as a vector for delivery of exogenous genetic material (e.g. for gene therapy). However, the size of the AAV capsid imposes a limit on the amount of DNA that can be packaged within it. The AAV genome is approximately 4.7 kilobases (kb) in size

The methods and compositions described herein permit the delivery of large proteins (e.g., greater than 4.7 kb) by administering two (or more) AAV vectors, each having a portion of an exogenous polypeptide to be expressed and a portion of a split intein. In one embodiment, the methods and compositions described herein use at least two different adeno-associated viral (AAV) vectors. The first AAV vector comprises an N-terminal portion of a split intein fused to a first portion of an exogenous polypeptide (e.g., dystrophin, dysferlin, utrophin or other desired therapeutic protein, e.g., for a muscular or other disease or disorder) and a second AAV vector comprises a C-terminal portion of a split intein fused to a second portion of the exogenous polypeptide. Upon expression of the first and second fusion polypeptides in the cell, the first and second portions of the split intein promote joining of the first portion of the exogenous polypeptide to the second portion of the exogenous polypeptide, thereby delivering the exogenous polypeptide to the cell. This system or arrangement permits delivery of an exogenous polypeptide that is larger than can be encoded by a single AAV vector particle.

Embodiments of the first and second AAV vectors are provided herein and include the following non-limiting embodiments. An AAV vector as used herein can be in the form of a mature AAV particle or virion, i.e. nucleic acid surrounded by an AAV protein capsid. The AAV vector can comprise an AAV genome or a portion or derivative thereof. An AAV genome is a polynucleotide which encodes functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, an AAV genome of a vector as used herein is typically replication-deficient.

The AAV genome can be in single-stranded form, either positive or negative-sense, or alternatively in double-stranded form. The use of a double-stranded form allows bypass of the DNA replication step in the target cell and so can accelerate transgene expression. In one embodiment, the AAV genome is in single-stranded form. The AAV genome can be from any naturally derived serotype, isolate or clade of AAV. Thus, the AAV genome can be the full genome of a naturally occurring AAV or a recombinant, engineered AAV. As is known to the skilled person, AAVs occurring in nature may be classified according to various biological systems.

Commonly, AAVs are referred to in terms of their serotype. A serotype corresponds to a variant subspecies of AAV which, owing to its profile of expression of capsid surface antigens, has a distinctive reactivity which can be used to distinguish it from other variant subspecies. Typically, a virus having a particular AAV serotype does not efficiently cross-react with neutralizing antibodies specific for any other AAV serotype. AAV serotypes include AAV1, AAV2, AAV3, AAV4, AAV5, AAV6, AAV7, AAV8, AAV9, AAV10 and AAV11, and also recombinant serotypes, such as Rec2 and Rec3. Any of these AAV serotypes can be used with the methods and compositions described herein. Reviews of AAV serotypes can be found in Choi et al. (2005) Curr. Gene Ther. 5:299-310 and Wu et al. (2006) Molecular Therapy 14:316-27. The sequences of AAV genomes or of elements of AAV genomes including ITR sequences, rep or cap genes can be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV can also be referred to in terms of clades or clones. This refers to the phylogenetic relationship of naturally derived AAVs, and typically to a phylogenetic group of AAVs which can be traced back to a common ancestor, and includes all descendants thereof.

Additionally, AAVs can be referred to in terms of a specific isolate, i.e. a genetic isolate of a specific AAV found in nature. The term genetic isolate describes a population of AAVs which has undergone limited genetic mixing with other naturally occurring AAVs, thereby defining a recognizably distinct population at a genetic level.

The skilled person can select an appropriate serotype, clade, clone or isolate of AAV for use with the methods and compositions described herein on the basis of their common general knowledge of the particular AAV characteristics.

The AAV serotype determines the tissue specificity of infection (or tropism) of an AAV virus. Accordingly, preferred AAV serotypes for use in AAVs administered to patients in accordance with the methods and compositions described herein are those which, for example, have natural tropism for or a high efficiency of infection of target cells within a muscle.

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell.

The AAV genome typically also comprises packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV particle. Capsid variants are discussed below. A promoter can be operably linked to each of the packaging genes. Specific examples of such promoters include the p5, p19 and p40 promoters (Laughlin et al. (1979) Proc. Natl. Acad. Sci. USA 76:5567-5571). For example, the p5 and p19 promoters are generally used to express the rep gene, while the p40 promoter is generally used to express the cap gene.

Typically, the AAV genome for use with the methods and compositions described herein will be derivatized for the purpose of administration to patients. Such derivatization is standard in the art (see e.g., Coura and Nardi (2007) Virology Journal 4:99). Derivatives of an AAV genome include any truncated or modified forms of an AAV genome which allow for expression of a transgene in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative of an AAV genome will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome, which contains both coding and complementary sequences, i.e. a self-complementary AAV genome. This allows for bypass of DNA replication in the target cell, and so enables accelerated transgene expression.

The inclusion of one or more ITRs is preferred to aid concatamer formation of the vector in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In some embodiments, ITR elements are the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. The following portions could therefore be removed in a derivative: one inverted terminal repeat (ITR) sequence, the replication (rep) and capsid (cap) genes. However, in some embodiments, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the vector may be tolerated in a therapeutic setting. Where a derivative comprises capsid proteins i.e. VP1, VP2 and/or VP3, the derivative can be a chimeric, shuffled or capsid-modified derivative of one or more naturally occurring AAVs. In particular, the methods and compositions described herein encompass the provision of capsid protein sequences from different serotypes, clades, clones, or isolates of AAV within the same vector (i.e. a pseudotyped vector).

Chimeric, shuffled or capsid-modified derivatives are typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV vector comprising a naturally occurring AAV genome, such as that of AAV2. Increased efficiency of gene delivery can be effected by improved receptor or co-receptor binding at the cell surface, improved internalization, improved trafficking within the cell and into the nucleus, improved uncoating of the viral particle and/or improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This can be performed, for example, by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins can also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes can also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence.

The vectors used herein can encompass the provision of sequences of an AAV genome in a different order and configuration to that of a native AAV genome. The vector(s) can also include the replacement of one or more AAV sequences or genes with sequences from another virus or with chimeric genes composed of sequences from more than one virus. Such chimeric genes can be composed of sequences from two or more related viral proteins of different viral species.

AAV vectors for use as described herein can include transcapsidated forms wherein an AAV genome or derivative having an ITR of one serotype is packaged in the capsid of a different serotype. Such AAV vectors can also include mosaic forms wherein a mixture of unmodified capsid proteins from two or more different serotypes makes up the viral capsid. An AAV vector can also include chemically modified forms bearing ligands adsorbed to the capsid surface. For example, such ligands may include antibodies for targeting a particular cell surface receptor.

The first and second AAV vectors of the AAV vector system as described herein together comprise all of the components necessary for a fully functional exogenous polypeptide to be re-assembled in a target cell following transduction by both vectors. A skilled person will be aware of additional genetic elements commonly used to ensure transgene expression in a viral vector-transduced cell. These may be referred to as expression control sequences. Thus, the AAV vectors of the AAV viral vector system described herein typically comprise expression control sequences (e.g. comprising a promoter sequence) operably linked to the nucleotide sequences encoding the desired exogenous polypeptide (e.g., dystrophin, utrophin, dysferlin and the like).

Any suitable promoter can be used. The promoter sequence can be constitutively active (i.e. operational in any host cell background), or alternatively may be active only in a specific host cell environment, thus allowing for targeted expression of the transgene in a particular cell type (e.g. a tissue-specific promoter). The promoter can show inducible expression in response to presence of another factor, for example a factor present in a host cell. In any event, where the vector is administered for therapy, it is preferred that the promoter should be functional in the target cell background.

In some embodiments, it is preferred that the promoter is highly efficacious in muscle cells in order to allow for the transgene to be preferentially or only expressed in muscle cell populations. Thus, expression from the promoter may be muscle-cell specific. In one embodiment, a muscle-specific promoter is comprised by a muscle-specific expression cassette, as that term is used herein.

At least one of the vectors described herein can comprise an untranslated region (UTR) located between the promoter and the upstream polypeptide-encoding nucleic acid sequence (i.e. a 5′ UTR). Any suitable UTR sequence can be used. The UTR can comprise one or more of the following elements: a Gallus gallus β-actin (CBA) intron 1 fragment, an Oryctolagus cuniculus β-globin (RBG) intron 2 fragment, and an Oryctolagus cuniculus β-globin exon 3 fragment. The UTR can comprise a Kozak consensus sequence. Any suitable Kozak consensus sequence can be used.

At least one of the vectors described herein can further comprise a post-transcriptional response element (also known as post-transcriptional regulatory element) or PRE. Any suitable PRE can be used. The presence of a suitable PRE can enhance expression of the desired transgene. In one embodiment, the PRE is a Woodchuck Hepatitis Virus PRE (WPRE). The one or more vectors can also comprise a poly-adenylation sequence located 3′ to the protein-encoding nucleic acid sequence. Any suitable poly-adenylation sequence can be used. In one embodiment, the poly-adenylation sequence is a bovine Growth Hormone (bGH) poly-adenylation sequence.

Expression of a given exogenous protein requires that the target cell be transduced with both the first AAV vector and the second AAV vector; however, the order is not important. Thus, the target cell can be transduced with the first AAV vector and the second AAV vector in any order (first AAV vector followed by second AAV vector, or second AAV vector followed by first AAV vector) or simultaneously. Methods for transducing target cells with AAV vectors are known in the art and will be familiar to a skilled person. The target cell is preferably a muscular cell, preferably a skeletal muscle cell or cardiac muscle cell.

While the methods and compositions described herein relate to the use of at least two adeno-associated vectors, the methods and compositions can utilize alternative vectors including, e.g., second generation adenoviral vectors, lentiviral vectors, or retroviral vectors.

Second generation adenoviral vectors delete the early regions of the Ad genome (E2A, E2B, and E4). Highly modified second generation adenoviral vectors are less likely to generate replication-competent virus during large-scale vector preparation. Host immune response against late viral proteins is thus reduced (See Amalfitano et al., “Production and Characterization of Improved Adenovirus Vectors With the E1, E2b, and E3 Genes Deleted,” J. Virol. 72:926-933 (1998)). The elimination of E2A, E2B, and E4 genes from the adenoviral genome also provides increased cloning capacity. This, combined with the split intein approach described herein can further increase the size of the exogenously-encoded polypeptide introduced.

Lentivirus-based vectors infect non-dividing cells as part of their normal life cycles, and are produced by expression of a package-able vector construct in a cell line that expresses viral proteins. The small size of lentiviral particles constrains the amount of exogenous DNA they are able to carry to about 10 kb.

Vectors based on Moloney murine leukemia viruses (MMLV) and other retroviruses have emerged as useful for gene therapy applications. These vectors stably transduce actively dividing cells as part of their normal life cycles, and integrate into host cell chromosomes. Retroviruses can be employed as described herein, for example, in the context of infection and transduction of muscle precursor cells such as myoblasts, satellite cells, or other muscle stem cells.

### Split Inteins

Inteins are naturally occurring, self-splicing protein subdomains that are capable of excising out their own protein subdomain from a larger protein structure while simultaneously joining the two formerly flanking peptide regions (“exteins”) together to form a mature host protein.

The ability of inteins to rearrange flanking peptide bonds, and retain activity when in fusion to proteins other than their native exteins, has led to a number of intein-based biotechnologies. These include various types of protein ligation and activation applications, as well as protein labeling and tracing applications. An important application of inteins is in the production of purified recombinant proteins. In particular, inteins have the ability to impart self-cleaving activity to a number of conventional affinity and purification tags, and thus provide a major advance in the production of recombinant protein products for research, medical and other commercial applications.

The use of split inteins with the methods and compositions provided herein permits large protein-encoding sequences to be divided amongst two (or more) different vectors, such as AAV vectors, which, upon expression in a cell, are ligated together to form the full protein. Given that AAV vectors are limited by the size of protein-encoding sequence they can carry, the use of split inteins permits the delivery of large proteins to a cell, which could not be encoded on a single AAV vector alone.

Any catalytically active intein, or fragment thereof, can be used to derive a split intein for use in the methods of the invention. For example, in one aspect the split intein can be derived from a eukaryotic intein. In another aspect, the split intein can be derived from a bacterial intein. In another aspect, the split intein can be derived from an archaeal intein. Preferably, the split intein so-derived will possess only the amino acid sequences essential for catalyzing trans-splicing reactions.

The N-terminal split intein, as that term is used herein, can comprise a sequence that is a modification of the N-terminal portion of a naturally occurring intein sequence. For example, an N-terminal split intein sequence can comprise additional amino acid residues and/or mutated residues so long as the inclusion of such additional and/or mutated residues does not render the intein non-functional with respect to splicing of two portions of the exogenous polypeptide. Preferably, the inclusion of the additional and/or mutated residues improves or enhances the splicing activity of the intein.

A C-terminal split intein for use with the methods and compositions described herein can be any intein sequence that comprises a C-terminal amino acid sequence that is functional for trans-splicing reactions. In one aspect, the C-terminal split intein comprises 4 to 7 contiguous amino acid residues, at least 4 amino acids of which are from the last β-strand of the intein from which it was derived. A C-terminal split intein region thus also comprises a sequence that is spliced out when trans-splicing occurs. A C-terminal split intein region can comprise a sequence that is a modification of the C-terminal portion of a naturally occurring intein sequence. For example, an C-terminal split intein region can comprise additional amino acid residues and/or mutated residues so long as the inclusion of such additional and/or mutated residues does not render the intein non-functional with respect to splicing. Preferably, the inclusion of the additional and/or mutated residues improves or enhances the splicing activity of the C-terminal split intein region.

In some embodiments, a peptide linked to a C-terminal or N-terminal split intein region can comprise an additional chemical moiety including, among others, fluorescence groups, biotin, polyethylene glycol (PEG), amino acid analogs, unnatural amino acids, phosphate groups, glycosyl groups, radioisotope labels, and pharmaceutical molecules. In other embodiments, a peptide linked to a C-terminal split intein region can comprise one or more chemically reactive groups including, among others, ketone, aldehyde, Cys residues and Lys residues. The N-intein and C-intein of a split intein can associate non-covalently to form an active intein and catalyze the splicing reaction when an “intein-splicing polypeptide (ISP)” is present. An “intein-splicing polypeptide (ISP)” is a portion of the amino acid sequence of a split intein that remains when the C-terminal or N-terminal split intein region or both, are removed from the split intein. In certain embodiments, the N-terminal split intein region comprises the ISP. In another embodiment, the C-terminal split intein region comprises the ISP. In yet another embodiment, the ISP is a separate peptide that is not covalently linked to either the C-terminal or N-terminal split intein region.

In protein trans-splicing, one precursor protein consists of an N-extein part followed by the N-intein, another precursor protein consists of the C-intein followed by a C-extein part, and a trans-splicing reaction (catalyzed by the N- and C-inteins together) excises the two intein sequences and links the two extein sequences with a peptide bond. Protein trans-splicing, being an enzymatic reaction, can work with very low (e.g. micromolar) concentrations of proteins and can be carried out under physiological conditions.

In some embodiments, the split intein sequences used herein are codon optimized for expression in particular cells, such as eukaryotic cells (e.g., eukaryotic muscle cells). The eukaryotic cells can be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected RNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.).

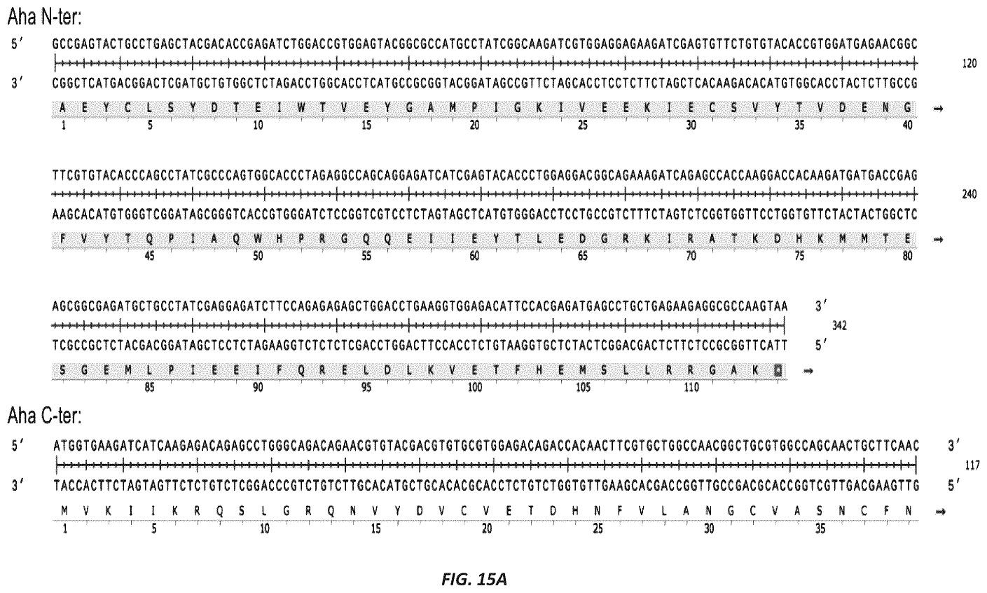

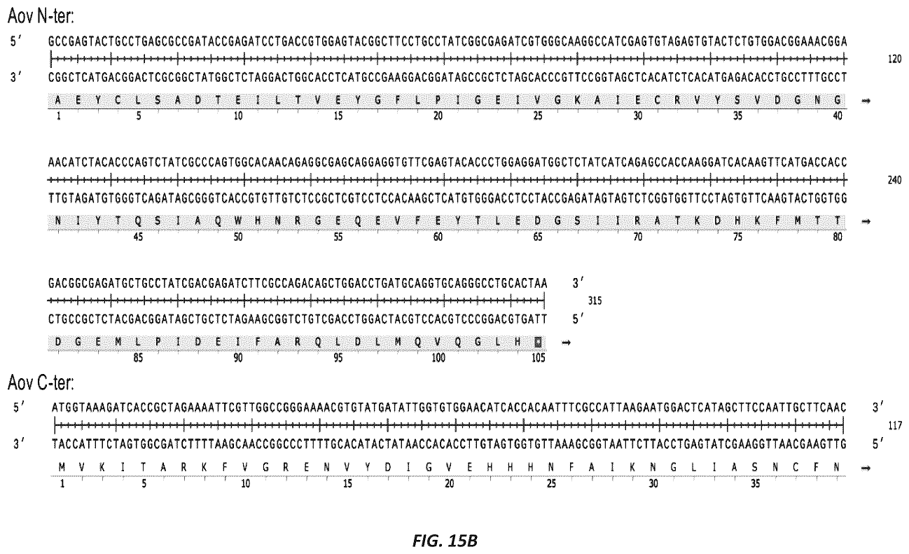

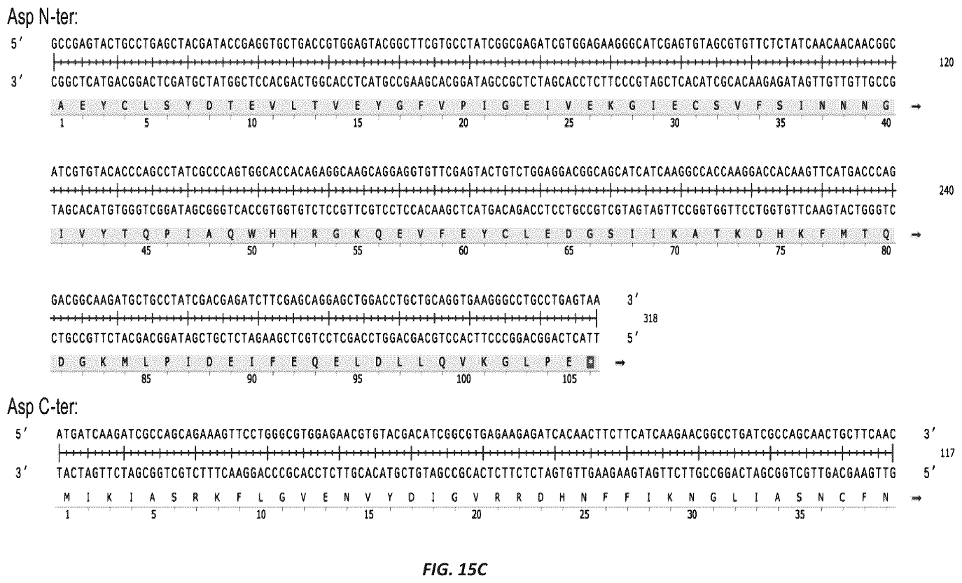

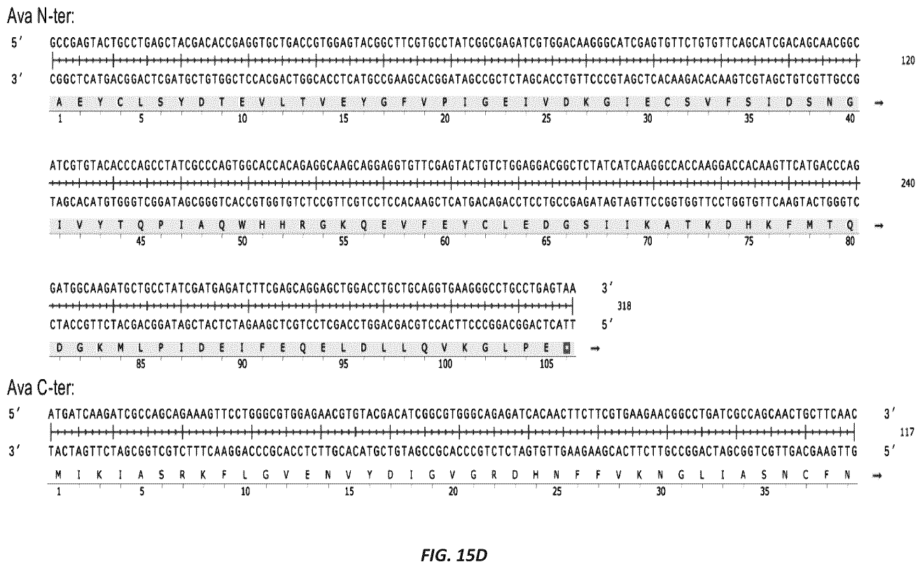

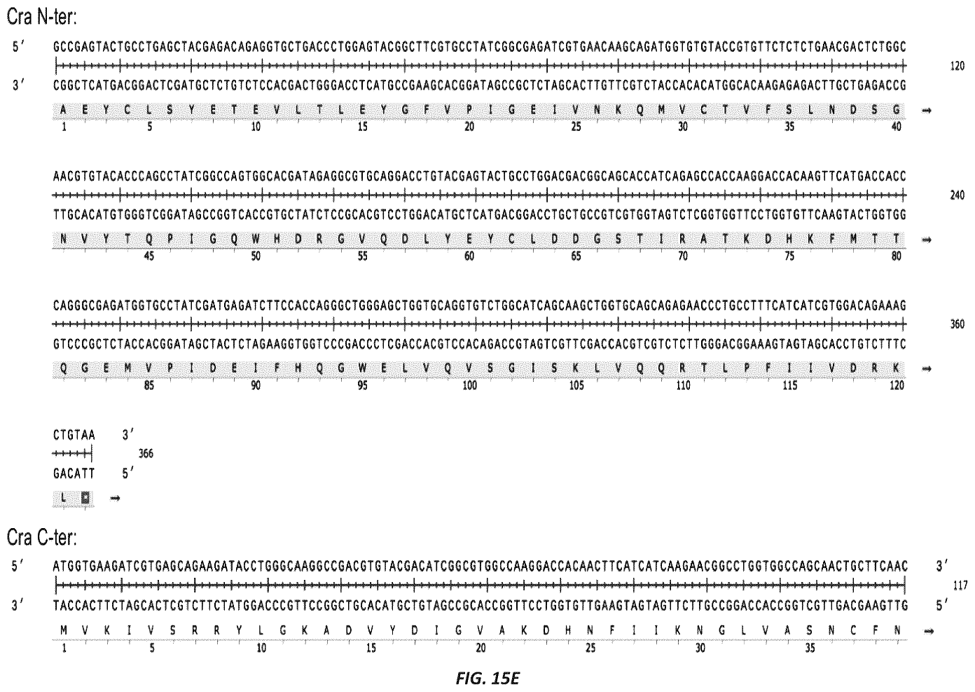

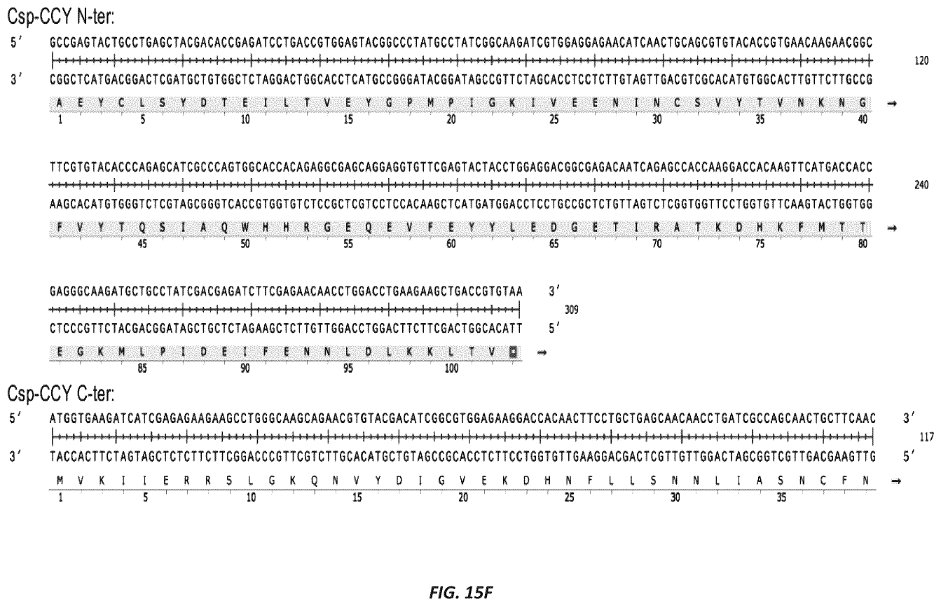

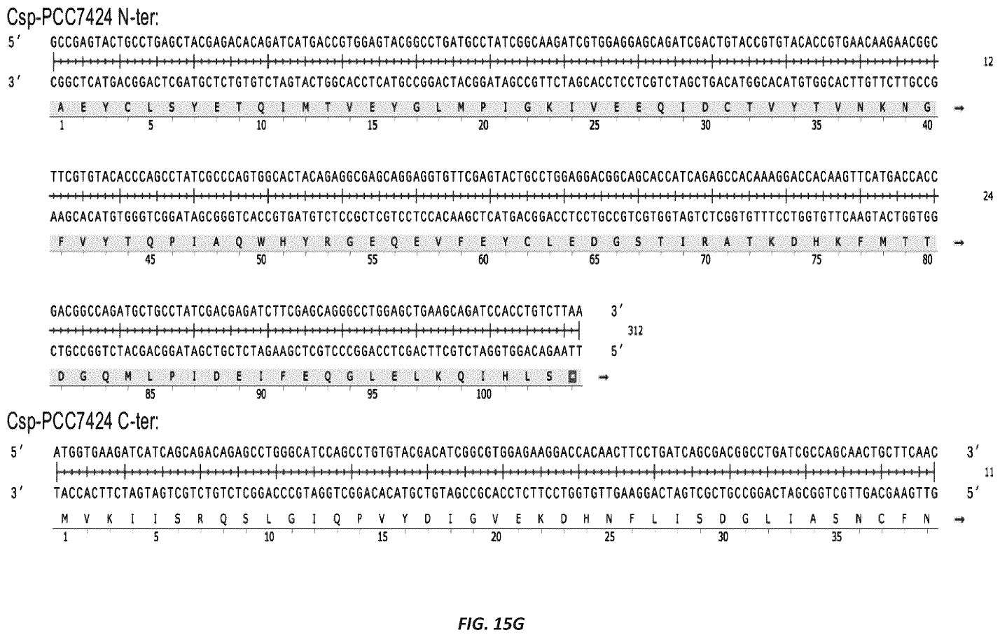

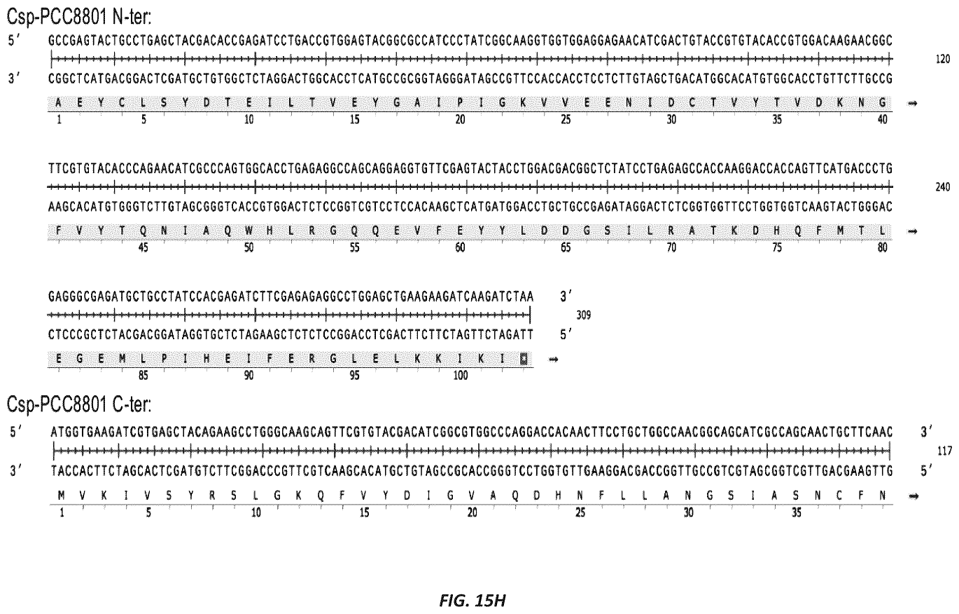

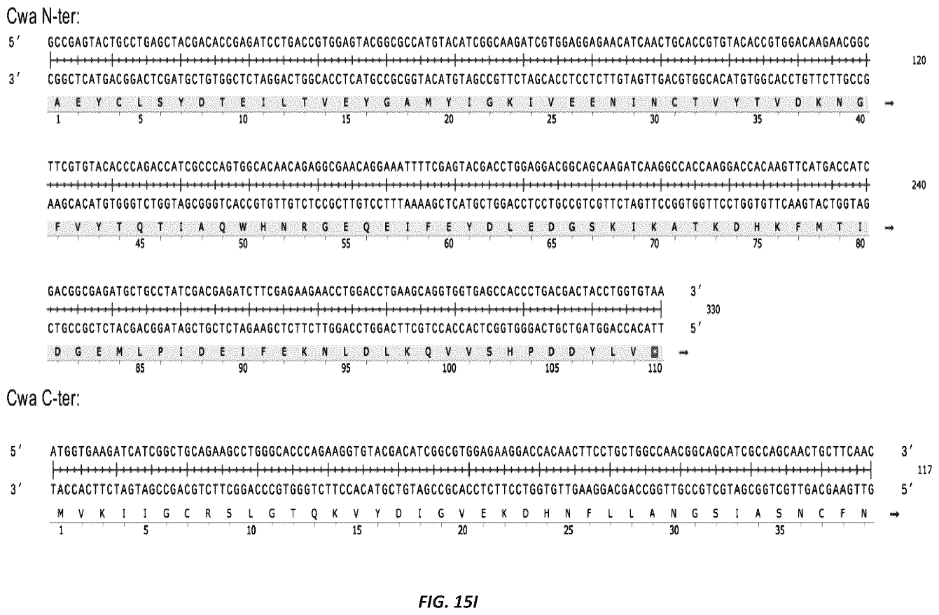

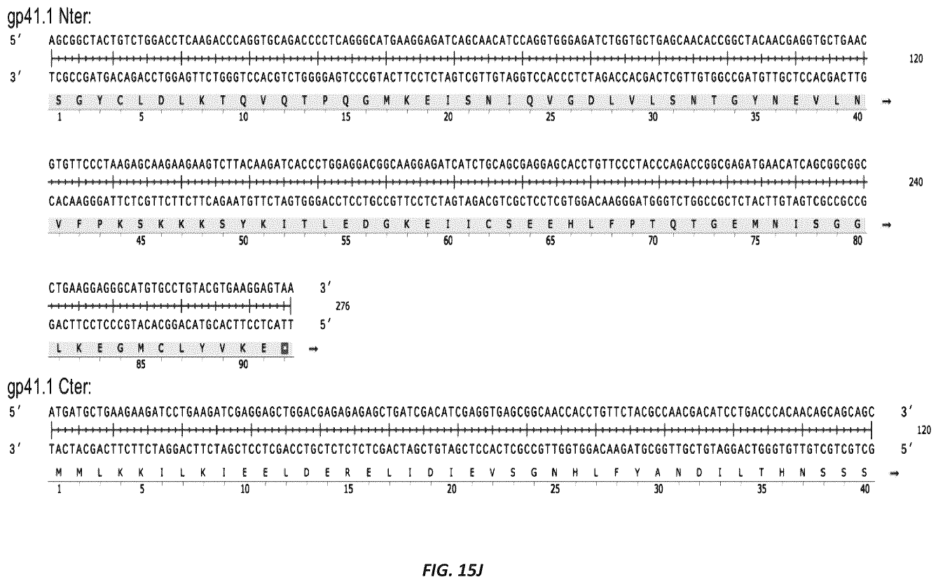

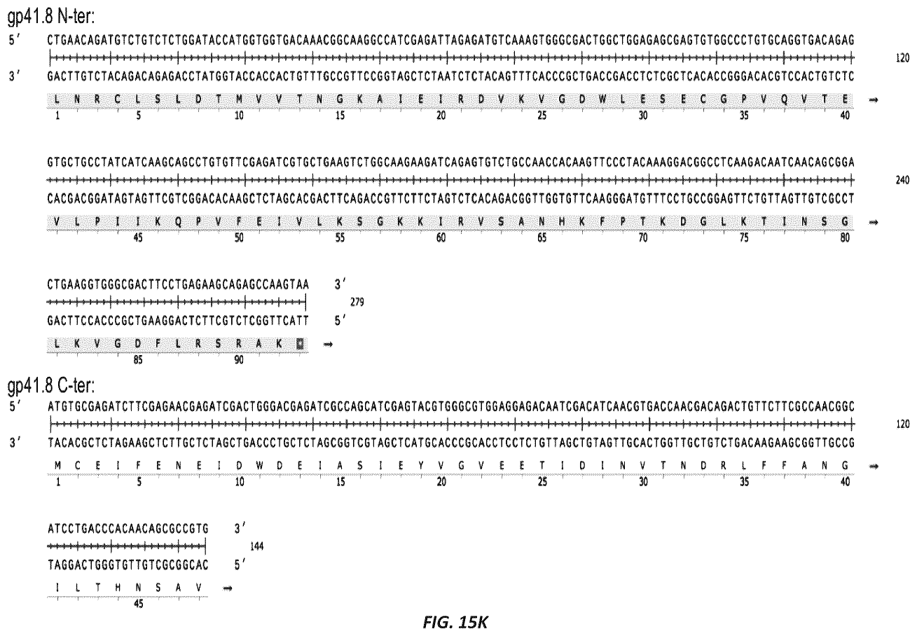

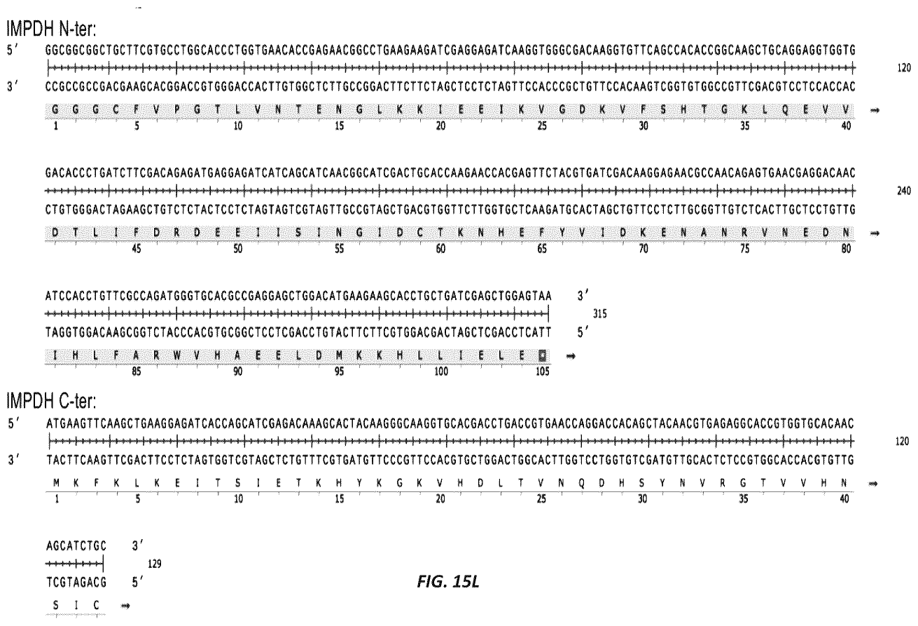

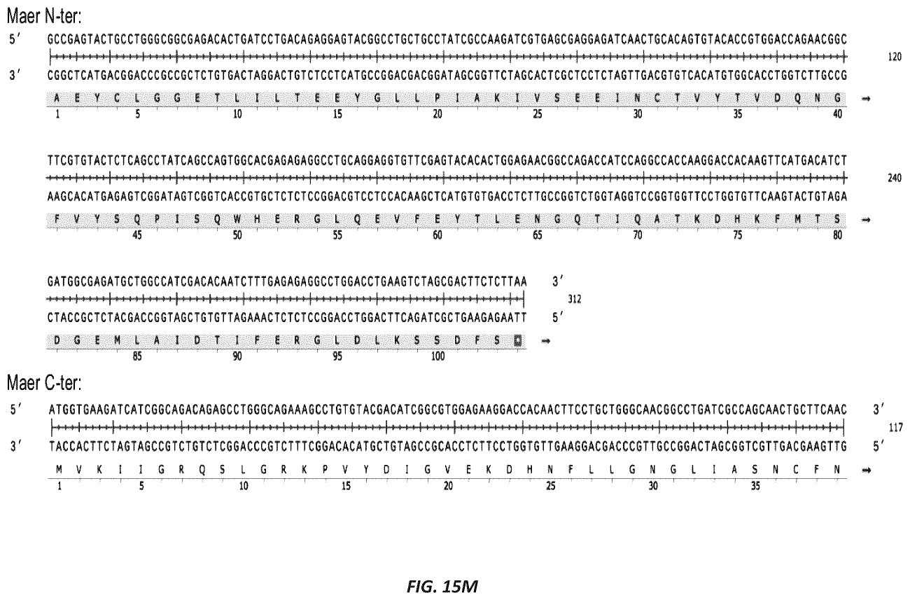

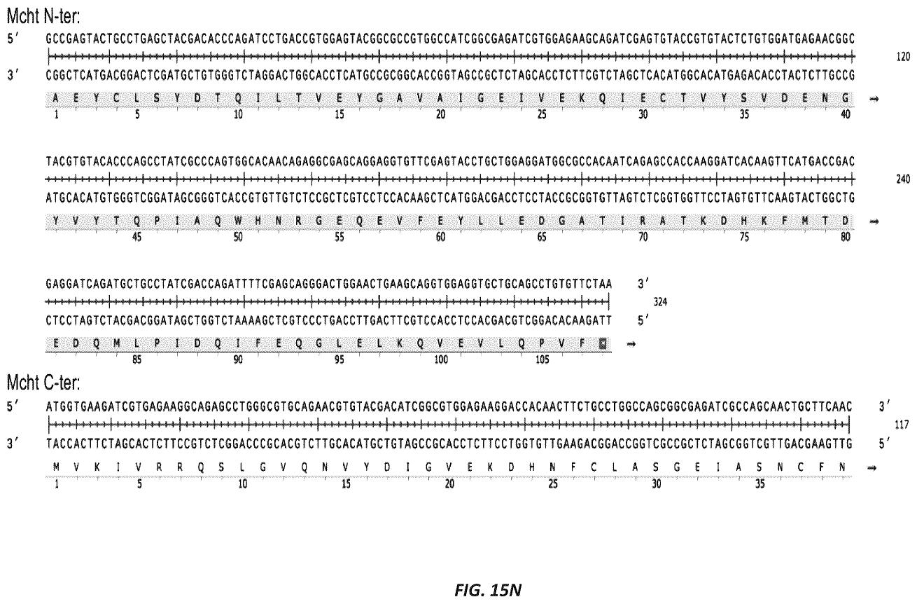

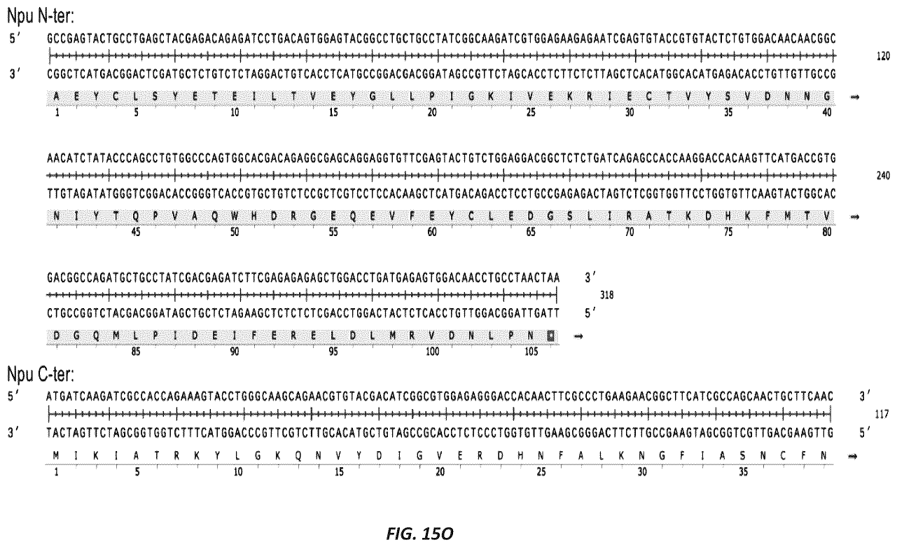

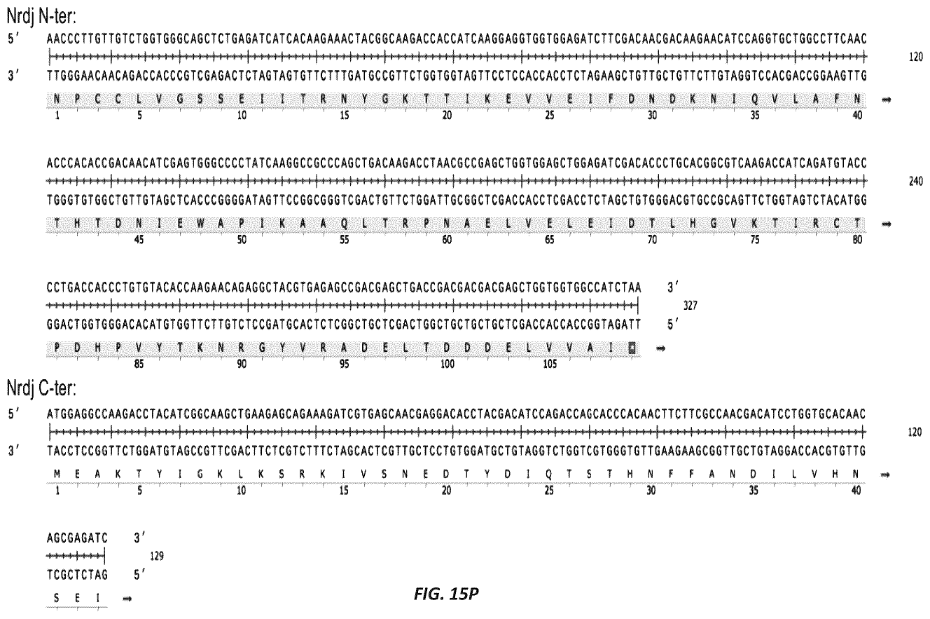

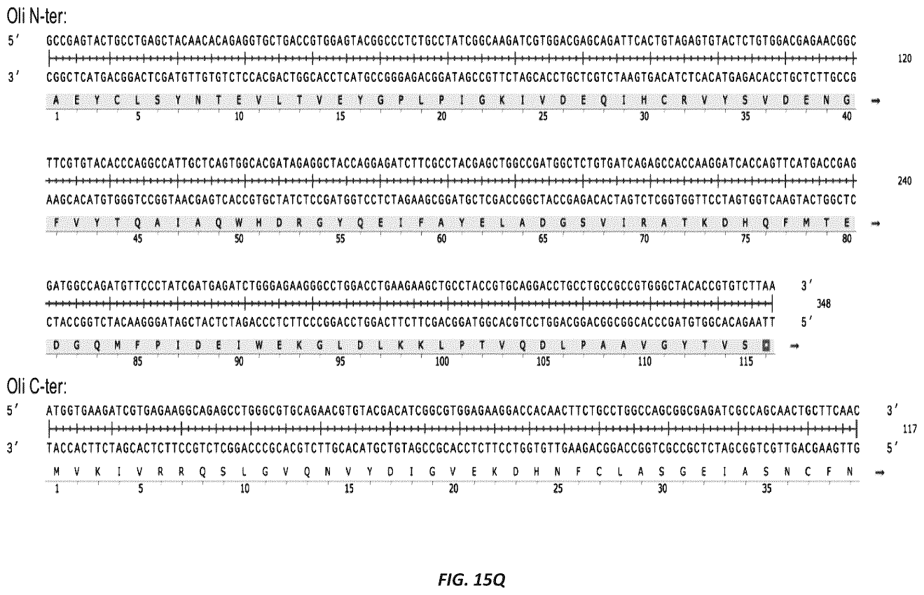

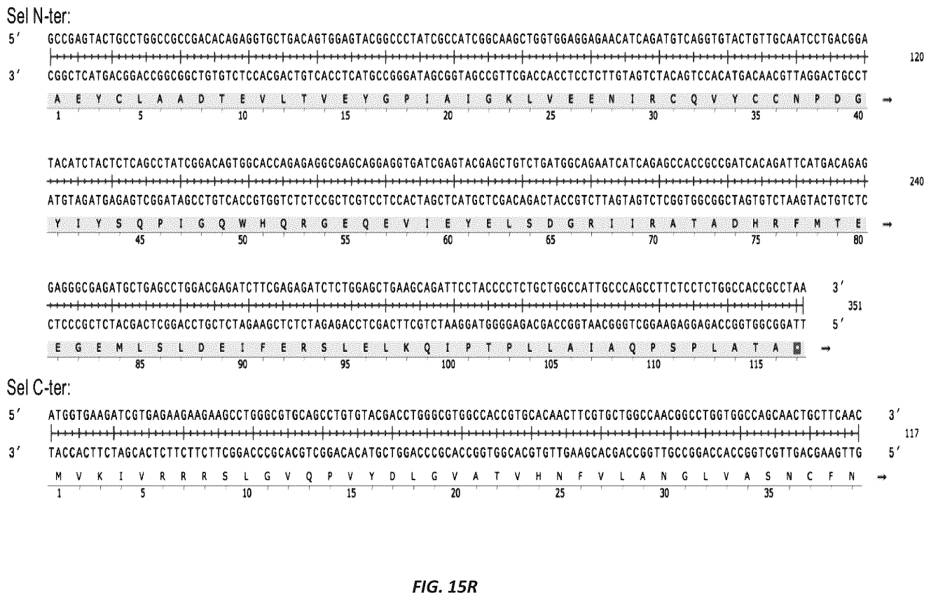

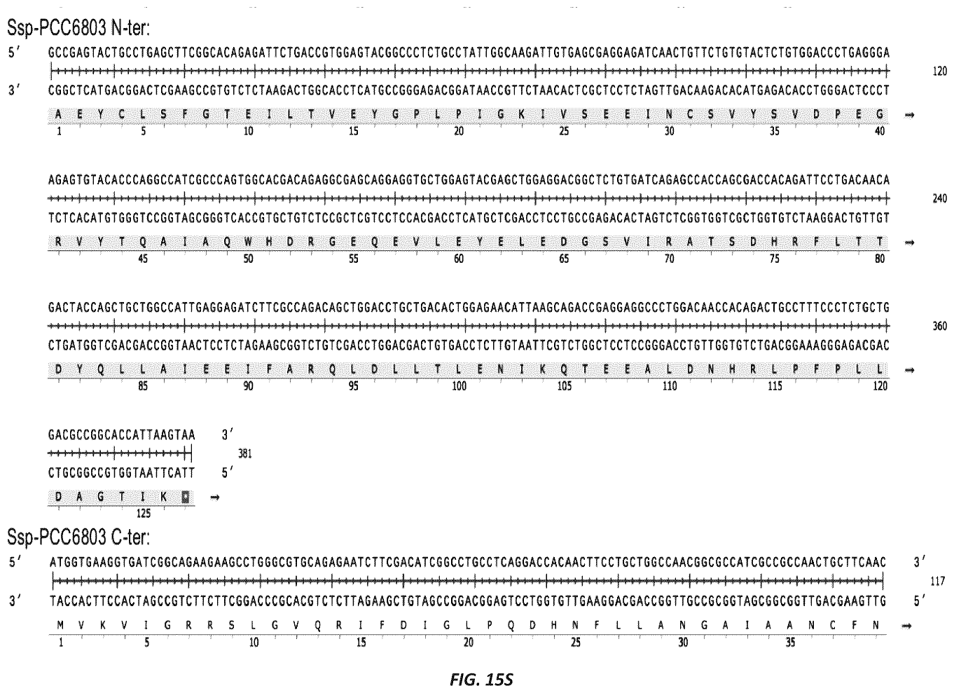

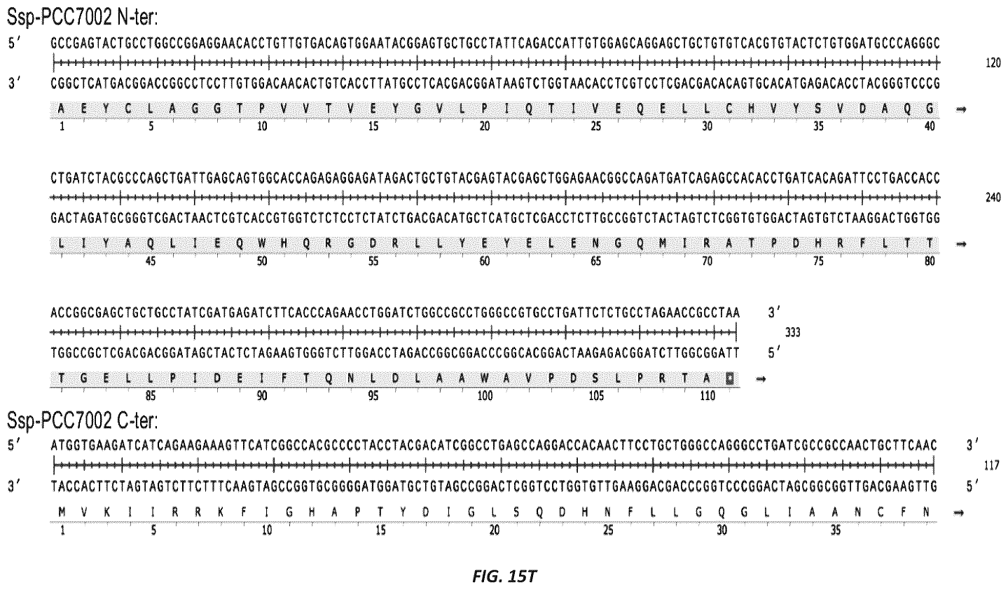

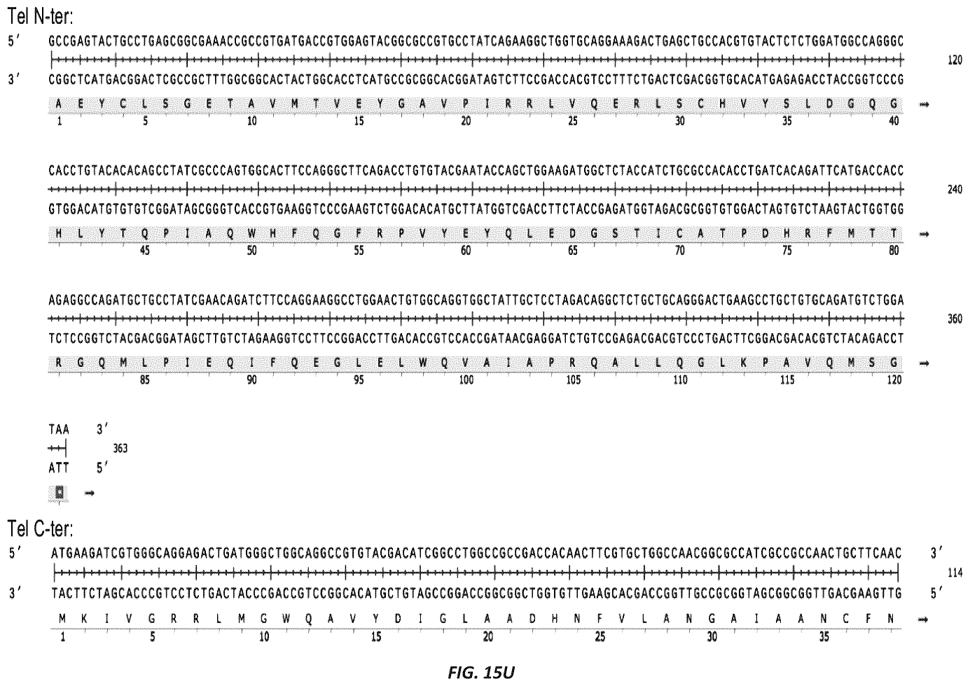

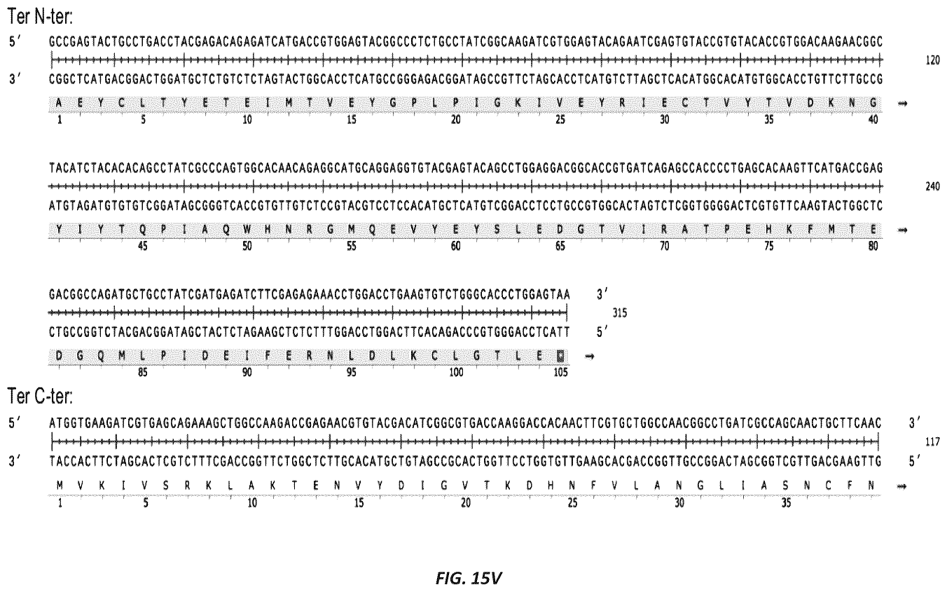

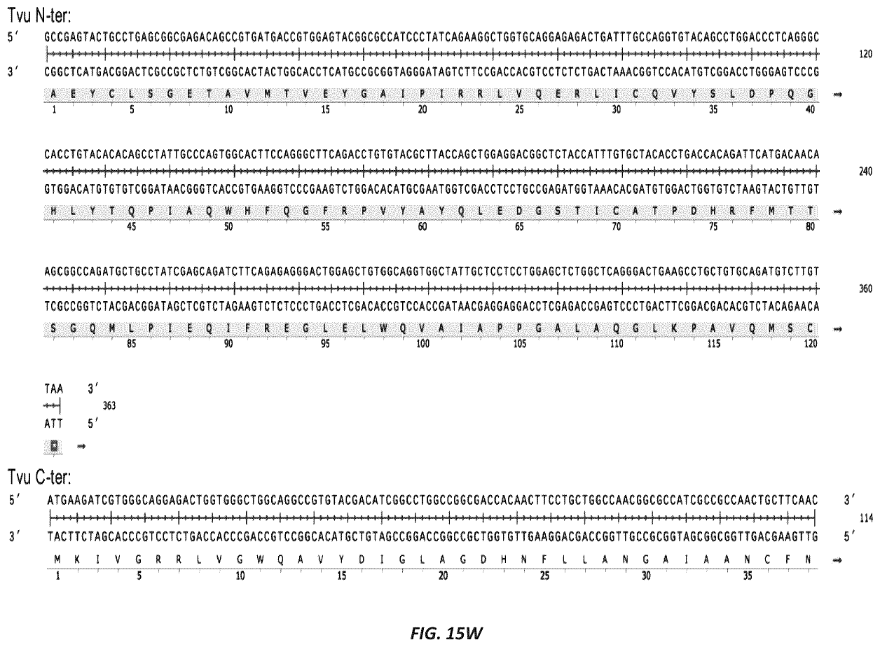

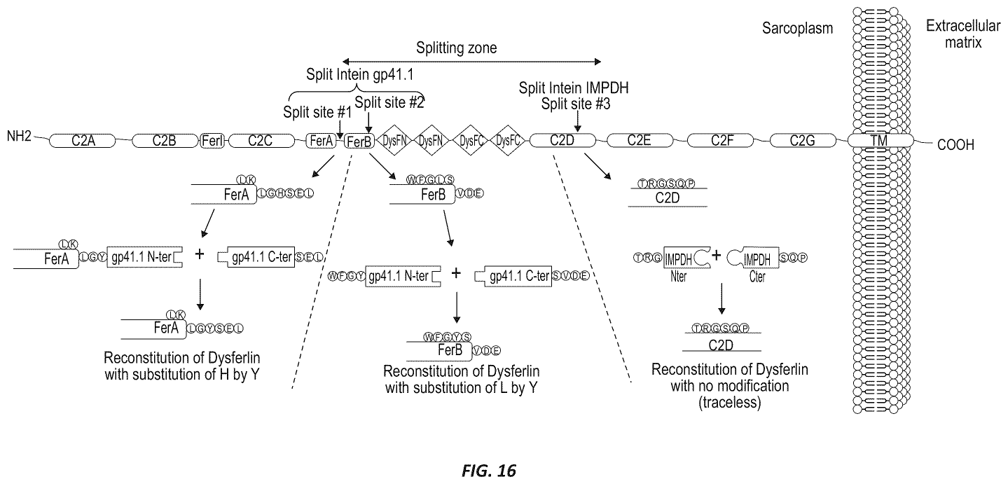

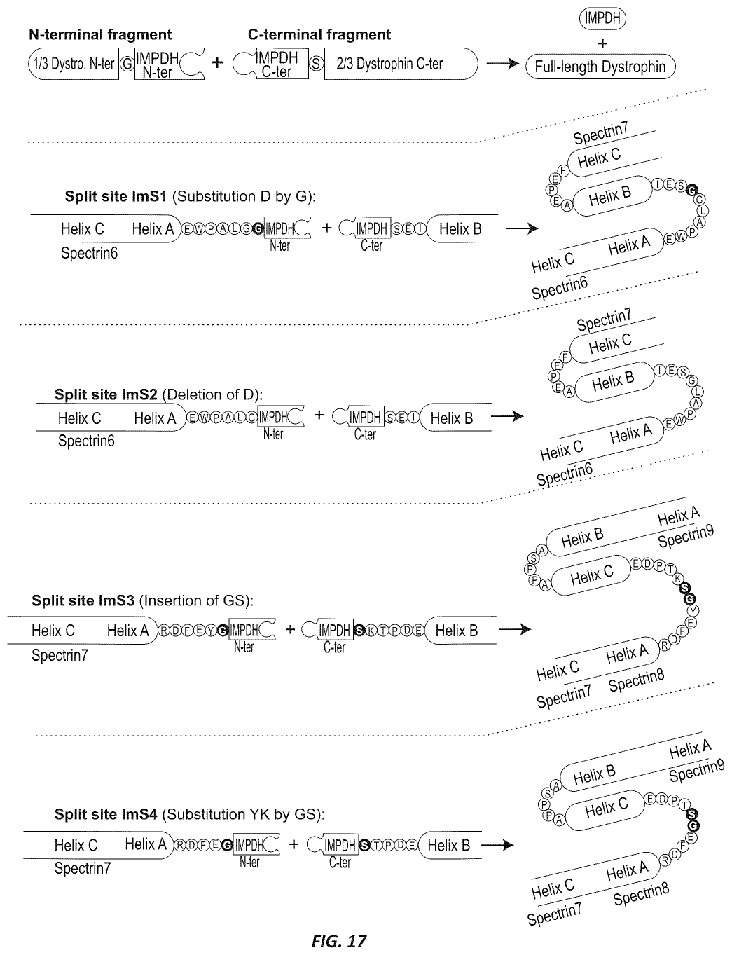

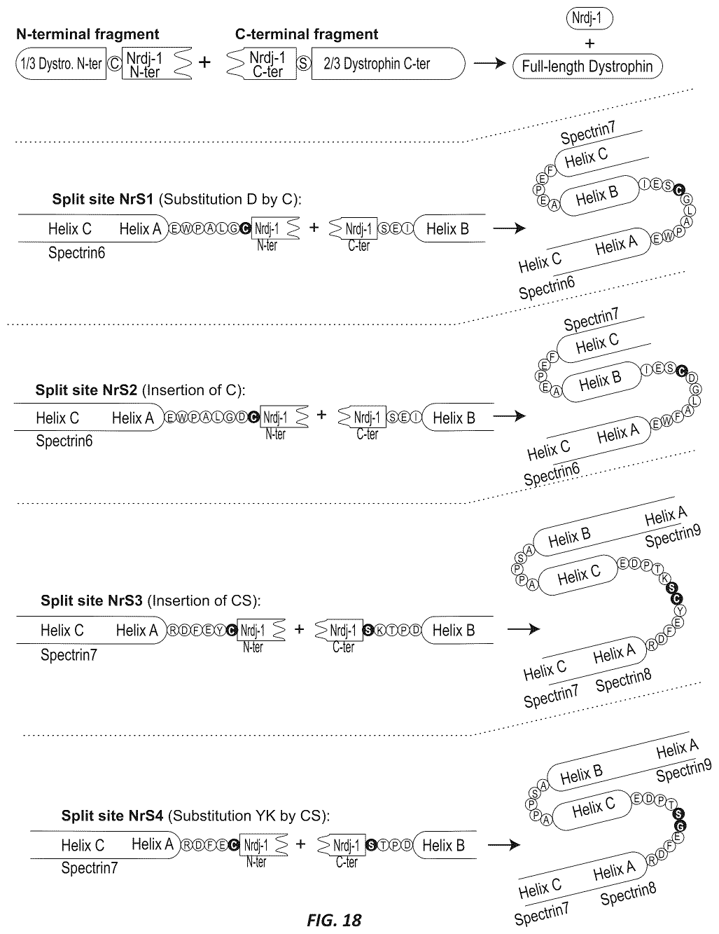

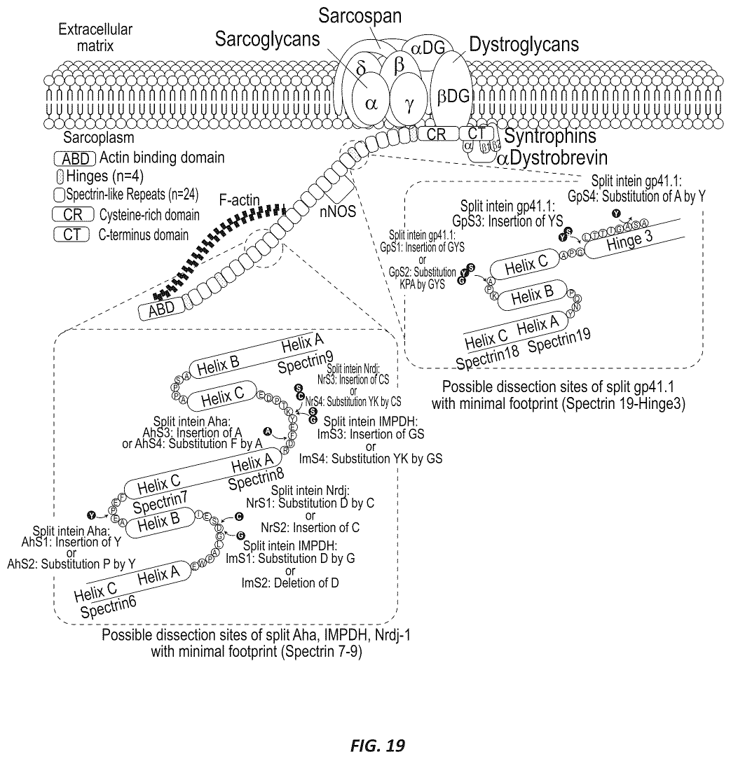

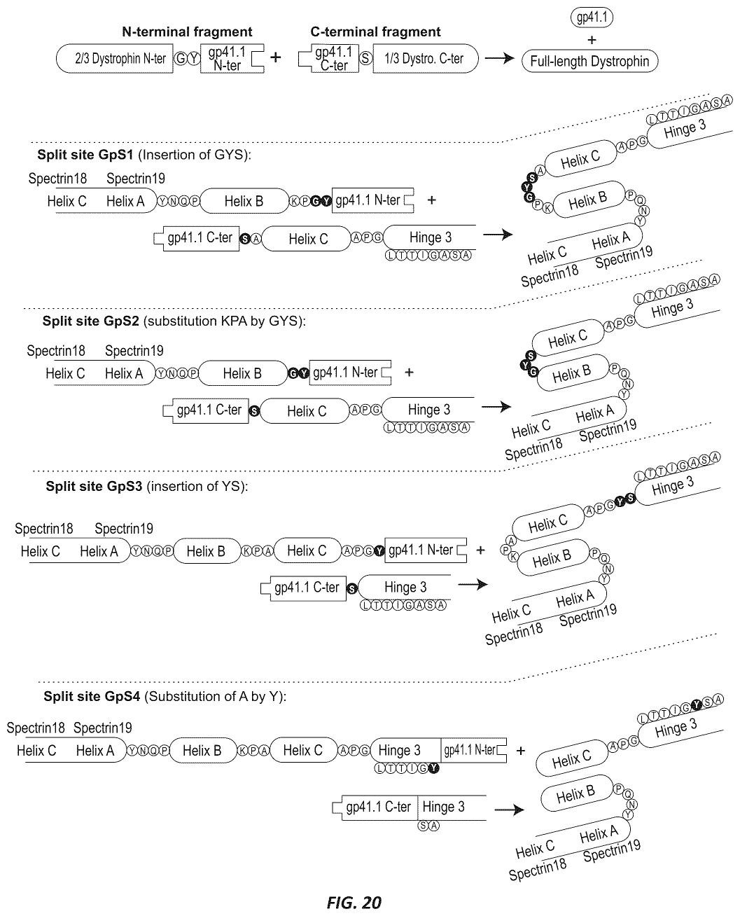

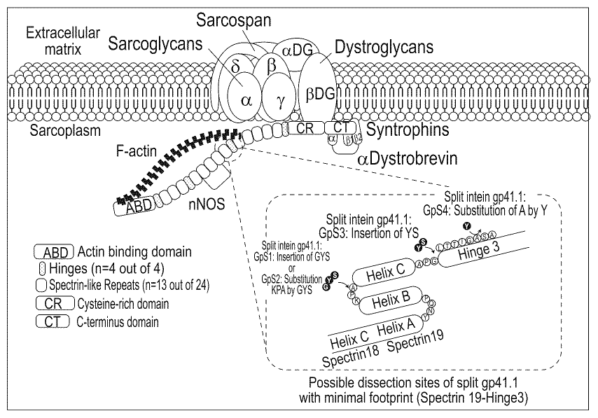

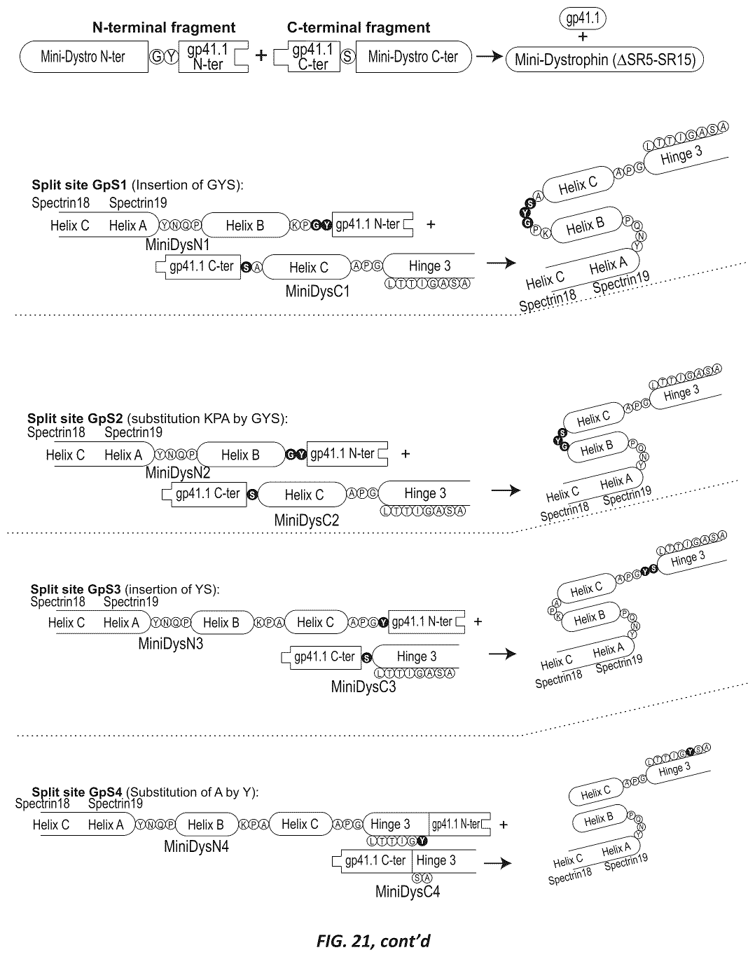

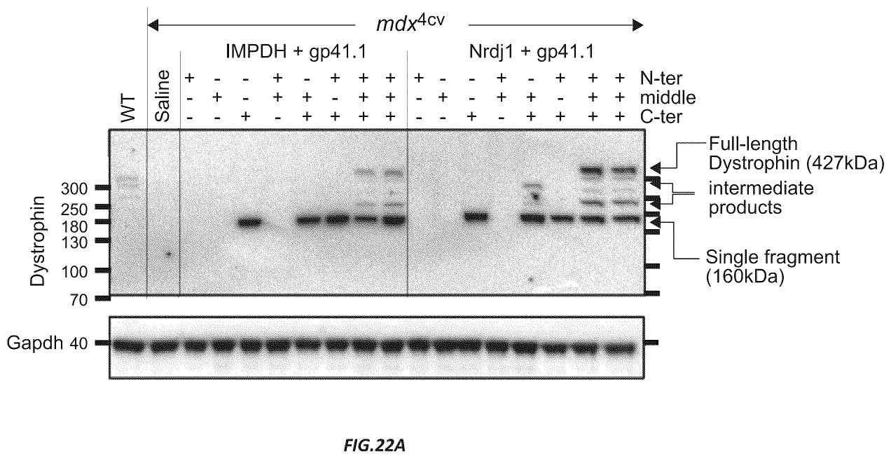

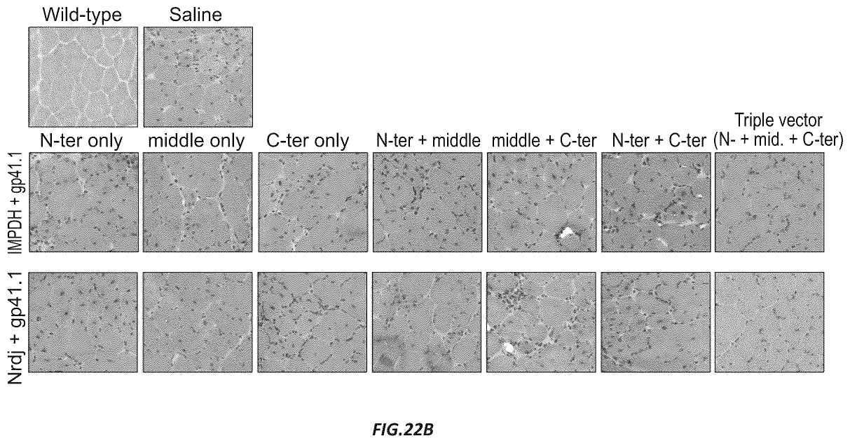

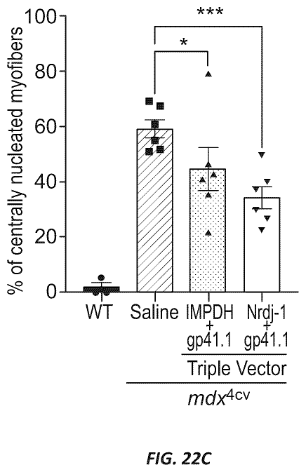

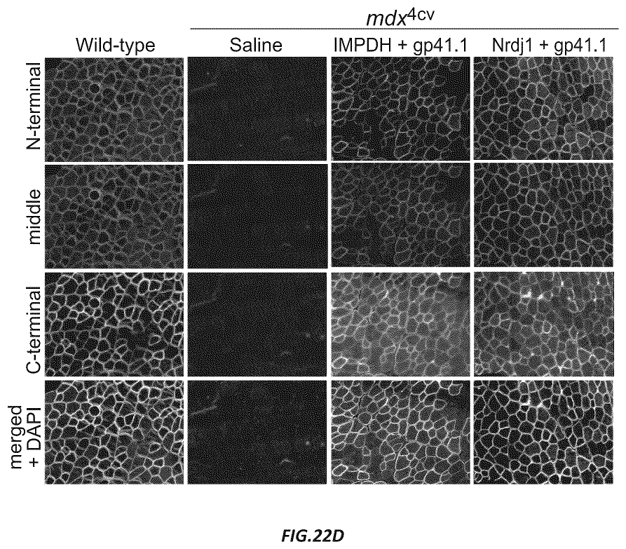

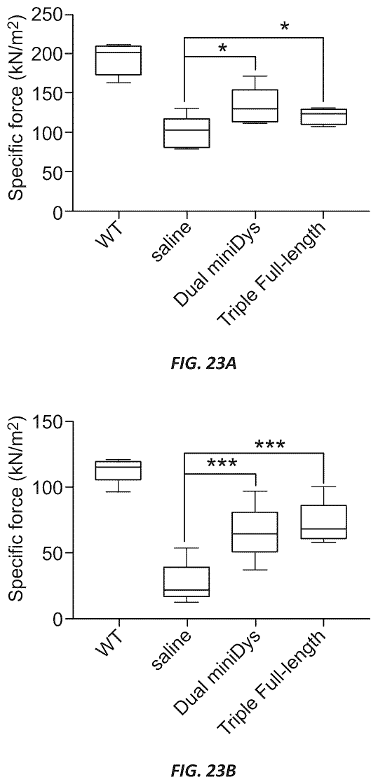

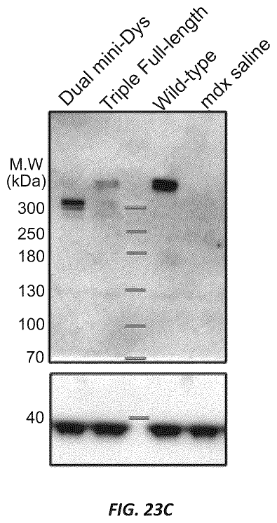

In some embodiments, the methods and compositions described herein utilize one or more split inteins present in the following Table. Exemplary split inteins for use herein are shown herein in FIGS. 15A-15U.

TABLE 3
Exemplary Split Inteins

| Intein | | |
|---|---|---|
| Name | Organism Name | Organism Description |
| Eucarya | | |
| APMV Pol | Acanthomoeba | isolate = “Rowbotham- |
| | polyphaga Mimivirus | Bradford”, Virus, infects |
| | | Amoebae, taxon: 212035 |
| Abr PRP8 | Aspergillus brevipes | Fungi, ATCC 16899, |
| | FRR2439 | taxon: 75551 |
| Aca- | Ajellomyces | Taxon: 447093, strain |
| G186AR | capsulatus G186AR | G186AR |
| PRP8 | | |
| Aca-H143 | Ajellomyces capsulatus H143 | Taxon: 544712 |
| PRP8 | | |
| Aca- | Ajellomyces | strain = JER2004, taxon: |
| JER2004 | capsulatus (anamorph: | 5037, |
| PRP8 | Histoplasma capsulatum) | Fungi |
| Aca-NAm1 | Ajellomyces capsulatus | strain = “NAm1”, taxon: |
| PRP8 | NAm1 | 339724 |
| Ade-ER3 | Ajellomyces dermatitidis | Human fungal |
| PRP8 | ER-3 | pathogen. taxon: 559297 |
| Ade- | Ajellomyces | Human fungal pathogen |
| SLH14081 | dermatitidis SLH14081, | |
| PRP8 | | |
| Afu-Af293 | Aspergillus | Human pathogenic |
| PRP8 | fumigatus var. ellipticus, | fungus, taxon: 330879 |
| | strain Af293 | |
| Afu- | Aspergillus fumigatus strain | Human pathogenic |
| FRR0163 | FRR0163 | fungus, taxon: 5085 |
| PRP8 | | |
| Afu- | Aspergillus | Human pathogenic |
| NRRL5109 | fumigatus var. ellipticus, | fungus, taxon: 41121 |
| PRP8 | strain NRRL 5109 | |
| Agi- | Aspergillus giganteus Strain | Fungus, taxon: 5060 |
| NRRL6136 | NRRL | |
| PRP8 | 6136 | |
| Ani- | Aspergillus nidulans FGSC A | Filamentous fungus, |
| FGSCA4 | | taxon: 227321 |
| PRP8 | | |
| Avi PRP8 | Aspergillus viridinutans | Fungi, ATCC 16902, |
| | strain FRR0577 | taxon: 75553 |
| Bci PRP8 | Botrytis cinerea (teleomorph | Plant fungal pathogen |
| | of | |
| | Botryotinia fuckeliana | |
| | B05.10) | |
| Bde-JEL197 | Batrachochytrium | Chytrid fungus, |
| RPB2 | dendrobatidis | isolate = “AFTOL-ID 21”, |
| | JEL197 | taxon: 109871 |
| Bde-JEL423 | Batrachochytrium | Chytrid fungus, isolate |
| PRP8-1 | dendrobatidis | JEL423, taxon 403673 |
| | JEL423 | |
| Bde-JEL423 | Batrachochytrium | Chytrid fungus, isolate |
| PRP8-2 | dendrobatidis | JEL423, taxon 403673 |
| | JEL423 | |
| Bde-JEL423 | Batrachochytrium | Chytrid fungus, isolate |
| RPC2 | dendrobatidis | JEL423, taxon 403673 |
| | JEL423 | |
| Bde-JEL423 | Batrachochytrium | Chytrid fungus, isolate |
| eIF-5B | dendrobatidis | JEL423, taxon 403673 |
| | JEL423 | |
| Bfu-B05 | Botryotinia fuckeliana | Taxon: 332648 |
| PRP8 | B05.10 | |
| CIV RIR1 | Chilo iridescent virus | dsDNA eucaryotic virus, |
| | | taxon: 10488 |
| CV-NY2A | Chlorella virus NY2A infects | dsDNA eucaryotic |
| ORF212392 | Chlorella NC64A, which | virus, taxon: 46021, |
| | infects | Family |
| CV-NY2A | Paramecium bursaria | Phycodnaviridae |
| RIR1 | Chlorella virus NY2A infects | dsDNA eucaryotic |
| | Chlorella NC64A, which | virus, taxon: 46021, |
| | infects | Family |
| | Paramecium bursaria | Phycodnaviridae |
| CZIV RIR1 | Costelytra | dsDNA eucaryotic virus, |
| | zealandica iridescent virus | Taxon: 68348 |
| Cba- | Cryptococcus | Yeast, human pathogen, |
| WM02.98 | bacillisporus strain | taxon: 37769 |
| PRP8 | WM02.98 (aka Cryptococcus | |
| | neoformans gattii) | |
| Cba-WM728 | Cryptococcus | Yeast, human pathogen, |
| PRP8 | bacillisporus strain | taxon: 37769 |
| | WM728 | |
| Ceu ClpP | Chlamydomonas eugametos | Green alga, taxon: 3053 |
| | (chloroplast) | |
| Cga PRP8 | Cryptococcus gattii (aka | Yeast, human pathogen |
| | Cryptococcus bacillisporus) | |
| Cgl VMA | Candida glabrata | Yeast, taxon: 5478 |
| Cla PRP8 | Cryptococcus laurentii strain | Fungi, Basidiomycete |
| | CBS139 | yeast, |
| | | taxon: 5418 |
| Cmo ClpP | Chlamydomonas moewusii | Green alga, chloroplast |
| | strain | gene, |
| | UTEX 97 | taxon: 3054 |
| Cmo RPB2 | Chlamydomonas moewusii, | Green alga, chloroplast |
| (RpoBb) | strain | gene, |
| | UTEX 97 | taxon: 3054 |
| Cne-A | Filobasidiella neoformans | Yeast, human pathogen |
| PRP8 (Fne- | (Cryptococcus neoformans) | |
| A | Serotype | |
| PRP8) | A, PHLS_8104 | |
| Cne-AD | Cryptococcus neoformans | Yeast, human pathogen, |
| PRP8 (Fne- | (Filobasidiella neoformans), | ATCC32045, taxon: 5207 |
| AD PRP8) | Serotype AD, CBS132). | |
| Cne-JEC21 | Cryptococcus | Yeast, human pathogen, |
| PRP8 | neoformans var. | serotype = “D” taxon: |
| | neoformans JEG21 | 214684 |
| Cpa ThrRS | Candida parapsilosis, strain | Yeast, Fungus, taxon: |
| | CLIB214 | 5480 |
| Cre RPB2 | Chlamydomonas reinhardtii | Green algae, taxon: 3055 |
| | (nucleus) | |
| CroV Pol | Cafeteria roenbergensis virus | taxon: 693272, Giant |
| | BV- | virus infecting marine |
| | PW1 | heterotrophic |
| | | nanoflagellate |
| CroV RIR1 | Cafeteria roenbergensis virus | taxon: 693272, Giant |
| | BV- | virus infecting marine |
| | PW1 | heterotrophic |
| | | nanoflagellate |
| CroV RPB2 | Cafeteria roenbergensis virus | taxon: 693272, Giant |
| | BV- | virus infecting marine |
| | PW1 | heterotrophic |
| | | nanoflagellate |
| CroV Top2 | Cafeteria roenbergensis virus | taxon: 693272, Giant |
| | BV- | virus infecting marine |
| | PW1 | heterotrophic |
| | | nanoflagellate |
| Cst RPB2 | Coelomomyces stegomyiae | Chytrid fungus, |
| | | isolate = “AFTOL-ID |
| | | 18”, taxon: 143960 |
| Ctr ThrRS | Candida tropicalis ATCC750 | Yeast |
| Ctr VMA | Candida tropicalis (nucleus) | Yeast |
| Ctr- | Candida tropicalis | Taxon: 294747 |
| MYA3404 | MYA-3404 | |
| VMA | | |
| Ddi RPC2 | Dictyostelium | Mycetozoa (a social |
| | discoldeum strain | amoeba) |
| | AX4 (nucleus) | |
| Dhan GLT1 | Debaryomyces | Fungi, Anamorph: |
| | hansenii CBS767 | Candida famata, |
| | | taxon: 4959 |
| Dhan VMA | Debaryomyces | Fungi, taxon: 284592 |
| | hansenii CBS767 | |
| Eni PRP8 | Emericella nidulans R20 | taxon: 162425 |
| | (anamorph: | |
| | Aspergillus nidulans) | |
| Eni- | Emericella | Filamentous fungus, |
| FGSCA4 | nidulans (anamorph: | taxon: 162425 |
| PRP8 | Aspergillus nidulans) FGSC | |
| | A4 | |
| Fte RPB2 | Floydiella terrestris, strain | Green alga, chloroplast |
| (RpoB) | UTEX | gene, |
| | 1709 | taxon: 51328 |
| Gth DnaB | Guillardia theta (plastid) | Cryptophyte Algae |
| HaV01 Pol | Heterosigma akashiwo virus | Algal virus, taxon: 97195, |
| | 01 | strain HaV01 |
| Hca PRP8 | Histoplasma | Fungi, human pathogen |
| | capsulatum (anamorph; | |
| | Ajellomyces capsulatus) | |
| IIV6 RIR1 | Invertebrate iridescent virus 6 | dsDNA eucaryotic |
| | | virus, taxon: 176652 |
| Kex- | Kazachstania exigua, | Yeast, taxon: 34358 |
| CBS379 | formerly Saccharomyces | |
| VMA | exiguus, strain | |
| | CBS379 | |
| Kla-CBS683 | Kluyveromyces lactis, strain | Yeast, taxon: 28985 |
| VMA | CBS683 | |
| Kla- | Kluyveromyces lactis | Fungi, taxon: 28985 |
| IFO1267 | IFO1267 | |
| VMA | | |
| Kla- | Kluyveromyces lactis NRRL | Fungi, taxon: 284590 |
| NRRLY1140 | Y-1140 | |
| VMA | | |
| Lel VMA | Lodderomyces elongisporus | Yeast |
| Mca- | Microsporum canis CBS | Taxon: 554155 |
| CBS113480 | 113480 | |
| PRP8 | | |
| Nau PRP8 | Neosartorya aurata NRRL | Fungus, taxon: 41051 |
| | 4378 | |
| Nfe- | Neosartorya fennelliae | Fungus, taxon: 41048 |
| NRRL5534 | NRRL 5534 | |
| PRP8 | | |
| Nfi PRP8 | Neosartorya fischeri | Fungi |
| Ngl-FR2163 | Neosartorya glabra | Fungi, ATCC 16909, |
| PRP8 | FRR2163 | taxon: 41049 |
| Ngl- | Neosartorya glabra | Fungi, taxon: 41049, |
| FRR1833 | FRR1833 | (preliminary |
| PRP8 | | identification) |
| Nqu PRP8 | Neosartorya quadricincta, | taxon: 41053 |
| | strain | |
| | NRRL 4175 | |
| Nspi PRP8 | Neosartorya | Fungi, taxon: 36631 |
| | spinosa FRR4595 | |
| Pabr-Pb01 | Paracoccidioides | Taxon: 502779 |
| PRP8 | brasiliensis Pb01 | |
| Pabr-Pb03 | Paracoccidioides | Taxon: 482561 |
| PRP8 | brasiliensis Pb03 | |
| Pan CHS2 | Podospora anserina | Fungi, Taxon 5145 |
| Pan GLT1 | Podospora anserina | Fungi, Taxon 5145 |
| Pbl PRP8-a | Phycomyces blakesleeanus | Zygomycete fungus, |
| | | strain NRRL155 |
| Pbl PRP8-b | Phycomyces blakesleeanus | Zygomycete fungus, |
| | | strain NRRL155 |
| Pbr-Pb18 | Paracoccidioides | Fungi, taxon: 121759 |
| PRP8 | brasiliensis Pb18 | |
| Pch PRP8 | Penicillium chrysogenum | Fungus, taxon: 5076 |
| Pex PRP8 | Penicillium expansum | Fungus, taxon27334 |
| Pgu GLT1 | Pichia (Candida) | Fungi, Taxon 294746 |
| | guilliermondii | |
| Pgu-alt | Pichia (Candida) | Fungi |
| GLT1 | guilliermondii | |
| Pno GLT1 | Phaeosphaeria | Fungi, taxon: 321614 |
| | nodorum SN15 | |
| Pno RPA2 | Phaeosphaeria | Fungi, taxon: 321614 |
| | nodorum SN15 | |
| Ppu DnaB | Porphyra | Red Alga |
| | purpurea (chloroplast) | |
| Pst VMA | Pichia stipitis CBS 6054, | Yeast |
| | taxon: 322104 | |
| Ptr PRP8 | Pyrenophora tritici-repentis | Ascomycete |
| | Pt-1C- | |
| | BF | fungus, taxon: 426418 |
| Pvu PRP8 | Penicillium vulpinum | Fungus |
| | (formerly P. claviforme) | |
| Pye DnaB | Porphyra | Red alga, |
| | yezoensis chloroplast, | organelle = “plastid: |
| | cultivar U-51 | chloroplast”, |
| | | “taxon: 2788 |
| Sas RPB2 | Spiromyces aspiralis NRRL | Zygomycete fungus, |
| | 22631 | Isolate = “AFTOL-ID |
| | | 185”, taxon: 68401 |
| Sca- | Saccharomyces castellii, | Yeast, taxon: 27288 |
| CBS4309 | strain | |
| VMA | CBS4309 | |
| Sca- | Saccharomyces castellii, | Yeast, taxon: 27288 |
| IFO1992 | strain | |
| VMA | IFO1992 | |
| Scar VMA | Saccharomyces cariocanus, | Yeast, taxon: 114526 |
| | strain = ″UFRJ 50791 | |
| Sce VMA | Saccharomyces | Yeast, also in Sce strains |
| | cerevisiae (nucleus) | |
| Sce-DH1-1A | Saccharomyces | OUT7163, OUT7045, |
| VMA | cerevisiae strain | OUT7163, IFO1992 |
| | DH1-1A | Yeast, taxon: 173900, |
| | | also in |
| | | Sce strains |
| | | OUT7900, OUT7903, |
| | | OUT7112 |
| Sce-JAY291 | Saccharomyces | Taxon: 574961 |
| VMA | cerevisiae JAY291 | |
| Sce- | Saccharomyces | Yeast, taxon: 4932, also |
| OUT7091 | cerevisiae OUT7091 | in Sce |
| VMA | | strains OUT7043, |
| | | OUT7064 |
| Sce- | Saccharomyces | Yeast, taxon: 4932, also |
| OUT7112 | cerevisiae OUT7112 | in Sce |
| VMA | | strains OUT7900 |
| | | OUT7903 |
| Sce- | Saccharomyces | Yeast, taxon: 307796 |
| YJM789 | cerevisiae strain | |
| VMA | YJM789 | |
| Sda VMA | Saccharomyces dairenensis, | Yeast, taxon: 27289, Also |
| | strain | in |
| | CBS 421 | Sda strain IFO0211 |
| Sex- | Saccharomyces exiguus, | Yeast, taxon: 34358 |
| IFO1128 | strain = ″IFO1128″ | |
| VMA | | |
| She RPB2 | Stigeoclonium helveticum, | Green alga, chloroplast |
| (RpoB) | strain | gene, |
| | UTEX 441 | taxon: 55999 |
| Sja VMA | Schizosaccharomyces | Ascomycete fungus, |
| | japonicus | taxon: 402676 |
| | yFS275 | |
| Spa VMA | Saccharomyces pastorianus | Yeast, taxon: 27292 |
| | IFO11023 | |
| Spu PRP8 | Spizellomyces punctatus | Chytrid fungus, |
| Sun VMA | Saccharomyces unisporus, | Yeast, taxon: 27294 |
| | strain | |
| | CBS 398 | |
| Tgl VMA | Torulaspora globosa, strain | Yeast, taxon: 48254 |
| | CBS 764 | |
| Tpr VMA | Torulaspora pretoriensis, | Yeast, taxon: 35629 |
| | strain CBS | |
| | 5080 | |
| Ure-1704 | Uncinocarpus reesii | Filamentous fungus |
| PRP8 | | |
| Vpo VMA | Vanderwaltozyma polyspora, | Yeast, taxon: 36033 |
| | formerly Kluyveromyces | |
| | polysporus, | |
| | strain CBS 2163 | |
| WIV RIR1 | Wiseana iridescent virus | dsDNA eucaryotic |
| | | virus, taxon: 68347 |
| Zba VMA | Zygosaccharomyces bailii, | Yeast, taxon: 4954 |
| | strain | |
| | CBS 685 | |
| Zbi VMA | Zygosaccharomyces bisporus, | Yeast, taxon: 4957 |
| | strain | |
| | CBS 702 | |
| Zro VMA | Zygosaccharomyces rouxii, | Yeast, taxon: 4956 |
| | strain | |
| | CBS 688 | |
| Eubacteria | | |
| AP-APSE1 | Acyrthosiphon | Bacteriophage, taxon: |
| dpol | pisum secondary | 67571 |
| | endosymbiot phage 1 | |
| AP-APSE2 | Bacteriophage APSE-2, | Bacteriophage |
| dpol | isolate = T5A | of Candidatus |
| | | Hamiltonella defensa, |
| | | endosymbiot of |
| | | Acyrthosiphon pisum, |
| | | Bacteriophage, taxon: |
| | | taxon: 340054 |
| AP-APSE4 | Bacteriophage of Candidatus | Bacteriophage, taxon: |
| dpol | Hamiltonella defensa strain | 568990 |
| | 5ATac, | |
| | endosymbiot of | |
| | Acyrthosiphon pisum | |
| AP-APSE5 | Bacteriophage APSE-5 | Bacteriophage |
| dpol | | of Candidatus |
| | | Hamiltonella defensa, |
| | | endosymbiot of |
| | | Uroleucon rudbeckiae, |
| | | taxon: 568991 |
| AP-Aaphi23 | Bacteriophage Aaphi23, | Actinobacillus |
| MupF | Haemophilus phage Aaphi23 | actinomycetemcomitans |
| | | Bacteriophage, taxon: |
| | | 230158 |
| Aae RIR2 | Aquifex aeolicus strain VF5 | Thermophilic |
| | | chemolithoautotroph, |
| | | taxon: 63363 |
| Aave- | Acidovorax | taxon: 397945 |
| AAC001 | avenae subsp. citrulli | |
| Aave1721 | AAC00-1 | |
| Aave- | Acidovorax | taxon: 397945 |
| AAC001 | avenae subsp. citrulli | |
| RIR1 | AAC00-1 | |
| Aave- | Acidovorax | Taxon: 643561 |
| ATCC19860 | avenae subsp. avenae | |
| RIR1 | ATCC 19860 | |
| Aba Hyp- | Acinetobacter | taxon: 405416 |
| 02185 | baumannii ACICU | |
| Ace RIR1 | Acidothermus | taxon: 351607 |
| | cellulolyticus 11B | |
| Aeh DnaB-1 | Alkalilimnicola | taxon: 187272 |
| | ehrlichei MLHE-1 | |
| Aeh DnaB-2 | Alkalilimnicola | taxon: 187272 |
| | ehrlichei MLHE-1 | |
| Aeh RIR1 | Alkalilimnicola | taxon: 187272 |
| | ehrlichei MLHE-1 | |
| AgP-S1249 | Aggregatibacter phage S1249 | Taxon: 683735 |
| MupF | | |
| Aha DnaE-c | Aphanothece halophytica | Cyanobacterium, taxon: |
| | | 72020 |
| Aha DnaE-n | Aphanothece halophytica | Cyanobacterium, taxon: |
| | | 72020 |
| Alvi- | Allochromatium vinosum | Taxon: 572477 |
| DSM180 | DSM 180 | |
| GyrA | | |
| Ama | phage uncharacterized | Probably prophage gene, |
| MADE823 | protein | taxon: 314275 |
| | [Alteromonas macleodii | |
| | ‘Deep ecotype’] | |
| Amax- | Arthrospira maxima CS-328 | Taxon: 513049 |
| CS328 | | |
| DnaX | | |
| Aov DnaE-c | Aphanizomenon ovalisporum | Cyanobacterium, taxon: |
| | | 75695 |
| Aov DnaE-n | Aphanizomenon ovalisporum | Cyanobacterium, taxon: |
| | | 75695 |
| Apl-C1 | Arthrospira platensis | Taxon: 118562, strain C1 |
| DnaX | | |
| Arsp-FB24 | Arthrobacter species FB24 | taxon: 290399 |
| DnaB | | |
| Asp DnaE-c | Anabaena species PCC7120, | Cyanobacterium, |
| | (Nostoc | Nitrogen-fixing, |
| | sp. PCC7120) | taxon: 103690 |
| Asp DnaE-n | Anabaena species PCC7120, | Cyanobacterium, |
| | (Nostoc | Nitrogen-fixing, |
| | sp. PCC7120) | taxon: 103690 |
| Ava DnaE-c | Anabaena | Cyanobacterium, taxon: |
| | variabilis ATCC29413 | 240292 |
| Ava DnaE-n | Anabaena | Cyanobacterium, taxon: |
| | variabilis ATCC29413 | 240292 |
| Avin RIR1 | Azotobacter vinelandii | taxon: 354 |
| BIL | | |
| Bce-MCO3 | Burkholderia | taxon: 406425 |
| DnaB | cenocepacia MC0-3 | |
| Bce-PC184 | Burkholderia | taxon: 350702 |
| DnaB | cenocepacia PC184 | |
| Bse-MLS10 | Bacillus | Probably prophage gene, |
| TerA | selenitireducens MLS10 | Taxon: 439292 |
| BsuP- | B. subtilis M1918 (prophage) | Prophage in B. |
| M1918 RIR1 | | subtilis M1918. |
| | | taxon: 157928 |
| BsuP- | B. subtilis strain 168 Sp beta | B. subtilis taxon 1423, |
| SPBc2 RIR1 | c2 | SPbeta |
| | prophage | c2 phage, taxon: 66797 |
| Bvi IcmO | Burkholderia | plasmid = “pBVIE03”. |
| | vietnamiensis G4 | taxon: 269482 |
| CP-P1201 | Corynebacterium phage | lytic bacteriophage P1201 |
| Thy1 | P1201 | from Corynebacterium |
| | | glutamicum NCHU |
| | | 87078. Viruses; dsDNA |
| | | viruses, taxon: 384848 |
| Cag RIR1 | Chlorochromatium | Motile, phototrophic |
| | aggregatum | consortia |
| Cau SpoVR | Chloroflexus aurantiacus J- | Anoxygenic |
| | 10-fl | |
| CbP-C-St | Clostridium botulinum phage | phototroph, taxon: |
| RNR | C-St | 324602 Phage, specific_ |
| | | host = “Clostridium |
| | | botulinum type C strain |
| | | C-Stockholm, taxon: |
| | | 12336 |
| CbP-D1873 | Clostridium botulinum | Ssp. phage |
| RNR | phage D | from Clostridium |
| | | botulinum type D strain, |
| | | 1873, |
| | | taxon: 29342 |
| Cbu- | Coxiella burnetii Dugway | Proteobacteria; |
| Dugway | 5J108-111 | Legionellales, |
| DnaB | | taxon: 434922 |
| Cbu-Goat | Coxiella burnetii ‘MSU Goat | Proteobacteria; |
| DnaB | Q177’ | Legionellales; |
| | | taxon: 360116 |
| Cbu- | Coxiella burnetii RSA 334 | Proteobacteria; |
| RSA334 | | Legionellales; |
| DnaB | | taxon: 360117 |
| Cbu- | Coxiella burnetii RSA 493 | Proteobacteria; |
| RSA493 | | Legionellales; |
| DnaB | | taxon: 227377 |
| Cce Hyp1- | Cyanothece sp. ATCC 51142 | Marine unicellular |
| Csp-2 | | diazotrophic |
| | | cyanobacterium, |
| | | taxon: 43989 |
| Cch RIR1 | Chlorobium | taxon: 340177 |
| | chlorochromatii CaD3 | |
| Ccy Hyp1- | Cyanothece sp. CCY0110 | Cyanobacterium, |
| Csp-1 | | taxon: 391612 |
| Ccy Hyp1- | Cyanothece sp. CCY0110 | Cyanobacterium, |
| Csp-2 | | taxon: 391612 |
| Cfl- | Cellulomonas flavigena | Taxon: 446466 |
| DSM20109 | DSM 20109 | |
| DnaB | | |
| Chy RIR1 | Carboxydothermus | Thermophile, taxon = |
| | hydrogenoformans Z-2901 | 246194 |
| Ckl PTerm | Clostridium kluyveri DSM | plasmid = “pCKL555A”, |
| | 555 | taxon: 431943 |
| Cra-CS505 | Cylindrospermopsis | Taxon: 533240 |
| DnaE-c | raciborskii CS-505 | |
| Cra-CS505 | Cylindrospermopsis | Taxon: 533240 |
| DnaE-n | raciborskii CS-505 | |
| Cra-CS505 | Cylindrospermopsis | Taxon: 533240 |
| GyrB | raciborskii CS-505 | |
| Csp- | Cyanothece sp. CCY0110 | Taxon: 391612 |
| CCY0110 | | |
| DnaE-c | | |
| Csp- | Cyanothece sp. CCY0110 | Taxon: 391612 |
| CCY0110 | | |
| DnaE-n | | |
| Csp- | Cyanothece sp. PCC 7424 | Cyanobacterium, taxon: |
| PCC7424 | | 65393 |
| DnaE-c | | |
| Csp- | Cyanothece sp. PCC7424 | Cyanobacterium, taxon: |
| PCC7424 | | 65393 |
| DnaE-n | | |
| Csp- | | |
| PCC7425 | Cyanothece sp. PCC 7425 | Taxon: 395961 |
| DnaB | | |
| Csp- | | |
| PCC7822 | Cyanothece sp. PCC 7822 | Taxon: 497965 |
| DnaE-n | | |
| Csp- | | |
| PCC8801 | Cyanothece sp. PCC 8801 | Taxon: 41431 |
| DnaE-c | | |
| Csp- | | |
| PCC8801 | Cyanothece sp. PCC 8801 | Taxon: 41431 |
| DnaE-n | | |
| Cth ATPase | Clostridium thermocellum | ATCC27405, taxon: |
| BIL | | 203119 |
| Cth- | Clostridium thermocellum | Probable prophage, |
| ATCC27405 | ATCC27405 | ATCC27405, taxon: |
| TerA | | 203119 |
| Cth- | Clostridium | Probably prophage |
| DSM2360 | thermocellum DSM | gene, Taxon: 572545 |
| TerA | 2360 | |
| Cwa DnaB | Crocosphaera watsonii WH | taxon: 165597 |
| | 8501 | |
| | (Synechocystis sp. WH 8501) | |
| Cwa DnaE-c | Crocosphaera watsonii WH | Cyanobacterium, |
| | 8501 | taxon: 165597 |
| | (Synechocystis sp. WH 8501) | |
| Cwa DnaE- | Crocosphaera watsonii WH | Cyanobacterium, |
| n | 8501 | taxon: 165597 |
| | (Synechocystis sp. WH 8501) | |
| Cwa PEP | Crocosphaera watsonii WH | taxon: 165597 |
| | 8501 | |
| | (Synechocystis sp. WH 8501) | |
| Cwa RIR1 | Crocosphaera watsonii WH | taxon: 165597 |
| | 8501 | |
| | (Synechocystis sp. WH 8501) | |
| Daud RIR1 | Candidatus Desulforudis | taxon: 477974 |
| | audaxviator | |
| | MP104C | |
| Dge DnaB | Deinococcus geothermalis | Thermophilic, radiation |
| | DSM11300 | resistant |
| Dha-DCB2 | Desulfitobacterium | Anaerobic |
| RIR1 | hafniense DCB-2 | dehalogenating bacteria, |
| | | taxon: 49338 |
| Dha-Y51 | Desulfitobacterium | Anaerobic |
| RIR1 | hafniense Y51 | dehalogenating bacteria, |
| | | taxon: 138119 |
| Dpr-MLMSI | delta proteobacterium | Taxon: 262489 |
| RIR1 | MLMS-1 | |
| Dra RIR1 | Deinococcus radiodurans R1, | Radiation resistant, |
| | TIGR | |
| | strain | taxon: 1299 |
| Dra Snf2-c | Deinococcus radiodurans R1, | Radiation and DNA |
| | TIGR | damage |
| | strain | resistent, taxon: 1299 |
| Dra Snf2-n | Deinococcus radiodurans R1, | Radiation and DNA |
| | TIGR | damage |
| | strain | resistent, taxon: 1299 |
| Dra- | Deinococcus radiodurans R1, | Radiation and DNA |
| ATCC13939 | ATCC13939/Brooks & | damage |
| Snf2 | Murray strain | resistent, taxon: 1299 |
| Dth UDP | Dictyoglomus | strain = “H-6-12; ATCC |
| GD | thermophilum H-6-12 | 35947, |
| | | taxon: 309799 |
| Dvul ParB | Desulfovibrio vulgaris subsp. | taxon: 391774 |
| | vulgaris DP4 | |
| EP-Min27 | Enterobacteria phage Min27 | bacteriophage of |
| Primase | | host = “Escherichia coli |
| | | O157: H7 str. Min27” |
| Fal DnaB | Frankia alni ACN14a | Plant symbiot, taxon: |
| | | 326424 |
| Fsp-Ccl3 | Frankia species Ccl3 | taxon: 106370 |
| RIR1 | | |
| Gob DnaE | Gemmata | Taxon 114, TIGR |
| | obscuriglobus UQM2246 | genome strain, |
| | | budding bacteria |
| Gob Hyp | Gemmata | Taxon 114, TIGR |
| | obscuriglobus UQM2246 | genome strain, |
| | | budding bacteria |
| Gvi DnaB | Gloeobacter violaceus, PCC | taxon: 33072 |
| | 7421 | |
| Gvi RIR1-1 | Gloeobacter violaceus, PCC | taxon: 33072 |
| | 7421 | |
| Gvi RIR1-2 | Gloeobacter violaceus, PCC | taxon: 33072 |
| | 7421 | |
| Hhal DnaB | Halorhodospira halophila | taxon: 349124 |
| Kfl- | SL1 | |
| DSM17836 | Kribbella flavida DSM 17836 | Taxon: 479435 |
| DnaB | | |
| Kra DnaB | Kineococcus radiotolerans | Radiation resistant |
| | SRS30216 | |
| LLP-KSY1 | Lactococcus phage KSY1 | Bacteriophage, taxon: |
| PolA | | 388452 |
| LP-phiHSIC | Listonella pelagia phage | taxon: 310539, a |
| Helicase | phiHSIC | pseudotemperate marine |
| | | phage of Listonella |
| | | pelagia |
| Lsp- | Lyngbya sp. PCC 8106 | Taxon: 313612 |
| PCC8106 | | |
| GyrB | | |
| MP-Be | Mycobacteriophage | Bacteriophage, taxon: |
| DnaB | Bethlehem | 260121 |
| MP-Be gp51 | Mycobacteriophage | Bacteriophage, taxon: |
| | Bethlehem | 260121 |
| MP-Catera | Mycobacteriophage Catera | Mycobacteriophage, |
| gp206 | | taxon: 373404 |
| MP-KBG | Mycobacterium phage KBG | Taxon: 540066 |
| gp53 | | |
| MP-Mcjw1 | Mycobacteriophage CJW1 | Bacteriophage, taxon: |
| DnaB | | 205869 |
| MP-Omega | Mycobacteriophage Omega | Bacteriophage, taxon: |
| DnaB | | 205879 |
| MP-U2 gp50 | Mycobacteriophage U2 | Bacteriophage, taxon: |
| | | 260120 |
| Maer- | Microcystis aeruginosa | Bloom-forming toxic |
| NIES843 | NIES-843 | cyanobacterium, taxon: |
| DnaB | | 449447 |
| Maer- | Microcystis aeruginosa | Bloom-forming toxic |
| NIES843 | NIES-843 | cyanobacterium, taxon: |
| DnaE-c | | 449447 |
| Maer- | Microcystis aeruginosa | Bloom-forming toxic |
| NIES843 | NIES-843 | cyanobacterium, taxon: |
| DnaE-n | | 449447 |
| Mau- | Micromonospora | Taxon: 644283 |
| ATCC27029 | aurantiaca ATCC | |
| GyrA | 27029 | |
| Mav-104 | Mycobacterium avium 104 | taxon: 243243 |
| DnaB | | |
| Mav- | Mycobacterium | Taxon: 553481 |
| ATCC25291 | avium subsp. avium | |
| DnaB | ATCC 25291 | |
| May- | Mycobacterium avium | ATCC35712, taxon 1764 |
| ATCC35712 | | |
| DnaB | | |
| Mav-PT | Mycobacterium avium subsp. | taxon: 262316 |
| DnaB | paratuberculosis str. k10 | |
| Mbo Pps1 | Mycobacterium | strain = “AF2122/97”, |
| | bovis subsp. bovis | |
| | AF2122/97 | taxon: 233413 |
| Mbo RecA | Mycobacterium | taxon: 233413 |
| | bovis subsp. bovis | |
| | AF2122/97 | |
| Mbo SufB | Mycobacterium | taxon: 233413 |
| (Mbo Pps1) | bovis subsp. bovis | |
| | AF2122/97 | |
| Mbo-1173P | Mycobacterium bovis BCG | strain = BCG Pasteur |
| DnaB | Pasteur | 1173P2,, taxon: 410289 |
| | 1173P | |
| Mbo- | Mycobacterium | strain = “AF2122/97”, |
| AF2122 | bovis subsp. bovis | |
| DnaB | AF2122/97 | taxon: 233413 |
| Mca MupF | Methylococcus | prophage MuMc02, |
| | capsulatus Bath, | taxon: 243233 |
| | prophage MuMc02 | |
| Mca RIR1 | Methylococcus | taxon: 243233 |
| | capsulatus Bath | |
| Mch RecA | Mycobacterium chitae | IP14116003, taxon: 1792 |
| Mcht- | Microcoleus chthonoplastes | Cyanobacterium, |
| PCC7420 | PCC7420 | taxon: 118168 |
| DnaE-1 | | |
| Mcht- | Microcoleus chthonoplastes | Cyanobacterium, |
| PCC7420 | PCC7420 | taxon: 118168 |
| DnaE-2c | | |
| Mcht- | Microcoleus chthonoplastes | Cyanobacterium, |
| PCC7420 | PCC7420 | taxon: 118168 |
| DnaE-2n | | |
| Mcht- | Microcoleus | Taxon: 118168 |
| PCC7420 | chthonoplastes PCC | |
| GyrB | 7420 | |
| Mcht- | Microcoleus | Taxon: 118168 |
| PCC7420 | chthonoplastes PCC | |
| RIR1-1 | 7420 | |
| Mcht- | Microcoleus | Taxon: 118168 |
| PCC7420 | chthonoplastes PCC | |
| RIR1-2 | 7420 | |
| Mex | Methylobacterium | Alphaproteobacteria |
| Helicase | extorquens AM1 | |
| Mex TrbC | Methylobacterium | Alphaproteobacteria |
| | extorquens AM1 | |
| Mfa RecA | Mycobacterium fallax | CITP8139, taxon: 1793 |
| Mfl GyrA | Mycobacterium | taxon: 1776, reference |
| | flavescens Fla0 | #930991 |
| Mfl RecA | Mycobacterium | strain = Fla0, taxon: |
| | flavescens Fla0 | 1776, ref. |
| | | #930991 |
| Mfi- | Mycobacterium flavescens, | strain = ATCC14474, |
| ATCC14474 | ATCC14474 | taxon: 1776, |
| RecA | | ref #930991 |
| Mfl-PYR- | Mycobacterium | taxon: 350054 |
| GCK DnaB | flavescens PYR- | |
| | GCK | |
| Mga GyrA | Mycobacterium gastri | HP4389, taxon: 1777 |
| Mga RecA | Mycobacterium gastri | HP4389, taxon: 1777 |
| Mga SufB | Mycobacterium gastri | HP4389, taxon: 1777 |
| (Mga Pps1) | | |
| Mgi-PYR- | Mycobacterium gilvum PYR- | taxon: 350054 |
| GCK DnaB | GCK | |
| Mgi-PYR- | Mycobacterium gilvum PYR- | taxon: 350054 |
| GCK GyrA | GCK | |
| Mgo GyrA | Mycobacterium gordonae | taxon: 1778, reference |
| | | number |
| | | 930835 |
| Min-1442 | Mycobacterium intracellulare | strain 1442, taxon: 1767 |
| DnaB | | |
| Min- | Mycobacterium | Taxon: 487521 |
| ATCC13950 | intracellulare ATCC | |
| GyrA | 13950 | |
| Mkas GyrA | Mycobacterium kansasii | taxon: 1768 |
| Mkas- | Mycobacterium | Taxon: 557599 |
| ATCC12478 | kansasii ATCC | |
| GyrA | 12478 | |
| Mle-Br4923 | Mycobacterium leprae | Taxon: 561304 |
| GyrA | Br4923 | |
| Mle-TN | Mycobacterium leprae, strain | Human pathogen, taxon: |
| DnaB | TN | 1769 |
| Mle-TN | Mycobacterium leprae TN | Human pathogen, |
| GyrA | | STRAIN = TN, taxon: |
| | | 1769 |
| Mle-TN | Mycobacterium leprae, strain | Human pathogen, taxon: |
| RecA | TN | 1769 |
| Mle-TN | Mycobacterium leprae | Human pathogen, taxon: |
| SufB (Mle | | 1769 |
| Pps1) | | |
| Mma GyrA | Mycobacterium malmoense | taxon: 1780 |
| Mmag | Magnetospirillum | Gram negative, taxon: |
| Magn8951 | magnetofacticum | 272627 |
| BIL | MS-1 | |
| Msh RecA | Mycobacterium shimodei | ATCC27962, |
| | | taxon: 29313 |
| Msm DnaB- | Mycobacterium | MC2 155, taxon: 246196 |
| 1 | smegmatis MC2 155 | |
| Msm DnaB- | Mycobacterium | MC2 155, taxon: 246196 |
| 2 | smegmatis MC2 155 | |
| Msp-KMS | Mycobacterium species KMS | taxon: 189918 |
| DnaB | | |
| Msp-KMS | Mycobacterium species KMS | taxon: 189918 |
| GyrA | | |
| Msp-MCS | Mycobacterium species MCS | taxon: 164756 |
| DnaB | | |
| Msp-MCS | Mycobacterium species MCS | taxon: 164756 |
| GyrA | | |
| Mthe RecA | Mycobacterium | ATCC19527, taxon: 1797 |
| | thermoresistibile | |
| Mtu SufB | Mycobacterium | Human pathogen, taxon: |
| (Mtu Pps1) | tuberculosis strains | 83332 |
| | H37Rv & CDC1551 | |
| Mtu-C RecA | Mycobacterium tuberculosis | Taxon: 348776 |
| | C | |
| Mtu- | Mycobacterium tuberculosis, | Human pathogen, taxon: |
| CDC1551 | CDC1551 | 83332 |
| DnaB | | |
| Mtu-CPHL | Mycobacterium tuberculosis | Taxon: 611303 |
| RecA | CPHL_A | |
| Mtu-Canetti | Mycobacterium tuberculosisi | Taxon: 1773 |
| RecA | strain = “Canetti” | |
| Mtu- | Mycobacterium | Taxon: 520140 |
| EAS054 | tuberculosis EAS054 | |
| RecA | | |
| Mtu-F11 | Mycobacterium tuberculosis, | taxon: 336982 |
| DnaB | strain | |
| | F11 | |
| Mtu-H37Ra | Mycobacterium | ATCC 25177, taxon: |
| DnaB | tuberculosis H37Ra | 419947 |
| Mtu-H37Rv | Mycobacterium | Human pathogen, taxon: |
| DnaB | tuberculosis H37Rv | 83332 |
| Mtu-H37Rv | Mycobacterium tuberculosis | Human pathogen, taxon: |
| RecA | H37Rv, Also CDC 1551 | 83332 |
| Mtu- | Mycobacterium | Taxon: 395095 |
| Haarlem | tuberculosis str. | |
| DnaB | Haarlem | |
| Mtu-K85 | Mycobacterium | Taxon: 611304 |
| RecA | tuberculosis K85 | |
| Mtu-R604 | Mycobacterium | Taxon: 555461 |
| RecA-n | tuberculosis ‘98- | |
| | R604 INH-RIF-EM’ | |
| Mtu-So93 | Mycobacterium tuberculosis | Human pathogen, taxon: |
| RecA | So93/sub_species = “Canetti” | 1773 |
| Mtu-T17 | Mycobacterium | Taxon: 537210 |
| RecA-c | tuberculosis T17 | |
| Mtu-T17 | Mycobacterium | Taxon: 537210 |
| RecA-n | tuberculosis T17 | |
| Mtu-T46 | Mycobacterium | Taxon: 611302 |
| RecA | tuberculosis T46 | |
| Mtu-T85 | Mycobacterium | Taxon: 520141 |
| RecA | tuberculosis T85 | |
| Mtu-T92 | Mycobacterium | Taxon: 515617 |
| RecA | tuberculosis T92 | |
| Mvan DnaB | Mycobacterium | taxon: 350058 |
| | vanbaalenii PYR-1 | |
| Mvan GyrA | Mycobacterium | taxon: 350058 |
| | vanbaalenii PYR-1 | |
| Mxa RAD25 | Myxococcus xanthus DK1622 | Deltaproteobacteria |
| Mxe GyrA | Mycobacterium xenopi strain | taxon: 1789 |
| | IMM5024 | |
| Naz-0708 | Nostoc azollae 0708 | Taxon: 551115 |
| RIR1-1 | | |
| Naz-0708 | Nostoc azollae 0708 | Taxon: 551115 |
| RIR1-2 | | |
| Nfa DnaB | Nocardia farcinica | taxon: 247156 |
| Nfa | IFM 10152 | |
| Nfa15250 | Nocardia farcinica | taxon: 247156 |
| | IFM 10152 | |
| Nfa RIR1 | Nocardia farcinica | taxon: 247156 |
| | IFM 10152 | |
| Nosp- | Nodularia | Taxon: 313624 |
| CCY9414 | spumigena CCY9414 | |
| DnaE-n | | |
| Npu DnaB | Nostoc punctiforme | Cyanobacterium, taxon: |
| | | 63737 |
| Npu GyrB | Nostoc punctiforme | Cyanobacterium, taxon: |
| | | 63737 |
| Npu- | Nostoc | Cyanobacterium, taxon: |
| PCC73102 | punctiforme PCC73102 | 63737, |
| DnaE-c | | ATCC29133 |
| Npu- | Nostoc | Cyanobacterium, taxon: |
| PCC73102 | punctiforme PCC73102 | 63737, |
| DnaE-n | | ATCC29133 |
| Nsp-JS614 | Nocardioides species JS614 | taxon: 196162 |
| DnaB | | |
| Nsp-JS614 | Nocardioides species JS614 | taxon: 196162 |
| TOPRIM | | |
| Nsp- | Nostoc species PCC7120, | Cyanobacterium, |
| PCC7120 | (Anabaena | Nitrogen-fixing, |
| DnaB | sp. PCC7120) | taxon: 103690 |
| Nsp- | Nostoc species PCC7120, | Cyanobacterium, |
| PCC7120 | (Anabaena | Nitrogen-fixing, |
| DnaE-c | sp. PCC7120) | taxon: 103690 |
| Nsp- | Nostoc species PCC7120, | Cyanobacterium, |
| PCC7120 | (Anabaena | Nitrogen-fixing, |
| DnaE-n | sp. PCC7120) | taxon: 103690 |
| Nsp- | Nostoc species PCC7120, | Cyanobacterium, |
| PCC7120 | (Anabaena | Nitrogen-fixing, |
| RIR1 | sp. PCC7120) | taxon: 103690 |
| Oli DnaE-c | Oscillatoria limnetica str. | Cyanobacterium, taxon: |
| | ‘Solar Lake’ | 262926 |
| Oli DnaE-n | Oscillatoria limnetica str. | Cyanobacterium, taxon: |
| | ‘Solar Lake’ | 262926 |
| PP-PhiEL | Pseudomonas | Phage |
| Helicase | aeruginosa phage | infects Pseudomonas |
| | phiEL | aeruginosa, |
| | | taxon: 273133 |
| PP-PhiEL | Pseudomonas | phage |
| ORF11 | aeruginosa phage | infects Pseudomonas |
| | phiEL | aeruginosa, |
| | | taxon: 273133 |
| PP-PhiEL | Pseudomonas | Phage |
| ORF39 | aeruginosa phage | infects Pseudomonas |
| | phiEL | aeruginosa, |
| | | taxon: 273133 |
| PP-PhiEL | Pseudomonas | phage |
| ORF40 | aeruginosa phage | Infects Pseudomonas |
| | phiEL | aeruginosa, |
| | | taxon: 273133 |
| Pfl Fha BIL | Pseudomonas fluorescens Pf- | Plant commensal |
| | 5 | organism, |
| | | taxon: 220664 |
| Plut RIR1 | Pelodictyon luteolum DSM | Green sulfur bacteria, |
| | 273 | Taxon |
| | | 319225 |
| Pma-EXH1 | Persephonella marina EX- | Taxon: 123214 |
| GyrA | H1 | |
| Pma-ExH1 | Persephonella marina EX- | Taxon: 123214 |
| DnaE | H1 | |
| Pna RIR1 | Polaromonas | taxon: 365044 |
| | naphthalenivorans CJ2 | |
| Pnuc DnaB | Polynucleobacter sp. QLW- | taxon: 312153 |
| | P1DMWA-1 | |
| Posp-JS666 | Polaromonas species JS666 | taxon: 296591 |
| DnaB | | |
| Posp-JS666 | Polaromonas species JS666 | taxon: 296591 |
| RIR1 | | |
| Pssp-A1-1 | Pseudomonas species A1-1 | |
| Fha | | |
| Psy Fha | Pseudomonas syringae pv. | Plant (tomato) pathogen, |
| | tomato | taxon: 223283 |
| | str. DC3000 | |
| Rbr-D9 | Raphidiopsis brookii D9 | Taxon: 533247 |
| GyrB | | |
| Rce RIR1 | Rhodospirillum centenum | taxon: 414684, ATCC |
| | SW | 51521 |
| Rer-SK121 | Rhodococcus | Taxon: 596309 |
| DnaB | erythropolis SK121 | |
| Rma DnaB | Rhodothermus marinus | Thermophile, |
| | | taxon: 29549 |
| Rma- | Rhodothermus marinus DSM | Taxon: 518766 |
| DSM4252 | 4252 | |
| DnaB | | |
| Rma- | Rhodothermus marinus DSM | Thermophile, taxon: |
| DSM4252 | 4252 | 518766 |
| DnaE | | |
| Rsp RIR1 | Roseovarius species 217 | taxon: 314264 |
| SaP- | Salmonella phage SETP12 | Phage, taxon: 424946 |
| SETP12 | | |
| dpol | | |
| SaP-SETP3 | Salmonella phage SETP3 | Phage, taxon: 424944 |
| Helicase | | |
| SaP-SETP3 | Salmonella phage SETP3 | Phage, taxon: 424944 |
| dpol | | |
| SaP-SETP5 | Salmonella phage SETP5 | Phage, taxon: 424945 |
| dpol | | |
| Sare DnaB | Salinispora arenicola CNS- | taxon: 391037 |
| | 205 | |
| Sav RecG | Streptomyces avermitilis | taxon: 227882, ATCC |
| Helicase | MA-4680 | 31267 |
| Sel-PC6301 | Synechococcus | taxon: 269084 Berkely |
| RIR1 | elongatus PCC 6301 | strain |
| | and PCC7942 | 6301~equivalent name: |
| | | Ssp |
| | | PCC 6301~synonym: |
| | | Anacystis nudulans |
| Sel-PC7942 | Synechococcus | taxon: 1140 |
| DnaE-C | elongatus PC7942 | |
| Sel-PC7942 | Synechococcus | taxon: 1140 |
| DnaE-n | elongatus PC7942 | |
| Sel-PC7942 | Synechococcus | taxon: 1140 |
| RIR1 | elongatus PC7942 | |
| Sel- | Synechococcus | Cyanobacterium, |
| PCC6301 | elongatus PCC 6301 | |
| DnaE-C | and PCC7942 | taxon: 269084, “Berkely |
| | | strain |
| | | 6301~equivalent name: |
| | | Synechococcus sp. PCC |
| | | 6301~synonym: |
| | | Anacystis nudulans” |
| Sel- | Synechococcus | Cyanobacterium, |
| PCC6301 | elongatus PCC 6301 | taxon: 269084 “Berkely |
| DnaE-n | | strain |
| | | 6301~equivalent name: |
| | | Synechococcus sp. PCC |
| | | 6301~synonym: |
| | | Anacystis nudulans” |
| Sep RIR1 | Staphylococcus | taxon: 176279 |
| | epidermidis RP62A | |
| ShP-Sfv-2a- | Shigella flexneri 2a str. | Putative bacteriophage |
| 2457T-n | 2457T | |
| Primase | | |
| ShP-Sfv-2a- | Shigella flexneri 2a str. 301 | Putative bacteriophage |
| 301-n | | |
| Primase | | |
| ShP-Sfv-5 | Shigella flexneri 5 str. 8401 | Bacteriphage, |
| Primase | | isolation_source |
| | | epidemic, taxon: 373384 |
| SoP-SO1 | Sodalis phage SO-1 | Phage/isolation_source = |
| dpol | | “Sodalis |
| | | glossinidius strain GA- |
| | | SG, secondary symbiont |
| | | of Glossina |
| | | austeni (Newstead)” |
| Spl DnaX | Spirulina platensis, strain C1 | Cyanobacterium, taxon: |
| | | 1156 |
| Sru DnaB | Salinibacter ruber | taxon: 309807, strain = |
| | DSM 13855 | “DSM |
| | | 13855; M31” |
| Sru PolBc | Salinibacter ruber | taxon: 309807. strain = |
| | DSM 13855 | “DSM |
| | | 13855; M31” |
| Sru RIR1 | Salinibacter ruber | taxon: 309807, strain = |
| | DSM 13855 | “DSM |
| | | 13855, M31” |
| Ssp DnaB | Synechocystis species, strain | Cyanobacterium, taxon: |
| | PCC6803 | 1148 |
| Ssp DnaE-c | Synechocystis species, strain | Cyanobacterium, taxon: |
| | PCC6803 | 1148 |
| Ssp DnaE-n | Synechocystis species, strain | Cyanobacterium, taxon: |
| | PCC6803 | 1148 |
| Ssp DnaX | Synechocystis species, strain | Cyanobacterium, taxon: |
| | PCC6803 | 1148 |
| Ssp GyrB | Synechocystis species, strain | Cyanobacterium, taxon: |
| | PCC6803 | 1148 |
| Ssp-JA2 | Synechococcus species JA-2- | Cyanobacterium, Taxon: |
| DnaB | 3B'a(2-13) | 321332 |
| Ssp-JA2 | Synechococcus species JA-2- | Cyanobacterium, Taxon: |
| RIR1 | 3B'a(2-13) | 321332 |
| Ssp-JA3 | Synechococcus species JA-3- | Cyanobacterium, Taxon: |
| DnaB | 3Ab | 321327 |
| Ssp-JA3 | Synechococcus species JA-3- | Cyanobacterium, Taxon: |
| RIR1 | 3Ab | 321327 |
| Ssp- | Synechocystis species, strain | Cyanobacterium, taxon: |
| PCC7002 | PCC | 32049 |
| DnaE-c | 7002 | |
| Ssp- | Synechocystis species, strain | Cyanobacterium, taxon: |
| PCC7002 | PCC | 32049 |
| DnaE-n | 7002 | |
| Ssp- | Synechococcus sp. PCC 7335 | Taxon: 91464 |
| PCC7335 | | |
| RIR1 | | |
| StP-Twort | Staphylococcus phage Twort | Phage, taxon 55510 |
| ORF6 | | |
| Susp- | Sulfurovum sp. NBC37-1 | taxon: 387093 |
| NBC371 | | |
| DnaB | | |
| intein | | |
| Taq- | Thermus aquaticus | Taxon: 498848 |
| Y51MC23 | Y51MC23 | |
| DnaE | | |
| Taq- | Thermus aquaticus | Taxon: 498848 |
| Y51MC23 | Y51MC23 | |
| RIR1 | | |
| Tcu- | Thermomonospora | Taxon: 471852 |
| DSM43183 | curvata DSM | |
| RecA | 43183 | |
| Tel DnaE-c | Thermosynechococcus | Cyanobacterium, taxon: |
| | elongatus BP-1 | 197221 |
| Tel DnaE-n | Thermosynechococcus | Cyanobacterium, |
| | elongatus BP-1 | |
| Ter DnaB-1 | Trichodesmium | Cyanobacterium, taxon; |
| | erythraeum IMS101 | 203124 |
| Ter DnaB-2 | Trichodesmium | Cyanobacterium, taxon: |
| | erythraeum IMS101 | 203124 |
| Ter DnaE-1 | Trichodesmium | Cyanobacterium, taxon: |
| | erythraeum IMS101 | 203124 |
| Ter DnaE-2 | Trichodesmium | Cyanobacterium, taxon: |
| | erythraeum IMS101 | 203124 |
| Ter DnaE- | Trichodesmium | Cyanobacterium, taxon: |
| 3c | erythraeum IMS101 | 203124 |
| Ter DnaE- | Trichodesmium | Cyanobacterium, taxon: |
| 3n | erythraeum IMS101 | 203124 |
| Ter GyrB | Trichodesmium | Cyanobacterium, taxon: |
| | erythraeum IMS101 | 203124 |
| Ter Ndse-1 | Trichodesmium | Cyanobacterium, taxon: |
| | erythraeum IMS101 | 203124 |
| Ter Ndse-2 | Trichodesmium | Cyanobacterium, taxon: |
| | erythraeum IMS101 | 203124 |
| Ter RIR1-1 | Trichodesmium | Cyanobacterium, taxon: |
| | erythraeum IMS101 | 203124 |
| Ter RIR1-2 | Trichodesmium | Cyanobacterium, taxon: |
| | erythraeum IMS101 | 203124 |
| Ter RIR1-3 | Trichodesmium | Cyanobacterium, taxon: |
| | erythraeum IMS101 | 203124 |
| Ter RIR1-4 | Trichodesmium | Cyanobacterium, taxon: |
| | erythraeum IMS101 | 203124 |
| Ter Snf2 | Trichodesmium | Cyanobacterium, taxon: |
| | erythraeum IMS101 | 203124 |
| Ter ThyX | Trichodesmium | Cyanobacterium, taxon: |
| | erythraeum IMS101 | 203124 |
| Tfus RecA-1 | Thermobifida fusca YX | Thermophile, taxon: |
| | | 269800 |
| Tfus RecA-2 | Thermobifida fusca YX | Thermophile, taxon: |
| | | 269800 |
| Tfus | Thermobifida fusca YX | Thermophile, taxon: |
| Tfu2914 | | 269800 |
| Thsp-K90 | Thioalkalivibrio sp. K90mix | Taxon: 396595 |
| RIR1 | | |
| Tth- | Thermoanaerobacterium | Taxon: 580327 |
| DSM571 | thermosaccharolyticum DSM | |
| RIR1 | 571 | |
| Tth-HB27 | Thermus thermophilus HB27 | thermophile, taxon: |
| DnaE-1 | | 262724 |
| Tth-HB27 | Thermus thermophilus HB27 | thermophile, taxon: |
| DnaE-2 | | 262724 |
| Tth-HB27 | Thermus thermophilus HB27 | thermophile, taxon: |
| RIR1-1 | | 262724 |
| Tth-HB27 | Thermus thermophilus HB27 | thermophile, taxon: |
| RIR1-2 | | 262724 |
| Tth-HB8 | Thermus thermophilus HB8 | thermophile, taxon: |
| DnaE-1 | | 300852 |
| Tth-HB8 | Thermus thermophilus HB8 | thermophile, taxon: |
| DnaE-2 | | 300852 |
| Tth-HB8 | Thermus thermophilus HB8 | thermophile, taxon: |
| RIR1-1 | | 300852 |
| Tth-HB8 | Thermus thermophilus HB8 | thermophile, taxon: |
| RIR1-2 | | 300852 |
| Tvu DnaE-c | Thermosynechococcus | Cyanobacterium, taxon: |
| | vulcanus | 32053 |
| Tvu DnaE-n | Thermosynechococcus | Cyanobacterium, taxon: |
| | vulcanus | 32053 |
| Tye RNR-1 | Thermodesulfovibrio | taxon: 289376 |
| | yellowstonii | |
| | DSM 11347 | |
| Tye RNR-2 | Thermodesulfovibrio | taxon: 289376 |
| | yellowstonii | |
| | DSM 11347 | |
| Archaea | | |
| Ape | Aeropyrum pernix K1 | Thermophile, |
| APE0745 | | taxon: 56636 |
| Cme-boo | Candidatus Methanoregula | taxon: 456442 |
| Pol-II | boonei | |
| | 6A8 | |
| Fac-Fer1 | Ferroplasma acidarmanus, | strain Fer1, eats iron |
| RIR1 | taxon: 97393 and taxon | |
| | 261390 | |
| Fac-Fer1 | Ferroplasma acidarmanus | strain fer1, eats |
| SufB (Fac | | iron, taxon: 97393 |
| Pps1) | | |
| Fac-TypeI | Ferroplasma | Eats iron, taxon 261390 |
| RIR1 | acidarmanus type I, | |
| Fac-typeI | Ferroplasma acidarmanus | Eats iron, taxon: 261390 |
| SufB (Fac | | |
| Pps1) | | |
| Hma | Haloarcula marismortui | taxon: 272569, |
| CDC21 | ATCC 43049 | |
| Hma Pol-II | Haloarcula marismortui | taxon: 272569, |
| | ATCC 43049 | |
| Hma PolB | Haloarcula marismortui | taxon: 272569, |
| | ATCC 43049 | |
| Hma TopA | Haloarcula marismortui | taxon: 272569 |
| | ATCC 43049 | |
| Hmu- | Halomicrobium | taxon: 485914 |
| DSM12286 | mukohataei DSM | (Halobacteria) |
| MCM | 12286 | |
| Hmu- | Halomicrobium | Taxon: 485914 |
| DSM12286 | mukohataei DSM | |
| PolB | 12286 | |
| Hsa-R1 | Halobacterium salinarum R-1 | Halophile, |
| MCM | | taxon: 478009, strain = |
| | | “R1; |
| | | DSM 671” |
| Hsp-NRC1 | Halobacterium species | Halophile, taxon: 64091 |
| CDC21 | NRC-1 | |
| Hsp-NRC1 | Halobacterium | Halophile, taxon: 64091 |
| Pol-II | salinarum NRC-1 | |
| Hut MCM-2 | Halorhabdus utahensis DSM | taxon: 519442 |
| | 12940 | |
| Hut- | Halorhabdus utahensis DSM | taxon: 519442 |
| DSM12940 | 12940 | |
| MCM- | | |
| 1 | | |
| Hvo PolB | Haloferax volcanii DS70 | taxon: 2246 |
| Hwa GyrB | Haloquadratum walsbyi DSM | Halophile, taxon: 362976, |
| | 16790 | strain: DSM 16790 = |
| | | HBSQ001 |
| Hwa MCM-1 | Haloquadratum walsbyi DSM | Halophile, taxon: 362976, |
| | 16790 | strain: DSM 16790 = |
| | | HBSQ001 |
| Hwa MCM-2 | Haloquadratum walsbyi DSM | Halophile, taxon: 362976, |
| | 16790 | strain: DSM 16790 = |
| | | HBSQ001 |
| Hwa MCM-3 | Haloquadratum walsbyi DSM | Halophile, taxon: 362976, |
| | 16790 | strain: DSM 16790 = |
| | | HBSQ001 |
| Hwa MCM-4 | Haloquadratum walsbyi DSM | Halophile, taxon: 362976, |
| | 16790 | strain: DSM 16790 = |
| | | HBSQ001 |
| Hwa Pol-II-1 | Haloquadratum walsbyi DSM | Halophile, taxon: 362976, |
| | 16790 | strain: DSM 16790 = |
| | | HBSQ001 |
| Hwa Pol-II-2 | Haloquadratum walsbyi DSM | Halophile, taxon: 362976, |
| | 16790 | strain: DSM 16790 = |
| | | HBSQ001 |
| Hwa PolB-1 | Haloquadratum walsbyi DSM | Halophile, taxon: 362976, |
| | 16790 | strain: DSM 16790 = |
| | | HBSQ001 |
| Hwa PolB-2 | Haloquadratum walsbyi DSM | Halophile, taxon: 362976, |
| | 16790 | strain: DSM 16790 = |
| | | HBSQ001 |
| Hwa PolB-3 | Haloquadratum walsbyi DSM | Halophile, taxon: 362976, |
| | 16790 | strain: DSM 16790 = |
| | | HBSQ001 |
| Hwa RCF | Haloquadratum walsbyi DSM | Halophile, taxon: 362976, |
| | 16790 | strain: DSM 16790 = |
| | | HBSQ001 |
| Hwa RIR1-1 | Haloquadratum walsbyi DSM | Halophile, taxon: 362976, |
| | 16790 | strain: DSM 16790 = |
| | | HBSQ001 |
| Hwa RIR1-2 | Haloquadratum walsbyi DSM | Halophile, taxon: 362976, |
| | 16790 | strain: DSM 16790 = |
| | | HBSQ001 |
| Hwa Top6B | Haloquadratum walsbyi DSM | Halophile, taxon: 362976, |
| | 16790 | strain: DSM 16790 = |
| | | HBSQ001 |
| Hwa rPol A″ | Haloquadratum walsbyi DSM | Halophile, taxon: 362976, |
| | 16790 | strain: DSM 16790 = |
| | | HBSQ001 |
| Maeo Pol-II | Methanococcus | taxon: 419665 |
| | aeolicus Nankai-3 | |
| Maeo RFC | Methanococcus | taxon: 419665 |
| | aeolicus Nankai-3 | |
| Maeo RNR | Methanococcus | taxon: 419665 |
| | aeolicus Nankai-3 | |
| Maeo-N3 | Methanococcus | taxon: 419665 |
| Helicase | aeolicus Nankai-3 | |
| Maeo-N3 | Methanococcus | taxon: 419665 |
| RtcB | aeolicus Nankai-3 | |
| Maeo-N3 | Methanococcus | taxon: 419665 |
| UDP GD | aeolicus Nankai-3 | |
| Mein-ME | Methanocaldococcus | thermophile, Taxon: |
| PEP | infernus ME | 573063 |
| Mein-ME | Methanocaldococcus | |
| RFC | infernus ME | Taxon: 573063 |
| Memar | Methanoculleus | taxon: 368407 |
| MCM2 | marisnigri JR1 | |
| Memar Pol- | Methanoculleus | taxon: 368407 |
| II | marisnigri JR1 | |
| Mesp- | Methanocaldococcus sp. | Taxon: 644281 |
| FS406 | FS406-22 | |
| PolB-1 | | |
| Mesp- | Methanocaldococcus sp. | Taxon: 644281 |
| FS406 | FS406-22 | |
| PolB-2 | | |
| Mesp- | Methanocaldococcus sp. | Taxon: 644281 |
| FS406 | | |
| PolB-3 | FS406-22 | |
| Mesp- | Methanocaldococcus sp. | Taxon: 644281 |
| FS406-22 | FS406-22 | |
| LHR | | |
| Mfe-AG86 | Methanocaldococcus | Taxon: 573064 |
| Pol-1 | fervens AG86 | |
| Mfe-AG86 | Methanocaldococcus | Taxon: 573064 |
| Pol-2 | fervens AG86 | |
| Mhu Pol-II | Methanospirillum | taxon 323259 |
| | hungateii JF-1 | |
| Mja GF-6P | Methanococcus jannaschii | Thermophile, DSM |
| | (Methanocaldococcus | 2661, taxon: 2190 |
| | jannaschii | |
| | DSM 2661) | |
| Mja | Methanococcus jannaschii | Thermophile, DSM |
| Helicase | (Methanocaldococcus | 2661, taxon: 2190 |
| | jannaschii | |
| | DSM 2661) | |
| Mja Hyp-1 | Methanococcus jannaschii | Thermophile, DSM |
| | (Methanocaldococcus | 2661, taxon: 2190 |
| | jannaschii | |
| | DSM 2661) | |
| Mja IF2 | Methanococcus jannaschii | Thermophile, DSM |
| | (Methanocaldococcus | 2661, taxon: 2190 |
| | jannaschii | |
| | DSM 2661) | |
| Mja KlbA | Methanococcus jannaschii | Thermophile, DSM |
| | (Methanocaldococcus | 2661, taxon: 2190 |
| | jannaschii | |
| | DSM 2661) | |
| Mja PEP | Methanococcus jannaschii | Thermophile, DSM |
| | (Methanocaldococcus | 2661, taxon: 2190 |
| | jannaschii | |
| | DSM 2661) | |
| Mja Pol-1 | Methanococcus jannaschii | Thermophile, DSM |
| | (Methanocaldococcus | 2661, taxon: 2190 |
| | jannaschii | |
| | DSM 2661) | |
| Mja Pol-2 | Methanococcus jannaschii | Thermophile, DSM |
| | (Methanocaldococcus | 2661, taxon: 2190 |
| | jannaschii | |
| | DSM 2661) | |
| Mja RFC-1 | Methanococcus jannaschii | Thermophile, DSM |
| | (Methanocaldococcus | 2661, taxon: 2190 |
| | jannaschii | |
| | DSM 2661) | |
| Mja RFC-2 | Methanococcus jannaschi | Thermophile, DSM |
| | (Methanocaldococcus | 2661, taxon: 2190 |
| | jannaschii | |
| | DSM 2661) | |
| Mja RFC-3 | Methanococcus jannaschii | Thermophile, DSM |
| | (Methanocaldococcus | 2661, taxon: 2190 |
| | jannaschii | |
| | DSM 2661) | |
| Mja RNR-1 | Methanococcus jannaschii | Thermophile, DSM |
| | (Methanocaldococcus | 2661, taxon: 2190 |
| | jannaschii | |
| | DSM 2661) | |
| Mja RNR-2 | Methanococcus jannaschii | Thermophile, DSM |
| | (Methanocaldococcus | 2661, taxon: 2190 |
| | jannaschii | |
| | DSM 2661) | |
| Mja RtcB | Methanococcus jannaschii | Thermophile, DSM |
| (Mja Hyp-2) | (Methanocaldococcus | 2661, taxon: 2190 |
| | jannaschii | |
| | DSM 2661) | |
| Mja TFIIB | Methanococcus jannaschii | Thermophile, DSM |
| | (Methanocaldococcus | 2661, taxon: 2190 |
| | jannaschii | |
| | DSM 2661) | |
| Mja UDP | Methanococcus jannaschii | Thermophile, DSM |
| GD | (Methanocaldococcus | 2661, taxon: 2190 |
| | jannaschii | |
| | DSM 2661) | |
| Mja r-Gyr | Methanococcus jannaschii | Thermophile, DSM |
| | (Methanocaldococcus | 2661, taxon: 2190 |
| | jannaschii | |
| | DSM 2661) | |
| Mja rPol A′ | Methanococcus jannaschii | Thermophile, DSM |
| | (Methanocaldococcus | 2661, taxon: 2190 |
| | jannaschii | |
| | DSM 2661) | |
| Mja rPol A″ | Methanococcus jannaschii | Thermophile, DSM |
| | (Methanocaldococcus | 2661, taxon: 2190 |
| | jannaschii | |
| | DSM 2661) | |
| Mka CDC48 | Methanopyrus kandleri AV19 | Thermophile, taxon: |
| | | 190192 |
| Mka EF2 | Methanopyrus kandleri AV19 | Thermophile, taxon: |
| | | 190192 |
| Mka RFC | Methanopyrus kandleri AV19 | Thermophile, taxon: |
| | | 190192 |
| Mka RtcB | Methanopyrus kandleri AV19 | Thermophile, taxon: |
| | | 190192 |
| Mka VatB | Methanopyrus kandleri AV19 | Thermophile, taxon: |
| | | 190192 |
| Mth RIR1 | Methanothermobacter | Thermophile, delta |
| | thermautotrophicus | H strain |
| | (Methanobacterium | |
| | thermoautotrophicum) | |
| Mvu-M7 | Methanocaldococcus | Taxon: 579137 |
| Helicase | vulcanius M7 | |
| Mvu-M7 | Methanocaldococcus | Taxon: 579137 |
| Pol-1 | vulcanius M7 | |
| Mvu-M7 | Methanocaldococcus | Taxon: 579137 |
| Pol-2 | vulcanius M7 | |
| Mvu-M7 | Methanocaldococcus | Taxon: 579137 |
| Pol-3 | vulcanius M7 | |
| Mvu-M7 | Methanocaldococcus | Taxon: 579137 |
| UDP GD | vulcanius M7 | |
| Neq Pol-c | Nanoarchaeum equitans Kin4- | Thermophile, taxon: |
| | M | 228908 |
| Neq Pol-n | Nanoarchaeum equitans Kin4- | Thermophile, taxon: |
| | M | 228908 |
| Nma- | Natrialba magadii ATCC | Taxon: 547559 |
| ATCC43099 | 43099 | |
| MCM | | |
| Nma- | Natrialba magadii ATCC | Taxon: 547559 |
| ATCC43099 | 43099 | |
| PolB-1 | | |
| Nma- | Natrialba magadii ATCC | Taxon: 547559 |
| ATCC43099 | 43099 | |
| PolB-2 | | |
| Nph CDC21 | Natronomonas | taxon: 348780 |
| | pharaonis DSM 2160 | |
| Nph PolB-1 | Natronomonas | taxon: 348780 |
| | pharaonis DSM 2160 | |
| Nph PolB-2 | Natronomonas | taxon: 348780 |
| | pharaonis DSM 2160 | |
| Nph rPol A″ | Natronomonas | taxon: 348780 |
| | pharaonis DSM 2160 | |
| Pab | Pyrococcus abyssi | Thermophile, strain |
| CDC21-1 | | Orsay, taxon: 29292 |
| Pab | Pyrococcus abyssi | Thermophile, strain |
| CDC21-2 | | Orsay, taxon: 29292 |
| Pab IF2 | Pyrococcus abyssi | Thermophile, strain |
| | | Orsay, taxon: 29292 |
| Pab KlbA | Pyrococcus abyssi | Thermophile, strain |
| | | Orsay, taxon: 29292 |
| Pab Lon | Pyrococcus abyssi | Thermophile, strain |
| | | Orsay, taxon: 29292 |
| Pab Moaa | Pyrococcus abyssi | Thermophile, strain |
| | | Orsay, taxon: 29292 |
| Pab Pol-II | Pyrococcus abyssi | Thermophile, strain |
| | | Orsay, taxon: 29292 |
| Pab RFC-1 | Pyrococcus abyssi | Thermophile, strain |
| | | Orsay, taxon: 29292 |
| Pab RFC-2 | Pyrococcus abyssi | Thermophile, strain |
| | | Orsay, taxon: 29292 |
| Pab RIR1-1 | Pyrococcus abyssi | Thermophile, strain |
| | | Orsay, taxon: 29292 |
| Pab RIR1-2 | Pyrococcus abyssi | Thermophile, strain |
| | | Orsay, taxon: 29292 |
| Pab RIR1-3 | Pyrococcus abyssi | Thermophile, strain |
| | | Orsay, taxon: 29292 |
| Pab RtcB | Pyrococcus abyssi | Thermophile, strain |
| (Pab Hyp-2) | | Orsay, taxon: 29292 |
| Pab VMA | Pyrococcus abyssi | Thermophile, strain |
| | | Orsay, taxon: 29292 |
| Par RIR1 | Pyrobaculum | taxon: 340102 |
| | arsenaticum DSM | |
| | 13514 | |
| Pfu CDC21 | Pyrococcus furiosus | Thermophile, taxon: |
| | | 186497, |
| | | DSM3638 |
| Pfu IF2 | Pyrococcus furiosus | Thermophile, taxon: |
| | | 186497, |
| | | DSM3638 |
| Pfu KlbA | Pyrococcus furiosus | Thermophile, taxon: |
| | | 186497, |
| | | DSM3638 |
| Pfu Lon | Pyrococcus furiosus | Thermophile, taxon: |
| | | 186497, |
| | | DSM3638 |
| Pfu RFC | Pyrococcus furiosus | Thermophile, DSM3638, |
| | | taxon: 186497 |
| Pfu RIR1-1 | Pyrococcus furiosus | Thermophile, taxon: |
| | | 186497, |
| | | DSM3638 |
| Pfu RIR1-2 | Pyrococcus furiosus | Thermophile, taxon: |
| | | 186497, |
| | | DSM3638 |
| Pfu RtcB | Pyrococcus furiosus | Thermophile, taxon: |
| (Pfu Hyp-2) | | 186497, |
| | | DSM3638 |
| Pfu TopA | Pyrococcus furiosus | Thermophile, taxon: |
| | | 186497, |
| | | DSM3638 |
| Pfu VMA | Pyrococcus furiosus | Thermophile, taxon: |
| | | 186497, |
| | | DSM3638 |
| Pho | Pyrococcus horikoshii OT3 | Thermophile, taxon: 53953 |
| CDC21-1 | | |
| Pho | | |
| CDC21-2 | Pyrococcus horikoshii OT3 | Thermophile, taxon: 53953 |
| Pho IF2 | Pyrococcus horikoshii OT3 | Thermophile, taxon: 53953 |
| Pho KlbA | Pyrococcus horikoshii OT3 | Thermophile, taxon: 53953 |
| Pho LHR | Pyrococcus horikoshii OT3 | Thermophile, taxon: 53953 |
| Pho Lon | Pyrococcus horikoshii OT3 | Thermophile, taxon: 53953 |
| Pho Pol I | Pyrococcus horikoshii OT3 | Thermophile, taxon: 53953 |
| Pho Pol-II | Pyrococcus horikoshii OT3 | Thermophile, taxon: 53953 |
| Pho RFC | Pyrococcus horikoshii OT3 | Thermophile, taxon: 53953 |
| Pho RIR1 | Pyrococcus horikoshii OT3 | Thermophile, taxon: 53953 |
| Pho RadA | Pyrococcus horikoshii OT3 | Thermophile, taxon: 53953 |
| Pho RtcB | Pyrococcus horikoshii OT3 | Thermophile, taxon: 53953 |
| (Pho Hyp-2) | | |
| Pho VMA | Pyrococcus horikoshii OT3 | Thermophile, taxon: 53953 |
| Pho r-Gyr | Pyrococcus horikoshii OT3 | Thermophile, taxon: 53953 |
| Psp-GBD | Pyrococcus species GB-D | Thermophile |
| Pol | | |
| Pto VMA | Picrophilus torridus, DSM | DSM 9790, taxon: 263820, |
| | 9790 | Thermoacidophile |
| Smar 1471 | Staphylothermus marinus F1 | taxon: 399550 |
| Smar MCM2 | Staphylothermus marinus F1 | taxon: 399550 |
| Tac- | Thermoplasma acidophilum, | Thermophile, taxon: 2303 |
| ATCC25905 | ATCC | |
| VMA | 25905 | |
| Tac- | Thermoplasma acidophilum, | Thermophile, taxon: 2303 |
| DSM1728 | DSM1728 | |
| VMA | | |
| Tag Pol-1 | Thermococcus aggregans | Thermophile, taxon: |
| (Tsp-TY | | 110163 |
| Pol-1) | | |
| Tag Pol-2 | Thermococcus aggregans | Thermophile, taxon: |
| (Tsp-TY | | 110163 |
| Pol-2) | | |
| Tag Pol-3 | Thermococcus aggregans | Thermophile, taxon: |
| (Tsp-TY | | 110163 |
| Pol-3) | | |
| Tba Pol-II | Thermococcus barophilus MP | taxon: 391623 |
| Tfu Pol-1 | Thermococcus fumicolans | Thermophilem, taxon: |
| | | 46540 |
| Tfu Pol-2 | Thermococcus fumicolans | Thermophile, taxon: 46540 |
| Thy Pol-1 | Thermococcus hydrothermalis | Thermophile, taxon: 46539 |
| Thy Pol-2 | Thermococcus hydrothermalis | Thermophile, taxon: 46539 |
| Tko CDC21- | Thermococcus | Thermophile, taxon: 69014 |
| 1 | kodakaraensis KOD1 | |
| Tko CDC21- | Thermococcus | Thermophile, taxon: 69014 |
| 2 | kodakaraensis KOD1 | |
| Tko | Thermococcus | Thermophile, taxon: 69014 |
| Helicase | kodakaraensis KOD1 | |
| Tko IF2 | Thermococcus | Thermophile, taxon: 69014 |
| | kodakaraensis KOD1 | |
| Tko KlbA | Thermococcus | Thermophile, taxon: 69014 |
| | kodakaraensis KOD1 | |
| Tko LHR | Thermococcus | Thermophile, taxon: 69014 |
| | kodakaraensis KOD1 | |
| Tko Pol-1 | Pyrococcus/Thermococcus | Thermophile, taxon: 69014 |
| (Pko Pol-1) | kodakaraensis KOD1 | |
| Tko Pol-2 | Pyrococcus/Thermococcus | Thermophile, taxon: 69014 |
| (Pko Pol-2) | kodakaraensis KOD1 | |
| Tko PoI-II | Thermococcus | Thermophile, taxon: 69014 |
| | kodakaraensis KOD1 | |
| Tko RFC | Thermococcus | Thermophile, taxon: 69014 |
| | kodakaraensis KOD1 | |
| Tko RIR1-1 | Thermococcus | Thermophile, taxon: 69014 |
| | kodakaraensis KOD1 | |
| Tko RIR 1-2 | Thermococcus | Thermophile, taxon: 69014 |
| | kodakaraensis KOD1 | |
| Tko RadA | Thermococcus | Thermophile, taxon: 69014 |
| | kodakaraensis KOD1 | |
| Tko TopA | Thermococcus | Thermophile, taxon: 69014 |
| | kodakaraensis KOD1 | |
| Tko r-Gyr | Thermococcus | Thermophile, taxon: 69014 |
| | kodakaraensis KOD1 | |
| Tli Pol-1 | Thermococcus litoralis | Thermophile, taxon: 2265 |
| Tli Pol-2 | Thermococcus litoralis | Thermophile, taxon: 2265 |
| Tma Pol | Thermococcus marinus | taxon: 187879 |
| Ton-NA1 | Thermococcus | Taxon: 523850 |
| LHR | onnurineus NA1 | |
| Ton-NA1 | Thermococcus | taxon: 342948 |
| Pol | onnurineus NA1 | |
| Tpe Pol | Thermococcus | taxon: 32644 |
| | peptonophilus strain | |
| | SM2 | |
| Tsi-MM739 | Thermococcus sibiricus MM | Thermophile, Taxon. |
| Lon | 739 | 604354 |
| Tsi-MM739 | Thermococcus sibiricus MM | Taxon: 604354 |
| Pol-1 | 739 | |
| Tsi-MM739 | Thermococcus sibiricus MM | Taxon: 604354 |
| Pol-2 | 739 | |
| Tsi-MM739 | Thermococcus sibiricus MM | Taxon: 604354 |
| RFC | 739 | |
| Tsp AM4 | Thermococcus sp. AM4 | Taxon: 246969 |
| RtcB | | |
| Tsp-AM4 | Thermococcus sp. AM4 | Taxon: 246969 |
| LHR | | |
| Tsp-AM4 | Thermococcus sp. AM4 | Taxon: 246969 |
| Lon | | |
| Tsp-AM4 | Thermococcus sp. AM4 | Taxon: 246969 |
| RIR1 | | |
| Tsp-GE8 | Thermococcus species GE8 | Thermophile, taxon: |
| Pol-1 | | 105583 |
| Tsp-GE8 | Thermococcus species GE8 | Thermophile, taxon: |
| Pol-2 | | 105583 |
| Tsp-GT Pol- | Thermococcus species GT | taxon: 370106 |
| 1 | | |
| Tsp-GT Pol- | Thermococcus species GT | taxon: 370106 |
| 2 | | |
| Tsp-OGL- | Thermococcus sp. OGL-20P | taxon: 277988 |
| 20P Pol | | |
| Tthi Pol | Thermococcus thioreducens | Hyperthermophile |
| Tvo VMA | Thermoplasma | Thermophile, |
| | volcanium GSS1 | taxon: 50339 |
| Tzi Pol | Thermococcus zilligii | taxon: 54076 |
| Unc-ERS | uncultured archaeon | isolation_source = “Eel |
| PFL | Gzfos13E1 | River |
| | | sediment”, |
| | | clone = “GZfos13E1”, |
| | | taxon: 285397 |
| Unc-ERS | uncultured archaeon | isolation_source = “Eel |
| RIR1 | GZfos9C4 | River |
| | | sediment”, taxon: 285366, |
| | | clone = “GZfos9C4″ |
| Unc-ERS | uncultured archaeon | isolation_source = “Eel |
| RNR | GZfos10C7 | River |
| | | sediment”. |
| | | clone = “GZfos10C7”, |
| | | taxon: 285400 |
| Unc- | uncultured archaeon (Rice | Enriched methanogenic |
| MetRFS | Cluster I) | consortium from rice field |
| MCM2 | | soil, taxon: 198240 |

In some embodiments, the coding sequence of the exogenous polypeptide can be split into three (or more) portions.

Split inteins can mediate the efficient post-translational splicing of two or more heterologous extein polypeptides. However, the resulting spliced product generally includes three to five amino acids of intein sequence introduced at the junction of the spliced amino- and carboxy-terminal extein polypeptides. In some instances, these three to five “intein footprint” (or simply “footprint”) amino acids do not appreciably affect the function of the final spliced polypeptide, but in others, the presence of such inserted amino acids can negatively impact the structure and function of the final product. As such, there can be a benefit to minimizing or even altogether avoiding an intein footprint in the trans-spliced protein product.

In one aspect, an intein footprint insert can be minimized or even completely avoided in the methods and compositions as described herein. To achieve this, one can, for example, analyze the sequence of a target protein relative to known split intein footprints to identify sequences within the target that match or closely approximate a split intein's footprint.

TABLE 4
Exemplary sequences to minimize split intein footprints

| | 6A.A | | | |
|---|---|---|---|---|
| | conventional | | | Minimal footprint |
| | footprint | | | with high protein |
| Split Intein ID | N-ter | C-ter | Footprint tested | trans-splicing rate |
| Split Aha, | AEY | CFN | AEYCFN, AEYCF, AEYC, AEY | AEY (only for split |
| Split Sel | | | EYCFN, EYCF, EYC, EY, | Aha using GFP) |
| | | | YCFN, YCF, YC, Y | |
| | | | CFN, CF, C | |
| Split gp41.1 | SGY | SSS | SGYSSS, SGYSS, SGYS, SGY | GYS |
| | | | GYSSS, GYSS, GYS, GY | |
| | | | YSSS, YSS, YS, Y | |
| | | | SSS, SS, S | |
| Split IMPDH | GGG | SIC | GGGSIC, GGGSI, GGGS, GGG | GS |
| | | | GGSIC, GGSI, GGS, GG | |
| | | | GSIC, GSI, GS, G | |
| | | | SIC, SI, S | |
| Nrdj-1 | NPC | SEI | NPCSEI, NPCSE, NPCS, NPC | CS |
| | | | PCSEI, PCSE, PCS, PC | |
| | | | CSEI, CSE, CS, C | |
| | | | SEI, SE, S | |

Amino-acids abbreviation:
A: Alanine
C: Cysteine
E: Glutamic Acid
F: Phenylalanine
G: Glycine
I: Isoleucine
N: Asparagine
P: Proline
S: Serine
Y: Tyrosine

One can then use such a split intein that has a footprint naturally occurring in a given target protein to design the heterologous extein-intein fusions to be separately expressed, and thereby minimize or even avoid the insertion of non-naturally occurring amino acids in the spliced polypeptide product. For example, after screening the target protein sequence for sequences that match split intein footprint sequence, one can prepare sequences encoding amino- and carboxy-terminal fusions of the target polypeptide fragments to the respective amino and carboxy-terminal spit intein fragments in which the footprint amino acids are omitted from the extein fusion polypeptide sequences. In this situation, upon cleavage and joinder of the extein sequences, the intein footprint insert reconstitutes the native target polypeptide sequence, resulting in a spliced target polypeptide that does not differ in amino acid sequence from the natural target polypeptide. That is, while there is technically still a footprint insert characteristic of that split intein, its sequence matches sequence occurring in the target protein, such that there is no non-native footprint in the resulting spliced polypeptide product.

In some instances, a given target polypeptide may lack an exact match to a split intein footprint, or an exact match may be located so close to the amino or carboxy terminus of the target protein that splitting the sequence encoding the target protein at that point does not divide the target protein coding sequence into fragments that will each fit into a delivery vector. In such instances, it can still be beneficial to identify sequences within the target protein that are similar, but not identical to a split intein footprint sequence. Such similarity can be, for example, matching four out of five footprint amino acids, three out of five footprint amino acids, or even two out of five footprint amino acids. Similarity in this context can also include, for example, the inclusion of amino acids with similar properties to those in the footprint, e.g., amino acids that are conservative substitutions for the naturally-occurring amino acids, or a combination of matches and conservative substitutions. Used analogously to the situation in which an exact match to an intein footprint can be identified in a beneficial location in a target protein, such an approach based on footprint similarity can minimize the intein footprint and/or its impact on function of the spliced target protein. Thus, a spliced product with an intein footprint of four or fewer differences, three or fewer, two or fewer, one or fewer, or no differences relative to the naturally occurring or desired target protein sequence can be generated as described herein.

In some embodiments, an “engineered” split intein differs from a naturally occurring polypeptide or nucleic acid by one or more amino acid or nucleic acid deletions, additions, substitutions or side-chain modifications, yet retains one or more specific functions or biological activities of the naturally occurring split intein sequence. Amino acid substitutions include alterations in which an amino acid is replaced with a different naturally-occurring or a non-conventional amino acid residue. Some substitutions can be classified as “conservative,” in which case an amino acid residue contained in a polypeptide is replaced with another naturally occurring amino acid of similar character either in relation to polarity, side chain functionality or size. Substitutions encompassed by variants as described herein can also be “non-conservative,” in which an amino acid residue which is present in a peptide is substituted with an amino acid having different properties (e.g., substituting a charged or hydrophobic amino acid with an uncharged or hydrophilic amino acid), or alternatively, in which a naturally-occurring amino acid is substituted with a non-conventional amino acid.

In one embodiment, the split intein comprises at least two of SEQ ID Nos: 1-46. In another embodiment, the split intein comprises SEQ ID NO: 1 and SEQ ID NO: 2. In another embodiment, the split intein comprises SEQ ID NO: 3 and SEQ ID NO: 4. In another embodiment, the split intein comprises SEQ ID NO: 5 and SEQ ID NO: 6. In another embodiment, the split intein comprises SEQ ID NO: 7 and SEQ ID NO: 8. In another embodiment, the split intein comprises SEQ ID NO: 9 and SEQ ID NO: 10. In another embodiment, the split intein comprises SEQ ID NO: 11 and SEQ ID NO: 12. In another embodiment, the split intein comprises SEQ ID NO: 13 and SEQ ID NO: 14. In another embodiment, the split intein comprises SEQ ID NO: 15 and SEQ ID NO: 16. In another embodiment, the split intein comprises SEQ ID NO: 17 and SEQ ID NO: 18. In another embodiment, the split intein comprises SEQ ID NO: 19 and SEQ ID NO: 20. In another embodiment, the split intein comprises SEQ ID NO: 21 and SEQ ID NO: 22. In another embodiment, the split intein comprises SEQ ID NO: 23 and SEQ ID NO: 24. In another embodiment, the split intein comprises SEQ ID NO: 25 and SEQ ID NO: 26. In another embodiment, the split intein comprises SEQ ID NO: 27 and SEQ ID NO: 28. In another embodiment, the split intein comprises SEQ ID NO: 29 and SEQ ID NO: 30. In another embodiment, the split intein comprises SEQ ID NO: 31 and SEQ ID NO: 32. In another embodiment, the split intein comprises SEQ ID NO: 33 and SEQ ID NO: 34. In another embodiment, the split intein comprises SEQ ID NO: 35 and SEQ ID NO: 36. In another embodiment, the split intein comprises SEQ ID NO: 37 and SEQ ID NO: 38. In another embodiment, the split intein comprises SEQ ID NO: 39 and SEQ ID NO: 40. In another embodiment, the split intein comprises SEQ ID NO: 41 and SEQ ID NO: 42. In another embodiment, the split intein comprises SEQ ID NO: 43 and SEQ ID NO: 44. In another embodiment, the split intein comprises SEQ ID NO: 45 and SEQ ID NO: 46.

Exemplary sequences for specific dystrophin, dysferlin, utrophin, and mini-dystrophin fragments with corresponding inteins are provided herein in Example 2.

### MSECs

In certain embodiments, the split intein constructs described herein can benefit from cell-type-specific expression. Such a design can ensure expression, including high level, moderate level or low level or regulated expression of the target protein not only where it is most needed, but also avoid or limit potential negative impact of ectopic expression in non-target cells or tissues. Inclusion of a tissue-specific expression cassette can thus maximize therapeutic benefit of transgene introduction. Such a design can also, for example, facilitate or permit systemic administration of vectors, in that while infection may occur in non-target cells or tissues, expression of the transgene polypeptide(s) will substantially only occur in the desired cell or tissue type. When used in combination with, for example, a vector that has a tropism or enhanced tropism for transduction of a given tissue or cell type, the use of a tissue specific expression cassette to drive expression of each target protein-split intein construct as described herein can be highly beneficial. When used in the context of delivery of two or more vectors, multiple tissue specific expression cassettes can be used to generate balanced ratios of, for example, mRNA production or accumulation, or protein translation, production or accumulation.

A “tissue-specific expression cassette,” as the term is used herein, provides expression of a target protein in a manner restricted to a particular tissue or cell type. By “restricted to” or “in a restricted manner” in this context is meant that expression from the construct is at least 5-fold higher in the target tissue or cell type than in other tissues or cell types, e.g., at least 5-fold higher, 10-fold higher, 15-fold higher, 20-fold higher or more. Expression can be measured at the level of, for example, mRNA production or accumulation, or at the level of protein translation, production or accumulation. In one embodiment, a tissue-specific expression cassette is a “muscle-specific expression cassette,” or “MSEC” as described herein. An MSEC will drive expression of a linked construct in a muscle cell- or muscle tissue-restricted manner as that term is defined herein above.

MSECs generally include elements of muscle-specific promoters and enhancers. See, for example, Salva et al., Molecular Therapy 15:320-329 (2007), which is incorporated herein by reference, for examples and discussion of muscle-specific expression cassettes designed for use in rAAV vectors to drive heterologous protein expression in skeletal and cardiac muscle. Muscle-specific expression cassettes include, for example, promoter and enhancer sequence elements derived from muscle-specific genes including muscle creatine kinase (MCK), skeletal «-actin and «-myosin heavy-chain genes, among others. The murine MCK gene includes a 206 bp enhancer located approximately 1.2 kb upstream of the transcription start site, and a 358 bp proximal promoter. However, for use in gene therapy vectors such as AAV, the viral packaging limits as discussed herein require that regulatory elements designed to drive muscle-specific expression be kept to a minimum (about 800 bp or less) in order to maximize the amount of payload protein coding sequence for a given vector. Thus, muscle-specific expression cassettes useful in the methods and compositions described herein are comprised of truncated/modified muscle-specific regulatory elements that provide binding sites for myogenic regulatory factors, as well as Inr (initiator element) and/or TATA box sequences, and can include, for example, additional sequences from the 5′ untranslated region of muscle-specific genes. The MHCK7 cassette described by Salva et al. is but one example of an MSEC useful in the methods and compositions described herein. That cassette drives expression to a higher degree than the constitutively active CMV promoter in MM14 myocytes, but is essentially inactive in non-muscle cells (e.g., HEK 293 fibroblasts, murine L cell fibroblasts, and JAWSII dendritic cells). See also the expression cassettes described in U.S. Pat. No. 10,479,821, which is incorporated herein by reference. As but one example, SEQ ID NO: 19 described therein and referred to as CK8, is highly active in cardiac and skeletal muscle. It is contemplated that variants of such MSEC sequences can also provide highly active, muscle-specific expression of therapeutic transgenes. For example, a sequence having at least 80%, 85%, 90%, 95%, 96%, 97%, 98%, 99% or greater identity to such MSECs can also be of use in the methods and compositions described herein. One of skill in the art can determine the activity of a given MSEC in muscle cells or tissue, e.g., using assays as described in the Salva et al. publication.

### Pharmaceutical Compositions

Provided herein are vector compositions that are useful for treating or preventing a variety of different diseases and/or disorders in a subject. An important subset of disease and disorders is muscle diseases and disorders. In one embodiment, the composition is a pharmaceutical composition. The composition can comprise a therapeutically or prophylactically effective amount of at least two vectors encoding an exogenous polynucleotide or therapeutic agent. The at least two vectors utilize split inteins to aid in delivery of large protein-encoding nucleic acids to a given cell.

The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions. Formulations suitable for parenteral administration can be formulated, for example, for intravenous, intramuscular, intradermal, intraperitoneal, and subcutaneous routes. Carriers can include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

In one embodiment, the composition is formulated for intramuscular delivery.

Therapeutic compositions contain a physiologically tolerable carrier together with the vectors described herein, dissolved or dispersed therein as an active ingredient. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmaceutical composition that contains active ingredients dissolved or dispersed therein is understood in the art and need not be limited based on formulation. Typically, such compositions are prepared as injectable either as liquid solutions or suspensions; however, solid forms suitable for solution, or suspension in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition for use with the methods described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Examples of such additional liquid phases are glycerin, vegetable oils such as cottonseed oil, and water-oil emulsions. The amount of a vector to be administered herein that will be effective in the treatment of a particular disorder or condition will depend on the nature of the disorder or condition, the expression of the therapeutic agent, and can be determined by standard clinical techniques.

While any suitable carrier known to those of ordinary skill in the art can be employed in the pharmaceutical composition, the type of carrier will vary depending on the mode of administration. Compositions for use as described herein can be formulated for any appropriate manner of administration, including for example, topical, oral, nasal, intravenous, intracranial, intraperitoneal, subcutaneous or intramuscular administration. For parenteral administration, such as intramuscular or subcutaneous injection, the carrier preferably comprises water, saline, alcohol, a fat, a wax or a buffer. Alternatively, compositions as described herein can be formulated as a lyophilizate. Compounds can also be encapsulated within liposomes.

### Dosage and Administration

Treatment using the methods and compositions described herein includes both prophylaxis/prevention of disease onset and therapy of an active disease. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals as well as other veterinary subjects. Preferably, the patients or subjects are human.

In one aspect, the methods described herein provide a method for treating a disease or disorder in a subject (e.g., a muscle disease or disorder). In one embodiment, the subject can be a mammal. In another embodiment, the mammal can be a human, although the approach is effective with respect to all mammals. The method comprises administering to the subject an effective amount of a pharmaceutical composition comprising vector as described herein in a pharmaceutically acceptable carrier.

The dosage range for the agent depends upon the potency, the expression level of the therapeutic protein and includes amounts large enough to produce the desired effect, e.g., reduction in at least one symptom of the disease to be treated. The dosage should not be so large as to cause unacceptable adverse side effects. Generally, the dosage will vary with the type of exogenous protein expressed from the vector (e.g., recombinant polypeptide, peptide, peptidomimetic, small molecule, etc.), the therapeutic protein characteristics (e.g., dystrophin, utrophin, dysferlin, etc) and with the age, condition, and sex of the patient. The dosage can be determined by one of skill in the art and can also be adjusted by the individual physician in the event of any complication.

In some embodiments, the vectors are administered at a multiplicity of infection (MOI) of at least 5, at least 10, at least 20, at least 30, at least 40, at least 50, at least 100, at least 200, at least 500 or more.

In certain embodiments, the vectors are administered at a titer of at least 1×10⁵, 1×10⁶, 1×10⁷, 1×10⁸, 1×10⁹, 1×10¹⁰, 1×10¹¹, 1×10¹² viral particles or more.

Repeated administration can be performed as necessary to maintain therapeutic efficacy. As used herein, the term “therapeutically effective amount” refers to an amount of a vector or expressed therapeutic agent that is sufficient to produce a statistically significant, measurable change in at least one symptom of a disease (see “Efficacy Measurement” below). Alternatively, a therapeutically effective amount is an amount of a vector or expressed therapeutic protein that is sufficient to produce a statistically significant, measurable change in the expression level of a biomarker associated with the disease in the subject. Such effective amounts can be gauged in clinical trials as well as animal studies for a given agent.

The vector compositions can be administered directly to a particular site (e.g., intramuscular injection, intravenous infusion, into a specific organ) or can be administered orally. It is also contemplated herein that the agents can also be delivered intravenously (by bolus or continuous infusion), by inhalation, intranasally, intraperitoneally, intramuscularly, subcutaneously, intracavity, and can be delivered by peristaltic means, if desired, or by other means known by those skilled in the art. The agent can be administered systemically, if so desired.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

### Efficacy Measurement

The efficacy of a given treatment for a disease can be determined by the skilled clinician. However, a treatment is considered “effective treatment,” as the term is used herein, if any one or all of the signs or symptoms of the disease to be treated is/are altered in a beneficial manner, other clinically accepted symptoms or markers of disease are improved, or even ameliorated, e.g., by at least 10% following treatment with a vector as described herein. Efficacy can also be measured by failure of an individual to worsen as assessed by stabilization of the disease, hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the disease; or (2) relieving the disease, e.g., causing regression of symptoms; and (3) preventing or reducing the likelihood of the development of the disease or preventing secondary issues associated with the disease.

In some embodiments, efficacy of treatment of a muscle disease or disorder can be determined by assessing one or more parameters of muscle function including, but not limited to, specific force generation, mobility, spasticity, tension, stability etc. In some embodiments, clinical tests for determining an improvement in muscle function, such as electromyography, magnetic resonance imaging (MRI) or muscle biopsies, can be used to assess efficacy of a method of treatment as described herein.

It is understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

The following provides non-limiting Examples demonstrating and supporting the technology as described herein.

### Example 1

### Summary

Duchenne muscular dystrophy (DMD) is among the most common human genetic disorders, affecting approximately 1 in 5,000 newborn males (Emery 2003). It results from genetic mutations in the DMD gene that prevent expression of functional dystrophin (Monaco 1985, Kunkel 1986), which is one of the largest proteins made by the human cells. Adeno-associated viral (AAV) vector-based gene delivery has been actively used to treat DMD (Crudele 2019). However, the main limitation associated with the delivery of the DMD gene is its large coding sequences (11 kb) (Koenig 1989, Chamberlain 1989), while the maximum AAV cargo capacity is less than 5 kb (Srivastava 1983). Previous work by the inventors included the development of miniaturized ‘micro-dystrophin’ (μDys) that can be packaged within a single AAV vector (Harper 2002, Gregorevic 2006). These truncated, albeit functional, dystrophins have been shown to improve the skeletal muscle force and histology when administrated to dystrophin-deficient animal models despite missing critical domains of dystrophin required for its mechanical and signaling functions and the impossibility of transporting them all in one AAV vector.

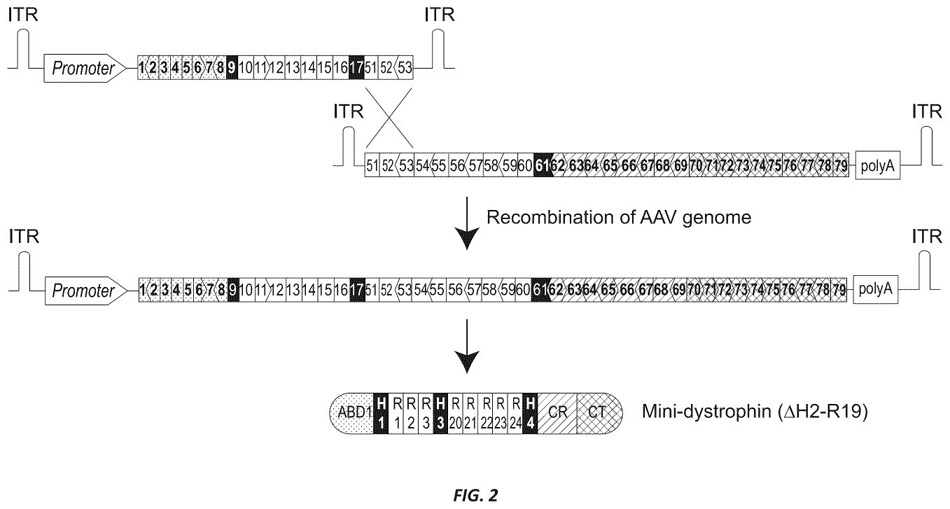

Gene therapies using AAV vectors hold a promising future for treating different loss-of-function genetic disorders (Li 2020). However, this therapeutic modality has been challenged by the small packaging capacity of this viral vector (−5 kb). Due to the large size of the DMD coding sequences (−11.2 kb), the current therapeutic approaches in development for DMD using AAV vectors aim to express either a miniaturized μDys or genetic elements that restore the open-reading frame (CRISPR/Cas9-mediated gene editing or exon-skipping induced by U7 small nuclear RNA) (Harper 2002, Goyenvalle 2004, Long 2016, Nelson 2016, Bengtsson 2017). In both strategies, smaller than normal dystrophins are produced. In addition, very low expression and unstable secondary structures are observed when one or multiple exons are skipped using CRISPR/Cas9 or U7 tools (most likely because of improperly ‘phased’ repeats; Harper 2002). Previous work has shown the feasibility of reconstituting larger mini- or full-length dystrophins via homologous recombination and following the administration of two or three vectors (FIG. 2) (Odom 2011, Koo 2014, Lostal 2014). However, these events were rare and, in some cases, led to unwanted and potentially toxic products that would jeopardize their clinical use.

Provided herein is a novel therapeutic approach that can deliver larger, possibly up to full-length, dystrophins with high expression and in a precise manner. This method is not limited by unwanted recombination products, and it can be adapted to clinical use for any patient with Duchenne or Becker muscular dystrophies (BMD). This improved strategy allows for the expression of large and stable proteins with high specificity and efficiency (SIMPLI-GT (Split Intein-Mediated Protein Ligation for Gene Therapy). This approach takes advantage of the intrinsic ability of split inteins to mediate protein trans-splicing, and therefore to reconstitute larger therapeutic constructs, which extends the usage of AAV-based gene replacement approach to any gene exceeding the maximum cargo capacity of AAV vectors.

### Study

Gene replacement therapies using AAV vectors hold a great promise for treating genetic disorders caused by loss-of-function mutations. Currently, hundreds of primate serotypes have been isolated and more serotypes are in development (Li 2020). The inventors have previously shown that AAV can be used for systemic gene delivery to both cardiac and skeletal muscles at high efficiency (Gregorevic 2004). Numerous studies have also demonstrated very stable expression lasting up to 8 years in different animal models (Rivera 2005, Niemeyer 2009). In the clinic, several patients affected by neuromuscular disorders such as myotubular myopathy or spinal muscular atrophy have been recently treated with a single dose of AAV vectors to replace the defective gene, MTM or SMN respectively (NCT03199469, NCT02122952) and the clinical data have shown very promising physiological outcomes with the lack of cellular immunogenicity to both transgene and vector.

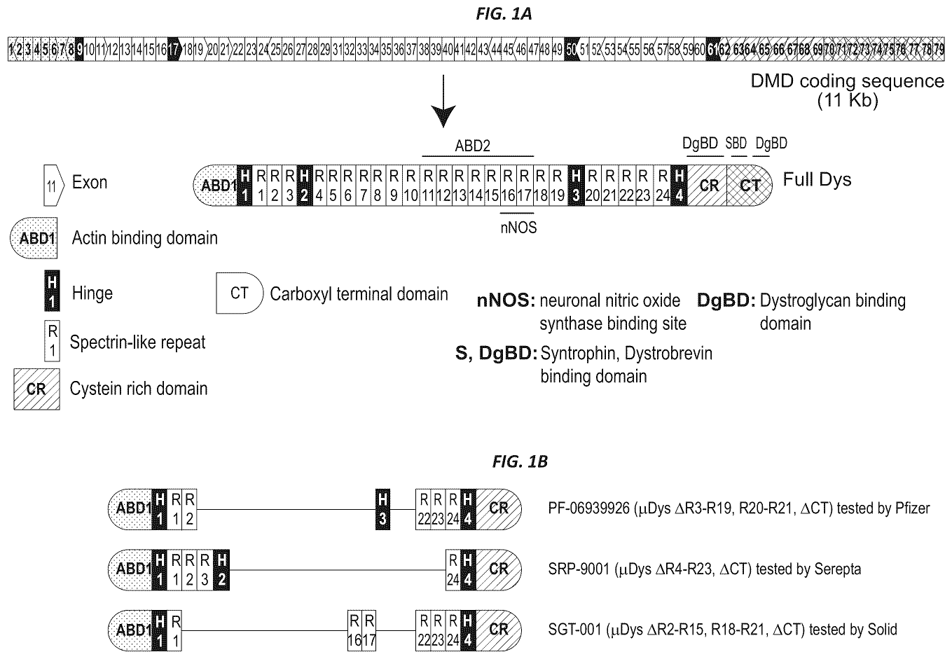

However, one of the main drawbacks of AAV vectors is their limited packaging capacity (˜5 kb), which excludes many genetic disorders from using these vectors as a gene transporter. Due to the large coding sequences of the defective gene in muscular dystrophies like Duchenne or limb-girdle type 2B, a single AAV vector cannot be used to deliver the DMD, or DYSF genes respectively, to the affected muscles. For DMD, a series of miniaturized μDys were previously developed that can be delivered by single AAV vector (FIG. 1) (Harper 2002, Gregorevic 2006, Banks 2010, Ramos 2019). These truncated, albeit functional, dystrophins have been shown to improve the skeletal muscle force and histology when administrated to dystrophin-deficient animal models, and three constructs are currently being tested in human clinical trials, by Sarepta and Solid Biosciences (NCT03375164, NCT03368742).

It is noteworthy that most of the μDys developed to date contain 4 to 6 Spectrin-repeats (SR) with 2 or 3 Hinges, plus the N-terminal actin-binding domain (ABD) and the dystroglycan-binding domain (DgBD) (Ramos, 2019). Thousands of combinations are possible to generate μDys from the 24 SR and 4 Hinges domains that originally constitute the full-length dystrophin. This high number of possible combinations together with the limited AAV carrying capacities make it challenging to predict which μDys would have the best physiological outcomes. Indeed, previous studies revealed that many μDys are not functional and the best constructs cloned so far exhibit incomplete cardiac and skeletal rescue (Wasla 2018, Ramos 2019). This suggests that the therapeutic candidate should include more domains to stabilize the dystrophin structure and provide additional protein functions.

On the other hand, two patients carrying a deletion of 46% (Aexon17-48) of the DMD gene were reported to express a truncated but highly functional dystrophin lacking most of the region between hinge 2 and the middle of SR19, and present a very mild phenotype with normal life span (England 1990), which inspired the development of larger ‘mini-dystrophin’ (mini-Dys) constructs (Phelps 1995, Harper, 2002, Odom 2011). Simultaneous administration of two AAV vectors that express two halves of a mini-Dys (ΔH2-SR19) flanked by a short region of homology allows the recombination of AAV genomes, which leads to the reconstitution the mini-Dys (FIG. 2). This highly functional protein showed a great therapeutic potential when tested in mdx^{4cv} mouse model with improvements in muscle histology and physiological performances. Nonetheless, unwanted products, obtained most likely from unspecific annealing of single-stranded genomes and the formation of concatamers, were detected by Southern blot analysis and by polymerase chain reaction. These unspecific products could potentially be toxic and trigger an immunoreaction which prevents its clinical use.

In the present study, the inventors describe a novel method named SIMPLI-GT (abbreviation of Split Intein-Mediated Protein Ligation for Gene Therapy), which aims to reconstitute larger therapeutic constructs using the protein trans-splicing mechanism mediated by inteins. This approach can overcome the main hurdle related to AAV-based gene replacement and extends its applications to numerous genetic disorders caused by loss-of-function in large genes.

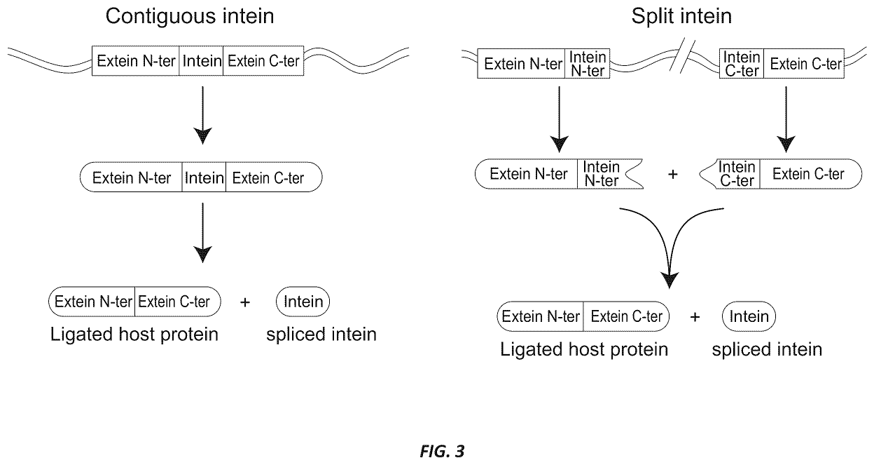

Inteins are genetic elements that are found in unicellular organisms. They are embedded within essential genes that are involved in DNA transcription, replication and maintenance (e.g. DNA or RNA polymerase subunits, helicases, gyrases, and ribonucleotide reductase) or in other housekeeping genes including essential proteases and metabolic enzymes (Shah 2014). Following their in-frame transcription and translation with the host gene, the intein polypeptides (size varies between 138 to 844 amino acids) are self-excised from the precursor protein (also called extein) and join the adjacent peptides. This post-translational modification, known as protein splicing, does not require energy supply, cofactors or exogenous protease intervention. Over 600 inteins have been identified to date and around 30 have the particularity to be encoded by two separate genes. Unlike the more common contiguous inteins, these split inteins are transcribed and translated separately in N- and C-intein fragments. Then, they associate and form one reconstituted complex (N-extein/N-intein/C-intein/C-extein) before spontaneous splicing of the intein, resulting in reconstituted and fully functional extein (host protein) (FIG. 3).

This protein trans-splicing mechanism is used in biotechnological applications including protein purification and labeling steps (Li 2015). The inventors propose to utilize split inteins to reconstitute larger proteins that cannot be delivered by a single AAV vector due to its packaging limitation. Therefore, they have generated a library of 23 split inteins in order to screen for their ability to reconstitute two polypeptide fragments into one functional protein. This pre-screening will be performed using the green fluorescent protein (GFP) as screening platform, which will permit testing of several inteins under the same conditions and in an unbiased and reliable manner. GFP is a widely used protein that has revolutionized different biology fields due to its small size (238 amino acids), easiness, specificity and lack of cell toxicity. It was previously adapted as a scaffold to screen aptamers and small anti-bacterial peptides (Abedi 1988, Soundrarajan 2016).

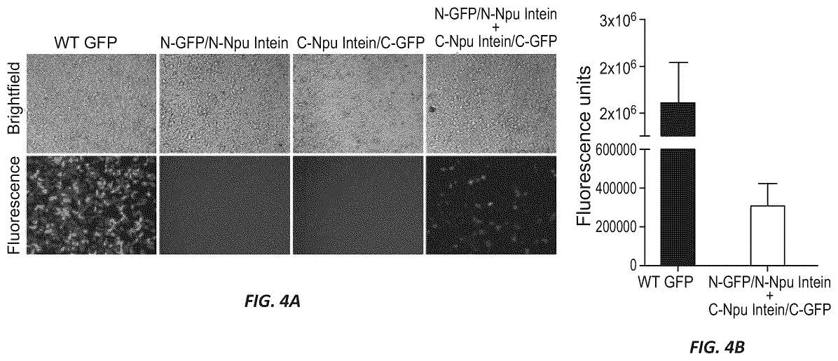

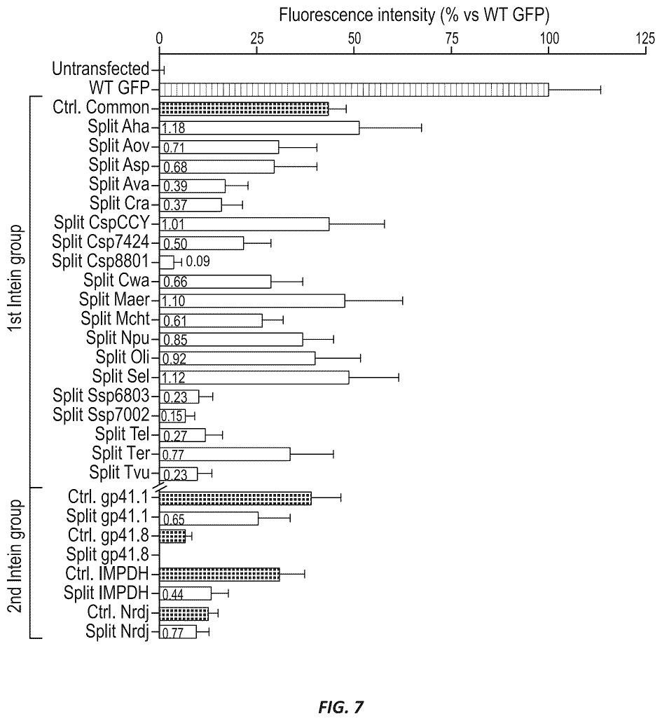

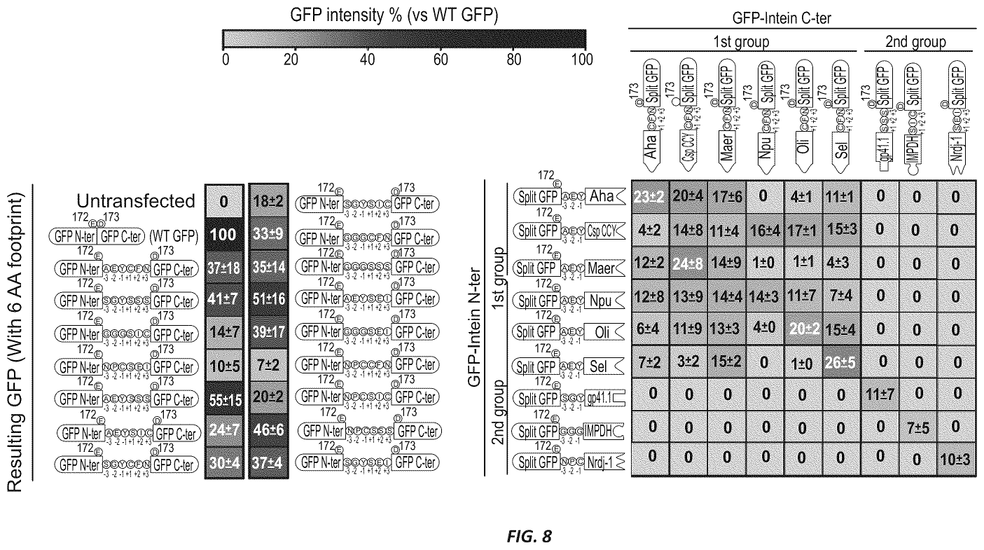

To begin, the inventors identified a splitting site in the GFP protein sequence where N- and C-terminal inteins can be inserted. In a preliminary test, two plasmids were cloned that encode either the N- or the C-terminal half of GFP fused to the N- or the C-terminal half of Npu intein (one of the most studied intein, which is found in nostoc punctiforme cyanobacterium). Then, human embryonic kidney 293 (HEK293) cells were co-transfected with both N- and/or C-terminal GFP/intein plasmids. 24 hours later, GFP fluorescence was detected only in cells transfected with either WT GFP (full-length GFP expressed from one plasmid) or dual split GFP/intein plasmids but not with the single N- or C-terminal plasmid (FIG. 4A). These data indicate that GFP was efficiently reassembled through protein trans-splicing mediated by Npu intein. The GFP fluorescence intensity was measured in living cells using a spectrophotometer. It was found that the GFP signal from the reconstituted protein was lower than that from WT GFP (FIG. 4B). This could be due to the short transfection period and the time required by the different steps prior to the formation of functional GFP (i.e., transcription, translation, fusion of N- and C-terminal fragments, intein splicing and ligation of the two N- and C-terminal GFP). In addition, the inventors observed other inteins that are currently being screened have a higher activity. The initial test showed the feasibility of using split GFP to test the ability of inteins to reconstitute a full-length GFP that is able to emit a measurable fluorescence. The inventors continued to screen the intein library using this accurate system, and results from a variety of additional split inteins is shown in FIG. 7.

Next, the ability of inteins to join two halves of mini-Dys was tested. With the small size of the split inteins, a dual AAV vector approach allows the expression of the largest mini-Dys construct tested so far. In silico modeling showed that two AAV vectors can transport the coding sequences of two dystrophin fragments: an N-terminal clone encoding proteins from N-terminus to the end of the SR19, but lacking SRs 5-15, while the C-terminal clone encodes sequences from Hinge3 through the C-terminus domain. Thus, the reconstituted mini-Dys mediated by the split intein trans-splicing contains 4 hinges, 13 SRs, the ABD, CR and CT domains. Unlike the improperly-phased-repeats in dystrophins that are frequently generated with exon skipping or gene editing (Harper, 2002), this novel mini-Dys (ΔSR5-15) carries only full spectrin-like repeats that will stabilize its secondary structure and molecular folding. More importantly, this mini-Dys (ΔSR5-15) is larger than the highly functional ΔH2-SR19 dystrophin found in very mild Becker patients (discussed in the background section). The new mini-Dys harbors several functional domains including actin, dystrotroglycan, dystrobrevin, syntrophin, and the neuronal nitric oxide synthase (nNOS) binding sites, which are important for its mechanical and signaling roles.

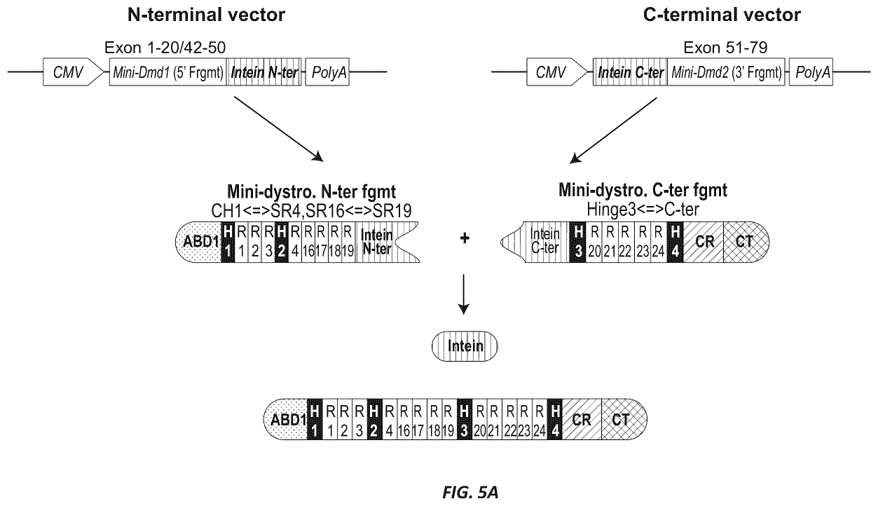

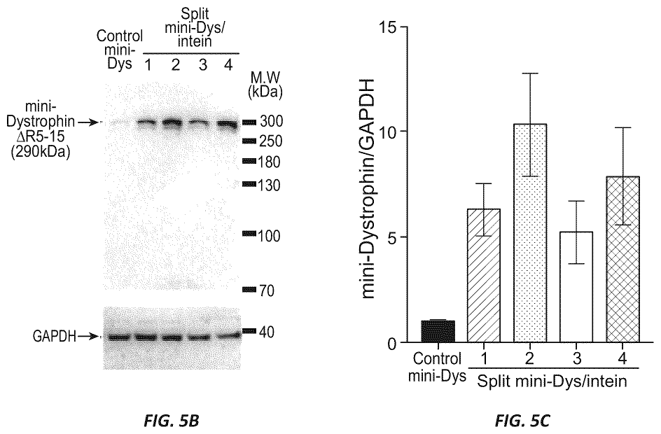

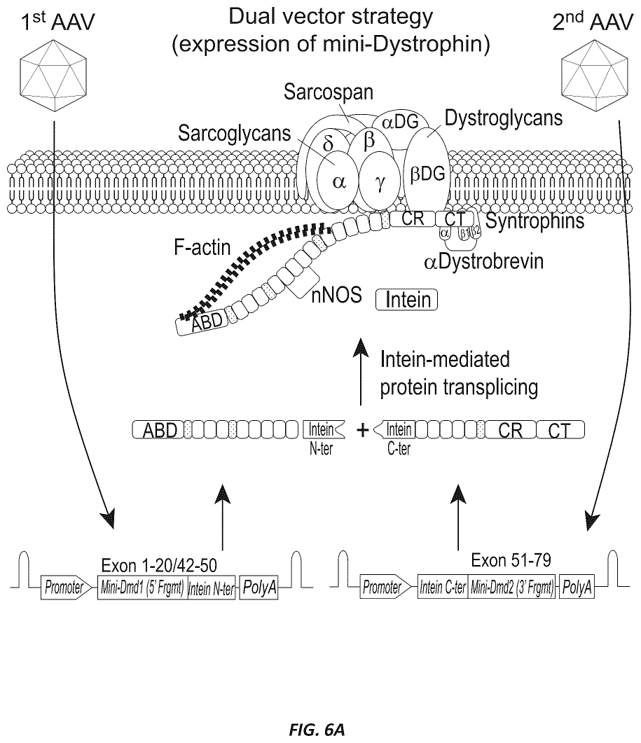

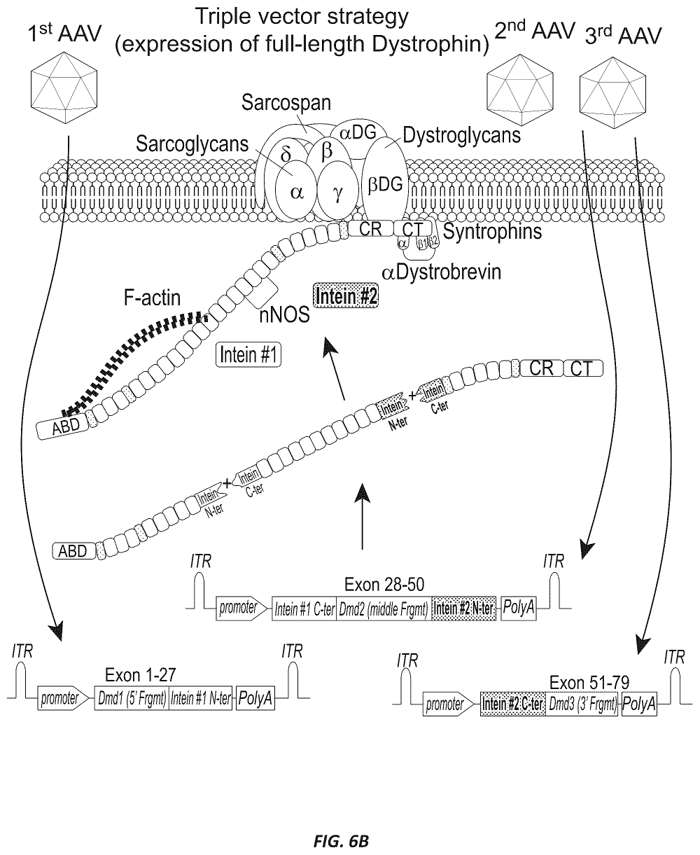

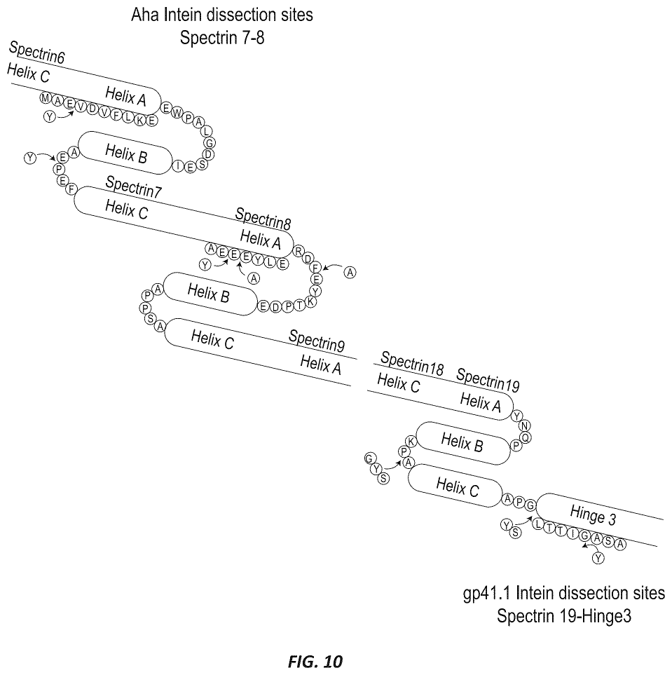

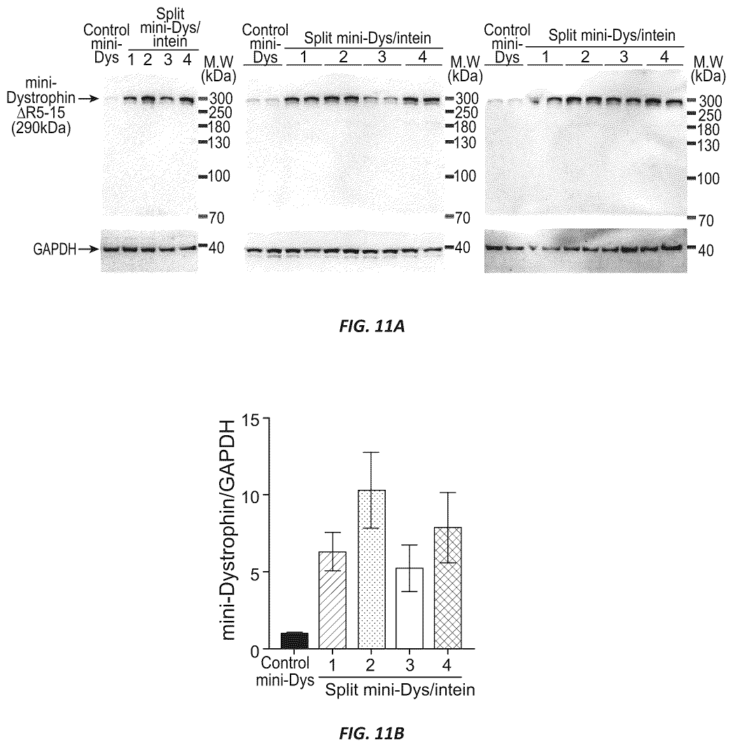

In a test, the inventors cloned two plasmids expressing either the N- or C-terminal mini-Dys/intein fragments (FIG. 5A). The inventors have tested four different splitting sites located in the linker between SR19 and Hinge3. (See, for example, FIG. 10). HEK293 cells were transfected with control plasmid encoding the entire mini-Dys (ΔSR5-15) or N and C-terminal vectors which encode for split mini-Dys (ΔSR5-15) fused to split intein. 48 hours later, total proteins were harvested for western blot analysis. Surprisingly, it was found that mini-Dys protein level was higher (5 to 11 fold) with the split vectors compared to the control plasmid (FIGS. 5B & 5C). This can possibly be explained by the short time required to simultaneously process two halves encoded by two vectors versus a long construct expressed by a single vector, or by transfection efficiencies. Interestingly, the inventors have noticed that all four selected splitting sites result in efficient mini-Dys formation, and the highest expression was obtained with site #2. In addition, no other bands were detected on the western blot membrane even after overexposure, which highlights the specificity of this method.

These in vitro data showed that this new split mini-Dys system can be used to validate the preselected inteins in vitro. Once the most efficient inteins are identified, the split mini-Dys/intein sets are cloned in AAV vectors for in vivo validation.

### Pre-Screening of Intein Library Using Split GFP System.

Numerous split intein pairs have been described, and the inventors compared the splicing of more of these (compared with the data in FIGS. 4 and 5) using a split intein GFP system. Plasmids expressing CMV-eGFP (control), dual plasmids carrying the N- or C-terminal half of eGFP with the N- or C-terminal half of codon optimized split inteins, & single plasmids carrying CMV-eGFP with the footprint that would be left by split intein joining of the dual vectors were cloned and transfected into HEK293 cells & the relative fluorescence was monitored (FIG. 7). Data show that this system worked well for comparing split intein splicing. While no pairs generated fluorescence as strong as that of WT GFP, several split intein vectors generated levels similar to that from a single vector carrying the corresponding footprint left behind after extein joining (e.g. the split Aha Intein; FIG. 7).

To evaluate whether 2 sets of split inteins could be used in 3 vectors to make full-length Dys, combinations of split intein halves were tested to see whether some might cross-splice with different split inteins, which could prevent joining of the exteins from 3 vectors by skipping the middle extein. It was observed that many split inteins do cross-splice, but that newer classes, ‘group 2 inteins’ generally do not (FIG. 8).

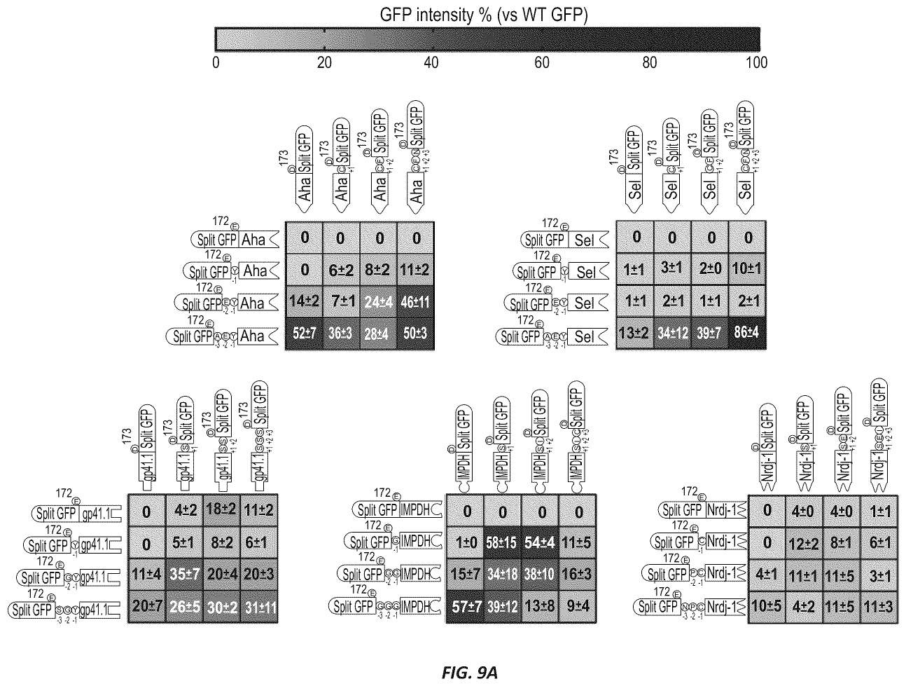

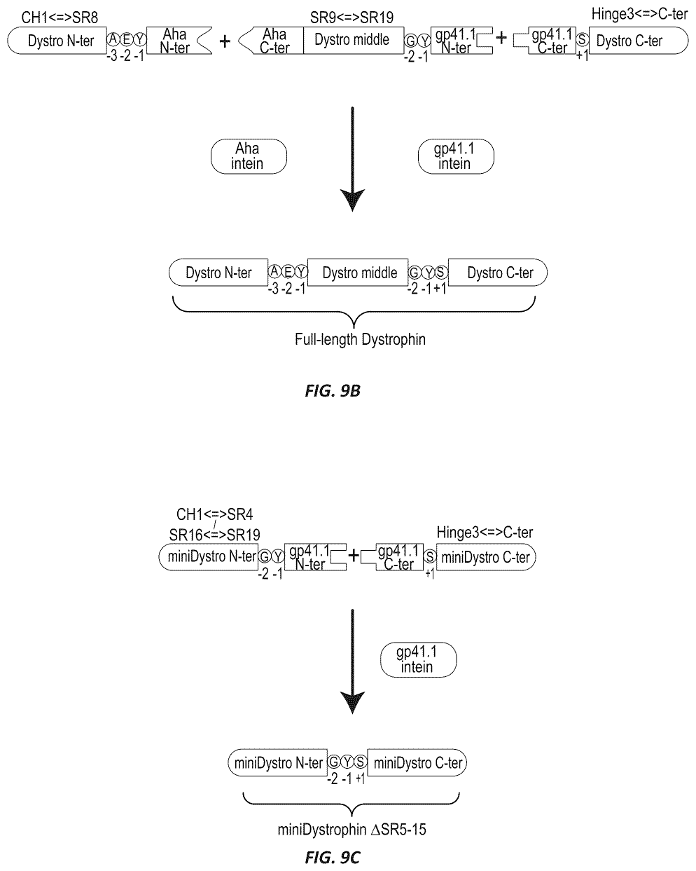

It was next tested whether the footprint could be reduced in size from its normal 6 amino acids (AA), and found that some split inteins splice efficiently when modified to leave behind only a 3 AA footprint (FIG. 9A). These results show the one can efficiently screen ‘wild-type’ & synthetic split inteins using a rapid GFP assay, and that some split intein pairs display efficient & specific splicing, while leaving minimal footprints after extein joining.

### In Vitro Validation of the Pre-Selected Inteins Using Split Mini-Dys System

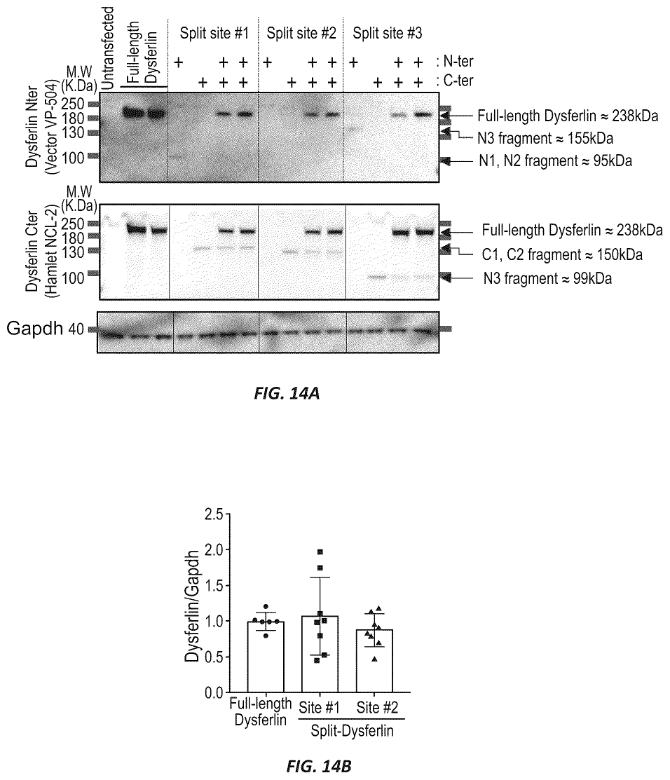

This intein system was adapted for mini-& full-length dystrophin (Dys). Two or three vectors were prepared with one or two sets of split inteins & tested in HEK293 cells. Controls used single plasmids expressing the corresponding mini-(ΔSR5-15) or full-Dys. All split intein vectors made the correct protein, at levels higher than with the single vector (perhaps reflecting reduced transfection efficiency by the larger plasmid; FIGS. 9B, 9C; FIGS. 5A-5C; FIG. 13). Some smaller products accumulated in the cells (dual vector) and some ‘mini-Dys’ in the triple vector studies, the latter suggesting splicing of the N-terminal extein directly to the C-terminal extein.

### In Vivo Validation of the Best Mini-Dys/Intein Sets by Intramuscular Injection

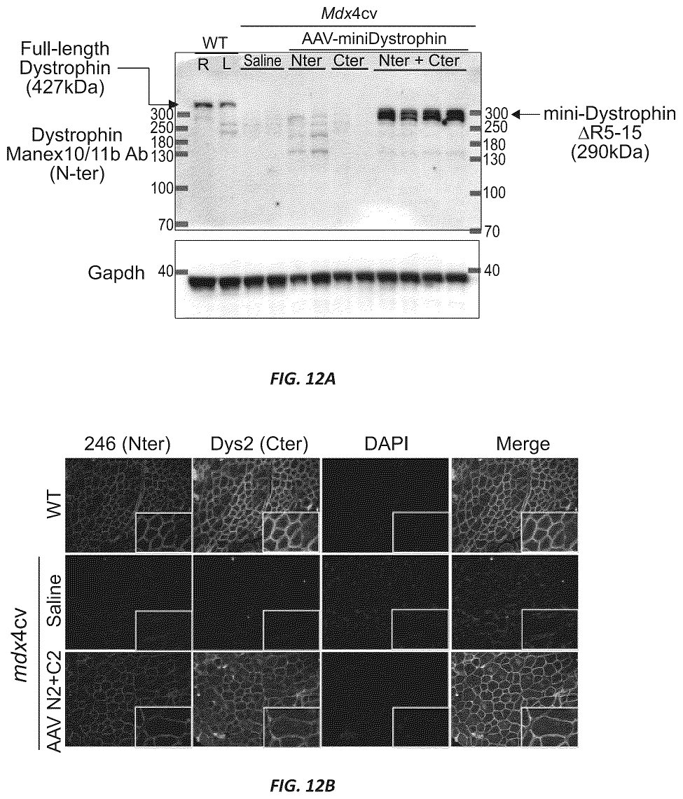

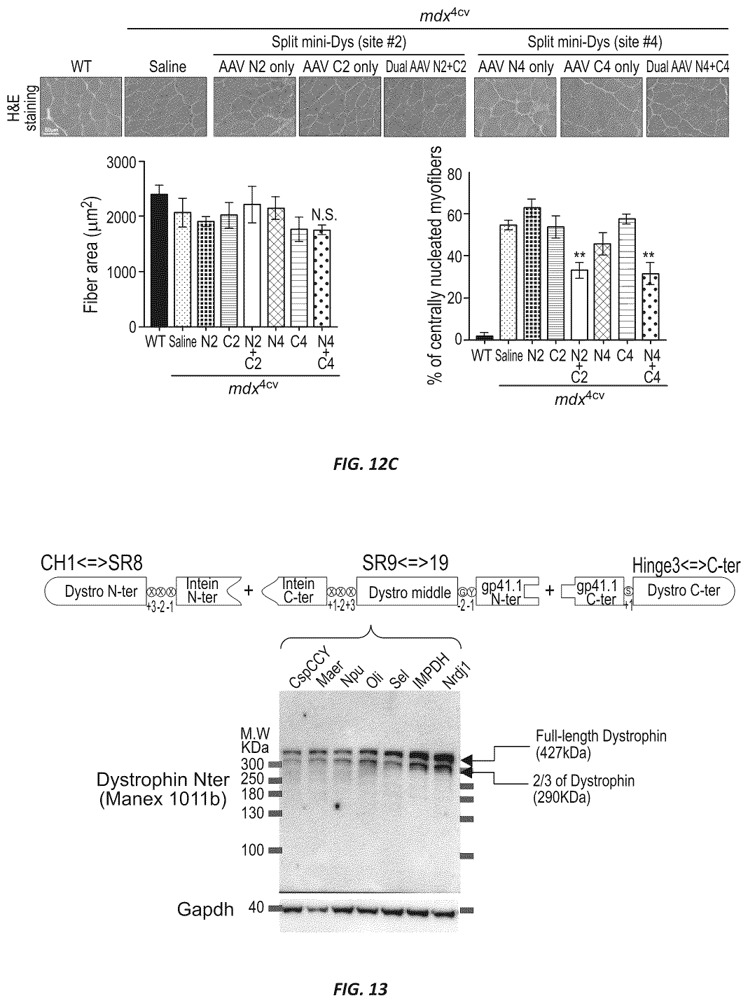

An example of one set of dual vectors that has been tested in mdx^{4cv} muscles reveals efficient expression of the ΔSR5-15 mini-dystrophin (FIG. 12). In this example, the split mini-Dystrophin/intein clones were inserted into AAV plasmid containing the muscle-specific creatine kinase 8 (CK8) regulatory cassette and small synthetic polyA flanked by two AAV serotype 2 inverted terminal repeats (ITRs) and used to make AAV vectors. A dose of 5×10¹⁰ viral genome (v.g) of AAV encoding the N- and/or C-terminal split mini-Dystrophin/intein was injected into tibialis anterior muscles (T.A) of three-week-old C57BL/6-mdx^{4cv}. Four weeks post-injection, the injected muscles were harvested and analyzed. Strong expression of mini-Dystrophin ΔSR5-15 was detected in 4 T.A muscle tested, highlighting the efficacy of SIMPLI-GT approach (FIG. 12A). Muscles were also cryo-sectioned and immunostained for dystrophin or stained with Hematoxylin and Eosin. The reconstituted mini-Dystrophin ΔSR5-15 was correctly localized at the myofiber sarcolemma of mdx^{4cv} injected with dual AAV N- and C-terminal vectors (FIG. 12B). These muscles exhibit a general muscle histology improvement with absence of inflammation (FIG. 12C).

For any given split-intein, there is flexibility on precisely where to split the mini-Dys exteins, which can affect splicing efficiency & minimize the footprint remaining after extein joining (FIGS. B-E). Some of our efforts have focused on the ΔSR5-15 mini-Dys (FIGS. 10 & 9C) in which spectrin-like repeat (SR) 4 is joined to SR16. This mini-Dys is modified from the Dys made in a mildly-affected patient with an exon 17-48 deletion, who remained ambulatory until his late 70s (England, 1989). However, the design described herein removes a partial SR encoded on exon 49 (Harper, 2002) and adds the nNOS-localization domain in SRs 16-17. (Adams et al, 2018; Lai et al., 2009) This ΔSR5-15 mini-Dys is expected to assemble the entire DGC. Unlike exon-skipping or Cas9 cutting methods, it is not needed to split the Dys sequence at a precise exon (which often leaves behind partial domains). Instead, one can put portions of the SR16-19 region in either vector followed by the 5′ split intein, while the 3′ vector could carry the 3′ split intein joined to the remaining SRs. The only difference is the location of the footprint. Multiple splitting sites have and can be tested to minimize footprints while monitoring splicing efficiency in 293 cells (FIGS. 10, 9B, C & 13). Engineered split inteins have also been developed to generate smaller inteins with a closer footprint match to candidate splitting sites (e.g. near the middle of mini-Dys clones; FIG. 10. The point here is that there are a wide choice of splitting targets such that the intein footprint can be matched to the Dys sequence while maximizing efficiency of splicing (& protein stability/function). Ideally, a Dys region that contains a perfect 6 AA match with the intein footprint would be used. While the inventors have not found such 6 AA stretches in a useful location, they have found regions where the footprint differs from Dys at only 1 or 2 amino acids. Given the size of the ideal mini-Dys proteins, there at least 600 bps in the middle of Dys to target the split inteins, as each extein vector must fit within the AAV cloning capacity (<5 kb) which includes the ITRs (360 bp), a polyA site (˜60 bp) and an MSEC. An additional way to minimize footprints is to target Dys sequences that display conservative amino acid differences from a given footprint.

To adapt this system to genes other than dystrophin, the inventors split the dysferlin cDNA into two pieces, using 3 different split sites, and cloned 3 sets of plasmids each carrying one of the sets of split inteins, similar to what was done with the dual dystrophin vector studies. In this study, the three sets of split intein dysferlin plasmids were separately co-transfected into HEK293 cells followed by harvesting of the cells and analysis by western blot against dysferlin protein (FIG. 14). As shown in FIG. 14A, the full-length dysferlin protein was produced in the HEK293 cells with both sets of split-intein dysferlin clones. Both sets produced similar levels of dysferlin as did a control plasmid carrying the full-length dysferlin cDNA. FIG. 14B shows quantitation of the protein levels, illustrating the similar efficiencies that were obtained.

The new SIMPLI-GT approach presents several advantages and can be applied to any genetic disorder with a defective gene larger than the packaging capacities of AAV vectors. It relies on the usage of AAV vectors, which are widely used in gene therapy field due to their efficiency, serotype diversity, and tissue tropism. Unlike CRISPR-Cas9 gene editing and U7 exon skipping methods, this method will promote high expression of larger dystrophin with properly phased domains, which will stabilize the dystrophin structure. This strategy can be applied to any DMD or BMD patient regardless of their genetic mutations, and ultimately, will lead to the manufacturing of one therapeutic candidate with less variability and regulatory hurdles.

### LITERATURE CITED

Banks G. B., Judge L. M., Allen J. M., Chamberlain J. S. (2010). The polyproline site in hinge 2 influences the functional capacity of truncated dystrophins, PLOS Genet., 6 (5): e1000958
Bengtsson N. E., Hall J. K., Odom G. L., Phelps M. P., Andrus C. R., Hawkins R. D., Hauschka S. D., Chamberlain J. R., Chamberlain J. S. (2017). Muscle-specific CRISPR/Cas9 dystrophin gene editing ameliorates pathophysiology in a mouse model for Duchenne muscular dystrophy, Nat Commun., 14; 8:14454
Chamberlain J. S., Gibbs R. A., Ranier J. E., Nguyen P. N., Caskey C. T. (1988). Deletion screening of the Duchenne muscular dystrophy locus via multiplex DNA amplification, Nucleic Acids Res., 16 (23): 11141-11156
Crudele J. M., Chamberlain J. S. (2019). AAV-based gene therapies for the muscular dystrophies, Human Molecular Genetics, Volume 28, Issue R1, Pages R102-R107
Emery E., Muntoni F. (2003). Duchenne muscular dystrophy, Oxford University Press, New York, 3rd edition, ISBN No. 0198515316
England S. B., Nicholson L. V., Johnson M. A., Forrest S. M., Love D. R., Zubrzycka-Gaarn E. E., Bulman D. E., Harris J. B., Davies K. E. (1990). Very mild muscular dystrophy associated with the deletion of 46% of dystrophin, Nature, 343 (6254): 180-2
Goyenvalle A., Vulin A., Fougerousse F., Leturcq F., Kaplan J., Garcia L., Danos O. (2004). Rescue of dystrophic muscle through U7 snRNA-mediated exon skipping, Science, 306 (5702): 1796-9
Gregorevic P., Allen J. M., Minami E., Blankinship M. J., Haraguchi M., Meuse L., Finn E., Adams M. E., Froehner S. C., Murry C. E., Chamberlain J. S. (2006). rAAV6-microdystrophin preserves muscle function and extends lifespan in severely dystrophic mice, Nat Med. 12 (7): 787-9
Gregorevic P., Blankinship M. J., Allen J. M., Crawford R. W., Meuse L., Miller D. G., Russell D. W., Chamberlain J. S. (2004). Systemic delivery of genes to striated muscles using adeno-associated viral vectors, Nat Med., 10 (8): 828-34
Harper S. Q., Hauser M. A., DelloRusso C., Duan D., Crawford R. W., Phelps S. F., Harper H. A., Robinson A. S., Engelhardt J. F., Brooks S. V., Chamberlain J. S. (2002). Modular flexibility of dystrophin: implications for gene therapy of Duchenne muscular dystrophy, Nat Med., 8 (3): 253-61
Koenig M., Beggs A. H., Moyer M., Scherpf S., Heindrich K., Bettecken T., Meng G., Müller C. R., Lindlöf M., Kaariainen H., de la Chapellet A., Kiuru A., Savontaus M. L., Gilgenkrantz H., Récan D., Chelly J., Kaplan J. C., Covone A. E., Archidiacono N., Romeo G., Liechti-Gailati S., Schneider V., Braga S., Moser H., Darras B. T., Murphy P., Francke U., Chen J. D., Morgan G., Denton M., Greenberg C. R., Wrogemann, Blonden L. A., van Paassen M. B., van Ommen G. J., Kunkel L. M. (1989). The molecular basis for Duchenne versus Becker muscular dystrophy: correlation of severity with type of deletion, Am J Hum Genet., 45 (4): 498-506
Koo T., Popplewell L., Athanasopoulos T., Dickson G. (2014). Triple trans-splicing adeno-associated virus vectors capable of transferring the coding sequence for full-length dystrophin protein into dystrophic mice, Hum Gene Ther, 25 (2): 98-108
Kunkel L. M. & co-authors (1986). Analysis of deletions in DNA from patients with Becker and Duchenne muscular dystrophy, Nature, 322, pages 73-77
Li C & Samulski R. J. (2020). Engineering adeno-associated virus vectors for gene therapy, Nature Reviews Genetics, 21, p255-272
Li Y. (2015). Split-inteins and their bioapplications, Biotechnol Lett., 37 (11): 2121-37
Abedi M. R., Caponigro G., Kamb A. (1998). Green fluorescent protein as a scaffold for intracellular presentation of peptides, Nucleic Acids Res., 26 (2): 623-630
Long C., Amoasii L., Mireault A. A., McAnally J. R., Li H., Sanchez-Ortiz E., Bhattacharyya S., Shelton J. M., Bassel-Duby R., Olson E. N (2016). Postnatal genome editing partially restores dystrophin expression in a mouse model of muscular dystrophy, Science, Vol. 351, Issue 6271, pp. 400-403
Lostal W., Kodippili K., Yue Y., Duan D. (2014). Full-length dystrophin reconstitution with adeno-associated viral vectors, Hum Gene Ther., 25 (6): 552-62
Monaco A. P., Bertelson C. J., Middlesworth W., Colletti C., Aldridge J., Fischbeck K. H., Bartlett R., Pericak-Vance M. A., Roses A. D. & Kunkel L. M. (1985), Detection of deletions spanning the Duchenne muscular dystrophy locus using a tightly linked DNA segment, Nature, 316, pages 842-845
Nelson C. E., Hakim C. H., Ousterout D. G., Thakore P. I., Moreb E. A., Castellanos Rivera R. M., Madhavan S., Pan X., Ann Ran F., Yan W. X., Asokan A., Zhang F., Duan D., Gersbach C. A. (2016). In vivo genome editing improves muscle function in a mouse model of Duchenne muscular dystrophy, Science, Vol. 351, Issue 6271, pp. 403-407
Niemeyer G. P., Herzog R. W., Mount J., Arruda V. R., Tillson D. M., Hathcock J., van Ginkel F. W., High K. A., Lothrop Jr C. D. (2009). Long-term correction of inhibitor-prone hemophilia B dogs treated with liver-directed AAV2-mediated factor IX gene therapy, Blood, 113 (4): 797-806
Ramos J. N., Hollinger K., Bengtsson N. E., Allen J. M., Hauschka S. D., Chamberlain J. S. (2019). Development of Novel Micro-dystrophins with Enhanced Functionality, Mol Ther., 27 (3): 623-635
Rivera V. M., Gao G., Grant R. L., Schnell M. A., Zoltick P. W., Rozamus L. W., Clackson T., Wilson J. M. (2005). Long-term pharmacologically regulated expression of erythropoietin in primates following AAV-mediated gene transfer, Blood, 105 (4): 1424-30
Odom G. L., Gregorevic P., Allen J. M., Chamberlain J. S. (2011). Gene therapy of mdx mice with large truncated dystrophins generated by recombination using rAAV6, Mol Ther., 19 (1): 36-45
Shah N. H., Muir T. W. (2015). Inteins: nature's gift to protein chemists, 2014, Chemical Science, 5 (2), 446-461
Phelps S. F., Hauser M. A., Cole N. M., Rafael J. A., Hinkle R. T., Faulkner J. A., Chamberlain J. S. (1995). Expression of full-length and truncated dystrophin mini-genes in transgenic mdx mice, Hum. mol. gen., Volume 4, Issue 8, Pages 1251-1258
Srivastava A., Lusby E. W., Berns K. I. (1983). Nucleotide sequence and organization of the adeno-associated virus 2 genome, J Virol, 45 (2): 555-64
Soundrarajan N., Cho H., Ahn B., Choi M., Thong L. M., Choi H., Cha S., Kim J., Park C., Seo K., Park C. (2016). Green fluorescent protein as a scaffold for high efficiency production of functional bacteriotoxic proteins in Escherichia coli, Sci Rep., 6:20661
Wasala N. B., Shin J. H., Lai Y., Yue Y., Montanaro F., Duan D. (2018). Cardiac specific expression of ΔH2-R15 mini-dystrophin normalized all ECG abnormalities and the end-diastolic volume in a 23-m-old mouse model of Duchenne dilated cardiomyopathy, Hum. Gene Ther., Vol. 29, No. 7
Adams M E, Odom G L, Kim M J, Chamberlain J S and Froehner S C: Syntrophin binds directly to multiple spectrin-like repeats in dystrophin and mediates binding of nNOS to repeats 16-17. Hum Mol Genet 2018; 27:2978-2985.
Lai Y, Thomas G D, Yue Y, Yang H T, Li D, Long C, Judge L, Bostick B, Chamberlain J S, Terjung R L and Duan D: Dystrophins carrying spectrin-like repeats 16 and 17 anchor nNOS to the sarcolemma and enhance exercise performance in a mouse model of muscular dystrophy. J Clin Invest 2009; 119:624-635.

### Example 2: Exemplary Sequences of Split Inteins with Mini-Dystrophin, Dystrophin, Dysferlin or Utrophin

MiniDys ΔSR5-15:
1) Split site #1:
•MiniDys-gp41.1 N1 (SEQ ID NO. 82):
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVMK
NIMAGLQQTNSEKILLSWVRQSTRNYPQVNVINFTTSWSDGLALNALIHSHRPDLFDWNSVVCQ
QSATQRLEHAFNIARYQLGIEKLLDPEDVDTTYPDKKSILMYITSLFQVLPQQVSIEAIQEVEMLP
RPPKVTKEEHFQLHHQMHYSQQITVSLAQGYERTSSPKPRFKSYAYTQAAYVTTSDPTRSPFPSQ
HLEAPEDKSFGSSLMESEVNLDRYQTALEEVLSWLLSAEDTLQAQGEISNDVEVVKDQFHTHEG
YMMDLTAHQGRVGNILQLGSKLIGTGKLSEDEETEVQEQMNLLNSRWECLRVASMEKQSNLH
RVLMDLQNQKLKELNDWLTKTEERTRKMEEEPLGPDLEDLKRQVQQHKVLQEDLEQEQVRVN
SLTHMVVVVDESSGDHATAALEEQLKVLGDRWANICRWTEDRWVLLQDILLKWQRLTEEQCL
FSAWLSEKEDAVNKIHTTGFKDQNEMLSSLQKLAVLKADLEKKKQSMGKLYSLKQDLLSTLKN
KSVTQKTEAWLDNFARCWDNLVQKLEKSTAQISQAVTTTQPSLTQTTVMETVTTVTTREQILVK
HAQEELPPPPPQKKRQITVDSEIRKRLDVDITELHSWITRSEAVLQSPEFAIFRKEGNFSDLKEKVN
AIEREKAEKFRKLQDASRSAQALVEQMVNEGVNADSIKQASEQLNSRWIEFCQLLSERLNWLEY
SYVPSTYLTEITHVSQALLEVEQLLNAPDLCAKDFEDLFKQEESLKNIKDSLQQSSGRIDIIHSKKT
AALQSATPVERVKLQEALSQLDFQWEKVNKMYKDRQGRFDRSVEKWRRFHYDIKIFNQWLTE
AEQFLRKTQIPENWEHAKYKWYLKELQDGIGQRQTVVRTLNATGEEIIQQSSKTDASILQEKLGS
LNLRWQEVCKQLSDRKKRLEEQKNILSEFQRDLNEFVLWLEEADNIASIPLEPGKEQQLKEKLEQ
VKLLVEELPLRQGILKQLNETGGPVLVSAPISPEEQDKLENKLKQTNLQWIKVSRALPEKQGEIE
AQIKDLGQLEKKLEDLEEQLNHLLLWLSPIRNQLEIYNQPNQEGPFDVQETEIAVQAKQPDVEEI
LSKGQHLYKEKPGYCLDLKTQVQTPQGMKEISNIQVGDLVLSNTGYNEVLNVFPKSKKKSYKIT
LEDGKEIICSEEHLFPTQTGEMNISGGLKEGMCLYVKE*
•MiniDys-gp41.1 C1 (SEQ ID NO. 83):
MMLKKILKIEELDERELIDIEVSGNHLFYANDILTHNSATQPVKRKLEDLSSEWKAVNRLLQELR
AKQPDLAPGLTTIGASPTQTVTLVTQPVVTKETAISKLEMPSSLMLEVPALADFNRAWTELTDW
LSLLDQVIKSQRVMVGDLEDINEMIIKQKATMQDLEQRRPQLEELITAAQNLKNKTSNQEARTII
TDRIERIQNQWDEVQEHLQNRRQQLNEMLKDSTQWLEAKEEAEQVLGQARAKLESWKEGPYT
VDAIQKKITETKQLAKDLRQWQTNVDVANDLALKLLRDYSADDTRKVHMITENINASWRSIHK
RVSEREAALEETHRLLQQFPLDLEKFLAWLTEAETTANVLQDATRKERLLEDSKGVKELMKQW
QDLQGEIEAHTDVYHNLDENSQKILRSLEGSDDAVLLQRRLDNMNFKWSELRKKSLNIRSHLEA
SSDQWKRLHLSLQELLVWLQLKDDELSRQAPIGGDFPAVQKQNDVHRAFKRELKTKEPVIMST
LETVRIFLTEQPLEGLEKLYQEPRELPPEERAQNVTRLLRKQAEEVNTEWEKLNLHSADWQRKI
DETLERLQELQEATDELDLKLRQAEVIKGSWQPVGDLLIDSLQDHLEKVKALRGEIAPLKENVS
HVNDLARQLTTLGIQLSPYNLSTLEDLNTRWKLLQVAVEDRVRQLHEAHRDFGPASQHELSTSV
QGPWERAISPNKVPYYINHETQTTCWDHPKMTELYQSLADLNNVRFSAYRTAMKLRRLQKALC
LDLLSLSAACDALDQHNLKQNDQPMDILQIINCLTTIYDRLEQEHNNLVNVPLCVDMCLNWLLN
VYDTGRTGRIRVLSFKTGIISLCKAHLEDKYRYLFKQVASSTGFCDQRRLGLLLHDSIQIPRQLGE
VASFGGSNIEPSVRSCFQFANNKPEIEAALFLDWMRLEPQSMVWLPVLHRVAAAETAKHQAKC
NICKECPIIGFRYRSLKHFNYDICQSCFFSGRVAKGHKMHYPMVEYCTPTTSGEDVRDFAKVLKN
KFRTKRYFAKHPRMGYLPVQTVLEGDNMETPVTLINFWPVDSAPASSPQLSHDDTHSRIEHYAS
RLAEMENSNGSYLNDSISPNESIDDEHLLIQHYCQSLNQDSPLSQPRSPAQILISLESEERGELERIL
ADLEEENRNLQAEYDRLKQQHEHKGLSPLPSPPEMMPTSPQSPRDAELIAEAKLLRQHKGRLEA
RMQILEDHNKQLESQLHRLRQLLEQPQAEAKVNGTTVSSPSTSLQRSDSSQPMLLRVVGSQTSD
SMGEEDLLSPPQDTSTGLEEVMEQLNNSFPSSRGRNTPGKPMREDTM*
2) Split site #2:
•MiniDys-gp41.1 N2 (SEQ ID NO. 84):
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVMK
NIMAGLQQTNSEKILLSWVRQSTRNYPQVNVINFTTSWSDGLALNALIHSHRPDLFDWNSVVCQ
QSATQRLEHAFNIARYQLGIEKLLDPEDVDTTYPDKKSILMYITSLFQVLPQQVSIEAIQEVEMLP
RPPKVTKEEHFQLHHQMHYSQQITVSLAQGYERTSSPKPRFKSYAYTQAAYVTTSDPTRSPFPSQ
HLEAPEDKSFGSSLMESEVNLDRYQTALEEVLSWLLSAEDTLQAQGEISNDVEVVKDQFHTHEG
YMMDLTAHQGRVGNILQLGSKLIGTGKLSEDEETEVQEQMNLLNSRWECLRVASMEKQSNLH
RVLMDLQNQKLKELNDWLTKTEERTRKMEEEPLGPDLEDLKRQVQQHKVLQEDLEQEQVRVN
SLTHMVVVVDESSGDHATAALEEQLKVLGDRWANICRWTEDRWVLLQDILLKWQRLTEEQCL
FSAWLSEKEDAVNKIHTTGFKDQNEMLSSLQKLAVLKADLEKKKQSMGKLYSLKQDLLSTLKN
KSVTQKTEAWLDNFARCWDNLVQKLEKSTAQISQAVTTTQPSLTQTTVMETVTTVTTREQILVK
HAQEELPPPPPQKKRQITVDSEIRKRLDVDITELHSWITRSEAVLQSPEFAIFRKEGNFSDLKEKVN
AIEREKAEKFRKLQDASRSAQALVEQMVNEGVNADSIKQASEQLNSRWIEFCQLLSERLNWLEY
SYVPSTYLTEITHVSQALLEVEQLLNAPDLCAKDFEDLFKQEESLKNIKDSLQQSSGRIDIIHSKKT
AALQSATPVERVKLQEALSQLDFQWEKVNKMYKDRQGRFDRSVEKWRRFHYDIKIFNQWLTE
AEQFLRKTQIPENWEHAKYKWYLKELQDGIGQRQTVVRTLNATGEEIIQQSSKTDASILQEKLGS
LNLRWQEVCKQLSDRKKRLEEQKNILSEFQRDLNEFVLWLEEADNIASIPLEPGKEQQLKEKLEQ
VKLLVEELPLRQGILKQLNETGGPVLVSAPISPEEQDKLENKLKQTNLQWIKVSRALPEKQGEIE
AQIKDLGQLEKKLEDLEEQLNHLLLWLSPIRNQLEIYNQPNQEGPFDVQETEIAVQAKQPDVEEI
LSKGQHLYKEGYCLDLKTQVQTPQGMKEISNIQVGDLVLSNTGYNEVLNVFPKSKKKSYKITLE
DGKEIICSEEHLFPTQTGEMNISGGLKEGMCLYVKE*
•MiniDys-gp41.1 C2 (SEQ ID NO. 85):
MMLKKILKIEELDERELIDIEVSGNHLFYANDILTHNSTQPVKRKLEDLSSEWKAVNRLLQELRA
KQPDLAPGLTTIGASPTQTVTLVTQPVVTKETAISKLEMPSSLMLEVPALADFNRAWTELTDWLS
LLDQVIKSQRVMVGDLEDINEMIIKQKATMQDLEQRRPQLEELITAAQNLKNKTSNQEARTIITD
RIERIQNQWDEVQEHLQNRRQQLNEMLKDSTQWLEAKEEAEQVLGQARAKLESWKEGPYTVD
AIQKKITETKQLAKDLRQWQTNVDVANDLALKLLRDYSADDTRKVHMITENINASWRSIHKRV
SEREAALEETHRLLQQFPLDLEKFLAWLTEAETTANVLQDATRKERLLEDSKGVKELMKQWQD
LQGEIEAHTDVYHNLDENSQKILRSLEGSDDAVLLQRRLDNMNFKWSELRKKSLNIRSHLEASS
DQWKRLHLSLQELLVWLQLKDDELSRQAPIGGDFPAVQKQNDVHRAFKRELKTKEPVIMSTLE
TVRIFLTEQPLEGLEKLYQEPRELPPEERAQNVTRLLRKQAEEVNTEWEKLNLHSADWQRKIDE
TLERLQELQEATDELDLKLRQAEVIKGSWQPVGDLLIDSLQDHLEKVKALRGEIAPLKENVSHV
NDLARQLTTLGIQLSPYNLSTLEDLNTRWKLLQVAVEDRVRQLHEAHRDFGPASQHFLSTSVQG
PWERAISPNKVPYYINHETQTTCWDHPKMTELYQSLADLNNVRFSAYRTAMKLRRLQKALCLD
LLSLSAACDALDQHNLKQNDQPMDILQIINCLTTIYDRLEQEHNNLVNVPLCVDMCLNWLLNV
YDTGRTGRIRVLSFKTGIISLCKAHLEDKYRYLFKQVASSTGFCDQRRLGLLLHDSIQIPRQLGEV
ASFGGSNIEPSVRSCFQFANNKPEIEAALFLDWMRLEPQSMVWLPVLHRVAAAETAKHQAKCNI
CKECPIIGFRYRSLKHFNYDICQSCFFSGRVAKGHKMHYPMVEYCTPTTSGEDVRDFAKVLKNK
FRTKRYFAKHPRMGYLPVQTVLEGDNMETPVTLINFWPVDSAPASSPQLSHDDTHSRIEHYASR
LAEMENSNGSYLNDSISPNESIDDEHLLIQHYCQSLNQDSPLSQPRSPAQILISLESEERGELERILA
DLEEENRNLQAEYDRLKQQHEHKGLSPLPSPPEMMPTSPQSPRDAELIAEAKLLRQHKGRLEAR
MQILEDHNKQLESQLHRLRQLLEQPQAEAKVNGTTVSSPSTSLQRSDSSQPMLLRVVGSQTSDS
MGEEDLLSPPQDTSTGLEEVMEQLNNSFPSSRGRNTPGKPMREDTM*
3) Split site #3:
•MiniDys-gp41.1 N3 (SEQ ID NO. 86):
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVMK
NIMAGLQQTNSEKILLSWVRQSTRNYPQVNVINFTTSWSDGLALNALIHSHRPDLFDWNSVVCQ
QSATQRLEHAFNIARYQLGIEKLLDPEDVDTTYPDKKSILMYITSLFQVLPQQVSIEAIQEVEMLP
RPPKVTKEEHFQLHHQMHYSQQITVSLAQGYERTSSPKPRFKSYAYTQAAYVTTSDPTRSPFPSQ
HLEAPEDKSFGSSLMESEVNLDRYQTALEEVLSWLLSAEDTLQAQGEISNDVEVVKDQFHTHEG
YMMDLTAHQGRVGNILQLGSKLIGTGKLSEDEETEVQEQMNLLNSRWECLRVASMEKQSNLH
RVLMDLQNQKLKELNDWLTKTEERTRKMEEEPLGPDLEDLKRQVQQHKVLQEDLEQEQVRVN
SLTHMVVVVDESSGDHATAALEEQLKVLGDRWANICRWTEDRWVLLQDILLKWQRLTEEQCL
FSAWLSEKEDAVNKIHTTGFKDQNEMLSSLQKLAVLKADLEKKKQSMGKLYSLKQDLLSTLKN
KSVTQKTEAWLDNFARCWDNLVQKLEKSTAQISQAVTTTQPSLTQTTVMETVTTVTTREQILVK
HAQEELPPPPPQKKRQITVDSEIRKRLDVDITELHSWITRSEAVLQSPEFAIFRKEGNFSDLKEKVN
AIEREKAEKFRKLQDASRSAQALVEQMVNEGVNADSIKQASEQLNSRWIEFCQLLSERLNWLEY
SYVPSTYLTEITHVSQALLEVEQLLNAPDLCAKDFEDLFKQEESLKNIKDSLQQSSGRIDIIHSKKT
AALQSATPVERVKLQEALSQLDFQWEKVNKMYKDRQGRFDRSVEKWRRFHYDIKIFNQWLTE
AEQFLRKTQIPENWEHAKYKWYLKELQDGIGQRQTVVRTLNATGEEIIQQSSKTDASILQEKLGS
LNLRWQEVCKQLSDRKKRLEEQKNILSEFQRDLNEFVLWLEEADNIASIPLEPGKEQQLKEKLEQ
VKLLVEELPLRQGILKQLNETGGPVLVSAPISPEEQDKLENKLKQTNLQWIKVSRALPEKQGEIE
AQIKDLGQLEKKLEDLEEQLNHLLLWLSPIRNQLEIYNQPNQEGPFDVQETEIAVQAKQPDVEEI
LSKGQHLYKEKPATQPVKRKLEDLSSEWKAVNRLLQELRAKQPDLAPGYCLDLKTQVQTPQG
MKEISNIQVGDLVLSNTGYNEVLNVFPKSKKKSYKITLEDGKEIICSEEHLFPTQTGEMNISGGLK
EGMCLYVKE*
•MiniDys-gp41.1 C3 (SEQ ID NO. 87):
MMLKKILKIEELDERELIDIEVSGNHLFYANDILTHNSLTTIGASPTQTVTLVTQPVVTKETAISKL
EMPSSLMLEVPALADFNRAWTELTDWLSLLDQVIKSQRVMVGDLEDINEMIIKQKATMQDLEQ
RRPQLEELITAAQNLKNKTSNQEARTIITDRIERIQNQWDEVQEHLQNRRQQLNEMLKDSTQWL
EAKEEAEQVLGQARAKLESWKEGPYTVDAIQKKITETKQLAKDLRQWQTNVDVANDLALKLL
RDYSADDTRKVHMITENINASWRSIHKRVSEREAALEETHRLLQQFPLDLEKFLAWLTEAETTA
NVLQDATRKERLLEDSKGVKELMKQWQDLQGEIEAHTDVYHNLDENSQKILRSLEGSDDAVLL
QRRLDNMNFKWSELRKKSLNIRSHLEASSDQWKRLHLSLQELLVWLQLKDDELSRQAPIGGDFP
AVQKQNDVHRAFKRELKTKEPVIMSTLETVRIFLTEQPLEGLEKLYQEPRELPPEERAQNVTRLL
RKQAEEVNTEWEKLNLHSADWQRKIDETLERLQELQEATDELDLKLRQAEVIKGSWQPVGDLL
IDSLQDHLEKVKALRGEIAPLKENVSHVNDLARQLTTLGIQLSPYNLSTLEDLNTRWKLLQVAV
EDRVRQLHEAHRDFGPASQHELSTSVQGPWERAISPNKVPYYINHETQTTCWDHPKMTELYQSL
ADLNNVRFSAYRTAMKLRRLQKALCLDLLSLSAACDALDQHNLKQNDQPMDILQIINCLTTIYD
RLEQEHNNLVNVPLCVDMCLNWLLNVYDTGRTGRIRVLSFKTGIISLCKAHLEDKYRYLFKQV
ASSTGFCDQRRLGLLLHDSIQIPRQLGEVASFGGSNIEPSVRSCFQFANNKPEIEAALFLDWMRLE
PQSMVWLPVLHRVAAAETAKHQAKCNICKECPIIGFRYRSLKHFNYDICQSCFFSGRVAKGHKM
HYPMVEYCTPTTSGEDVRDFAKVLKNKFRTKRYFAKHPRMGYLPVQTVLEGDNMETPVTLINF
WPVDSAPASSPQLSHDDTHSRIEHYASRLAEMENSNGSYLNDSISPNESIDDEHLLIQHYCQSLNQ
DSPLSQPRSPAQILISLESEERGELERILADLEEENRNLQAEYDRLKQQHEHKGLSPLPSPPEMMPT
SPQSPRDAELIAEAKLLRQHKGRLEARMQILEDHNKQLESQLHRLRQLLEQPQAEAKVNGTTVS
SPSTSLQRSDSSQPMLLRVVGSQTSDSMGEEDLLSPPQDTSTGLEEVMEQLNNSFPSSRGRNTPG
KPMREDTM*
4) Split site #4:
•MiniDys-gp41.1 N4 (SEQ ID NO. 88):
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVMK
NIMAGLQQTNSEKILLSWVRQSTRNYPQVNVINFTTSWSDGLALNALIHSHRPDLFDWNSVVCQ
QSATQRLEHAFNIARYQLGIEKLLDPEDVDTTYPDKKSILMYITSLFQVLPQQVSIEAIQEVEMLP
RPPKVTKEEHFQLHHQMHYSQQITVSLAQGYERTSSPKPRFKSYAYTQAAYVTTSDPTRSPFPSQ
HLEAPEDKSFGSSLMESEVNLDRYQTALEEVLSWLLSAEDTLQAQGEISNDVEVVKDQFHTHEG
YMMDLTAHQGRVGNILQLGSKLIGTGKLSEDEETEVQEQMNLLNSRWECLRVASMEKQSNLH
RVLMDLQNQKLKELNDWLTKTEERTRKMEEEPLGPDLEDLKRQVQQHKVLQEDLEQEQVRVN
SLTHMVVVVDESSGDHATAALEEQLKVLGDRWANICRWTEDRWVLLQDILLKWQRLTEEQCL
FSAWLSEKEDAVNKIHTTGFKDQNEMLSSLQKLAVLKADLEKKKQSMGKLYSLKQDLLSTLKN
KSVTQKTEAWLDNFARCWDNLVQKLEKSTAQISQAVTTTQPSLTQTTVMETVTTVTTREQILVK
HAQEELPPPPPQKKRQITVDSEIRKRLDVDITELHSWITRSEAVLQSPEFAIFRKEGNFSDLKEKVN
AIEREKAEKFRKLQDASRSAQALVEQMVNEGVNADSIKQASEQLNSRWIEFCQLLSERLNWLEY
SYVPSTYLTEITHVSQALLEVEQLLNAPDLCAKDFEDLFKQEESLKNIKDSLQQSSGRIDIIHSKKT
AALQSATPVERVKLQEALSQLDFQWEKVNKMYKDRQGRFDRSVEKWRRFHYDIKIFNQWLTE
AEQFLRKTQIPENWEHAKYKWYLKELQDGIGQRQTVVRTLNATGEEIIQQSSKTDASILQEKLGS
LNLRWQEVCKQLSDRKKRLEEQKNILSEFQRDLNEFVLWLEEADNIASIPLEPGKEQQLKEKLEQ
VKLLVEELPLRQGILKQLNETGGPVLVSAPISPEEQDKLENKLKQTNLQWIKVSRALPEKQGEIE
AQIKDLGQLEKKLEDLEEQLNHLLLWLSPIRNQLEIYNQPNQEGPFDVQETEIAVQAKQPDVEEI
LSKGQHLYKEKPATQPVKRKLEDLSSEWKAVNRLLQELRAKQPDLAPGLTTIGYCLDLKTQVQ
TPQGMKEISNIQVGDLVLSNTGYNEVLNVFPKSKKKSYKITLEDGKEIICSEEHLFPTQTGEMNIS
GGLKEGMCLYVKE*
•MiniDys-gp41.1 C4 (SEQ ID NO. 89):
MMLKKILKIEELDERELIDIEVSGNHLFYANDILTHNSPTQTVTLVTQPVVTKETAISKLEMPSSL
MLEVPALADFNRAWTELTDWLSLLDQVIKSQRVMVGDLEDINEMIIKQKATMQDLEQRRPQLE
ELITAAQNLKNKTSNQEARTIITDRIERIQNQWDEVQEHLQNRRQQLNEMLKDSTQWLEAKEEA
EQVLGQARAKLESWKEGPYTVDAIQKKITETKQLAKDLRQWQTNVDVANDLALKLLRDYSAD
DTRKVHMITENINASWRSIHKRVSEREAALEETHRLLQQFPLDLEKFLAWLTEAETTANVLQDA
TRKERLLEDSKGVKELMKQWQDLQGEIEAHTDVYHNLDENSQKILRSLEGSDDAVLLQRRLDN
MNFKWSELRKKSLNIRSHLEASSDQWKRLHLSLQELLVWLQLKDDELSRQAPIGGDFPAVQKQ
NDVHRAFKRELKTKEPVIMSTLETVRIFLTEQPLEGLEKLYQEPRELPPEERAQNVTRLLRKQAE
EVNTEWEKLNLHSADWQRKIDETLERLQELQEATDELDLKLRQAEVIKGSWQPVGDLLIDSLQD
HLEKVKALRGEIAPLKENVSHVNDLARQLTTLGIQLSPYNLSTLEDLNTRWKLLQVAVEDRVRQ
LHEAHRDFGPASQHELSTSVQGPWERAISPNKVPYYINHETQTTCWDHPKMTELYQSLADLNNV
RFSAYRTAMKLRRLQKALCLDLLSLSAACDALDQHNLKQNDQPMDILQIINCLTTIYDRLEQEH
NNLVNVPLCVDMCLNWLLNVYDTGRTGRIRVLSFKTGIISLCKAHLEDKYRYLFKQVASSTGFC
DQRRLGLLLHDSIQIPRQLGEVASFGGSNIEPSVRSCFQFANNKPEIEAALFLDWMRLEPQSMVW
LPVLHRVAAAETAKHQAKCNICKECPIIGFRYRSLKHFNYDICQSCFFSGRVAKGHKMHYPMVE
YCTPTTSGEDVRDFAKVLKNKFRTKRYFAKHPRMGYLPVQTVLEGDNMETPVTLINFWPVDSA
PASSPQLSHDDTHSRIEHYASRLAEMENSNGSYLNDSISPNESIDDEHLLIQHYCQSLNQDSPLSQ
PRSPAQILISLESEERGELERILADLEEENRNLQAEYDRLKQQHEHKGLSPLPSPPEMMPTSPQSPR
DAELIAEAKLLRQHKGRLEARMQILEDHNKQLESQLHRLRQLLEQPQAEAKVNGTTVSSPSTSL
QRSDSSQPMLLRVVGSQTSDSMGEEDLLSPPQDTSTGLEEVMEQLNNSFPSSRGRNTPGKPMRE
DTM*
Full-length Dystrophin:
a. Using IMPDH and gp41.1:
1) Split site ImS1:
•N-ter fragment (SEQ ID NO. 90):
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVM
KNIMAGLQQTNSEKILLSWVRQSTRNYPQVNVINFTTSWSDGLALNALIHSHRPDLFDWNSVV
CQQSATQRLEHAFNIARYQLGIEKLLDPEDVDTTYPDKKSILMYITSLFQVLPQQVSIEAIQEVE
MLPRPPKVTKEEHFQLHHQMHYSQQITVSLAQGYERTSSPKPRFKSYAYTQAAYVTTSDPTRSP
FPSQHLEAPEDKSFGSSLMESEVNLDRYQTALEEVLSWLLSAEDTLQAQGEISNDVEVVKDQFH
THEGYMMDLTAHQGRVGNILQLGSKLIGTGKLSEDEETEVQEQMNLLNSRWECLRVASMEKQ
SNLHRVLMDLQNQKLKELNDWLTKTEERTRKMEEEPLGPDLEDLKRQVQQHKVLQEDLEQEQ
VRVNSLTHMVVVVDESSGDHATAALEEQLKVLGDRWANICRWTEDRWVLLQDILLKWQRLT
EEQCLFSAWLSEKEDAVNKIHTTGFKDQNEMLSSLQKLAVLKADLEKKKQSMGKLYSLKQDL
LSTLKNKSVTQKTEAWLDNFARCWDNLVQKLEKSTAQISQAVTTTQPSLTQTTVMETVTTVTT
REQILVKHAQEELPPPPPQKKRQITVDSEIRKRLDVDITELHSWITRSEAVLQSPEFAIFRKEGNFS
DLKEKVNAIEREKAEKFRKLQDASRSAQALVEQMVNEGVNADSIKQASEQLNSRWIEFCQLLS
ERLNWLEYQNNIIAFYNQLQQLEQMTTTAENWLKIQPTTPSEPTAIKSQLKICKDEVNRLSGLQP
QIERLKIQSIALKEKGQGPMFLDADFVAFTNHFKQVFSDVQAREKELQTIFDTLPPMRYQETMS
AIRTWVQQSETKLSIPQLSVTDYEIMEQRLGELQALQSSLQEQQSGLYYLSTTVKEMSKKAPSEI
SRKYQSEFEEIEGRWKKLSSQLVEHCQKLEEQMNKLRKIQNHIQTLKKWMAEVDVFLKEEWP
ALGGCFVPGTLVNTENGLKKIEEIKVGDKVFSHTGKLQEVVDTLIFDRDEEIISINGIDCTKNHEF
YVIDKENANRVNEDNIHLFARWVHAEELDMKKHLLIELE*
•Middle fragment (SEQ ID NO. 91):
MKFKLKEITSIETKHYKGKVHDLTVNQDHSYNVRGTVVHNSEILKKQLKQCRLLVSDIQTIQPS
LNSVNEGGQKIKNEAEPEFASRLETELKELNTQWDHMCQQVYARKEALKGGLEKTVSLQKDL
SEMHEWMTQAEEEYLERDFEYKTPDELQKAVEEMKRAKEEAQQKEAKVKLLTESVNSVIAQA
PPVAQEALKKELETLTTNYQWLCTRLNGKCKTLEEVWACWHELLSYLEKANKWLNEVEFKL
KTTENIPGGAEEISEVLDSLENLMRHSEDNPNQIRILAQTLTDGGVMDELINEELETFNSRWREL
HEEAVRRQKLLEQSIQSAQETEKSLHLIQESLTFIDKQLAAYIADKVDAAQMPQEAQKIQSDLTS
HEISLEEMKKHNQGKEAAQRVLSQIDVAQKKLQDVSMKFRLFQKPANFELRLQESKMILDEVK
MHLPALETKSVEQEVVQSQLNHCVNLYKSLSEVKSEVEMVIKTGRQIVQKKQTENPKELDERV
TALKLHYNELGAKVTERKQQLEKCLKLSRKMRKEMNVLTEWLAATDMELTKRSAVEGMPSN
LDSEVAWGKATQKEIEKQKVHLKSITEVGEALKTVLGKKETLVEDKLSLLNSNWIAVTSRAEE
WLNLLLEYQKHMETFDQNVDHITKWIIQADTLLDESEKKKPQQKEDVLKRLKAELNDIRPKVD
STRDQAANLMANRGDHCRKLVEPQISELNHRFAAISHRIKTGKASIPLKELEQFNSDIQKLLEPL
EAEIQQGVNLKEEDFNKDMNEDNEGTVKELLQRGDNLQQRITDERKREEIKIKQQLLQTKHNA
LKDLRSQRRKKALEISHQWYQYKRQADDLLKCLDDIEKKLASLPEPRDERKIKEIDRELQKKKE
ELNAVRRQAEGLSEDGAAMAVEPTQIQLSKRWREIESKFAQFRRLNFAQIHTVREETMMVMTE
DMPLEISYVPSTYLTEITHVSQALLEVEQLLNAPDLCAKDFEDLFKQEESLKNIKDSLQQSSGRI
DIIHSKKTAALQSATPVERVKLQEALSQLDFQWEKVNKMYKDRQGRFDRSVEKWRRFHYDIKI
FNQWLTEAEQFLRKTQIPENWEHAKYKWYLKELQDGIGQRQTVVRTLNATGEEIIQQSSKTDA
SILQEKLGSLNLRWQEVCKQLSDRKKRLEEQKNILSEFQRDLNEFVLWLEEADNIASIPLEPGKE
QQLKEKLEQVKLLVEELPLRQGILKQLNETGGPVLVSAPISPEEQDKLENKLKQTNLQWIKVSR
ALPEKQGEIEAQIKDLGQLEKKLEDLEEQLNHLLLWLSPIRNQLEIYNQPNQEGPFDVQETEIAV
QAKQPDVEEILSKGQHLYKEGYCLDLKTQVQTPQGMKEISNIQVGDLVLSNTGYNEVLNVFPK
SKKKSYKITLEDGKEIICSEEHLFPTQTGEMNISGGLKEGMCLYVKE*
•C-ter fragment (SEQ ID NO. 92):
MMLKKILKIEELDERELIDIEVSGNHLFYANDILTHNSTQPVKRKLEDLSSEWKAVNRLLQELR
AKQPDLAPGLTTIGASPTQTVTLVTQPVVTKETAISKLEMPSSLMLEVPALADFNRAWTELTDW
LSLLDQVIKSQRVMVGDLEDINEMIIKQKATMQDLEQRRPQLEELITAAQNLKNKTSNQEARTII
TDRIERIQNQWDEVQEHLQNRRQQLNEMLKDSTQWLEAKEEAEQVLGQARAKLESWKEGPYT
VDAIQKKITETKQLAKDLRQWQTNVDVANDLALKLLRDYSADDTRKVHMITENINASWRSIH
KRVSEREAALEETHRLLQQFPLDLEKFLAWLTEAETTANVLQDATRKERLLEDSKGVKELMKQ
WQDLQGEIEAHTDVYHNLDENSQKILRSLEGSDDAVLLQRRLDNMNFKWSELRKKSLNIRSHL
EASSDQWKRLHLSLQELLVWLQLKDDELSRQAPIGGDFPAVQKQNDVHRAFKRELKTKEPVI
MSTLETVRIFLTEQPLEGLEKLYQEPRELPPEERAQNVTRLLRKQAEEVNTEWEKLNLHSADW
QRKIDETLERLQELQEATDELDLKLRQAEVIKGSWQPVGDLLIDSLQDHLEKVKALRGEIAPLK
ENVSHVNDLARQLTTLGIQLSPYNLSTLEDLNTRWKLLQVAVEDRVRQLHEAHRDFGPASQHF
LSTSVQGPWERAISPNKVPYYINHETQTTCWDHPKMTELYQSLADLNNVRFSAYRTAMKLRRL
QKALCLDLLSLSAACDALDQHNLKQNDQPMDILQIINCLTTIYDRLEQEHNNLVNVPLCVDMC
LNWLLNVYDTGRTGRIRVLSFKTGIISLCKAHLEDKYRYLFKQVASSTGFCDQRRLGLLLHDSI
QIPRQLGEVASFGGSNIEPSVRSCFQFANNKPEIEAALFLDWMRLEPQSMVWLPVLHRVAAAET
AKHQAKCNICKECPIIGFRYRSLKHFNYDICQSCFFSGRVAKGHKMHYPMVEYCTPTTSGEDVR
DFAKVLKNKFRTKRYFAKHPRMGYLPVQTVLEGDNMETPVTLINFWPVDSAPASSPQLSHDDT
HSRIEHYASRLAEMENSNGSYLNDSISPNESIDDEHLLIQHYCQSLNQDSPLSQPRSPAQILISLES
EERGELERILADLEEENRNLQAEYDRLKQQHEHKGLSPLPSPPEMMPTSPQSPRDAELIAEAKLL
RQHKGRLEARMQILEDHNKQLESQLHRLRQLLEQPQAEAKVNGTTVSSPSTSLQRSDSSQPML
LRVVGSQTSDSMGEEDLLSPPQDTSTGLEEVMEQLNNSFPSSRGRNTPGKPMREDTM*
2) Split site ImS2:
•N-ter fragment (SEQ ID NO. 93):
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVMK
NIMAGLQQTNSEKILLSWVRQSTRNYPQVNVINFTTSWSDGLALNALIHSHRPDLFDWNSVVCQ
QSATQRLEHAFNIARYQLGIEKLLDPEDVDTTYPDKKSILMYITSLFQVLPQQVSIEAIQEVEMLP
RPPKVTKEEHFQLHHQMHYSQQITVSLAQGYERTSSPKPRFKSYAYTQAAYVTTSDPTRSPFPSQ
HLEAPEDKSFGSSLMESEVNLDRYQTALEEVLSWLLSAEDTLQAQGEISNDVEVVKDQFHTHEG
YMMDLTAHQGRVGNILQLGSKLIGTGKLSEDEETEVQEQMNLLNSRWECLRVASMEKQSNLH
RVLMDLQNQKLKELNDWLTKTEERTRKMEEEPLGPDLEDLKRQVQQHKVLQEDLEQEQVRVN
SLTHMVVVVDESSGDHATAALEEQLKVLGDRWANICRWTEDRWVLLQDILLKWQRLTEEQCL
FSAWLSEKEDAVNKIHTTGFKDQNEMLSSLQKLAVLKADLEKKKQSMGKLYSLKQDLLSTLKN
KSVTQKTEAWLDNFARCWDNLVQKLEKSTAQISQAVTTTQPSLTQTTVMETVTTVTTREQILVK
HAQEELPPPPPQKKRQITVDSEIRKRLDVDITELHSWITRSEAVLQSPEFAIFRKEGNFSDLKEKVN
AIEREKAEKFRKLQDASRSAQALVEQMVNEGVNADSIKQASEQLNSRWIEFCQLLSERLNWLEY
QNNIIAFYNQLQQLEQMTTTAENWLKIQPTTPSEPTAIKSQLKICKDEVNRLSGLQPQIERLKIQSI
ALKEKGQGPMFLDADFVAFTNHFKQVFSDVQAREKELQTIFDTLPPMRYQETMSAIRTWVQQS
ETKLSIPQLSVTDYEIMEQRLGELQALQSSLQEQQSGLYYLSTTVKEMSKKAPSEISRKYQSEFEE
IEGRWKKLSSQLVEHCQKLEEQMNKLRKIQNHIQTLKKWMAEVDVFLKEEWPALGCFVPGTLV
NTENGLKKIEEIKVGDKVFSHTGKLQEVVDTLIFDRDEEIISINGIDCTKNHEFYVIDKENANRVN
EDNIHLFARWVHAEELDMKKHLLIELE*
•Middle fragment (SEQ ID NO. 94):
MKFKLKEITSIETKHYKGKVHDLTVNQDHSYNVRGTVVHNSEILKKQLKQCRLLVSDIQTIQPSL
NSVNEGGQKIKNEAEPEFASRLETELKELNTQWDHMCQQVYARKEALKGGLEKTVSLQKDLSE
MHEWMTQAEEEYLERDFEYKTPDELQKAVEEMKRAKEEAQQKEAKVKLLTESVNSVIAQAPP
VAQEALKKELETLTTNYQWLCTRLNGKCKTLEEVWACWHELLSYLEKANKWLNEVEFKLKTT
ENIPGGAEEISEVLDSLENLMRHSEDNPNQIRILAQTLTDGGVMDELINEELETFNSRWRELHEEA
VRRQKLLEQSIQSAQETEKSLHLIQESLTFIDKQLAAYIADKVDAAQMPQEAQKIQSDLTSHEISL
EEMKKHNQGKEAAQRVLSQIDVAQKKLQDVSMKFRLFQKPANFELRLQESKMILDEVKMHLP
ALETKSVEQEVVQSQLNHCVNLYKSLSEVKSEVEMVIKTGRQIVQKKQTENPKELDERVTALKL
HYNELGAKVTERKQQLEKCLKLSRKMRKEMNVLTEWLAATDMELTKRSAVEGMPSNLDSEVA
WGKATQKEIEKQKVHLKSITEVGEALKTVLGKKETLVEDKLSLLNSNWIAVTSRAEEWLNLLLE
YQKHMETFDQNVDHITKWIIQADTLLDESEKKKPQQKEDVLKRLKAELNDIRPKVDSTRDQAA
NLMANRGDHCRKLVEPQISELNHRFAAISHRIKTGKASIPLKELEQFNSDIQKLLEPLEAEIQQGV
NLKEEDFNKDMNEDNEGTVKELLQRGDNLQQRITDERKREEIKIKQQLLQTKHNALKDLRSQR
RKKALEISHQWYQYKRQADDLLKCLDDIEKKLASLPEPRDERKIKEIDRELQKKKEELNAVRRQ
AEGLSEDGAAMAVEPTQIQLSKRWREIESKFAQFRRLNFAQIHTVREETMMVMTEDMPLEISYV
PSTYLTEITHVSQALLEVEQLLNAPDLCAKDFEDLFKQEESLKNIKDSLQQSSGRIDIIHSKKTAAL
QSATPVERVKLQEALSQLDFQWEKVNKMYKDRQGRFDRSVEKWRRFHYDIKIFNQWLTEAEQ
FLRKTQIPENWEHAKYKWYLKELQDGIGQRQTVVRTLNATGEEIIQQSSKTDASILQEKLGSLNL
RWQEVCKQLSDRKKRLEEQKNILSEFQRDLNEFVLWLEEADNIASIPLEPGKEQQLKEKLEQVK
LLVEELPLRQGILKQLNETGGPVLVSAPISPEEQDKLENKLKQTNLQWIKVSRALPEKQGEIEAQI
KDLGQLEKKLEDLEEQLNHLLLWLSPIRNQLEIYNQPNQEGPFDVQETEIAVQAKQPDVEEILSK
GQHLYKEGYCLDLKTQVQTPQGMKEISNIQVGDLVLSNTGYNEVLNVFPKSKKKSYKITLEDGK
EIICSEEHLFPTQTGEMNISGGLKEGMCLYVKE*
•C-ter fragment (SEQ ID NO. 95):
MMLKKILKIEELDERELIDIEVSGNHLFYANDILTHNSTQPVKRKLEDLSSEWKAVNRLLQELRA
KQPDLAPGLTTIGASPTQTVTLVTQPVVTKETAISKLEMPSSLMLEVPALADFNRAWTELTDWLS
LLDQVIKSQRVMVGDLEDINEMIIKQKATMQDLEQRRPQLEELITAAQNLKNKTSNQEARTIITD
RIERIQNQWDEVQEHLQNRRQQLNEMLKDSTQWLEAKEEAEQVLGQARAKLESWKEGPYTVD
AIQKKITETKQLAKDLRQWQTNVDVANDLALKLLRDYSADDTRKVHMITENINASWRSIHKRV
SEREAALEETHRLLQQFPLDLEKFLAWLTEAETTANVLQDATRKERLLEDSKGVKELMKQWQD
LQGEIEAHTDVYHNLDENSQKILRSLEGSDDAVLLQRRLDNMNFKWSELRKKSLNIRSHLEASS
DQWKRLHLSLQELLVWLQLKDDELSRQAPIGGDFPAVQKQNDVHRAFKRELKTKEPVIMSTLE
TVRIFLTEQPLEGLEKLYQEPRELPPEERAQNVTRLLRKQAEEVNTEWEKLNLHSADWQRKIDE
TLERLQELQEATDELDLKLRQAEVIKGSWQPVGDLLIDSLQDHLEKVKALRGEIAPLKENVSHV
NDLARQLTTLGIQLSPYNLSTLEDLNTRWKLLQVAVEDRVRQLHEAHRDFGPASQHFLSTSVQG
PWERAISPNKVPYYINHETQTTCWDHPKMTELYQSLADLNNVRFSAYRTAMKLRRLQKALCLD
LLSLSAACDALDQHNLKQNDQPMDILQIINCLTTIYDRLEQEHNNLVNVPLCVDMCLNWLLNV
YDTGRTGRIRVLSFKTGIISLCKAHLEDKYRYLFKQVASSTGFCDQRRLGLLLHDSIQIPRQLGEV
ASFGGSNIEPSVRSCFQFANNKPEIEAALFLDWMRLEPQSMVWLPVLHRVAAAETAKHQAKCNI
CKECPIIGFRYRSLKHFNYDICQSCFFSGRVAKGHKMHYPMVEYCTPTTSGEDVRDFAKVLKNK
FRTKRYFAKHPRMGYLPVQTVLEGDNMETPVTLINFWPVDSAPASSPQLSHDDTHSRIEHYASR
LAEMENSNGSYLNDSISPNESIDDEHLLIQHYCQSLNQDSPLSQPRSPAQILISLESEERGELERILA
DLEEENRNLQAEYDRLKQQHEHKGLSPLPSPPEMMPTSPQSPRDAELIAEAKLLRQHKGRLEAR
MQILEDHNKQLESQLHRLRQLLEQPQAEAKVNGTTVSSPSTSLQRSDSSQPMLLRVVGSQTSDS
MGEEDLLSPPQDTSTGLEEVMEQLNNSFPSSRGRNTPGKPMREDTM*
3) Split site ImS3:
•N-ter fragment (SEQ ID NO. 96):
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVMK
NIMAGLQQTNSEKILLSWVRQSTRNYPQVNVINFTTSWSDGLALNALIHSHRPDLFDWNSVVCQ
QSATQRLEHAFNIARYQLGIEKLLDPEDVDTTYPDKKSILMYITSLFQVLPQQVSIEAIQEVEMLP
RPPKVTKEEHFQLHHQMHYSQQITVSLAQGYERTSSPKPRFKSYAYTQAAYVTTSDPTRSPFPSQ
HLEAPEDKSFGSSLMESEVNLDRYQTALEEVLSWLLSAEDTLQAQGEISNDVEVVKDQFHTHEG
YMMDLTAHQGRVGNILQLGSKLIGTGKLSEDEETEVQEQMNLLNSRWECLRVASMEKQSNLH
RVLMDLQNQKLKELNDWLTKTEERTRKMEEEPLGPDLEDLKRQVQQHKVLQEDLEQEQVRVN
SLTHMVVVVDESSGDHATAALEEQLKVLGDRWANICRWTEDRWVLLQDILLKWQRLTEEQCL
FSAWLSEKEDAVNKIHTTGFKDQNEMLSSLQKLAVLKADLEKKKQSMGKLYSLKQDLLSTLKN
KSVTQKTEAWLDNFARCWDNLVQKLEKSTAQISQAVTTTQPSLTQTTVMETVTTVTTREQILVK
HAQEELPPPPPQKKRQITVDSEIRKRLDVDITELHSWITRSEAVLQSPEFAIFRKEGNFSDLKEKVN
AIEREKAEKFRKLQDASRSAQALVEQMVNEGVNADSIKQASEQLNSRWIEFCQLLSERLNWLEY
QNNIIAFYNQLQQLEQMTTTAENWLKIQPTTPSEPTAIKSQLKICKDEVNRLSGLQPQIERLKIQSI
ALKEKGQGPMFLDADFVAFTNHFKQVFSDVQAREKELQTIFDTLPPMRYQETMSAIRTWVQQS
ETKLSIPQLSVTDYEIMEQRLGELQALQSSLQEQQSGLYYLSTTVKEMSKKAPSEISRKYQSEFEE
IEGRWKKLSSQLVEHCQKLEEQMNKLRKIQNHIQTLKKWMAEVDVFLKEEWPALGDSEILKKQ
LKQCRLLVSDIQTIQPSLNSVNEGGQKIKNEAEPEFASRLETELKELNTQWDHMCQQVYARKEA
LKGGLEKTVSLQKDLSEMHEWMTQAEEEYLERDFEYGCFVPGTLVNTENGLKKIEEIKVGDKV
FSHTGKLQEVVDTLIFDRDEEIISINGIDCTKNHEFYVIDKENANRVNEDNIHLFARWVHAEELD
MKKHLLIELE*
•Middle fragment (SEQ ID NO. 97):
MKFKLKEITSIETKHYKGKVHDLTVNQDHSYNVRGTVVHNSKTPDELQKAVEEMKRAKEEAQ
QKEAKVKLLTESVNSVIAQAPPVAQEALKKELETLTTNYQWLCTRLNGKCKTLEEVWACWHEL
LSYLEKANKWLNEVEFKLKTTENIPGGAEEISEVLDSLENLMRHSEDNPNQIRILAQTLTDGGVM
DELINEELETFNSRWRELHEEAVRRQKLLEQSIQSAQETEKSLHLIQESLTFIDKQLAAYIADKVD
AAQMPQEAQKIQSDLTSHEISLEEMKKHNQGKEAAQRVLSQIDVAQKKLQDVSMKFRLFQKPA
NFELRLQESKMILDEVKMHLPALETKSVEQEVVQSQLNHCVNLYKSLSEVKSEVEMVIKTGRQI
VQKKQTENPKELDERVTALKLHYNELGAKVTERKQQLEKCLKLSRKMRKEMNVLTEWLAATD
MELTKRSAVEGMPSNLDSEVAWGKATQKEIEKQKVHLKSITEVGEALKTVLGKKETLVEDKLS
LLNSNWIAVTSRAEEWLNLLLEYQKHMETFDQNVDHITKWIIQADTLLDESEKKKPQQKEDVL
KRLKAELNDIRPKVDSTRDQAANLMANRGDHCRKLVEPQISELNHRFAAISHRIKTGKASIPLKE
LEQFNSDIQKLLEPLEAEIQQGVNLKEEDFNKDMNEDNEGTVKELLQRGDNLQQRITDERKREEI
KIKQQLLQTKHNALKDLRSQRRKKALEISHQWYQYKRQADDLLKCLDDIEKKLASLPEPRDER
KIKEIDRELQKKKEELNAVRRQAEGLSEDGAAMAVEPTQIQLSKRWREIESKFAQFRRLNFAQIH
TVREETMMVMTEDMPLEISYVPSTYLTEITHVSQALLEVEQLLNAPDLCAKDFEDLFKQEESLK
NIKDSLQQSSGRIDIIHSKKTAALQSATPVERVKLQEALSQLDFQWEKVNKMYKDRQGRFDRSV
EKWRRFHYDIKIFNQWLTEAEQFLRKTQIPENWEHAKYKWYLKELQDGIGQRQTVVRTLNATG
EEIIQQSSKTDASILQEKLGSLNLRWQEVCKQLSDRKKRLEEQKNILSEFQRDLNEFVLWLEEAD
NIASIPLEPGKEQQLKEKLEQVKLLVEELPLRQGILKQLNETGGPVLVSAPISPEEQDKLENKLKQ
TNLQWIKVSRALPEKQGEIEAQIKDLGQLEKKLEDLEEQLNHLLLWLSPIRNQLEIYNQPNQEGP
FDVQETEIAVQAKQPDVEEILSKGQHLYKEKPATQPVKRKLEDLSSEWKAVNRLLQELRAKQPD
LAPGLTTIGYCLDLKTQVQTPQGMKEISNIQVGDLVLSNTGYNEVLNVFPKSKKKSYKITLEDGK
EIICSEEHLFPTQTGEMNISGGLKEGMCLYVKE*
•C-ter fragment (SEQ ID NO. 98):
MMLKKILKIEELDERELIDIEVSGNHLFYANDILTHNSPTQTVTLVTQPVVTKETAISKLEMPSSL
MLEVPALADFNRAWTELTDWLSLLDQVIKSQRVMVGDLEDINEMIIKQKATMQDLEQRRPQLE
ELITAAQNLKNKTSNQEARTIITDRIERIQNQWDEVQEHLQNRRQQLNEMLKDSTQWLEAKEEA
EQVLGQARAKLESWKEGPYTVDAIQKKITETKQLAKDLRQWQTNVDVANDLALKLLRDYSAD
DTRKVHMITENINASWRSIHKRVSEREAALEETHRLLQQFPLDLEKFLAWLTEAETTANVLQDA
TRKERLLEDSKGVKELMKQWQDLQGEIEAHTDVYHNLDENSQKILRSLEGSDDAVLLQRRLDN
MNFKWSELRKKSLNIRSHLEASSDQWKRLHLSLQELLVWLQLKDDELSRQAPIGGDFPAVQKQ
NDVHRAFKRELKTKEPVIMSTLETVRIFLTEQPLEGLEKLYQEPRELPPEERAQNVTRLLRKQAE
EVNTEWEKLNLHSADWQRKIDETLERLQELQEATDELDLKLRQAEVIKGSWQPVGDLLIDSLQD
HLEKVKALRGEIAPLKENVSHVNDLARQLTTLGIQLSPYNLSTLEDLNTRWKLLQVAVEDRVRQ
LHEAHRDFGPASQHELSTSVQGPWERAISPNKVPYYINHETQTTCWDHPKMTELYQSLADLNNV
RFSAYRTAMKLRRLQKALCLDLLSLSAACDALDQHNLKQNDQPMDILQIINCLTTIYDRLEQEH
NNLVNVPLCVDMCLNWLLNVYDTGRTGRIRVLSFKTGIISLCKAHLEDKYRYLFKQVASSTGFC
DQRRLGLLLHDSIQIPRQLGEVASFGGSNIEPSVRSCFQFANNKPEIEAALFLDWMRLEPQSMVW
LPVLHRVAAAETAKHQAKCNICKECPIIGFRYRSLKHFNYDICQSCFFSGRVAKGHKMHYPMVE
YCTPTTSGEDVRDFAKVLKNKFRTKRYFAKHPRMGYLPVQTVLEGDNMETPVTLINFWPVDSA
PASSPQLSHDDTHSRIEHYASRLAEMENSNGSYLNDSISPNESIDDEHLLIQHYCQSLNQDSPLSQ
PRSPAQILISLESEERGELERILADLEEENRNLQAEYDRLKQQHEHKGLSPLPSPPEMMPTSPQSPR
DAELIAEAKLLRQHKGRLEARMQILEDHNKQLESQLHRLRQLLEQPQAEAKVNGTTVSSPSTSL
QRSDSSQPMLLRVVGSQTSDSMGEEDLLSPPQDTSTGLEEVMEQLNNSFPSSRGRNTPGKPMRE
DTM*
4) Split site ImS4:
•N-ter fragment (SEQ ID NO. 99):
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVMK
NIMAGLQQTNSEKILLSWVRQSTRNYPQVNVINFTTSWSDGLALNALIHSHRPDLFDWNSVVCQ
QSATQRLEHAFNIARYQLGIEKLLDPEDVDTTYPDKKSILMYITSLFQVLPQQVSIEAIQEVEMLP
RPPKVTKEEHFQLHHQMHYSQQITVSLAQGYERTSSPKPRFKSYAYTQAAYVTTSDPTRSPFPSQ
HLEAPEDKSFGSSLMESEVNLDRYQTALEEVLSWLLSAEDTLQAQGEISNDVEVVKDQFHTHEG
YMMDLTAHQGRVGNILQLGSKLIGTGKLSEDEETEVQEQMNLLNSRWECLRVASMEKQSNLH
RVLMDLQNQKLKELNDWLTKTEERTRKMEEEPLGPDLEDLKRQVQQHKVLQEDLEQEQVRVN
SLTHMVVVVDESSGDHATAALEEQLKVLGDRWANICRWTEDRWVLLQDILLKWQRLTEEQCL
FSAWLSEKEDAVNKIHTTGFKDQNEMLSSLQKLAVLKADLEKKKQSMGKLYSLKQDLLSTLKN
KSVTQKTEAWLDNFARCWDNLVQKLEKSTAQISQAVTTTQPSLTQTTVMETVTTVTTREQILVK
HAQEELPPPPPQKKRQITVDSEIRKRLDVDITELHSWITRSEAVLQSPEFAIFRKEGNFSDLKEKVN
AIEREKAEKFRKLQDASRSAQALVEQMVNEGVNADSIKQASEQLNSRWIEFCQLLSERLNWLEY
QNNIIAFYNQLQQLEQMTTTAENWLKIQPTTPSEPTAIKSQLKICKDEVNRLSGLQPQIERLKIQSI
ALKEKGQGPMFLDADFVAFTNHFKQVFSDVQAREKELQTIFDTLPPMRYQETMSAIRTWVQQS
ETKLSIPQLSVTDYEIMEQRLGELQALQSSLQEQQSGLYYLSTTVKEMSKKAPSEISRKYQSEFEE
IEGRWKKLSSQLVEHCQKLEEQMNKLRKIQNHIQTLKKWMAEVDVFLKEEWPALGDSEILKKQ
LKQCRLLVSDIQTIQPSLNSVNEGGQKIKNEAEPEFASRLETELKELNTQWDHMCQQVYARKEA
LKGGLEKTVSLQKDLSEMHEWMTQAEEEYLERDFEGCFVPGTLVNTENGLKKIEEIKVGDKVFS
HTGKLQEVVDTLIFDRDEEIISINGIDCTKNHEFYVIDKENANRVNEDNIHLFARWVHAEELDMK
KHLLIELE*
•Middle fragment (SEQ ID NO. 100):
MKFKLKEITSIETKHYKGKVHDLTVNQDHSYNVRGTVVHNSTPDELQKAVEEMKRAKEEAQQ
KEAKVKLLTESVNSVIAQAPPVAQEALKKELETLTTNYQWLCTRLNGKCKTLEEVWACWHELL
SYLEKANKWLNEVEFKLKTTENIPGGAEEISEVLDSLENLMRHSEDNPNQIRILAQTLTDGGVMD
ELINEELETFNSRWRELHEEAVRRQKLLEQSIQSAQETEKSLHLIQESLTFIDKQLAAYIADKVDA
AQMPQEAQKIQSDLTSHEISLEEMKKHNQGKEAAQRVLSQIDVAQKKLQDVSMKFRLFQKPAN
FELRLQESKMILDEVKMHLPALETKSVEQEVVQSQLNHCVNLYKSLSEVKSEVEMVIKTGRQIV
QKKQTENPKELDERVTALKLHYNELGAKVTERKQQLEKCLKLSRKMRKEMNVLTEWLAATD
MELTKRSAVEGMPSNLDSEVAWGKATQKEIEKQKVHLKSITEVGEALKTVLGKKETLVEDKLS
LLNSNWIAVTSRAEEWLNLLLEYQKHMETFDQNVDHITKWIIQADTLLDESEKKKPQQKEDVL
KRLKAELNDIRPKVDSTRDQAANLMANRGDHCRKLVEPQISELNHRFAAISHRIKTGKASIPLKE
LEQFNSDIQKLLEPLEAEIQQGVNLKEEDFNKDMNEDNEGTVKELLQRGDNLQQRITDERKREEI
KIKQQLLQTKHNALKDLRSQRRKKALEISHQWYQYKRQADDLLKCLDDIEKKLASLPEPRDER
KIKEIDRELQKKKEELNAVRRQAEGLSEDGAAMAVEPTQIQLSKRWREIESKFAQFRRLNFAQIH
TVREETMMVMTEDMPLEISYVPSTYLTEITHVSQALLEVEQLLNAPDLCAKDFEDLFKQEESLK
NIKDSLQQSSGRIDIIHSKKTAALQSATPVERVKLQEALSQLDFQWEKVNKMYKDRQGRFDRSV
EKWRRFHYDIKIFNQWLTEAEQFLRKTQIPENWEHAKYKWYLKELQDGIGQRQTVVRTLNATG
EEIIQQSSKTDASILQEKLGSLNLRWQEVCKQLSDRKKRLEEQKNILSEFQRDLNEFVLWLEEAD
NIASIPLEPGKEQQLKEKLEQVKLLVEELPLRQGILKQLNETGGPVLVSAPISPEEQDKLENKLKQ
TNLQWIKVSRALPEKQGEIEAQIKDLGQLEKKLEDLEEQLNHLLLWLSPIRNQLEIYNQPNQEGP
FDVQETEIAVQAKQPDVEEILSKGQHLYKEKPATQPVKRKLEDLSSEWKAVNRLLQELRAKQPD
LAPGLTTIGYCLDLKTQVQTPQGMKEISNIQVGDLVLSNTGYNEVLNVFPKSKKKSYKITLEDGK
EIICSEEHLFPTQTGEMNISGGLKEGMCLYVKE*
•C-ter fragment (SEQ ID NO. 101):
MMLKKILKIEELDERELIDIEVSGNHLFYANDILTHNSPTQTVTLVTQPVVTKETAISKLEMPSSL
MLEVPALADFNRAWTELTDWLSLLDQVIKSQRVMVGDLEDINEMIIKQKATMQDLEQRRPQLE
ELITAAQNLKNKTSNQEARTIITDRIERIQNQWDEVQEHLQNRRQQLNEMLKDSTQWLEAKEEA
EQVLGQARAKLESWKEGPYTVDAIQKKITETKQLAKDLRQWQTNVDVANDLALKLLRDYSAD
DTRKVHMITENINASWRSIHKRVSEREAALEETHRLLQQFPLDLEKFLAWLTEAETTANVLQDA
TRKERLLEDSKGVKELMKQWQDLQGEIEAHTDVYHNLDENSQKILRSLEGSDDAVLLQRRLDN
MNFKWSELRKKSLNIRSHLEASSDQWKRLHLSLQELLVWLQLKDDELSRQAPIGGDFPAVQKQ
NDVHRAFKRELKTKEPVIMSTLETVRIFLTEQPLEGLEKLYQEPRELPPEERAQNVTRLLRKQAE
EVNTEWEKLNLHSADWQRKIDETLERLQELQEATDELDLKLRQAEVIKGSWQPVGDLLIDSLQD
HLEKVKALRGEIAPLKENVSHVNDLARQLTTLGIQLSPYNLSTLEDLNTRWKLLQVAVEDRVRQ
LHEAHRDFGPASQHELSTSVQGPWERAISPNKVPYYINHETQTTCWDHPKMTELYQSLADLNNV
RFSAYRTAMKLRRLQKALCLDLLSLSAACDALDQHNLKQNDQPMDILQIINCLTTIYDRLEQEH
NNLVNVPLCVDMCLNWLLNVYDTGRTGRIRVLSFKTGIISLCKAHLEDKYRYLFKQVASSTGFC
DQRRLGLLLHDSIQIPRQLGEVASFGGSNIEPSVRSCFQFANNKPEIEAALFLDWMRLEPQSMVW
LPVLHRVAAAETAKHQAKCNICKECPIIGFRYRSLKHFNYDICQSCFFSGRVAKGHKMHYPMVE
YCTPTTSGEDVRDFAKVLKNKFRTKRYFAKHPRMGYLPVQTVLEGDNMETPVTLINFWPVDSA
PASSPQLSHDDTHSRIEHYASRLAEMENSNGSYLNDSISPNESIDDEHLLIQHYCQSLNQDSPLSQ
PRSPAQILISLESEERGELERILADLEEENRNLQAEYDRLKQQHEHKGLSPLPSPPEMMPTSPQSPR
DAELIAEAKLLRQHKGRLEARMQILEDHNKQLESQLHRLRQLLEQPQAEAKVNGTTVSSPSTSL
QRSDSSQPMLLRVVGSQTSDSMGEEDLLSPPQDTSTGLEEVMEQLNNSFPSSRGRNTPGKPMRE
DTM*
b. Using Nrdj-1:
1) Split site NrS1:
•N-ter fragment (SEQ ID NO. 102):
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVM
KNIMAGLQQTNSEKILLSWVRQSTRNYPQVNVINFTTSWSDGLALNALIHSHRPDLFDWNSVV
CQQSATQRLEHAFNIARYQLGIEKLLDPEDVDTTYPDKKSILMYITSLFQVLPQQVSIEAIQEVE
MLPRPPKVTKEEHFQLHHQMHYSQQITVSLAQGYERTSSPKPRFKSYAYTQAAYVTTSDPTRSP
FPSQHLEAPEDKSFGSSLMESEVNLDRYQTALEEVLSWLLSAEDTLQAQGEISNDVEVVKDQFH
THEGYMMDLTAHQGRVGNILQLGSKLIGTGKLSEDEETEVQEQMNLLNSRWECLRVASMEKQ
SNLHRVLMDLQNQKLKELNDWLTKTEERTRKMEEEPLGPDLEDLKRQVQQHKVLQEDLEQEQ
VRVNSLTHMVVVVDESSGDHATAALEEQLKVLGDRWANICRWTEDRWVLLQDILLKWQRLT
EEQCLFSAWLSEKEDAVNKIHTTGFKDQNEMLSSLQKLAVLKADLEKKKQSMGKLYSLKQDL
LSTLKNKSVTQKTEAWLDNFARCWDNLVQKLEKSTAQISQAVTTTQPSLTQTTVMETVTTVTT
REQILVKHAQEELPPPPPQKKRQITVDSEIRKRLDVDITELHSWITRSEAVLQSPEFAIFRKEGNFS
DLKEKVNAIEREKAEKFRKLQDASRSAQALVEQMVNEGVNADSIKQASEQLNSRWIEFCQLLS
ERLNWLEYQNNIIAFYNQLQQLEQMTTTAENWLKIQPTTPSEPTAIKSQLKICKDEVNRLSGLQP
QIERLKIQSIALKEKGQGPMFLDADFVAFTNHFKQVFSDVQAREKELQTIFDTLPPMRYQETMS
AIRTWVQQSETKLSIPQLSVTDYEIMEQRLGELQALQSSLQEQQSGLYYLSTTVKEMSKKAPSEI
SRKYQSEFEEIEGRWKKLSSQLVEHCQKLEEQMNKLRKIQNHIQTLKKWMAEVDVFLKEEWP
ALGCCLVGSSEIITRNYGKTTIKEVVEIFDNDKNIQVLAFNTHTDNIEWAPIKAAQLTRPNAELV
ELEIDTLHGVKTIRCTPDHPVYTKNRGYVRADELTDDDELVVAI*
•Middle fragment (SEQ ID NO. 103):
MEAKTYIGKLKSRKIVSNEDTYDIQTSTHNFFANDILVHNSEILKKQLKQCRLLVSDIQTIQPSLN
SVNEGGQKIKNEAEPEFASRLETELKELNTQWDHMCQQVYARKEALKGGLEKTVSLQKDLSE
MHEWMTQAEEEYLERDFEYKTPDELQKAVEEMKRAKEEAQQKEAKVKLLTESVNSVIAQAPP
VAQEALKKELETLTTNYQWLCTRLNGKCKTLEEVWACWHELLSYLEKANKWLNEVEFKLKT
TENIPGGAEEISEVLDSLENLMRHSEDNPNQIRILAQTLTDGGVMDELINEELETFNSRWRELHE
EAVRRQKLLEQSIQSAQETEKSLHLIQESLTFIDKQLAAYIADKVDAAQMPQEAQKIQSDLTSHE
ISLEEMKKHNQGKEAAQRVLSQIDVAQKKLQDVSMKFRLFQKPANFELRLQESKMILDEVKM
HLPALETKSVEQEVVQSQLNHCVNLYKSLSEVKSEVEMVIKTGRQIVQKKQTENPKELDERVT
ALKLHYNELGAKVTERKQQLEKCLKLSRKMRKEMNVLTEWLAATDMELTKRSAVEGMPSNL
DSEVAWGKATQKEIEKQKVHLKSITEVGEALKTVLGKKETLVEDKLSLLNSNWIAVTSRAEEW
LNLLLEYQKHMETFDQNVDHITKWIIQADTLLDESEKKKPQQKEDVLKRLKAELNDIRPKVDS
TRDQAANLMANRGDHCRKLVEPQISELNHRFAAISHRIKTGKASIPLKELEQFNSDIQKLLEPLE
AEIQQGVNLKEEDFNKDMNEDNEGTVKELLQRGDNLQQRITDERKREEIKIKQQLLQTKHNAL
KDLRSQRRKKALEISHQWYQYKRQADDLLKCLDDIEKKLASLPEPRDERKIKEIDRELQKKKEE
LNAVRRQAEGLSEDGAAMAVEPTQIQLSKRWREIESKFAQFRRLNFAQIHTVREETMMVMTED
MPLEISYVPSTYLTEITHVSQALLEVEQLLNAPDLCAKDFEDLFKQEESLKNIKDSLQQSSGRIDII
HSKKTAALQSATPVERVKLQEALSQLDFQWEKVNKMYKDRQGRFDRSVEKWRRFHYDIKIFN
QWLTEAEQFLRKTQIPENWEHAKYKWYLKELQDGIGQRQTVVRTLNATGEEIIQQSSKTDASIL
QEKLGSLNLRWQEVCKQLSDRKKRLEEQKNILSEFQRDLNEFVLWLEEADNIASIPLEPGKEQQ
LKEKLEQVKLLVEELPLRQGILKQLNETGGPVLVSAPISPEEQDKLENKLKQTNLQWIKVSRAL
PEKQGEIEAQIKDLGQLEKKLEDLEEQLNHLLLWLSPIRNQLEIYNQPNQEGPFDVQETEIAVQA
KQPDVEEILSKGQHLYKEGYCLDLKTQVQTPQGMKEISNIQVGDLVLSNTGYNEVLNVFPKSK
KKSYKITLEDGKEIICSEEHLFPTQTGEMNISGGLKEGMCLYVKE*
•C-ter fragment (SEQ ID NO. 104):
MMLKKILKIEELDERELIDIEVSGNHLFYANDILTHNSTQPVKRKLEDLSSEWKAVNRLLQELRA
KQPDLAPGLTTIGASPTQTVTLVTQPVVTKETAISKLEMPSSLMLEVPALADFNRAWTELTDWLS
LLDQVIKSQRVMVGDLEDINEMIIKQKATMQDLEQRRPQLEELITAAQNLKNKTSNQEARTIITD
RIERIQNQWDEVQEHLQNRRQQLNEMLKDSTQWLEAKEEAEQVLGQARAKLESWKEGPYTVD
AIQKKITETKQLAKDLRQWQTNVDVANDLALKLLRDYSADDTRKVHMITENINASWRSIHKRV
SEREAALEETHRLLQQFPLDLEKFLAWLTEAETTANVLQDATRKERLLEDSKGVKELMKQWQD
LQGEIEAHTDVYHNLDENSQKILRSLEGSDDAVLLQRRLDNMNFKWSELRKKSLNIRSHLEASS
DQWKRLHLSLQELLVWLQLKDDELSRQAPIGGDFPAVQKQNDVHRAFKRELKTKEPVIMSTLE
TVRIFLTEQPLEGLEKLYQEPRELPPEERAQNVTRLLRKQAEEVNTEWEKLNLHSADWQRKIDE
TLERLQELQEATDELDLKLRQAEVIKGSWQPVGDLLIDSLQDHLEKVKALRGEIAPLKENVSHV
NDLARQLTTLGIQLSPYNLSTLEDLNTRWKLLQVAVEDRVRQLHEAHRDFGPASQHFLSTSVQG
PWERAISPNKVPYYINHETQTTCWDHPKMTELYQSLADLNNVRFSAYRTAMKLRRLQKALCLD
LLSLSAACDALDQHNLKQNDQPMDILQIINCLTTIYDRLEQEHNNLVNVPLCVDMCLNWLLNV
YDTGRTGRIRVLSFKTGIISLCKAHLEDKYRYLFKQVASSTGFCDQRRLGLLLHDSIQIPRQLGEV
ASFGGSNIEPSVRSCFQFANNKPEIEAALFLDWMRLEPQSMVWLPVLHRVAAAETAKHQAKCNI
CKECPIIGFRYRSLKHFNYDICQSCFFSGRVAKGHKMHYPMVEYCTPTTSGEDVRDFAKVLKNK
FRTKRYFAKHPRMGYLPVQTVLEGDNMETPVTLINFWPVDSAPASSPQLSHDDTHSRIEHYASR
LAEMENSNGSYLNDSISPNESIDDEHLLIQHYCQSLNQDSPLSQPRSPAQILISLESEERGELERILA
DLEEENRNLQAEYDRLKQQHEHKGLSPLPSPPEMMPTSPQSPRDAELIAEAKLLRQHKGRLEAR
MQILEDHNKQLESQLHRLRQLLEQPQAEAKVNGTTVSSPSTSLQRSDSSQPMLLRVVGSQTSDS
MGEEDLLSPPQDTSTGLEEVMEQLNNSFPSSRGRNTPGKPMREDTM*
2) Split site NrS2:
•N-ter fragment (SEQ ID NO. 105):
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVMK
NIMAGLQQTNSEKILLSWVRQSTRNYPQVNVINFTTSWSDGLALNALIHSHRPDLFDWNSVVCQ
QSATQRLEHAFNIARYQLGIEKLLDPEDVDTTYPDKKSILMYITSLFQVLPQQVSIEAIQEVEMLP
RPPKVTKEEHFQLHHQMHYSQQITVSLAQGYERTSSPKPRFKSYAYTQAAYVTTSDPTRSPFPSQ
HLEAPEDKSFGSSLMESEVNLDRYQTALEEVLSWLLSAEDTLQAQGEISNDVEVVKDQFHTHEG
YMMDLTAHQGRVGNILQLGSKLIGTGKLSEDEETEVQEQMNLLNSRWECLRVASMEKQSNLH
RVLMDLQNQKLKELNDWLTKTEERTRKMEEEPLGPDLEDLKRQVQQHKVLQEDLEQEQVRVN
SLTHMVVVVDESSGDHATAALEEQLKVLGDRWANICRWTEDRWVLLQDILLKWQRLTEEQCL
FSAWLSEKEDAVNKIHTTGFKDQNEMLSSLQKLAVLKADLEKKKQSMGKLYSLKQDLLSTLKN
KSVTQKTEAWLDNFARCWDNLVQKLEKSTAQISQAVTTTQPSLTQTTVMETVTTVTTREQILVK
HAQEELPPPPPQKKRQITVDSEIRKRLDVDITELHSWITRSEAVLQSPEFAIFRKEGNFSDLKEKVN
AIEREKAEKFRKLQDASRSAQALVEQMVNEGVNADSIKQASEQLNSRWIEFCQLLSERLNWLEY
QNNIIAFYNQLQQLEQMTTTAENWLKIQPTTPSEPTAIKSQLKICKDEVNRLSGLQPQIERLKIQSI
ALKEKGQGPMFLDADFVAFTNHFKQVFSDVQAREKELQTIFDTLPPMRYQETMSAIRTWVQQS
ETKLSIPQLSVTDYEIMEQRLGELQALQSSLQEQQSGLYYLSTTVKEMSKKAPSEISRKYQSEFEE
IEGRWKKLSSQLVEHCQKLEEQMNKLRKIQNHIQTLKKWMAEVDVFLKEEWPALGDCCLVGSS
EIITRNYGKTTIKEVVEIFDNDKNIQVLAFNTHTDNIEWAPIKAAQLTRPNAELVELEIDTLHGVK
TIRCTPDHPVYTKNRGYVRADELTDDDELVVAI*
•Middle fragment (SEQ ID NO. 106):
MEAKTYIGKLKSRKIVSNEDTYDIQTSTHNFFANDILVHNSEILKKQLKQCRLLVSDIQTIQPSLNS
VNEGGQKIKNEAEPEFASRLETELKELNTQWDHMCQQVYARKEALKGGLEKTVSLQKDLSEM
HEWMTQAEEEYLERDFEYKTPDELQKAVEEMKRAKEEAQQKEAKVKLLTESVNSVIAQAPPVA
QEALKKELETLTTNYQWLCTRLNGKCKTLEEVWACWHELLSYLEKANKWLNEVEFKLKTTENI
PGGAEEISEVLDSLENLMRHSEDNPNQIRILAQTLTDGGVMDELINEELETFNSRWRELHEEAVR
RQKLLEQSIQSAQETEKSLHLIQESLTFIDKQLAAYIADKVDAAQMPQEAQKIQSDLTSHEISLEE
MKKHNQGKEAAQRVLSQIDVAQKKLQDVSMKFRLFQKPANFELRLQESKMILDEVKMHLPAL
ETKSVEQEVVQSQLNHCVNLYKSLSEVKSEVEMVIKTGRQIVQKKQTENPKELDERVTALKLHY
NELGAKVTERKQQLEKCLKLSRKMRKEMNVLTEWLAATDMELTKRSAVEGMPSNLDSEVAW
GKATQKEIEKQKVHLKSITEVGEALKTVLGKKETLVEDKLSLLNSNWIAVTSRAEEWLNLLLEY
QKHMETFDQNVDHITKWIIQADTLLDESEKKKPQQKEDVLKRLKAELNDIRPKVDSTRDQAAN
LMANRGDHCRKLVEPQISELNHRFAAISHRIKTGKASIPLKELEQFNSDIQKLLEPLEAEIQQGVN
LKEEDFNKDMNEDNEGTVKELLQRGDNLQQRITDERKREEIKIKQQLLQTKHNALKDLRSQRR
KKALEISHQWYQYKRQADDLLKCLDDIEKKLASLPEPRDERKIKEIDRELQKKKEELNAVRRQA
EGLSEDGAAMAVEPTQIQLSKRWREIESKFAQFRRLNFAQIHTVREETMMVMTEDMPLEISYVP
STYLTEITHVSQALLEVEQLLNAPDLCAKDFEDLFKQEESLKNIKDSLQQSSGRIDIIHSKKTAAL
QSATPVERVKLQEALSQLDFQWEKVNKMYKDRQGRFDRSVEKWRRFHYDIKIFNQWLTEAEQ
FLRKTQIPENWEHAKYKWYLKELQDGIGQRQTVVRTLNATGEEIIQQSSKTDASILQEKLGSLNL
RWQEVCKQLSDRKKRLEEQKNILSEFQRDLNEFVLWLEEADNIASIPLEPGKEQQLKEKLEQVK
LLVEELPLRQGILKQLNETGGPVLVSAPISPEEQDKLENKLKQTNLQWIKVSRALPEKQGEIEAQI
KDLGQLEKKLEDLEEQLNHLLLWLSPIRNQLEIYNQPNQEGPFDVQETEIAVQAKQPDVEEILSK
GQHLYKEGYCLDLKTQVQTPQGMKEISNIQVGDLVLSNTGYNEVLNVFPKSKKKSYKITLEDGK
EIICSEEHLFPTQTGEMNISGGLKEGMCLYVKE*
•C-ter fragment (SEQ ID NO. 107):
MMLKKILKIEELDERELIDIEVSGNHLFYANDILTHNSTQPVKRKLEDLSSEWKAVNRLLQELRA
KQPDLAPGLTTIGASPTQTVTLVTQPVVTKETAISKLEMPSSLMLEVPALADFNRAWTELTDWLS
LLDQVIKSQRVMVGDLEDINEMIIKQKATMQDLEQRRPQLEELITAAQNLKNKTSNQEARTIITD
RIERIQNQWDEVQEHLQNRRQQLNEMLKDSTQWLEAKEEAEQVLGQARAKLESWKEGPYTVD
AIQKKITETKQLAKDLRQWQTNVDVANDLALKLLRDYSADDTRKVHMITENINASWRSIHKRV
SEREAALEETHRLLQQFPLDLEKFLAWLTEAETTANVLQDATRKERLLEDSKGVKELMKQWQD
LQGEIEAHTDVYHNLDENSQKILRSLEGSDDAVLLQRRLDNMNFKWSELRKKSLNIRSHLEASS
DQWKRLHLSLQELLVWLQLKDDELSRQAPIGGDFPAVQKQNDVHRAFKRELKTKEPVIMSTLE
TVRIFLTEQPLEGLEKLYQEPRELPPEERAQNVTRLLRKQAEEVNTEWEKLNLHSADWQRKIDE
TLERLQELQEATDELDLKLRQAEVIKGSWQPVGDLLIDSLQDHLEKVKALRGEIAPLKENVSHV
NDLARQLTTLGIQLSPYNLSTLEDLNTRWKLLQVAVEDRVRQLHEAHRDFGPASQHFLSTSVQG
PWERAISPNKVPYYINHETQTTCWDHPKMTELYQSLADLNNVRFSAYRTAMKLRRLQKALCLD
LLSLSAACDALDQHNLKQNDQPMDILQIINCLTTIYDRLEQEHNNLVNVPLCVDMCLNWLLNV
YDTGRTGRIRVLSFKTGIISLCKAHLEDKYRYLFKQVASSTGFCDQRRLGLLLHDSIQIPRQLGEV
ASFGGSNIEPSVRSCFQFANNKPEIEAALFLDWMRLEPQSMVWLPVLHRVAAAETAKHQAKCNI
CKECPIIGFRYRSLKHFNYDICQSCFFSGRVAKGHKMHYPMVEYCTPTTSGEDVRDFAKVLKNK
FRTKRYFAKHPRMGYLPVQTVLEGDNMETPVTLINFWPVDSAPASSPQLSHDDTHSRIEHYASR
LAEMENSNGSYLNDSISPNESIDDEHLLIQHYCQSLNQDSPLSQPRSPAQILISLESEERGELERILA
DLEEENRNLQAEYDRLKQQHEHKGLSPLPSPPEMMPTSPQSPRDAELIAEAKLLRQHKGRLEAR
MQILEDHNKQLESQLHRLRQLLEQPQAEAKVNGTTVSSPSTSLQRSDSSQPMLLRVVGSQTSDS
MGEEDLLSPPQDTSTGLEEVMEQLNNSFPSSRGRNTPGKPMREDTM*
3) Split site NrS3:
•N-ter fragment (SEQ ID NO. 108):
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVMK
NIMAGLQQTNSEKILLSWVRQSTRNYPQVNVINFTTSWSDGLALNALIHSHRPDLFDWNSVVCQ
QSATQRLEHAFNIARYQLGIEKLLDPEDVDTTYPDKKSILMYITSLFQVLPQQVSIEAIQEVEMLP
RPPKVTKEEHFQLHHQMHYSQQITVSLAQGYERTSSPKPRFKSYAYTQAAYVTTSDPTRSPFPSQ
HLEAPEDKSFGSSLMESEVNLDRYQTALEEVLSWLLSAEDTLQAQGEISNDVEVVKDQFHTHEG
YMMDLTAHQGRVGNILQLGSKLIGTGKLSEDEETEVQEQMNLLNSRWECLRVASMEKQSNLH
RVLMDLQNQKLKELNDWLTKTEERTRKMEEEPLGPDLEDLKRQVQQHKVLQEDLEQEQVRVN
SLTHMVVVVDESSGDHATAALEEQLKVLGDRWANICRWTEDRWVLLQDILLKWQRLTEEQCL
FSAWLSEKEDAVNKIHTTGFKDQNEMLSSLQKLAVLKADLEKKKQSMGKLYSLKQDLLSTLKN
KSVTQKTEAWLDNFARCWDNLVQKLEKSTAQISQAVTTTQPSLTQTTVMETVTTVTTREQILVK
HAQEELPPPPPQKKRQITVDSEIRKRLDVDITELHSWITRSEAVLQSPEFAIFRKEGNFSDLKEKVN
AIEREKAEKFRKLQDASRSAQALVEQMVNEGVNADSIKQASEQLNSRWIEFCQLLSERLNWLEY
QNNIIAFYNQLQQLEQMTTTAENWLKIQPTTPSEPTAIKSQLKICKDEVNRLSGLQPQIERLKIQSI
ALKEKGQGPMFLDADFVAFTNHFKQVFSDVQAREKELQTIFDTLPPMRYQETMSAIRTWVQQS
ETKLSIPQLSVTDYEIMEQRLGELQALQSSLQEQQSGLYYLSTTVKEMSKKAPSEISRKYQSEFEE
IEGRWKKLSSQLVEHCQKLEEQMNKLRKIQNHIQTLKKWMAEVDVFLKEEWPALGDSEILKKQ
LKQCRLLVSDIQTIQPSLNSVNEGGQKIKNEAEPEFASRLETELKELNTQWDHMCQQVYARKEA
LKGGLEKTVSLQKDLSEMHEWMTQAEEEYLERDFEYCCLVGSSEIITRNYGKTTIKEVVEIFDND
KNIQVLAFNTHTDNIEWAPIKAAQLTRPNAELVELEIDTLHGVKTIRCTPDHPVYTKNRGYVRAD
ELTDDDELVVAI*
•Middle fragment (SEQ ID NO. 109):
MEAKTYIGKLKSRKIVSNEDTYDIQTSTHNFFANDILVHNSKTPDELQKAVEEMKRAKEEAQQK
EAKVKLLTESVNSVIAQAPPVAQEALKKELETLTTNYQWLCTRLNGKCKTLEEVWACWHELLS
YLEKANKWLNEVEFKLKTTENIPGGAEEISEVLDSLENLMRHSEDNPNQIRILAQTLTDGGVMD
ELINEELETFNSRWRELHEEAVRRQKLLEQSIQSAQETEKSLHLIQESLTFIDKQLAAYIADKVDA
AQMPQEAQKIQSDLTSHEISLEEMKKHNQGKEAAQRVLSQIDVAQKKLQDVSMKFRLFQKPAN
FELRLQESKMILDEVKMHLPALETKSVEQEVVQSQLNHCVNLYKSLSEVKSEVEMVIKTGRQIV
QKKQTENPKELDERVTALKLHYNELGAKVTERKQQLEKCLKLSRKMRKEMNVLTEWLAATD
MELTKRSAVEGMPSNLDSEVAWGKATQKEIEKQKVHLKSITEVGEALKTVLGKKETLVEDKLS
LLNSNWIAVTSRAEEWLNLLLEYQKHMETFDQNVDHITKWIIQADTLLDESEKKKPQQKEDVL
KRLKAELNDIRPKVDSTRDQAANLMANRGDHCRKLVEPQISELNHRFAAISHRIKTGKASIPLKE
LEQFNSDIQKLLEPLEAEIQQGVNLKEEDFNKDMNEDNEGTVKELLQRGDNLQQRITDERKREEI
KIKQQLLQTKHNALKDLRSQRRKKALEISHQWYQYKRQADDLLKCLDDIEKKLASLPEPRDER
KIKEIDRELQKKKEELNAVRRQAEGLSEDGAAMAVEPTQIQLSKRWREIESKFAQFRRLNFAQIH
TVREETMMVMTEDMPLEISYVPSTYLTEITHVSQALLEVEQLLNAPDLCAKDFEDLFKQEESLK
NIKDSLQQSSGRIDIIHSKKTAALQSATPVERVKLQEALSQLDFQWEKVNKMYKDRQGRFDRSV
EKWRRFHYDIKIFNQWLTEAEQFLRKTQIPENWEHAKYKWYLKELQDGIGQRQTVVRTLNATG
EEIIQQSSKTDASILQEKLGSLNLRWQEVCKQLSDRKKRLEEQKNILSEFQRDLNEFVLWLEEAD
NIASIPLEPGKEQQLKEKLEQVKLLVEELPLRQGILKQLNETGGPVLVSAPISPEEQDKLENKLKQ
TNLQWIKVSRALPEKQGEIEAQIKDLGQLEKKLEDLEEQLNHLLLWLSPIRNQLEIYNQPNQEGP
FDVQETEIAVQAKQPDVEEILSKGQHLYKEKPATQPVKRKLEDLSSEWKAVNRLLQELRAKQPD
LAPGLTTIGYCLDLKTQVQTPQGMKEISNIQVGDLVLSNTGYNEVLNVFPKSKKKSYKITLEDGK
EIICSEEHLFPTQTGEMNISGGLKEGMCLYVKE*
•C-ter fragment (SEQ ID NO. 110):
MMLKKILKIEELDERELIDIEVSGNHLFYANDILTHNSPTQTVTLVTQPVVTKETAISKLEMPSSL
MLEVPALADFNRAWTELTDWLSLLDQVIKSQRVMVGDLEDINEMIIKQKATMQDLEQRRPQLE
ELITAAQNLKNKTSNQEARTIITDRIERIQNQWDEVQEHLQNRRQQLNEMLKDSTQWLEAKEEA
EQVLGQARAKLESWKEGPYTVDAIQKKITETKQLAKDLRQWQTNVDVANDLALKLLRDYSAD
DTRKVHMITENINASWRSIHKRVSEREAALEETHRLLQQFPLDLEKFLAWLTEAETTANVLQDA
TRKERLLEDSKGVKELMKQWQDLQGEIEAHTDVYHNLDENSQKILRSLEGSDDAVLLQRRLDN
MNFKWSELRKKSLNIRSHLEASSDQWKRLHLSLQELLVWLQLKDDELSRQAPIGGDFPAVQKQ
NDVHRAFKRELKTKEPVIMSTLETVRIFLTEQPLEGLEKLYQEPRELPPEERAQNVTRLLRKQAE
EVNTEWEKLNLHSADWQRKIDETLERLQELQEATDELDLKLRQAEVIKGSWQPVGDLLIDSLQD
HLEKVKALRGEIAPLKENVSHVNDLARQLTTLGIQLSPYNLSTLEDLNTRWKLLQVAVEDRVRQ
LHEAHRDFGPASQHELSTSVQGPWERAISPNKVPYYINHETQTTCWDHPKMTELYQSLADLNNV
RFSAYRTAMKLRRLQKALCLDLLSLSAACDALDQHNLKQNDQPMDILQIINCLTTIYDRLEQEH
NNLVNVPLCVDMCLNWLLNVYDTGRTGRIRVLSFKTGIISLCKAHLEDKYRYLFKQVASSTGFC
DQRRLGLLLHDSIQIPRQLGEVASFGGSNIEPSVRSCFQFANNKPEIEAALFLDWMRLEPQSMVW
LPVLHRVAAAETAKHQAKCNICKECPIIGFRYRSLKHFNYDICQSCFFSGRVAKGHKMHYPMVE
YCTPTTSGEDVRDFAKVLKNKFRTKRYFAKHPRMGYLPVQTVLEGDNMETPVTLINFWPVDSA
PASSPQLSHDDTHSRIEHYASRLAEMENSNGSYLNDSISPNESIDDEHLLIQHYCQSLNQDSPLSQ
PRSPAQILISLESEERGELERILADLEEENRNLQAEYDRLKQQHEHKGLSPLPSPPEMMPTSPQSPR
DAELIAEAKLLRQHKGRLEARMQILEDHNKQLESQLHRLRQLLEQPQAEAKVNGTTVSSPSTSL
QRSDSSQPMLLRVVGSQTSDSMGEEDLLSPPQDTSTGLEEVMEQLNNSFPSSRGRNTPGKPMRE
DTM*
4) Split site NrS4:
•N-ter fragment (SEQ ID NO. 111):
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVMK
NIMAGLQQTNSEKILLSWVRQSTRNYPQVNVINFTTSWSDGLALNALIHSHRPDLFDWNSVVCQ
QSATQRLEHAFNIARYQLGIEKLLDPEDVDTTYPDKKSILMYITSLFQVLPQQVSIEAIQEVEMLP
RPPKVTKEEHFQLHHQMHYSQQITVSLAQGYERTSSPKPRFKSYAYTQAAYVTTSDPTRSPFPSQ
HLEAPEDKSFGSSLMESEVNLDRYQTALEEVLSWLLSAEDTLQAQGEISNDVEVVKDQFHTHEG
YMMDLTAHQGRVGNILQLGSKLIGTGKLSEDEETEVQEQMNLLNSRWECLRVASMEKQSNLH
RVLMDLQNQKLKELNDWLTKTEERTRKMEEEPLGPDLEDLKRQVQQHKVLQEDLEQEQVRVN
SLTHMVVVVDESSGDHATAALEEQLKVLGDRWANICRWTEDRWVLLQDILLKWQRLTEEQCL
FSAWLSEKEDAVNKIHTTGFKDQNEMLSSLQKLAVLKADLEKKKQSMGKLYSLKQDLLSTLKN
KSVTQKTEAWLDNFARCWDNLVQKLEKSTAQISQAVTTTQPSLTQTTVMETVTTVTTREQILVK
HAQEELPPPPPQKKRQITVDSEIRKRLDVDITELHSWITRSEAVLQSPEFAIFRKEGNFSDLKEKVN
AIEREKAEKFRKLQDASRSAQALVEQMVNEGVNADSIKQASEQLNSRWIEFCQLLSERLNWLEY
QNNIIAFYNQLQQLEQMTTTAENWLKIQPTTPSEPTAIKSQLKICKDEVNRLSGLQPQIERLKIQSI
ALKEKGQGPMFLDADFVAFTNHFKQVFSDVQAREKELQTIFDTLPPMRYQETMSAIRTWVQQS
ETKLSIPQLSVTDYEIMEQRLGELQALQSSLQEQQSGLYYLSTTVKEMSKKAPSEISRKYQSEFEE
IEGRWKKLSSQLVEHCQKLEEQMNKLRKIQNHIQTLKKWMAEVDVFLKEEWPALGDSEILKKQ
LKQCRLLVSDIQTIQPSLNSVNEGGQKIKNEAEPEFASRLETELKELNTQWDHMCQQVYARKEA
LKGGLEKTVSLQKDLSEMHEWMTQAEEEYLERDFECCLVGSSEIITRNYGKTTIKEVVEIFDNDK
NIQVLAFNTHTDNIEWAPIKAAQLTRPNAELVELEIDTLHGVKTIRCTPDHPVYTKNRGYVRADE
LTDDDELVVAI*
•Middle fragment (SEQ ID NO. 112):
MEAKTYIGKLKSRKIVSNEDTYDIQTSTHNFFANDILVHNSTPDELQKAVEEMKRAKEEAQQKE
AKVKLLTESVNSVIAQAPPVAQEALKKELETLTTNYQWLCTRLNGKCKTLEEVWACWHELLSY
LEKANKWLNEVEFKLKTTENIPGGAEEISEVLDSLENLMRHSEDNPNQIRILAQTLTDGGVMDEL
INEELETFNSRWRELHEEAVRRQKLLEQSIQSAQETEKSLHLIQESLTFIDKQLAAYIADKVDAAQ
MPQEAQKIQSDLTSHEISLEEMKKHNQGKEAAQRVLSQIDVAQKKLQDVSMKFRLFQKPANFEL
RLQESKMILDEVKMHLPALETKSVEQEVVQSQLNHCVNLYKSLSEVKSEVEMVIKTGRQIVQKK
QTENPKELDERVTALKLHYNELGAKVTERKQQLEKCLKLSRKMRKEMNVLTEWLAATDMELT
KRSAVEGMPSNLDSEVAWGKATQKEIEKQKVHLKSITEVGEALKTVLGKKETLVEDKLSLLNS
NWIAVTSRAEEWLNLLLEYQKHMETFDQNVDHITKWIIQADTLLDESEKKKPQQKEDVLKRLK
AELNDIRPKVDSTRDQAANLMANRGDHCRKLVEPQISELNHRFAAISHRIKTGKASIPLKELEQF
NSDIQKLLEPLEAEIQQGVNLKEEDFNKDMNEDNEGTVKELLQRGDNLQQRITDERKREEIKIKQ
QLLQTKHNALKDLRSQRRKKALEISHQWYQYKRQADDLLKCLDDIEKKLASLPEPRDERKIKEI
DRELQKKKEELNAVRRQAEGLSEDGAAMAVEPTQIQLSKRWREIESKFAQFRRLNFAQIHTVRE
ETMMVMTEDMPLEISYVPSTYLTEITHVSQALLEVEQLLNAPDLCAKDFEDLFKQEESLKNIKDS
LQQSSGRIDIIHSKKTAALQSATPVERVKLQEALSQLDFQWEKVNKMYKDRQGRFDRSVEKWR
RFHYDIKIFNQWLTEAEQFLRKTQIPENWEHAKYKWYLKELQDGIGQRQTVVRTLNATGEEIIQ
QSSKTDASILQEKLGSLNLRWQEVCKQLSDRKKRLEEQKNILSEFQRDLNEFVLWLEEADNIASI
PLEPGKEQQLKEKLEQVKLLVEELPLRQGILKQLNETGGPVLVSAPISPEEQDKLENKLKQTNLQ
WIKVSRALPEKQGEIEAQIKDLGQLEKKLEDLEEQLNHLLLWLSPIRNQLEIYNQPNQEGPFDVQ
ETEIAVQAKQPDVEEILSKGQHLYKEKPATQPVKRKLEDLSSEWKAVNRLLQELRAKQPDLAPG
LTTIGYCLDLKTQVQTPQGMKEISNIQVGDLVLSNTGYNEVLNVFPKSKKKSYKITLEDGKEIICS
EEHLFPTQTGEMNISGGLKEGMCLYVKE*
•C-ter fragment (SEQ ID NO. 113):
MMLKKILKIEELDERELIDIEVSGNHLFYANDILTHNSPTQTVTLVTQPVVTKETAISKLEMPSSL
MLEVPALADFNRAWTELTDWLSLLDQVIKSQRVMVGDLEDINEMIIKQKATMQDLEQRRPQLE
ELITAAQNLKNKTSNQEARTIITDRIERIQNQWDEVQEHLQNRRQQLNEMLKDSTQWLEAKEEA
EQVLGQARAKLESWKEGPYTVDAIQKKITETKQLAKDLRQWQTNVDVANDLALKLLRDYSAD
DTRKVHMITENINASWRSIHKRVSEREAALEETHRLLQQFPLDLEKFLAWLTEAETTANVLQDA
TRKERLLEDSKGVKELMKQWQDLQGEIEAHTDVYHNLDENSQKILRSLEGSDDAVLLQRRLDN
MNFKWSELRKKSLNIRSHLEASSDQWKRLHLSLQELLVWLQLKDDELSRQAPIGGDFPAVQKQ
NDVHRAFKRELKTKEPVIMSTLETVRIFLTEQPLEGLEKLYQEPRELPPEERAQNVTRLLRKQAE
EVNTEWEKLNLHSADWQRKIDETLERLQELQEATDELDLKLRQAEVIKGSWQPVGDLLIDSLQD
HLEKVKALRGEIAPLKENVSHVNDLARQLTTLGIQLSPYNLSTLEDLNTRWKLLQVAVEDRVRQ
LHEAHRDFGPASQHELSTSVQGPWERAISPNKVPYYINHETQTTCWDHPKMTELYQSLADLNNV
RFSAYRTAMKLRRLQKALCLDLLSLSAACDALDQHNLKQNDQPMDILQIINCLTTIYDRLEQEH
NNLVNVPLCVDMCLNWLLNVYDTGRTGRIRVLSFKTGIISLCKAHLEDKYRYLFKQVASSTGFC
DQRRLGLLLHDSIQIPRQLGEVASFGGSNIEPSVRSCFQFANNKPEIEAALFLDWMRLEPQSMVW
LPVLHRVAAAETAKHQAKCNICKECPIIGFRYRSLKHFNYDICQSCFFSGRVAKGHKMHYPMVE
YCTPTTSGEDVRDFAKVLKNKFRTKRYFAKHPRMGYLPVQTVLEGDNMETPVTLINFWPVDSA
PASSPQLSHDDTHSRIEHYASRLAEMENSNGSYLNDSISPNESIDDEHLLIQHYCQSLNQDSPLSQ
PRSPAQILISLESEERGELERILADLEEENRNLQAEYDRLKQQHEHKGLSPLPSPPEMMPTSPQSPR
DAELIAEAKLLRQHKGRLEARMQILEDHNKQLESQLHRLRQLLEQPQAEAKVNGTTVSSPSTSL
QRSDSSQPMLLRVVGSQTSDSMGEEDLLSPPQDTSTGLEEVMEQLNNSFPSSRGRNTPGKPMRE
DTM*
Full-length Dysferlin:
1) Split site #1 (using split gp41.1):
•N-ter fragment (SEQ ID NO. 114):
MLRVFILYAENVHTPDTDISDAYCSAVFAGVKKRTKVIKNSVNPVWNEGFEWDLKGIPLDQGSE
LHVVVKDHETMGRNRFLGEAKVPLREVLATPSLSASFNAPLLDTKKQPTGASLVLQVSYTPLPG
AVPLFPPPTPLEPSPTLPDLDVVADTGGEEDTEDQGLTGDEAEPFLDQSGGPGAPTTPRKLPSRPP
PHYPGIKRKRSAPTSRKLLSDKPQDFQIRVQVIEGRQLPGVNIKPVVKVTAAGQTKRTRIHKGNS
PLFNETLFFNLFDSPGELFDEPIFITVVDSRSLRTDALLGEFRMDVGTIYREPRHAYLRKWLLLSD
PDDFSAGARGYLKTSLCVLGPGDEAPLERKDPSEDKEDIESNLLRPTGVALRGAHFCLKVFRAE
DLPQMDDAVMDNVKQIFGFESNKKNLVDPFVEVSFAGKMLCSKILEKTANPQWNQNITLPAMF
PSMCEKMRIRIIDWDRLTHNDIVATTYLSMSKISAPGGEIEEEPAGAVKPSKASDLDDYLGFLPTF
GPCYINLYGSPREFTGFPDPYTELNTGKGEGVAYRGRLLLSLETKLVEHSEQKVEDLPADDILRV
EKYLRRRKYSLFAAFYSATMLQDVDDAIQFEVSIGNYGNKFDMTCLPLASTTQYSRAVFDGCH
YYYLPWGNVKPVVVLSSYWEDISHRIETQNQLLGIADRLEAGLEQVHLALKAQCSTEDVDSLV
AQLTDELIAGCSQPLGDIHETPSATHLDQYLYQLRTHHLSQITEAALALKLGYCLDLKTQVQTPQ
GMKEISNIQVGDLVLSNTGYNEVLNVFPKSKKKSYKITLEDGKEIICSEEHLFPTQTGEMNISGGL
KEGMCLYVKE*
•C-ter fragment (SEQ ID NO. 115):
MMLKKILKIEELDERELIDIEVSGNHLFYANDILTHNSELPAALEQAEDWLLRLRALAEEPQNSLP
DIVIWMLQGDKRVAYQRVPAHQVLFSRRGANYCGKNCGKLQTIFLKYPMEKVPGARMPVQIR
VKLWFGLSVDEKEFNQFAEGKLSVFAETYENETKLALVGNWGTTGLTYPKFSDVTGKIKLPKDS
FRPSAGWTWAGDWFVCPEKTLLHDMDAGHLSFVEEVFENQTRLPGGQWIYMSDNYTDVNGEK
VLPKDDIECPLGWKWEDEEWSTDLNRAVDEQGWEYSITIPPERKPKHWVPAEKMYYTHRRRR
WVRLRRRDLSQMEALKRHRQAEAEGEGWEYASLFGWKFHLEYRKTDAFRRRRWRRRMEPLE
KTGPAAVFALEGALGGVMDDKSEDSMSVSTLSFGVNRPTISCIFDYGNRYHLRCYMYQARDLA
AMDKDSFSDPYAIVSFLHQSQKTVVVKNTLNPTWDQTLIFYEIEIFGEPATVAEQPPSIVVELYDH
DTYGADEFMGRCICQPSLERMPRLAWFPLTRGSQPSGELLASFELIQREKPAIHHIPGFEVQETSRI
LDESEDTDLPYPPPQREANIYMVPQNIKPALQRTAIEILAWGLRNMKSYQLANISSPSLVVECGG
QTVQSCVIRNLRKNPNFDICTLFMEVMLPREELYCPPITVKVIDNRQFGRRPVVGQCTIRSLESFL
CDPYSAESPSPQGGPDDVSLLSPGEDVLIDIDDKEPLIPIQEEEFIDWWSKFFASIGEREKCGSYLE
KDFDTLKVYDTQLENVEAFEGLSDFCNTFKLYRGKTQEETEDPSVIGEFKGLFKIYPLPEDPAIPM
PPRQFHQLAAQGPQECLVRIYIVRAFGLQPKDPNGKCDPYIKISIGKKSVSDQDNYIPCTLEPVFG
KMFELTCTLPLEKDLKITLYDYDLLSKDEKIGETVVDLENRLLSKFGARCGLPQTYCVSGPNQW
RDQLRPSQLLHLFCQQHRVKAPVYRTDRVMFQDKEYSIEEIEAGRIPNPHLGPVEERLALHVLQQ
QGLVPEHVESRPLYSPLQPDIEQGKLQMWVDLFPKALGRPGPPFNITPRRARRFFLRCIIWNTRD
VILDDLSLTGEKMSDIYVKGWMIGFEEHKQKTDVHYRSLGGEGNFNWRFIFPFDYLPAEQVCTI
AKKDAFWRLDKTESKIPARVVFQIWDNDKFSFDDFLGSLQLDLNRMPKPAKTAKKCSLDQLDD
AFHPEWFVSLFEQKTVKGWWPCVAEEGEKKILAGKLEMTLEIVAESEHEERPAGQGRDEPNMN
PKLEDPRRPDTSFLWFTSPYKTMKFILWRRFRWAIILFIILFILLLFLAIFIYAFPNYAAMKLVKPFS
*
2) Split site #2 (using split gp41.1):
•N-ter fragment (SEQ ID NO. 116):
MLRVFILYAENVHTPDTDISDAYCSAVFAGVKKRTKVIKNSVNPVWNEGFEWDLKGIPLDQGSE
LHVVVKDHETMGRNRFLGEAKVPLREVLATPSLSASFNAPLLDTKKQPTGASLVLQVSYTPLPG
AVPLFPPPTPLEPSPTLPDLDVVADTGGEEDTEDQGLTGDEAEPFLDQSGGPGAPTTPRKLPSRPP
PHYPGIKRKRSAPTSRKLLSDKPQDFQIRVQVIEGRQLPGVNIKPVVKVTAAGQTKRTRIHKGNS
PLFNETLFFNLFDSPGELFDEPIFITVVDSRSLRTDALLGEFRMDVGTIYREPRHAYLRKWLLLSD
PDDFSAGARGYLKTSLCVLGPGDEAPLERKDPSEDKEDIESNLLRPTGVALRGAHFCLKVFRAE
DLPQMDDAVMDNVKQIFGFESNKKNLVDPFVEVSFAGKMLCSKILEKTANPQWNQNITLPAMF
PSMCEKMRIRIIDWDRLTHNDIVATTYLSMSKISAPGGEIEEEPAGAVKPSKASDLDDYLGFLPTF
GPCYINLYGSPREFTGFPDPYTELNTGKGEGVAYRGRLLLSLETKLVEHSEQKVEDLPADDILRV
EKYLRRRKYSLFAAFYSATMLQDVDDAIQFEVSIGNYGNKFDMTCLPLASTTQYSRAVFDGCH
YYYLPWGNVKPVVVLSSYWEDISHRIETQNQLLGIADRLEAGLEQVHLALKAQCSTEDVDSLV
AQLTDELIAGCSQPLGDIHETPSATHLDQYLYQLRTHHLSQITEAALALKLGHSELPAALEQAED
WLLRLRALAEEPQNSLPDIVIWMLQGDKRVAYQRVPAHQVLFSRRGANYCGKNCGKLQTIFLK
YPMEKVPGARMPVQIRVKLWFGYCLDLKTQVQTPQGMKEISNIQVGDLVLSNTGYNEVLNVFP
KSKKKSYKITLEDGKEIICSEEHLFPTQTGEMNISGGLKEGMCLYVKE*
•C-ter fragment (SEQ ID NO. 117):
MMLKKILKIEELDERELIDIEVSGNHLFYANDILTHNSVDEKEFNQFAEGKLSVFAETYENETKL
ALVGNWGTTGLTYPKFSDVTGKIKLPKDSFRPSAGWTWAGDWFVCPEKTLLHDMDAGHLSFV
EEVFENQTRLPGGQWIYMSDNYTDVNGEKVLPKDDIECPLGWKWEDEEWSTDLNRAVDEQGW
EYSITIPPERKPKHWVPAEKMYYTHRRRRWVRLRRRDLSQMEALKRHRQAEAEGEGWEYASLF
GWKFHLEYRKTDAFRRRRWRRRMEPLEKTGPAAVFALEGALGGVMDDKSEDSMSVSTLSFGV
NRPTISCIFDYGNRYHLRCYMYQARDLAAMDKDSFSDPYAIVSFLHQSQKTVVVKNTLNPTWD
QTLIFYEIEIFGEPATVAEQPPSIVVELYDHDTYGADEFMGRCICQPSLERMPRLAWFPLTRGSQP
SGELLASFELIQREKPAIHHIPGFEVQETSRILDESEDTDLPYPPPQREANIYMVPQNIKPALQRTAI
EILAWGLRNMKSYQLANISSPSLVVECGGQTVQSCVIRNLRKNPNFDICTLFMEVMLPREELYCP
PITVKVIDNRQFGRRPVVGQCTIRSLESFLCDPYSAESPSPQGGPDDVSLLSPGEDVLIDIDDKEPLI
PIQEEEFIDWWSKFFASIGEREKCGSYLEKDFDTLKVYDTQLENVEAFEGLSDFCNTFKLYRGKT
QEETEDPSVIGEFKGLFKIYPLPEDPAIPMPPRQFHQLAAQGPQECLVRIYIVRAFGLQPKDPNGK
CDPYIKISIGKKSVSDQDNYIPCTLEPVFGKMFELTCTLPLEKDLKITLYDYDLLSKDEKIGETVV
DLENRLLSKFGARCGLPQTYCVSGPNQWRDQLRPSQLLHLFCQQHRVKAPVYRTDRVMFQDKE
YSIEEIEAGRIPNPHLGPVEERLALHVLQQQGLVPEHVESRPLYSPLQPDIEQGKLQMWVDLFPK
ALGRPGPPFNITPRRARRFFLRCIIWNTRDVILDDLSLTGEKMSDIYVKGWMIGFEEHKQKTDVH
YRSLGGEGNFNWRFIFPFDYLPAEQVCTIAKKDAFWRLDKTESKIPARVVFQIWDNDKFSFDDFL
GSLQLDLNRMPKPAKTAKKCSLDQLDDAFHPEWFVSLFEQKTVKGWWPCVAEEGEKKILAGK
LEMTLEIVAESEHEERPAGQGRDEPNMNPKLEDPRRPDTSFLWFTSPYKTMKFILWRRFRWAIIL
FIILFILLLFLAIFIYAFPNYAAMKLVKPFS*
3) Split site #3 (using split IMPDH):
VN-ter fragment (SEQ ID NO. 118):
MLRVFILYAENVHTPDTDISDAYCSAVFAGVKKRTKVIKNSVNPVWNEGFEWDLKGIPLDQGSE
LHVVVKDHETMGRNRFLGEAKVPLREVLATPSLSASFNAPLLDTKKQPTGASLVLQVSYTPLPG
AVPLFPPPTPLEPSPTLPDLDVVADTGGEEDTEDQGLTGDEAEPFLDQSGGPGAPTTPRKLPSRPP
PHYPGIKRKRSAPTSRKLLSDKPQDFQIRVQVIEGRQLPGVNIKPVVKVTAAGQTKRTRIHKGNS
PLFNETLFFNLFDSPGELFDEPIFITVVDSRSLRTDALLGEFRMDVGTIYREPRHAYLRKWLLLSD
PDDFSAGARGYLKTSLCVLGPGDEAPLERKDPSEDKEDIESNLLRPTGVALRGAHFCLKVFRAE
DLPQMDDAVMDNVKQIFGFESNKKNLVDPFVEVSFAGKMLCSKILEKTANPQWNQNITLPAMF
PSMCEKMRIRIIDWDRLTHNDIVATTYLSMSKISAPGGEIEEEPAGAVKPSKASDLDDYLGFLPTF
GPCYINLYGSPREFTGFPDPYTELNTGKGEGVAYRGRLLLSLETKLVEHSEQKVEDLPADDILRV
EKYLRRRKYSLFAAFYSATMLQDVDDAIQFEVSIGNYGNKFDMTCLPLASTTQYSRAVFDGCH
YYYLPWGNVKPVVVLSSYWEDISHRIETQNQLLGIADRLEAGLEQVHLALKAQCSTEDVDSLV
AQLTDELIAGCSQPLGDIHETPSATHLDQYLYQLRTHHLSQITEAALALKLGHSELPAALEQAED
WLLRLRALAEEPQNSLPDIVIWMLQGDKRVAYQRVPAHQVLFSRRGANYCGKNCGKLQTIFLK
YPMEKVPGARMPVQIRVKLWFGLSVDEKEFNQFAEGKLSVFAETYENETKLALVGNWGTTGLT
YPKFSDVTGKIKLPKDSFRPSAGWTWAGDWFVCPEKTLLHDMDAGHLSFVEEVFENQTRLPGG
QWIYMSDNYTDVNGEKVLPKDDIECPLGWKWEDEEWSTDLNRAVDEQGWEYSITIPPERKPKH
WVPAEKMYYTHRRRRWVRLRRRDLSQMEALKRHRQAEAEGEGWEYASLFGWKFHLEYRKTD
AFRRRRWRRRMEPLEKTGPAAVFALEGALGGVMDDKSEDSMSVSTLSFGVNRPTISCIFDYGNR
YHLRCYMYQARDLAAMDKDSFSDPYAIVSFLHQSQKTVVVKNTLNPTWDQTLIFYEIEIFGEPA
TVAEQPPSIVVELYDHDTYGADEFMGRCICQPSLERMPRLAWFPLTRGCFVPGTLVNTENGLKKI
EEIKVGDKVFSHTGKLQEVVDTLIFDRDEEIISINGIDCTKNHEFYVIDKENANRVNEDNIHLFAR
WVHAEELDMKKHLLIELE*
•C-ter fragment (SEQ ID NO. 119):
MKFKLKEITSIETKHYKGKVHDLTVNQDHSYNVRGTVVHNSQPSGELLASFELIQREKPAIHHIP
GFEVQETSRILDESEDTDLPYPPPQREANIYMVPQNIKPALQRTAIEILAWGLRNMKSYQLANISS
PSLVVECGGQTVQSCVIRNLRKNPNFDICTLFMEVMLPREELYCPPITVKVIDNRQFGRRPVVGQ
CTIRSLESFLCDPYSAESPSPQGGPDDVSLLSPGEDVLIDIDDKEPLIPIQEEEFIDWWSKFFASIGER
EKCGSYLEKDFDTLKVYDTQLENVEAFEGLSDFCNTFKLYRGKTQEETEDPSVIGEFKGLFKIYP
LPEDPAIPMPPRQFHQLAAQGPQECLVRIYIVRAFGLQPKDPNGKCDPYIKISIGKKSVSDQDNYI
PCTLEPVFGKMFELTCTLPLEKDLKITLYDYDLLSKDEKIGETVVDLENRLLSKFGARCGLPQTY
CVSGPNQWRDQLRPSQLLHLFCQQHRVKAPVYRTDRVMFQDKEYSIEEIEAGRIPNPHLGPVEE
RLALHVLQQQGLVPEHVESRPLYSPLQPDIEQGKLQMWVDLFPKALGRPGPPFNITPRRARRFFL
RCIIWNTRDVILDDLSLTGEKMSDIYVKGWMIGFEEHKQKTDVHYRSLGGEGNFNWRFIFPFDY
LPAEQVCTIAKKDAFWRLDKTESKIPARVVFQIWDNDKFSFDDFLGSLQLDLNRMPKPAKTAKK
CSLDQLDDAFHPEWFVSLFEQKTVKGWWPCVAEEGEKKILAGKLEMTLEIVAESEHEERPAGQ
GRDEPNMNPKLEDPRRPDTSFLWFTSPYKTMKFILWRRFRWAIILFIILFILLLFLAIFIYAFPNYAA
MKLVKPFS*
Mini-Utrophin ΔSR5-15 (Site #1) or ΔSR6-14 (site #2):
1) Split site #1 (using split IMPDH):
•N-ter fragment (SEQ ID NO. 120):
MAKYGEHEASPDNGQNEFSDIIKSRSDEHNDVQKKTFTKWINARFSKSGKPPINDMFTDLKDGR
KLLDLLEGLTGTSLPKERGSTRVHALNNVNRVLQVLHQNNVELVNIGGTDIVDGNHKLTLGLL
WSIILHWQVKDVMKDVMSDLQQTNSEKILLSWVRQTTRPYSQVNVLNFTTSWTDGLAFNAVL
HRHKPDLFSWDKVVKMSPIERLEHAFSKAQTYLGIEKLLDPEDVAVQLPDKKSIIMYLTSLFEVL
PQQVTIDAIREVETLPRKYKKECEEEAINIQSTAPEEEHESPRAETPSTVTEVDMDLDSYQIALEEV
LTWLLSAEDTFQEQDDISDDVEEVKDQFATHEAFMMELTAHQSSVGSVLQAGNQLITQGTLSDE
EEFEIQEQMTLLNARWEALRVESMDRQSRLHDVLMELQKKQLQQLSAWLTLTEERIQKMETCP
LDDDVKSLQKLLEEHKSLQSDLEAEQVKVNSLTHMVVIVDENSGESATAILEDQLQKLGERWT
AVCRWTEERWNRLQEINILWQELLEEQCLLKAWLTEKEEALNKVQTSNFKDQKELSVSVRRLAI
LKEDMEMKRQTLDQLSEIGQDVGQLLDNSKASKKINSDSEELTQRWDSLVQRLEDSSNQVTQA
VAKLGMSQIPQKDLLETVRVREQAITKKSKQELPPPPPPKKRQIHVDIEAKKKFDAISAELLNWIL
KWKTAIQTTEIKEYMKMQDTSEMKKKLKALEKEQRERIPRADELNQTGQILVEQMGKEGLPTE
EIKNVLEKVSSEWKNVSQHLEDLERKIQLEEWRQFHCDLNDLTQWITEAEELLVDTCAPGGSLD
LEKARIHQQELEVGISSHQPSFAALNRTGDGIVQKLSQADGSFLKEKLAGLNQRWDAIVAEVKD
RQPRLKGESKQVMKYRHQLDEIICWLTKAEHAMQKRSTTELGENLQELRDLTQEMEVHAEKLK
WLNRTELEMLSDKSLSLPERDKISESLRTVNMTWNKICREVPTTLKECIQEPSSVSQTRIAAHPNV
QKVVLVSSASDIPVQSHRTSEISIPADLDKTITELADWLVLIDQMLKSNIVTVGDVEEINKTVSRM
KITKADLEQRHPQLDYVFTLAQNLKNKASSSDMRTAITEKLERVKNQWDGTQHGVELRQQQLE
DMIIDSLQWDDHREETEELMRKYEARLYILQQARRDPLTKQISDNQILLQELGPGDGIVMAFDN
VLQKLLEEYGCFVPGTLVNTENGLKKIEEIKVGDKVFSHTGKLQEVVDTLIFDRDEEIISINGIDCT
KNHEFYVIDKENANRVNEDNIHLFARWVHAEELDMKKHLLIELE*
C-ter fragment (SEQ ID NO. 121):
MKFKLKEITSIETKHYKGKVHDLTVNQDHSYNVRGTVVHNSDDTRNVKETTEYLKTSWINLKQ
SIADRQNALEAEWRTVQASRRDLENFLKWIQEAETTVNVLVDASHRENALQDSILARELKQQM
QDIQAEIDAHNDIFKSIDGNRQKMVKALGNSEEATMLQHRLDDMNQRWNDLKAKSASIRAHLE
ASAEKWNRLLMSLEELIKWLNMKDEELKKQMPIGGDVPALQLQYDHCKALRRELKEKEYSVL
NAVDQARVFLADQPIEAPEEPRRNLQSKTELTPEERAQKIAKAMRKQSSEVKEKWESLNAVTSN
WQKQVDKALEKLRDLQGAMDDLDADMKEAESVRNGWKPVGDLLIDSLQDHIEKIMAFREEIA
PINFKVKTVNDLSSQLSPLDLHPSLKMSRQLDDLNMRWKLLQVSVDDRLKQLQEAHRDFGPSS
QHFLSTSVQLPWQRSISHNKVPYYINHQTQTTCWDHPKMTELFQSLADLNNVRFSAYRTAIKIRR
LQKALCLDLLELSTTNEIFKQHKLNQNDQLLSVPDVINCLTTTYDGLEQMHKDLVNVPLCVDM
CLNWLLNVYDTGRTGKIRVQSLKIGLMSLSKGLLEEKYRYLFKEVAGPTEMCDQRQLGLLLHD
AIQIPRQLGEVAAFGGSNIEPSVRSCFQQNNNKPEISVKEFIDWMHLEPQSMVWLPVLHRVAAAE
TAKHQAKCNICKECPIVGFRYRSLKHFNYDVCQSCFFSGRTAKGHKLHYPMVEYCIPTTSGEDV
RDFTKVLKNKFRSKKYFAKHPRLGYLPVQTVLEGDNLETPITLISMWPEHYDPSQSPQLFHDDT
HSRIEQYATRLAQMERTNGSFLTDSSSTTGSVEDEHALIQQYCQTLGGESPVSQPQSPAQILKSVE
REERGELERIIADLEEEQRNLQVEYEQLKDQHLRRGLPVGSPPESIISPHHTSEDSELIAEAKLLRQ
HKGRLEARMQILEDHNKQLESQLHRLRQLLEQPESDSRINGVSPWASPQHSALSYSLDPDASGP
QFHQAAGEDLLAPPHDTSTDLTEVMEQIHSTFPSCCPNVPSRPQAM*
2) Split site #2 (using split Nrdj-1):
•N-ter fragment (SEQ ID NO. 122):
MAKYGEHEASPDNGQNEFSDIIKSRSDEHNDVQKKTFTKWINARFSKSGKPPINDMFTDLKDGR
KLLDLLEGLTGTSLPKERGSTRVHALNNVNRVLQVLHQNNVELVNIGGTDIVDGNHKLTLGLL
WSIILHWQVKDVMKDVMSDLQQTNSEKILLSWVRQTTRPYSQVNVLNFTTSWTDGLAFNAVL
HRHKPDLFSWDKVVKMSPIERLEHAFSKAQTYLGIEKLLDPEDVAVQLPDKKSIIMYLTSLFEVL
PQQVTIDAIREVETLPRKYKKECEEEAINIQSTAPEEEHESPRAETPSTVTEVDMDLDSYQIALEEV
LTWLLSAEDTFQEQDDISDDVEEVKDQFATHEAFMMELTAHQSSVGSVLQAGNQLITQGTLSDE
EEFEIQEQMTLLNARWEALRVESMDRQSRLHDVLMELQKKQLQQLSAWLTLTEERIQKMETCP
LDDDVKSLQKLLEEHKSLQSDLEAEQVKVNSLTHMVVIVDENSGESATAILEDQLQKLGERWT
AVCRWTEERWNRLQEINILWQELLEEQCLLKAWLTEKEEALNKVQTSNFKDQKELSVSVRRLAI
LKEDMEMKRQTLDQLSEIGQDVGQLLDNSKASKKINSDSEELTQRWDSLVQRLEDSSNQVTQA
VAKLGMSQIPQKDLLETVRVREQAITKKSKQELPPPPPPKKRQIHVDIEAKKKFDAISAELLNWIL
KWKTAIQTTEIKEYMKMQDTSEMKKKLKALEKEQRERIPRADELNQTGQILVEQMGKEGLPTE
EIKNVLEKVSSEWKNVSQHLEDLERKIQLQEDINAYFKQLDELEKVIKTKEEWVKHTSISESSRQ
SLPSLKDSCQRELTNLLGLHPKIEMARASCSALMSQPSAPDFVQRGFDSFLGRYQAVQEAVEDR
QQHLENELKLLPTDYLVEINKILLCMDDVELSLNVPELNTAIYEDFSFQEDSLKNIKDQLDKLGE
QIAVIHEKQPDVILEASGPEAIQIRDTLTQLNAKWDRINRMYSDRKGCFDRAMEEWRQFHCDLN
DLTQWITEAEELLVDTCAPGGSLDLEKARIHQQELEVGISSHQPSFAALNRTGDGIVQKLSQADG
SFLKEKLAGLNQRWDAIVAEVKDRQPRLKGESKQVMKYRHQLDEIICWLTKAEHAMQKRSTTE
LGENLQELRDLTQEMEVHAEKLKWLNRTELEMLSDKSLSLPERDKISESLRTVNMTWNKICREV
PTTLKECIQEPSSVSQTRIAAHPNVQKVVLVSSASDIPVQSHRTCCLVGSSEIITRNYGKTTIKEVV
EIFDNDKNIQVLAFNTHTDNIEWAPIKAAQLTRPNAELVELEIDTLHGVKTIRCTPDHPVYTKNR
GYVRADELTDDDELVVAI*
•C-ter fragment (SEQ ID NO. 123):
MEAKTYIGKLKSRKIVSNEDTYDIQTSTHNFFANDILVHNSEISIPADLDKTITELADWLVLIDQM
LKSNIVTVGDVEEINKTVSRMKITKADLEQRHPQLDYVFTLAQNLKNKASSSDMRTAITEKLER
VKNQWDGTQHGVELRQQQLEDMIIDSLQWDDHREETEELMRKYEARLYILQQARRDPLTKQIS
DNQILLQELGPGDGIVMAFDNVLQKLLEEYGSDDTRNVKETTEYLKTSWINLKQSIADRQNALE
AEWRTVQASRRDLENFLKWIQEAETTVNVLVDASHRENALQDSILARELKQQMQDIQAEIDAH
NDIFKSIDGNRQKMVKALGNSEEATMLQHRLDDMNQRWNDLKAKSASIRAHLEASAEKWNRL
LMSLEELIKWLNMKDEELKKQMPIGGDVPALQLQYDHCKALRRELKEKEYSVLNAVDQARVF
LADQPIEAPEEPRRNLQSKTELTPEERAQKIAKAMRKQSSEVKEKWESLNAVTSNWQKQVDKA
LEKLRDLQGAMDDLDADMKEAESVRNGWKPVGDLLIDSLQDHIEKIMAFREEIAPINFKVKTVN
DLSSQLSPLDLHPSLKMSRQLDDLNMRWKLLQVSVDDRLKQLQEAHRDFGPSSQHELSTSVQLP
WQRSISHNKVPYYINHQTQTTCWDHPKMTELFQSLADLNNVRFSAYRTAIKIRRLQKALCLDLL
ELSTTNEIFKQHKLNQNDQLLSVPDVINCLTTTYDGLEQMHKDLVNVPLCVDMCLNWLLNVYD
TGRTGKIRVQSLKIGLMSLSKGLLEEKYRYLFKEVAGPTEMCDQRQLGLLLHDAIQIPRQLGEVA
AFGGSNIEPSVRSCFQQNNNKPEISVKEFIDWMHLEPQSMVWLPVLHRVAAAETAKHQAKCNIC
KECPIVGFRYRSLKHFNYDVCQSCFFSGRTAKGHKLHYPMVEYCIPTTSGEDVRDFTKVLKNKF
RSKKYFAKHPRLGYLPVQTVLEGDNLETPITLISMWPEHYDPSQSPQLFHDDTHSRIEQYATRLA
QMERTNGSFLTDSSSTTGSVEDEHALIQQYCQTLGGESPVSQPQSPAQILKSVEREERGELERIIA
DLEEEQRNLQVEYEQLKDQHLRRGLPVGSPPESIISPHHTSEDSELIAEAKLLRQHKGRLEARMQI
LEDHNKQLESQLHRLRQLLEQPESDSRINGVSPWASPQHSALSYSLDPDASGPQFHQAAGEDLL
APPHDTSTDLTEVMEQIHSTFPSCCPNVPSRPQAM*

1. A method for inducing the production of an exogenous polypeptide in a cell, the method comprising contacting the cell with:
a first adeno-associated virus (AAV) vector particle comprising a first nucleic acid encoding a first fusion polypeptide comprising a first portion of an exogenous polypeptide fused to a first portion of a split intein; and
a second AAV vector particle comprising a second nucleic acid encoding a second fusion polypeptide comprising a second portion of the exogenous polypeptide fused to a second portion of the split intein;
wherein the first and second fusion polypeptides are produced in the cell from the first and second nucleic acids, and wherein the first and second portions of the split intein promote joining of the first portion of the exogenous polypeptide to the second portion of the exogenous polypeptide to form a spliced exogenous polypeptide, thereby inducing the production of the exogenous polypeptide in the cell;
wherein the spliced exogenous polypeptide produced is larger than can be encoded by a single AAV vector particle, and wherein the spliced exogenous polypeptide comprises a footprint of less than four amino acids from the split intein.

2. The method of claim 1, wherein the first and second nucleic acids comprise a muscle-specific expression cassette (MSEC).

3. The method of claim 1, wherein the split intein is a naturally-occurring split intein.

4. The method of claim 1, wherein the split intein is a genetically modified split intein.

5. The method of claim 4, wherein the genetic modification of the split intein is selected from codon optimization for expression and/or stability in mammalian cells, shortening or lengthening of the split intein, or changing encoded amino acids in the split intein to more closely match the sequence of the exogenous protein to be produced.

6. The method of claim 1, wherein the first and second portions of the exogenous polypeptide are substantially the same size, or wherein the first and second portions of the exogenous polypeptide differ in size by no more than 50 amino acids.

7-8. (canceled)

9. The method of claim 1, wherein the exogenous polypeptide comprises a footprint of 3 or fewer amino acids from the split intein.

10. The method of claim 9, wherein the split site separating the first and second portions of the exogenous polypeptide is selected at a site having the same sequence as the split intein footprint, thereby producing the spliced exogenous polypeptide without extra amino acids from the split intein.

11. The method of claim 1, wherein the exogenous polypeptide is a therapeutic polypeptide.

12. The method of claim 11, wherein the therapeutic polypeptide is selected from dystrophin, mini-dystrophin, utrophin, dysferlin, nebulin, titin, myosin, spectrin repeat containing nuclear envelope protein 1 (Syne-1), dystroglycan, ATP synthase, clotting factor VIII, lamin A/C, thyroglobulin, epidermal growth factor receptor (EGFR), alpha- and/or beta spectrin, muscle target of rapamycin (mTOR), and ryanodine receptor 1.

13. The method of claim 12, wherein the mini-dystrophin is greater than 160 kDa and smaller than full-length dystrophin.

14. The method of claim 12, wherein the therapeutic polypeptide is dystrophin and the N-terminal portion of the dystrophin extein is joined to the N-terminal portion of a split intein within or adjacent to a dystrophin hinge domain.

15. The method of claim 14, wherein the hinge domain comprises hinge 1, 2, 3, or 4 of dystrophin.

16. The method of claim 12, wherein the therapeutic polypeptide is dystrophin and the N-terminal portion of the dystrophin extein is joined to a loop domain joining helix b to helix c, or helix c to helix a′ within one of the 24 dystrophin spectrin-like repeat domains.

17. The method of claim 12, wherein the therapeutic polypeptide is dystrophin and the C-terminal portion of the dystrophin extein is joined to the C-terminal portion of the split intein within or adjacent to a dystrophin hinge domain or to a loop domain joining helix b to helix c, or helix c to helix a′ within one of the 24 dystrophin spectrin-like repeat domains.

18. The method of claim 17, wherein the hinge domain comprises hinge 1, 2, 3, or 4 of dystrophin.

19. The method of claim 1, wherein the exogenous polypeptide is functional in the cell.

20. A method for inducing the production of an exogenous polypeptide in a cell, the method comprising contacting the cell with:
a first adeno-associated virus (AAV) vector particle comprising a first nucleic acid encoding a first fusion polypeptide comprising a first portion of an exogenous polypeptide fused to a first portion of a first split intein, wherein the first portion of the split intein is fused to the carboxy terminus of the first portion of the exogenous polypeptide;
a second AAV vector particle comprising a second nucleic acid encoding a second fusion polypeptide comprising a second portion of the exogenous polypeptide fused to (i) a second portion of the first split intein at the amino terminus of the second portion of the exogenous polypeptide and (ii) a first portion of a second split intein at the carboxy terminus of the second portion of the exogenous polypeptide; and
a third AAV vector particle comprising a third nucleic encoding a third fusion polypeptide comprising a third portion of the exogenous polypeptide fused to a second portion of the second split intein at the amino terminus of the third portion of the exogenous polypeptide,
wherein the first, second, and third fusion polypeptides are produced in the cell from the first, second and third nucleic acids, and wherein the respective portions of the first and second split inteins promote joining of (a) the carboxy terminus of the first portion of the exogenous polypeptide to the amino terminus of the second portion of the exogenous polypeptide and (b) the carboxy terminus of the second portion of the exogenous polypeptide to the amino terminus of the third portion of the exogenous polypeptide to form a spliced exogenous polypeptide, thereby inducing the production of the exogenous polypeptide in the cell;
wherein the spliced exogenous polypeptide produced is larger than can be encoded by a single AAV vector particle, and wherein the spliced exogenous polypeptide comprises a footprint of less than four amino acids from the first split intein and a footprint of less than four amino acids from the second split intein.

21. (canceled)

22. A protein expression system comprising a set of AAV vector particles comprising a first and second AAV particle,
wherein the first AAV vector particle comprises a first nucleic acid encoding a first fusion polypeptide comprising a first portion of an exogenous polypeptide fused to a first portion of a split intein;
wherein the second AAV vector particle comprises a second nucleic acid encoding a second fusion polypeptide comprising a second portion of the exogenous polypeptide fused to a second portion of the split intein; and
wherein, when the first and second fusion polypeptides are expressed, the first and second fusion polypeptides undergo splicing such that the first portion of the exogenous polypeptide is joined to the second portion of the exogenous polypeptide to form a spliced exogenous polypeptide, with removal of the first and second portions of the split intein, and wherein the spliced exogenous polypeptide comprises a footprint of less than four amino acids from the split intein.

23-28. (canceled)

29. A method of treating a disease or disorder in a subject in need thereof, the method comprising administering a protein expression system of claim 22, thereby treating the subject.

30-57. (canceled)
